SEQUENCE LISTING (324 sequences)

SEQ ID NO: 1 - protein, 132 aa, synthetic construct
  source 1..132: note = OX40L
VSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 152 aa, synthetic construct
  source 1..152: note = 4-1BBL
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 3 - protein, 14 aa, synthetic construct
  source 1..14: note = Linker
GGSGGSGGGSSGSG

SEQ ID NO: 4 - protein, 4 aa, synthetic construct
  source 1..4: note = Linker
SGSG

SEQ ID NO: 5 - skipped

SEQ ID NO: 6 - protein, 7 aa, synthetic construct
  source 1..7: note = Linker
SGSGGSG

SEQ ID NO: 7 - protein, 9 aa, synthetic construct
  source 1..9: note = Linker
SGSGGSSGG

SEQ ID NO: 8 - protein, 11 aa, synthetic construct
  source 1..11: note = Linker
SGSGGSSGGSG

SEQ ID NO: 9 - protein, 13 aa, synthetic construct
  source 1..13: note = Linker
GSGSGGSSGGSSG

SEQ ID NO: 10 - protein, 21 aa, synthetic construct
  source 1..21: note = Linker
LREGPELSPDDPAGLLDLRQG

SEQ ID NO: 11 - protein, 424 aa, synthetic construct
  source 1..424: note = OX40L trimer
VSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 12 - protein, 464 aa, synthetic construct
  source 1..464: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 13 - protein, 500 aa, synthetic construct
  source 1..500: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 14 - protein, 470 aa, synthetic construct
  source 1..470: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 15 - protein, 474 aa, synthetic construct
  source 1..474: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 16 - protein, 478 aa, synthetic construct
  source 1..478: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 17 - protein, 482 aa, synthetic construct
  source 1..482: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGGSSGGSSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGGSSGGSSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 18 - protein, 540 aa, synthetic construct
  source 1..540: note = 4-1BBL trimer
MFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 19 - protein, 5 aa, synthetic construct
  source 1..5: note = CD3 CDRH1
RYTMH

SEQ ID NO: 20 - protein, 17 aa, synthetic construct
  source 1..17: note = CD3 CDRH2
YINPSRGYTNYAQKFQG

SEQ ID NO: 21 - protein, 10 aa, synthetic construct
  source 1..10: note = CD3 CDRH3
YYDDHYSLDY

SEQ ID NO: 22 - protein, 10 aa, synthetic construct
  source 1..10: note = CD3 CDRL1
RASQSISYLN

SEQ ID NO: 23 - protein, 7 aa, synthetic construct
  source 1..7: note = CD3 CDRL2
DTSKLAS

SEQ ID NO: 24 - protein, 9 aa, synthetic construct
  source 1..9: note = CD3 CDRL3
QQWSSNPLT

SEQ ID NO: 25 - protein, 5 aa, synthetic construct
  source 1..5: note = CD3h34 CDRH1
TYAMN

SEQ ID NO: 26 - protein, 19 aa, synthetic construct
  source 1..19: note = CD3h34 CDRH2
RIRSKYNNYATYYADSVKD

SEQ ID NO: 27 - protein, 14 aa, synthetic construct
  source 1..14: note = CD3h34 CDRH3
HGNFGNSYVSWFAY

SEQ ID NO: 28 - protein, 14 aa, synthetic construct
  source 1..14: note = CD3h34 CDRL1
RSSTGAVTTSNYAN

SEQ ID NO: 29 - protein, 7 aa, synthetic construct
  source 1..7: note = CD3h34 CDRL2
GTNKRAP

SEQ ID NO: 30 - protein, 9 aa, synthetic construct
  source 1..9: note = CD3h34 CDRL3
ALWYSNLWV

SEQ ID NO: 31 - protein, 5 aa, synthetic construct
  source 1..5: note = CD28 CDRH1
SYYIH

SEQ ID NO: 32 - protein, 17 aa, synthetic construct
  source 1..17: note = CD28 CDRH2
SIYPGNVNTNYNEKFKD

SEQ ID NO: 33 - protein, 11 aa, synthetic construct
  source 1..11: note = CD28 CDRH3
SHYGLDWNFDV

SEQ ID NO: 34 - protein, 11 aa, synthetic construct
  source 1..11: note = CD28 CDRL1
HASQNIYVWLN

SEQ ID NO: 35 - protein, 7 aa, synthetic construct
  source 1..7: note = CD28 CDRL2
KASNLHT

SEQ ID NO: 36 - protein, 9 aa, synthetic construct
  source 1..9: note = CD28 CDRL3
QQGQTYPYT

SEQ ID NO: 37 - protein, 5 aa, synthetic construct
  source 1..5: note = HER2 CDRH1
DTYIH

SEQ ID NO: 38 - protein, 17 aa, synthetic construct
  source 1..17: note = HER2 CDRH2
RIYPTTGYTRYADSVKG

SEQ ID NO: 39 - protein, 11 aa, synthetic construct
  source 1..11: note = HER2 CDRH3
WGGTGFYAMDY

SEQ ID NO: 40 - protein, 11 aa, synthetic construct
  source 1..11: note = HER2 CDRL1
RASQDVNTAVA

SEQ ID NO: 41 - protein, 7 aa, synthetic construct
  source 1..7: note = HER2 CDRL2
SASFLYS

SEQ ID NO: 42 - protein, 9 aa, synthetic construct
  source 1..9: note = HER2 CDRL3
QQHYTTPPT

SEQ ID NO: 43 - protein, 119 aa, synthetic construct
  source 1..119: note = CD3 VH
QVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSS

SEQ ID NO: 44 - protein, 119 aa, synthetic construct
  source 1..119: note = CD3 VH
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSS

SEQ ID NO: 45 - protein, 106 aa, synthetic construct
  source 1..106: note = CD3 VL
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIK

SEQ ID NO: 46 - protein, 120 aa, synthetic construct
  source 1..120: note = CD28 VH
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 47 - protein, 107 aa, synthetic construct
  source 1..107: note = CD28 VL
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 48 - protein, 120 aa, synthetic construct
  source 1..120: note = HER2 VH
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSS

SEQ ID NO: 49 - protein, 108 aa, synthetic construct
  source 1..108: note = HER2 VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 50 - protein, 214 aa, synthetic construct
  source 1..214: note = HER2, light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - DNA, 642 bp, synthetic construct
  source 1..642: mol_type = other DNA; note = HER2, light chain
gacatccagatgacacagagccctagcagcctgtctgccagcgtgggagacagagtgaccatcacctgtagagccagccaggacgtgaacacagccgtggcttggtatcagcagaagcctggcaaggcccctaagctgctgatctacagcgccagctttctgtacagcggcgtgcccagcagattcagcggaagcagaagcggcaccgacttcaccctgaccataagcagtctgcagcccgaggacttcgccacctactactgtcagcagcactacaccacacctccaacctttggccagggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 52 - protein, 214 aa, synthetic construct
  source 1..214: note = CD28, light chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - DNA, 642 bp, synthetic construct
  source 1..642: mol_type = other DNA; note = CD28, light chain
gacattcagatgactcaatctccctcttccttgagcgccagcgttggggaccgtgtgacaatcacatgccacgcatcccaaaatatttatgtctggctgaactggtaccaacagaaaccgggtaaagcccctaaactcttgatatacaaagcatcaaatctccacactggcgtaccctctcggttcagtggtagcgggtccgggaccgactttacacttacgatatctagcctgcaaccagaggattttgcgacttactattgtcaacaaggacagacatatccttatacttttgggggggggacgaaagttgagatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 54 - protein, 213 aa, synthetic construct
  source 1..213: note = CD3, light chain
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 639 bp, synthetic construct
  source 1..639: mol_type = other DNA; note = CD3, light chain
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 56 - protein, 216 aa, synthetic construct
  source 1..216: note = CD3, heavy chain
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 648 bp, synthetic construct
  source 1..648: mol_type = other DNA; note = CD3, heavy chain
caggccgtggtcacccaagagcctagcctgacagttagccctggcggaaccgtgacactgacatgtcgctcttctacaggcgccgtgaccacctctaattacgcaaattgggtgcagcagaagcccggacaggctcccagaggactgatcggcggaacaaacaagagagccccttggacccctgccagattttctggctctctgctcggaggaaaggccgctctgacaatcacaggtgcccaggccgaggatgaggccgattactattgtgccctgtggtacagcaacctgtgggtgttcggcggaggcaccaaactgacagttctgcgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgt

SEQ ID NO: 58 - protein, 918 aa, synthetic construct
  source 1..918: note = ahCD28_ScFv_Kcc_hOX40LLALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 59 - DNA, 2754 bp, synthetic construct
  source 1..2754: mol_type = other DNA; note = ahCD28_ScFv_Kcc_hOX40LLALAPA
gacattcaaatgacacagtctccttcttccctgtccgccagcgtcggcgatagagttactatcacctgtcatgccagccaaaatatttacgtgtggctcaactggtatcaacagaaaccgggcaaggcgccaaagctgctgatatacaaagcctctaatctgcatacaggcgtgccctctcggttctccggcagtggcagcggaacagactttaccctgactatctctagcctgcaaccggaagatttcgccacatattactgccagcagggtcagacctatccttatactttcggcggcggaactaaagttgaaatcaaaggcggcggcggtagcggcggctccggcagcggtggtggtggtagtgcgtcaggatctggccaagttcaactcgtgcagtcaggtgcggaagtaaagaagccaggagccagcgtcaaggttagttgcaaagcaagcggttataccttcacgtcctattacatccattgggtgcgccaagcgcctggccaaggcctggagtggattggtagtatctacccggggaatgtcaacacaaactataatgaaaagttcaaagatagggccaccttgactgtggacacatctatctccactgcttatatggagctgtcacgtctccgtagtgacgataccgctgtctatttctgcactagaagccactacgggctggattggaactttgatgtatggggccaaggtacgactgtgactgtctcttccgcatctggtgggtccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 60 - protein, 894 aa, synthetic construct
  source 1..894: note = ahCD28Hcs_Kcc_hOX40L LALAPA
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 61 - DNA, 2682 bp, synthetic construct
  source 1..2682: mol_type = other DNA; note = ahCD28Hcs_Kcc_hOX40L LALAPA
caggtccaactcgtccaatcaggagcagaagtcaagaaaccaggggcctctgttaaagtaagttgtaaggcctctgggtacacatttacgtcttattacattcattgggtcagacaagcgcctgggcaaggtctggagtggataggaagtatctatccaggaaatgtgaataccaattataatgaaaagtttaaagaccgggccacgttgacagtggacacctcaatttccaccgcttatatggaactttcacgcttgcggtccgacgacacggccgtgtacttttgtactcggtcccactatggcctggattggaatttcgatgtatggggccaaggcactacggtcacagtttcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 62 - protein, 449 aa, synthetic construct
  source 1..449: note = ahCD28 _LALAPA_Kcc_HC
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - DNA, 1347 bp, synthetic construct
  source 1..1347: mol_type = other DNA; note = ahCD28 _LALAPA_Kcc_HC
caggtccaactcgtccaatcaggagcagaagtcaagaaaccaggggcctctgttaaagtaagttgtaaggcctctgggtacacatttacgtcttattacattcattgggtcagacaagcgcctgggcaaggtctggagtggataggaagtatctatccaggaaatgtgaataccaattataatgaaaagtttaaagaccgggccacgttgacagtggacacctcaatttccaccgcttatatggaactttcacgcttgcggtccgacgacacggccgtgtacttttgtactcggtcccactatggcctggattggaatttcgatgtatggggccaaggcactacggtcacagtttcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctgga

SEQ ID NO: 64 - protein, 916 aa, synthetic construct
  source 1..916: note = ahbCD3h_V14_scFv_LHFc_Hcc_hOX40L LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 65 - DNA, 2748 bp, synthetic construct
  source 1..2748: mol_type = other DNA; note = ahbCD3h_V14_scFv_LHFc_Hcc_hOX40L LALAPA
gatatccaactcacgcagtcaccatcatcactgtccgccagcgtcggtgatcgagtgaccattacctgtcgcgccagtcagagcatctcctatcttaattggtatcagcagaaacccggaaagagccctaaaagatggatttacgacaccagtaagctcgctagtggagtgccatcccgcttttccgggagtgggagtggaacggactatacattgactatctccagtatgcaacccgaagatgcagccacctactattgtcaacagtggtcctcaaacccactcacatttggggggggaaccaaagttgaaattaaaggcggtggtggttctggcggaagcggctccggcggcggtggatcagcaagtggttccggccaagtgcagctggtccaatcaggagcggaagttgccaaacctggagcctcagttaaagttagttgtaaagcctctggctacacgtttacccggtacactatgcactgggtgcgtcaagccccagggcaagggcttgagtggattggctatattaatccatcccgcggttacacgaattacgcccagaagtttcagggcagggcgactctcacgaccgacaaatctacaagtacagcttacatggagctcagtagtctgcgtagcgaagatacagccgtctattattgcgcccggtattacgacgatcattattccctggattactggggccaaggtacaacccttaccgtttccagcgcatctggtggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 66 - protein, 477 aa, synthetic construct
  source 1..477: note = ahbCD3h_V14_scFv_LHFc_LALAPA_Hcc
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 67 - DNA, 1431 bp, synthetic construct
  source 1..1431: mol_type = other DNA; note = ahbCD3h_V14_scFv_LHFc_LALAPA_Hcc
gatatccaactcacgcagtcaccatcatcactgtccgccagcgtcggtgatcgagtgaccattacctgtcgcgccagtcagagcatctcctatcttaattggtatcagcagaaacccggaaagagccctaaaagatggatttacgacaccagtaagctcgctagtggagtgccatcccgcttttccgggagtgggagtggaacggactatacattgactatctccagtatgcaacccgaagatgcagccacctactattgtcaacagtggtcctcaaacccactcacatttggggggggaaccaaagttgaaattaaaggcggtggtggttctggcggaagcggctccggcggcggtggatcagcaagtggttccggccaagtgcagctggtccaatcaggagcggaagttgccaaacctggagcctcagttaaagttagttgtaaagcctctggctacacgtttacccggtacactatgcactgggtgcgtcaagccccagggcaagggcttgagtggattggctatattaatccatcccgcggttacacgaattacgcccagaagtttcagggcagggcgactctcacgaccgacaaatctacaagtacagcttacatggagctcagtagtctgcgtagcgaagatacagccgtctattattgcgcccggtattacgacgatcattattccctggattactggggccaaggtacaacccttaccgtttccagcgcatctggtggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 68 - protein, 916 aa, synthetic construct
  source 1..916: note = ahbCD3h_V14_scFv_HLFc _Hcc_hOX40L LALAPA
QVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSGGGGSGGSGSGGGGSASGSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 69 - DNA, 2748 bp, synthetic construct
  source 1..2748: mol_type = other DNA; note = ahbCD3h_V14_scFv_HLFc _Hcc_hOX40L LALAPA
caagtccagctcgtccaatcaggcgccgaagtagctaagcctggagcttccgttaaagtgtcttgtaaggccagtggttacacattcaccagatacaccatgcattgggtccgccaagccccgggacaaggcctcgaatggattggttacattaatcctagccggggatacacaaattatgcacagaaatttcaagggagagctacgctcacgaccgacaagtctaccagcactgcctacatggaactctcttcactgagaagcgaagataccgccgtatattactgcgcaaggtattacgacgaccattattctctggactattggggacaaggcacaacacttacagtttccagcggtggtggtggtagcggcggctccgggtctggtggtggcgggagtgcctctggaagtggcgatatacaactgacacaatctcccagtagtctgagcgcatcagtcggggatcgggtgacgatcacgtgccgagcgagccaaagcatctcctatctgaattggtatcagcaaaagcctggaaagagtcccaagcggtggatctatgacacaagtaaactggctagcggggtgccctcaagatttagcggaagcggatctggaaccgattacacactcaccatttcctcaatgcagccagaagacgcagccacatattattgtcaacaatggagctcaaatcccctcacatttggcggcggtactaaggtggaaatcaaggccagcggcggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 70 - protein, 477 aa, synthetic construct
  source 1..477: note = ahbCD3h_V14_scFv_HLFc_LALAPA_Hcc
QVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSGGGGSGGSGSGGGGSASGSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 71 - DNA, 1431 bp, synthetic construct
  source 1..1431: mol_type = other DNA; note = ahbCD3h_V14_scFv_HLFc_LALAPA_Hcc
caagtccagctcgtccaatcaggcgccgaagtagctaagcctggagcttccgttaaagtgtcttgtaaggccagtggttacacattcaccagatacaccatgcattgggtccgccaagccccgggacaaggcctcgaatggattggttacattaatcctagccggggatacacaaattatgcacagaaatttcaagggagagctacgctcacgaccgacaagtctaccagcactgcctacatggaactctcttcactgagaagcgaagataccgccgtatattactgcgcaaggtattacgacgaccattattctctggactattggggacaaggcacaacacttacagtttccagcggtggtggtggtagcggcggctccgggtctggtggtggcgggagtgcctctggaagtggcgatatacaactgacacaatctcccagtagtctgagcgcatcagtcggggatcgggtgacgatcacgtgccgagcgagccaaagcatctcctatctgaattggtatcagcaaaagcctggaaagagtcccaagcggtggatctatgacacaagtaaactggctagcggggtgccctcaagatttagcggaagcggatctggaaccgattacacactcaccatttcctcaatgcagccagaagacgcagccacatattattgtcaacaatggagctcaaatcccctcacatttggcggcggtactaaggtggaaatcaaggccagcggcggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 72 - protein, 479 aa, synthetic construct
  source 1..479: note = ahcd28_ScFv_Kcc_LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - DNA, 1437 bp, synthetic construct
  source 1..1437: mol_type = other DNA; note = ahcd28_ScFv_Kcc_LALAPA
gacattcaaatgacacagtctccttcttccctgtccgccagcgtcggcgatagagttactatcacctgtcatgccagccaaaatatttacgtgtggctcaactggtatcaacagaaaccgggcaaggcgccaaagctgctgatatacaaagcctctaatctgcatacaggcgtgccctctcggttctccggcagtggcagcggaacagactttaccctgactatctctagcctgcaaccggaagatttcgccacatattactgccagcagggtcagacctatccttatactttcggcggcggaactaaagttgaaatcaaaggcggcggcggtagcggcggctccggcagcggtggtggtggtagtgcgtcaggatctggccaagttcaactcgtgcagtcaggtgcggaagtaaagaagccaggagccagcgtcaaggttagttgcaaagcaagcggttataccttcacgtcctattacatccattgggtgcgccaagcgcctggccaaggcctggagtggattggtagtatctacccggggaatgtcaacacaaactataatgaaaagttcaaagatagggccaccttgactgtggacacatctatctccactgcttatatggagctgtcacgtctccgtagtgacgataccgctgtctatttctgcactagaagccactacgggctggattggaactttgatgtatggggccaaggtacgactgtgactgtctcttccgcatctggtgggtccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 74 - protein, 448 aa, synthetic construct
  source 1..448: note = ahCD3_Hrw8_Hcc_LALAPA
QVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - DNA, 1344 bp, synthetic construct
  source 1..1344: mol_type = other DNA; note = ahCD3_Hrw8_Hcc_LALAPA
caggttcagctggttcagtctggcgccgaagtggctaaacctggcgcctccgtgaaggtgtcctgtaaagccagcggctacacattcacccggtacaccatgcactgggtccgacaggctccaggacaaggcttggagtggatcggctacatcaaccccagccggggctacaccaattacgcccagaagttccagggcagagccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 76 - protein, 924 aa, synthetic construct
  source 1..924: note = ahHER2_LALAPA_41BBLrw1
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 77 - DNA, 2772 bp, synthetic construct
  source 1..2772: mol_type = other DNA; note = ahHER2_LALAPA_41BBLrw1
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacacctagcggcagcggcatgttcgcacagctcgttgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgtctgctgcacctgagcgctggacaaagactcggagtgcacctccacaccgaagctagagcaagacacgcttggcaactcacccaaggcgcaactgtcctgggactgttccgcgtgacaccttctggctcaggcatgtttgcccaactcgtggcacaaaatgtgctgctcatcgatgggccactttcttggtactctgaccctgggcttgcaggggtgtcccttactggtggcctctcttacaaagaggatacaaaagaactcgttgtcgcaaaggcaggggtctactacgtgttcttccaactcgaactccggcgcgtcgtggcaggcgaaggcagtggttctgtttcactggccctccacttgcagccccttagaagcgcagctggcgcagcagctcttgcactgactgtcgatcttccaccagcctcttccgaagccagaaactccgcttttgggtttcaaggcaggctcctccacctgagtgcaggacagaggcttggcgtccacctccatactgaggctagggccagacatgcatggcaacttacacaaggtgccaccgtgctcggactgttcagagttactcct

SEQ ID NO: 78 - protein, 960 aa, synthetic construct
  source 1..960: note = ahHER2_LALAPA_41BBLrw2
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 79 - DNA, 2880 bp, synthetic construct
  source 1..2880: mol_type = other DNA; note = ahHER2_LALAPA_41BBLrw2
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacccctgagattcctgccggcctgccttctccaagatccgaaggctctggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgctttcggatttcaaggacggctgctgcacctgagcgctggacaaagactcggagtgcacctccacaccgaagctagagcaagacacgcttggcaactcacccaaggcgcaactgtcctgggactgttccgcgtgacacctgaaatccctgctgggctcccatctcctagaagcgaaggcagcggaatgttcgcacaactggttgctcaaaatgtgctcctcatcgatggcccactgtcttggtactccgatcctgggctcgcaggggtttcacttactggcgggttgtcatacaaagaggatacaaaagaattggtcgtcgcaaaggcaggggtttactacgtgttcttccaactcgagctgagaagagtcgttgcaggcgaaggctccggctcagtttctctcgccctgcatctccaaccactcagatccgcagctggcgcagcagctcttgcactgactgtcgatcttccaccagcctcttccgaagccagaaactccgccttcggttttcaaggccgcctccttcacctgtcagctggacagaggcttggcgtccacctccatactgaggctagggccagacatgcatggcaacttacacaaggtgccaccgtgctcggactgttcagagtcacaccagagatccccgcaggactgccaagtccaagaagcgagggatct

SEQ ID NO: 80 - protein, 1056 aa, synthetic construct
  source 1..1056: note = ahHER2_LALAPA_41BBLrw3
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 81 - DNA, 3168 bp, synthetic construct
  source 1..3168: mol_type = other DNA; note = ahHER2_LALAPA_41BBLrw3
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggaagcggagctagagcttctcctggatctgccgcctctccaagactgagagagggacctgagctgagccctgatgatcctgctggactgctggatctgagacagggcatgtttgcccagctggtggcccagaatgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctgccagagcctctccaggctctgctgctagccctaggcttagagaaggcccagagctgtccccagatgacccagcaggactgctcgaccttcggcagggaatgttcgctcagctcgtcgctcaaaacgtcctgctgatcgacggcccactgtcttggtactctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgcctgctccatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggaagcgccagagcaagtcctggaagtgccgctagtcctcgcctcagagaaggacccgaactgtctcctgacgatccagctggccttctcgacctgcgccaaggcatgttcgcacaactggtcgcacaaaatgtgctcctcatcgatggacccctgtcatggtatagcgacccaggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggcagtggttctgtttcactggccctccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagagcacggcacgcatggcagttgactcaaggtgccacagtcctcgggctgttcagagtcacacccgaaattcccgctgggctcccaagtcctagaagcgaaggctct

SEQ ID NO: 82 - protein, 930 aa, synthetic construct
  source 1..930: note = ahHER2_LALAPA_41BBLrw4
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 83 - DNA, 2790 bp, synthetic construct
  source 1..2790: mol_type = other DNA; note = ahHER2_LALAPA_41BBLrw4
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacacctagcggctctggcggcagcggaatgttcgcacaactggtcgctcagaatgtcctgctcatcgacggcccactgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgtctgctgcacctgagcgctggacaaagactcggagtgcacctccacaccgaagctagagcaagacacgcttggcaactcacccaaggcgcaactgtgctgggactcttccgcgtgacaccttctggttctggcggctcaggcatgttcgcccagctcgtggctcaaaatgttctcctgatcgatggaccgctgtcttggtactccgatcctgggctcgcaggggtttcacttactggcgggttgtcatacaaagaggatacaaaagaattggtcgtcgcaaaggcaggggtttactacgtgttcttccaactcgagctgagaagagtcgttgcaggcgaaggcagtggcagtgtgtcactggccctccatctccaaccactgagaagcgcagctggggctgcagctcttgctcttaccgtggatcttccaccagcctcttccgaagccagaaactccgcttttgggtttcaaggcaggctcctccacctgagtgcaggacagaggcttggcgtccacctccatactgaggctagggccagacatgcatggcaacttacacaaggtgccaccgtcctgggactgttcagagttactcct

SEQ ID NO: 84 - protein, 934 aa, synthetic construct
  source 1..934: note = ahHER2_LALAPA_41BBLrw5
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 85 - DNA, 2802 bp, synthetic construct
  source 1..2802: mol_type = other DNA; note = ahHER2_LALAPA_41BBLrw5
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacacctagcggcagcggaggatctagcggcggcatgtttgcccaactcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgtctgctgcacctgagcgctggacaaagactcggagtgcacctccacaccgaagctagagcaagacacgcttggcaactcacccaaggcgcaactgtcctgggactgttccgcgtgacaccttctggtagcggtggatcttctggcggaatgttcgcccagttggttgctcaaaatgtgctcctcatcgatggcccactgtcttggtactccgatcctgggctcgcaggggtttcacttactggcgggttgtcatacaaagaggatacaaaagaattggtcgtcgcaaaggcaggggtttactacgtgttcttccaactcgagctgagaagagtcgttgcaggcgaaggcagcggtagtgtgtcactggccctccatctccaaccactgagaagcgcagctggggctgcagctcttgctcttactgtcgatctcccaccagcctcttccgaagccagaaactccgcttttgggtttcaaggcaggctcctccacctgagtgcaggacagaggcttggcgtccacctccatactgaggctagggccagacatgcatggcaacttacacaaggtgccaccgtgctcggactgttcagagttactcct

SEQ ID NO: 86 - protein, 938 aa, synthetic construct
  source 1..938: note = ahHER2_LALAPA_41BBLrw6
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPSGSGGSSGGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 87 - DNA, 2814 bp, synthetic construct
  source 1..2814: mol_type = other DNA; note = ahHER2_LALAPA_41BBLrw6
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacacctagcggcagcggaggatcttctggcggctctggaatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgtctgctgcacctgagcgctggacaaagactcggagtgcacctccacaccgaagctagagcaagacacgcttggcaactcacccaaggcgcaactgtcctgggactgttccgcgtgacaccttctggttctggcggaagcagtggcggtagcggcatgtttgcccaactggtcgcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggcagtggttctgtttcactggccctccacttgcagccccttagaagcgcagctggcgcagcagctcttgcactgactgtcgatcttccaccagcctcttccgaagccagaaactccgcttttgggtttcaaggcaggctcctccacctgagtgcaggacagaggcttggcgtccacctccatactgaggctagggccagacatgcatggcaacttacacaaggtgccaccgtgctcggactgttcagagttactcct

SEQ ID NO: 88 - protein, 942 aa, synthetic construct
  source 1..942: note = ahHER2_LALAPA_41BBLrw7
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGGSSGGSSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGGSSGGSSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 89 - DNA, 2826 bp, synthetic construct
  source 1..2826: mol_type = other DNA; note = ahHER2_LALAPA_41BBLrw7
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacacctggctctggcagcggcggatctagcggaggatcttctggaatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggttcaggttctgttagcctggcactgcaccttcagccactcagaagtgctgcaggcgcagctgcactggctctcactgtggatttgcctccagccagctctgaggcccggaatagcgccttcggatttcaaggacgtctgctgcacctgagcgctggacaaagactcggagtgcacctccacaccgaagctagagcaagacacgcttggcaactcacccaaggcgcaactgtcctgggactgttcagagttaccccaggctcaggctctggtggaagctctggcggttcctctggcatgtttgcccaactggttgcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggcagtggttctgtttcactggccctccacttgcagccccttagaagcgcagctggcgcagcagctcttgcactgactgtcgatctcccaccagccagcagcgaggctaggaattccgcattcgggttccaaggacggctcctccatctcagtgcaggacagaggcttggcgtccacctccatactgaggctagggccagacatgcatggcaacttacacaaggtgccaccgtgctcggcctcttcagagtcactcct

SEQ ID NO: 90 - protein, 1020 aa, synthetic construct
  source 1..1020: note = ahHER2_LALAPA_41BBLrw8
EVQLVESGGGLVQPGGSLRLSCAASGFNIQDTYIHWVRQAPGKGLEWVARIYPTTGYTRYADSVKGRFTISADTSKNTAYLQMNSLRAEDTAVYYCSRWGGTGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 91 - DNA, 3060 bp, synthetic construct
  source 1..3060: mol_type = other DNA; note = ahHER2_LALAPA_41BBLrw8
gaggtgcagctggttgaatctggcggaggactggttcagcctggcggatctctgagactgtcttgtgccgccagcggattcaatattcaggacacctacatccactgggtccgacaggcccctggcaaaggacttgaatgggtcgccagaatctaccccaccaccggctacaccagatacgccgattctgtgaagggcagattcaccatcagcgccgacaccagcaagaacaccgcctacctgcagatgaacagcctgagagccgaggacaccgccgtgtactactgtagcagatggggcggcacaggcttctacgccatggattattggggccagggcaccctggtcaccgtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 92 - protein, 916 aa, synthetic construct
  source 1..916: note = ahCD3_scFv_Hcc_hOX40L LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 93 - DNA, 2748 bp, synthetic construct
  source 1..2748: mol_type = other DNA; note = ahCD3_scFv_Hcc_hOX40L LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaaaggcggcggaggatctggcggatctggaagcggaggcggaggttctgcttctggatctggacaggtgcagctggtgcagtctggcgccgaagttgctaaacctggcgcctctgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccacactgaccaccgacaagagcaccagcaccgcctacatggaactgtccagcctgagaagcgaggacaccgccgtgtactactgtgcccggtactacgacgaccactacagcctggattattggggccagggcacaaccctgacagtgtctagtgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 94 - protein, 714 aa, synthetic construct
  source 1..714: note = ahCD28xahCD3 L1 Hcc hIgG1LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 95 - DNA, 2142 bp, synthetic construct
  source 1..2142: mol_type = other DNA; note = ahCD28xahCD3 L1 Hcc hIgG1LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 96 - protein, 714 aa, synthetic construct
  source 1..714: note = ahCD3xahCD28 L1 Hcc hIgG1LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 97 - DNA, 2142 bp, synthetic construct
  source 1..2142: mol_type = other DNA; note = ahCD3xahCD28 L1 Hcc hIgG1LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggaagcagcggcgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggtggcggaggaagcggcggatcaggatctggcggcggaggatctgctagcggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggtggatctagtggccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 98 - protein, 448 aa, synthetic construct
  source 1..448: note = ahCD3_Hrw11_Hcc_LALAPA
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 99 - DNA, 1344 bp, synthetic construct
  source 1..1344: mol_type = other DNA; note = ahCD3_Hrw11_Hcc_LALAPA
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 100 - protein, 226 aa, synthetic construct
  source 1..226: note = hu IgG1 Fc_Kcc_LALAPA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - DNA, 678 bp, synthetic construct
  source 1..678: mol_type = other DNA; note = hu IgG1 Fc_Kcc_LALAPA
gataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctgga

SEQ ID NO: 102 - protein, 893 aa, synthetic construct
  source 1..893: note = ahCD3_Hrw8_Hcc_hOX40L LALAPA
QVQLVQSGAEVAKPGASVKVSCKASGYTFTRYTMHWVRQAPGQGLEWIGYINPSRGYTNYAQKFQGRATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 103 - DNA, 2679 bp, synthetic construct
  source 1..2679: mol_type = other DNA; note = ahCD3_Hrw8_Hcc_hOX40L LALAPA
caggttcagctggttcagtctggcgccgaagtggctaaacctggcgcctccgtgaaggtgtcctgtaaagccagcggctacacattcacccggtacaccatgcactgggtccgacaggctccaggacaaggcttggagtggatcggctacatcaaccccagccggggctacaccaattacgcccagaagttccagggcagagccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 104 - protein, 893 aa, synthetic construct
  source 1..893: note = ahCD3_HRw11_Hcc_hOX40L LALAPA
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - DNA, 2679 bp, synthetic construct
  source 1..2679: mol_type = other DNA; note = ahCD3_HRw11_Hcc_hOX40L LALAPA
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 106 - protein, 665 aa, synthetic construct
  source 1..665: note = hu IgG1 Fc stump_Kcc_hOX40L LALAPA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 107 - DNA, 1995 bp, synthetic construct
  source 1..1995: mol_type = other DNA; note = hu IgG1 Fc stump_Kcc_hOX40L LALAPA
gataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 108 - protein, 714 aa, synthetic construct
  source 1..714: note = ahCD28xahCD3 L1 hIgG1LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - DNA, 2142 bp, synthetic construct
  source 1..2142: mol_type = other DNA; note = ahCD28xahCD3 L1 hIgG1LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctgga

SEQ ID NO: 110 - protein, 1153 aa, synthetic construct
  source 1..1153: note = ahCD28xCD3_Hcc_hOX40L LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 111 - DNA, 3459 bp, synthetic construct
  source 1..3459: mol_type = other DNA; note = ahCD28xCD3_Hcc_hOX40L LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 112 - protein, 1153 aa, synthetic construct
  source 1..1153: note = CD3xCD28_Hcc_hOX40L LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 113 - DNA, 3459 bp, synthetic construct
  source 1..3459: mol_type = other DNA; note = CD3xCD28_Hcc_hOX40L LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggaagcagcggcgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggtggcggaggaagcggcggatcaggatctggcggcggaggatctgctagcggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggtggatctagtggccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 114 - protein, 1153 aa, synthetic construct
  source 1..1153: note = CD28xCD3_LALAPA_hOX40L
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 115 - DNA, 3459 bp, synthetic construct
  source 1..3459: mol_type = other DNA; note = CD28xCD3_LALAPA_hOX40L
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 116 - protein, 1153 aa, synthetic construct
  source 1..1153: note = CD3xCD28_LALAPA_hOX40L
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 117 - DNA, 3459 bp, synthetic construct
  source 1..3459: mol_type = other DNA; note = CD3xCD28_LALAPA_hOX40L
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggaagcagcggcgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggtggcggaggaagcggcggatcaggatctggcggcggaggatctgctagcggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggtggatctagtggccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggaggctccggagacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 118 - protein, 1050 aa, synthetic construct
  source 1..1050: note = ahCD28 TGNcs_ScFv_Kcc_41BBLrw8 LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 119 - DNA, 3150 bp, synthetic construct
  source 1..3150: mol_type = other DNA; note = ahCD28 TGNcs_ScFv_Kcc_41BBLrw8 LALAPA
gacattcaaatgacacagtctccttcttccctgtccgccagcgtcggcgatagagttactatcacctgtcatgccagccaaaatatttacgtgtggctcaactggtatcaacagaaaccgggcaaggcgccaaagctgctgatatacaaagcctctaatctgcatacaggcgtgccctctcggttctccggcagtggcagcggaacagactttaccctgactatctctagcctgcaaccggaagatttcgccacatattactgccagcagggtcagacctatccttatactttcggcggcggaactaaagttgaaatcaaaggcggcggcggtagcggcggctccggcagcggtggtggtggtagtgcgtcaggatctggccaagttcaactcgtgcagtcaggtgcggaagtaaagaagccaggagccagcgtcaaggttagttgcaaagcaagcggttataccttcacgtcctattacatccattgggtgcgccaagcgcctggccaaggcctggagtggattggtagtatctacccggggaatgtcaacacaaactataatgaaaagttcaaagatagggccaccttgactgtggacacatctatctccactgcttatatggagctgtcacgtctccgtagtgacgataccgctgtctatttctgcactagaagccactacgggctggattggaactttgatgtatggggccaaggtacgactgtgactgtctcttccgcatctggtgggtccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 120 - protein, 1025 aa, synthetic construct
  source 1..1025: note = CD3_HRw11_Hcc_41BBLrw LALAPA
QVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 121 - DNA, 3075 bp, synthetic construct
  source 1..3075: mol_type = other DNA; note = CD3_HRw11_Hcc_41BBLrw LALAPA
caggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 122 - protein, 797 aa, synthetic construct
  source 1..797: note = hu IgG1 Fc stump_Kcc_4-1BB LALAPA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 123 - DNA, 2391 bp, synthetic construct
  source 1..2391: mol_type = other DNA; note = hu IgG1 Fc stump_Kcc_4-1BB LALAPA
gataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 124 - protein, 1285 aa, synthetic construct
  source 1..1285: note = ahCD28xCD3_Hcc_4-1BB LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 125 - DNA, 3855 bp, synthetic construct
  source 1..3855: mol_type = other DNA; note = ahCD28xCD3_Hcc_4-1BB LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 126 - protein, 1285 aa, synthetic construct
  source 1..1285: note = CD3xCD28_Hcc_4-1BB LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 127 - DNA, 3855 bp, synthetic construct
  source 1..3855: mol_type = other DNA; note = CD3xCD28_Hcc_4-1BB LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggaagcagcggcgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggtggcggaggaagcggcggatcaggatctggcggcggaggatctgctagcggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggtggatctagtggccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 128 - protein, 1285 aa, synthetic construct
  source 1..1285: note = CD28xCD3_LALAPA_4-1BB
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 129 - DNA, 3855 bp, synthetic construct
  source 1..3855: mol_type = other DNA; note = CD28xCD3_LALAPA_4-1BB
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggcggaagcagcggcgacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggtggcggaggaagcggcggatcaggatctggcggcggaggatctgctagcggatctggacaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctggtggatctagtggccaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttctggaggctccggagacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 130 - protein, 1285 aa, synthetic construct
  source 1..1285: note = CD3xCD28_LALAPA_4-1BB
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGSSGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSGGSSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 131 - DNA, 3855 bp, synthetic construct
  source 1..3855: mol_type = other DNA; note = CD3xCD28_LALAPA_4-1BB
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagggcggaagcagcggcgacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagggtggcggaggaagcggcggatcaggatctggcggcggaggatctgctagcggatctggacaggttcagctggttcagtctggcgccgaagtgaagaaacctggcgcctctgtgaaggtgtcctgcaaggccagcggctacacctttaccagctactacatccactgggtccgacaggcccctggacaaggactggaatggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctggtggatctagtggccaggttcagctggttcagtctggcgccgaagtggctaaacctggcgcctccgtgaaggtgtcctgtaaagccagcggctacacattcacccggtacaccatgcactgggtcaagcaggctccaggacaaggcttggagtggatcggctacatcaaccctagccggggctacaccaattacgcccagaagttccagggcaaggccacactgaccaccgacaagagcacaagcaccgcctacatggaactgagcagcctgagaagcgaggacaccgccgtgtactactgtgcccggtactacgacgaccactacagcctggattattggggccagggcacaaccctgaccgtgtcatctgcttctggaggctccggagacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 132 - protein, 1146 aa, synthetic construct
  source 1..1146: note = SF_CD3_v23_Hcc_hOX40L LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 133 - DNA, 3438 bp, synthetic construct
  source 1..3438: mol_type = other DNA; note = SF_CD3_v23_Hcc_hOX40L LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcacactgaccctgagcaaggccgactacgagaagcacaaagtgtacgcctgcgaagtgacccaccagggcctttctagccctgtgaccaagagcttcaacagaggcgaatgtggcggcggaggaagcggaggcggaggttctggtggtggtggctctggtggcggaggatctggcggaggtggaagtggcggtggcggttcaggcggaggcggtagcggcggaggcggatctcaagttcagctggttcagtctggcgccgaggtggcaaaacctggcgcctctgtgaaagtgtcctgcaaggccagcggctacaccttcaccagatacaccatgcactgggtcaagcaggcccctggacaaggcttggagtggatcggctacatcaaccccagccggggctacaccaattacgcccagaagttccagggcaaagccactctgaccaccgacaagagcaccagcaccgcctacatggaactgtccagcctgagaagcgaggacaccgccgtgtactactgtgcccggtactacgacgaccactactccctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 134 - protein, 1148 aa, synthetic construct
  source 1..1148: note = SF_CD28_Kcc_hOX40L LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 135 - DNA, 3444 bp, synthetic construct
  source 1..3444: mol_type = other DNA; note = SF_CD28_Kcc_hOX40L LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaacagaggcgaatgtggcggaggcggatcaggcggcggaggtagcggtggtggtggtagcggaggtggcggaagtggtggcggaggaagcggaggcggaggatctggtggtggcggatctggcggaggtggaagtcaagttcagctggtgcagtctggcgccgaagtgaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 136 - protein, 1146 aa, synthetic construct
  source 1..1146: note = CD3_ScFvscCLCH1_Hcc_hOX40L LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 137 - DNA, 3438 bp, synthetic construct
  source 1..3438: mol_type = other DNA; note = CD3_ScFvscCLCH1_Hcc_hOX40L LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaaaggcggcggaggatctggcggatctggaagcggaggcggaggttctgcttctggatctggacaggtgcagctggtgcagtctggcgccgaagttgctaaacctggcgcctctgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccacactgaccaccgacaagagcaccagcaccgcctacatggaactgtccagcctgagaagcgaggacaccgccgtgtactactgtgcccggtactacgacgaccactacagcctggattattggggccagggcacaaccctgaccgtgtctagtagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgcactgcagagcggcaacagccaagagagcgtgacagagcaggacagcaaggactccacctatagcctgagcagcaccctgactctgagcaaggccgactacgagaagcacaaagtgtacgcctgcgaagtgacccaccagggcctttctagccctgtgaccaagagcttcaacaggggcgaatgcggtggcggtggaagtggtggttcaggttctggtggtggcggctctgctagtggttctggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 138 - protein, 1148 aa, synthetic construct
  source 1..1148: note = CD28_ScFv_CLCH1_Kcc_hOX40L LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSGGSGSGGGGSASGSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGSGSGGGGSASGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 139 - DNA, 3444 bp, synthetic construct
  source 1..3444: mol_type = other DNA; note = CD28_ScFv_CLCH1_Kcc_hOX40L LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaaaggcggcggaggctctggcggatctggatctggtggcggaggatctgcttctggaagtggacaggtgcagctggtgcagtctggcgccgaagttaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtgtctagtagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgcactgcagagcggcaacagccaagagagcgtgacagagcaggacagcaaggactccacctacagcctgagcagcaccctgacactgagcaaggccgactacgagaagcacaaagtgtacgcctgcgaagtgacccaccagggcctttctagccctgtgaccaagagcttcaacagaggcgaatgcggaggcggtggaagtggtggatctggaagcggaggcggcggttctgctagtggttctggagcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 140 - protein, 1278 aa, synthetic construct
  source 1..1278: note = SF_CD3_v23_Hcc_4-1BBL LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 141 - DNA, 3834 bp, synthetic construct
  source 1..3834: mol_type = other DNA; note = SF_CD3_v23_Hcc_4-1BBL LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcacactgaccctgagcaaggccgactacgagaagcacaaagtgtacgcctgcgaagtgacccaccagggcctttctagccctgtgaccaagagcttcaacagaggcgaatgtggcggcggaggaagcggaggcggaggttctggtggtggtggctctggtggcggaggatctggcggaggtggaagtggcggtggcggttcaggcggaggcggtagcggcggaggcggatctcaagttcagctggttcagtctggcgccgaggtggcaaaacctggcgcctctgtgaaagtgtcctgcaaggccagcggctacaccttcaccagatacaccatgcactgggtcaagcaggcccctggacaaggcttggagtggatcggctacatcaaccccagccggggctacaccaattacgcccagaagttccagggcaaagccactctgaccaccgacaagagcaccagcaccgcctacatggaactgtccagcctgagaagcgaggacaccgccgtgtactactgtgcccggtactacgacgaccactactccctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 142 - protein, 1280 aa, synthetic construct
  source 1..1280: note = SF_CD28_Kcc_4-1BBL LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 143 - DNA, 3840 bp, synthetic construct
  source 1..3840: mol_type = other DNA; note = SF_CD28_Kcc_4-1BBL LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaacagaggcgaatgtggcggaggcggatcaggcggcggaggtagcggtggtggtggtagcggaggtggcggaagtggtggcggaggaagcggaggcggaggatctggtggtggcggatctggcggaggtggaagtcaagttcagctggtgcagtctggcgccgaagtgaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 144 - protein, 899 aa, synthetic construct
  source 1..899: note = ahCD3_h34H_Hcc_hOX40L LALAPA
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 145 - DNA, 2697 bp, synthetic construct
  source 1..2697: mol_type = other DNA; note = ahCD3_h34H_Hcc_hOX40L LALAPA
gaggtgcagctggttgaatctggcggaggactggttcagcctggcggatctctgagactgtcttgtgccgccagcggcttcaccttcaacacctacgccatgaactgggtccgacaggcccctggcaaaggccttgaatgggtcgcccggatcagaagcaagtacaacaattacgccacctactacgccgacagcgtgaaggacagattcaccatcagccgggacgacagcaagaacagcctgtacctgcagatgaacagcctgaagaccgaggacaccgccgtgtactattgtgtgcggcacggcaacttcggcaacagctatgtgtcttggtttgcctactggggccagggcacactggtcacagttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 146 - protein, 1031 aa, synthetic construct
  source 1..1031: note = ahCD3_h34H_Hcc_41BBL LALAPA
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 147 - DNA, 3093 bp, synthetic construct
  source 1..3093: mol_type = other DNA; note = ahCD3_h34H_Hcc_41BBL LALAPA
gaggtgcagctggttgaatctggcggaggactggttcagcctggcggatctctgagactgtcttgtgccgccagcggcttcaccttcaacacctacgccatgaactgggtccgacaggcccctggcaaaggccttgaatgggtcgcccggatcagaagcaagtacaacaattacgccacctactacgccgacagcgtgaaggacagattcaccatcagccgggacgacagcaagaacagcctgtacctgcagatgaacagcctgaagaccgaggacaccgccgtgtactattgtgtgcggcacggcaacttcggcaacagctatgtgtcttggtttgcctactggggccagggcacactggtcacagttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 148 - protein, 10 aa, synthetic construct
  source 1..10: note = Linker
GGSGSGGGSG

SEQ ID NO: 149 - protein, 11 aa, synthetic construct
  source 1..11: note = Linker
GGSGSGGGSGG

SEQ ID NO: 150 - protein, 15 aa, synthetic construct
  source 1..15: note = Linker
GGSSGSGSGGSGSSG

SEQ ID NO: 151 - protein, 31 aa, synthetic construct
  source 1..31: note = Linker
GGSGSGGGSGLREGPELSPDDPAGLLDLRQG

SEQ ID NO: 152-154 - skipped (3 entries)

SEQ ID NO: 155 - protein, 5 aa, synthetic construct
  source 1..5: note = Linker
GGSSG

SEQ ID NO: 156 - protein, 5 aa, synthetic construct
  source 1..5: note = Linker
GGSGS

SEQ ID NO: 157 - protein, 5 aa, synthetic construct
  source 1..5: note = Linker
GSSGS

SEQ ID NO: 158 - protein, 5 aa, synthetic construct
  source 1..5: note = Linker
GTVAA

SEQ ID NO: 159 - protein, 5 aa, synthetic construct
  source 1..5: note = Linker
ASGGS

SEQ ID NO: 160 - protein, 5 aa, synthetic construct
  source 1..5: note = Linker
ASTGG

SEQ ID NO: 161 - protein, 6 aa, synthetic construct
  source 1..6: note = Linker
ASGGSG

SEQ ID NO: 162 - protein, 7 aa, synthetic construct
  source 1..7: note = Linker
GGSGSSG

SEQ ID NO: 163 - protein, 10 aa, synthetic construct
  source 1..10: note = Linker
GGSGGSSGSS

SEQ ID NO: 164 - protein, 10 aa, synthetic construct
  source 1..10: note = Linker
SGGSGSSGGS

SEQ ID NO: 165 - protein, 17 aa, synthetic construct
  source 1..17: note = Linker
GGSGGSGSGGGSASGSG

SEQ ID NO: 166 - protein, 18 aa, synthetic construct
  source 1..18: note = Linker
GGSGGSGSGGGGSASGSG

SEQ ID NO: 167 - protein, 20 aa, synthetic construct
  source 1..20: note = Linker
GGGSSGGGGSGGSGSGGSGS

SEQ ID NO: 168 - protein, 20 aa, synthetic construct
  source 1..20: note = Linker
GGGGSGGSGSGGGGSASGSG

SEQ ID NO: 169 - protein, 20 aa, synthetic construct
  source 1..20: note = Linker
GGGSSGGSGSGGSGSGGSGS

SEQ ID NO: 170 - protein, 22 aa, synthetic construct
  source 1..22: note = Linker
SGGSSGGSGSGGSGSGGSGSSG

SEQ ID NO: 171 - protein, 22 aa, synthetic construct
  source 1..22: note = Linker
GSGSSGGGGSGGSGSGGSGSSG

SEQ ID NO: 172 - protein, 40 aa, synthetic construct
  source 1..40: note = Linker
GGGGSGSGGSGGGSSGGGGSGGGGSGGGGSGGGGSGGGGS

SEQ ID NO: 173 - protein, 40 aa, synthetic construct
  source 1..40: note = Linker
GGGGSGGGGSGGGGSGGGGSGGGGSGGGGSGGGGSGGGGS

SEQ ID NO: 174 - protein, 43 aa, synthetic construct
  source 1..43: note = Linker
GGGGSGSGGSGGGSSGGGGSGGGGSGGGGSGGGGSGGGGSSSS

SEQ ID NO: 175 - protein, 45 aa, synthetic construct
  source 1..45: note = Linker
GGGGSGSGGSGGGSSGGGGSGGGGSGGGGSGGGGSGGGGSSSSGS

SEQ ID NO: 176 - protein, 216 aa, synthetic construct; identical to SEQ ID NO: 56
  source 1..216: note = ahCD3_h34L

SEQ ID NO: 177 - DNA, 648 bp, synthetic construct; identical to SEQ ID NO: 57
  source 1..648: mol_type = other DNA; note = ahCD3_h34L

SEQ ID NO: 178 - protein, 899 aa, synthetic construct; identical to SEQ ID NO: 144
  source 1..899: note = ahCD3_h34H_Hcc_hOx40L LALAPA

SEQ ID NO: 179 - DNA, 2697 bp, synthetic construct; identical to SEQ ID NO: 145
  source 1..2697: mol_type = other DNA; note = ahCD3_h34H_Hcc_hOx40L LALAPA

SEQ ID NO: 180 - protein, 1031 aa, synthetic construct; identical to SEQ ID NO: 146
  source 1..1031: note = ahCD3_h34H_Hcc_41BBL LALAPA

SEQ ID NO: 181 - DNA, 3093 bp, synthetic construct; identical to SEQ ID NO: 147
  source 1..3093: mol_type = other DNA; note = ahCD3_h34H_Hcc_41BBL LALAPA

SEQ ID NO: 182 - protein, 5 aa, synthetic construct
  source 1..5: note = CD3h34 CDRH1
TYAMN

SEQ ID NO: 183 - protein, 19 aa, synthetic construct
  source 1..19: note = CD3h34 CDRH2
RIRSKYNNYATYYADSVKD

SEQ ID NO: 184 - protein, 14 aa, synthetic construct
  source 1..14: note = CD3h34 CDRH3
HGNFGNSYVSWFAY

SEQ ID NO: 185 - protein, 14 aa, synthetic construct
  source 1..14: note = CD3h34 CDRL1
RSSTGAVTTSNYAN

SEQ ID NO: 186 - protein, 7 aa, synthetic construct
  source 1..7: note = CD3h34 CDRL2
GTNKRAP

SEQ ID NO: 187 - protein, 9 aa, synthetic construct
  source 1..9: note = CD3h34 CDRL3
ALWYSNLWV

SEQ ID NO: 188 - protein, 125 aa, synthetic construct
  source 1..125: note = CD3 heavy chain sequence
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 189 - protein, 1280 aa, synthetic construct; identical to SEQ ID NO: 142
  source 1..1280: note = ScFab_CD28_Kcc_4-1BBL LALAPA

SEQ ID NO: 190 - protein, 1155 aa, synthetic construct
  source 1..1155: note = ScFab_CD3_Glo_Hcc_hOx40L LALAPA
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 191 - protein, 1287 aa, synthetic construct
  source 1..1287: note = ScFab_CD3_Glo_Hcc_h4-1BBL LALAPA
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 192 - protein, 1154 aa, synthetic construct
  source 1..1154: note = ScFab_CD3_SAR_Hcc_hOx40L LALAPA
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 193 - protein, 1286 aa, synthetic construct
  source 1..1286: note = ScFab_CD3_SAR_Hcc_h4-1BBL LALAPA
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 194 - protein, 711 aa, synthetic construct
  source 1..711: note = ahTrop2_h20xCD3_hOKT_Kcc LALAPA
DIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 1282 aa, synthetic construct
  source 1..1282: note = ahTrop2_h20xCD3_hOKT_Kcc_41BBLrw8 LALAPA
DIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 196 - protein, 719 aa, synthetic construct
  source 1..719: note = ahcMetxahCD3_hOKT_ Kcc_LALAPA
DIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 1290 aa, synthetic construct
  source 1..1290: note = ahcMetxahCD3_hOKTKcc_LALAPA_4-1BBL
DIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGSSGDIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKGGGGSGGSGSGGGGSASGSGQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSGGSSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 198 - protein, 1289 aa, synthetic construct
  source 1..1289: note = ahcMetxahTrop2_h20_Kcc_41BBLrw8 LALAPA
DIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGSSGDIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGGGSGGSGSGGGGSASGSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSGGSSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 199 - protein, 1289 aa, synthetic construct
  source 1..1289: note = ahTrop2_h20xahcMet_Kcc_41BBLrw8 LALAPA
DIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGSSGDIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGGGSGGSGSGGGGSASGSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSGGSSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 200 - protein, 1024 aa, synthetic construct
  source 1..1024: note = ahTrop2_h20Hrw3_Hcc_41BBLrw8 LALAPA
QVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 201 - protein, 1025 aa, synthetic construct
  source 1..1025: note = ahcMet_Hrw5_Hcc_41BBLrw8 LALAPA
QVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 202 - protein, 1275 aa, synthetic construct
  source 1..1275: note = VRC01.23scFab_Kcc_4-1BBL LALAPA
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 203 - protein, 1028 aa, synthetic construct
  source 1..1028: note = VRC01.23H_Kcc_4-1BBL LALAPA
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 204 - protein, 919 aa, synthetic construct
  source 1..919: note = VRC01.23scFv_Kcc_hOx40L LALAPA
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 205 - protein, 1051 aa, synthetic construct
  source 1..1051: note = VRC01.23scFv_Kcc_4-1BBL LALAPA
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 206 - protein, 1295 aa, synthetic construct
  source 1..1295: note = ahCD20_hRitxahCD19_Kcc_41BBL LALAPA
EIQLTQSPSSLSASVGDRVTITCRASSSVSYIHWFQQKPGSAPKSLIYATSNLASGVPSRFSGSGSGTDYTLTISSLQPEDFATYYCQQWTSNPPTFGGGTKLEIKGGSSGDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSGGSGSGGGGSASGSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSGGSSGQVQLVQPGAEVKKPGASVKMSCKASGYTFTSYNMHWVRQAPGRGLEWMGAIYPGNGDTSYNQKFQGRVTLTADKSTSTAYMELSSLESEDTAVYYCARSTYYGGDWYFNVWGAGTTVTVSAASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 207 - protein, 1295 aa, synthetic construct
  source 1..1295: note = ahCD19xahCD20_hRit_Kcc_41BBL LALAPA
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIQLTQSPSSLSASVGDRVTITCRASSSVSYIHWFQQKPGSAPKSLIYATSNLASGVPSRFSGSGSGTDYTLTISSLQPEDFATYYCQQWTSNPPTFGGGTKLEIKGGGGSGGSGSGGGGSASGSGQVQLVQPGAEVKKPGASVKMSCKASGYTFTSYNMHWVRQAPGRGLEWMGAIYPGNGDTSYNQKFQGRVTLTADKSTSTAYMELSSLESEDTAVYYCARSTYYGGDWYFNVWGAGTTVTVSAGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 208 - protein, 709 aa, synthetic construct
  source 1..709: note = ScFab_CD28_TGNcs_LALAPA_Kcc
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 715 aa, synthetic construct
  source 1..715: note = ScFab_CD3_SAR_Hcc_LALAPA
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 210 - protein, 1161 aa, synthetic construct
  source 1..1161: note = CD3_SAR x VRC01.23_Hcc_hOx40L LALAPA
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGGSSGLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSGGSSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 211 - protein, 1156 aa, synthetic construct
  source 1..1156: note = CD28 x VRC01.23_Kcc_hOx40L LALAPA
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGSSGLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKGGGGSGGSGSGGGGSASGSGQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSGGSSGQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 212 - protein, 896 aa, synthetic construct
  source 1..896: note = VRC01.23H_Kcc_Ox40L LALAPA
QVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 213 - protein, 1143 aa, synthetic construct
  source 1..1143: note = VRC01.23scFab_Kcc_Ox40L LALAPA
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGGQMKKPGESMRISCRASGYEFIDCTLNWIRLAPGKRPEWMGWLKPRWGAVNYARPLQGRVTMTRQLSQDPDDPDWGTAFLELRSLTVDDTAVYFCTRGKNCDYNWDFEHWGRGTPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSSGSGSGGSGSSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGSGGSGGGSSGSGVSHRYSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 214 - protein, 1296 aa, synthetic construct
  source 1..1296: note = ahCD19axahCD20PS_Kcc_41BBL LALAPA
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGSSGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGSGSGGGGSASGSGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGSSGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 215 - protein, 1296 aa, synthetic construct
  source 1..1296: note = ahCD20_PSxahCD19_Kcc_41BBL LALAPA
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGSSGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGSGSGGGGSASGSGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGSSGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 216 - protein, 707 aa, synthetic construct
  source 1..707: note = ScFab_CD3_hOKT_v23_Hcc_LALAPA
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 217 - protein, 1296 aa, synthetic construct
  source 1..1296: note = CD20_PSxCD19 LALAPA_41BBL
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGSSGDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGSGSGGGGSASGSGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGSSGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 218 - protein, 1296 aa, synthetic construct
  source 1..1296: note = CD19 x CD20_PS LALAPA_41BBL
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGSSGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGSGSGGGGSASGSGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGSSGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 219 - protein, 1289 aa, synthetic construct
  source 1..1289: note = cMetxTrop2_h20_41BBL
DIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGSSGDIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGGGSGGSGSGGGGSASGSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSGGSSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 220 - protein, 1289 aa, synthetic construct
  source 1..1289: note = ahTrop2_h20xahcMet 41BBL
DIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIKGGSSGDIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIKGGGGSGGSGSGGGGSASGSGQVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSSGGSSGQVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 221 - protein, 904 aa, synthetic construct
  source 1..904: note = ScFab_CD3_Hcc_mono4-1BBL8
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 222 - protein, 906 aa, synthetic construct
  source 1..906: note = ScFab_CD28_Kcc_mono4-1BBL8
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 223 - protein, 870 aa, synthetic construct
  source 1..870: note = ScFab_CD3_Hcc_mono4-1BBL7
DIQLTQSPSSLSASVGDRVTITCRASQSISYLNWYQQKPGKSPKRWIYDTSKLASGVPSRFSGSGSGTDYTLTISSMQPEDAATYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVAKPGASVKVSCKASGYTFTRYTMHWVKQAPGQGLEWIGYINPSRGYTNYAQKFQGKATLTTDKSTSTAYMELSSLRSEDTAVYYCARYYDDHYSLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 224 - protein, 872 aa, synthetic construct
  source 1..872: note = ScFab_CD28_Kcc_mono4-1BBL7
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSQVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 225 - protein, 1297 aa, synthetic construct
  source 1..1297: note = ahCD20_ofxahCD19_Kcc_41BBL LALAPA
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGSSGDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSGGSGSGGGGSASGSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSGGSSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 226 - protein, 1297 aa, synthetic construct
  source 1..1297: note = ahCD20_ofx ahCD19_41BBLrw8
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGSSGDIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGGGSGGSGSGGGGSASGSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSGGSSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 227 - protein, 1297 aa, synthetic construct
  source 1..1297: note = ahCD19xahCD20_of_Kcc_41BBL LALAPA
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGSGSGGGGSASGSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 228 - protein, 1297 aa, synthetic construct
  source 1..1297: note = ahCD19xahCD20_of_41BBLrw8
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIKGGSSGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGSGSGGGGSASGSGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGSSGQVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSSASGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGSGGGSGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 229 - DNA, 3840 bp, synthetic construct; identical to SEQ ID NO: 143
  source 1..3840: mol_type = other DNA; note = ScFab_CD28_Kcc_4-1BBL LALAPA

SEQ ID NO: 230 - DNA, 3465 bp, synthetic construct
  source 1..3465: mol_type = other DNA; note = ScFab_CD3_Glo_Hcc_hOx40L LALAPA
caggccgtggttacacaagagcctagcctgacagtgtcccctggcggaacagtgacactgacatgtggcagctctacaggcgccgtgaccaccagcaattacgccaactgggtgcaagagaagcccggccaggctttcagaggactgatcggcggcaccaacaagagagcccctggaacaccagccagattcagcggatctctgctcggaggaaaggccgctctgacactgtctggtgcccagcctgaagatgaggccgagtactattgcgccctgtggtacagcaacctgtgggttttcggcggaggcaccaagctgaccgtgctgagaacagtggctgcccctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaacagaggcgaatgtggcggcggaggatctggcggaggtggaagcggaggcggaggaagcggcggaggcggttctggtggtggtggttctggcggtggcggcagtggtggtggcggctcaggtggcggtggatctgaagttcagctcctggaaagtggcggaggcctggttcaacctggtggcagtctgagactgagctgtgccgccagcggcttcacctttagcacctacgccatgaactgggtccgacaggctcctggcaaaggcctggaatgggtgtcccggatcagaagcaagtacaacaactacgccacctactacgccgacagcgtgaagggcagattcaccatcagccgggacgacagcaagaacaccctgtacctgcagatgaactccctgagagccgaggacaccgccgtgtattattgtgtgcggcacggcaacttcggcaacagctatgtgtcttggtttgcctactggggccagggcacactcgtgactgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 231 - DNA, 3861 bp, synthetic construct
  source 1..3861: mol_type = other DNA; note = ScFab_CD3_Glo_Hcc_h4-1BBL LALAPA
caggccgtggttacacaagagcctagcctgacagtgtcccctggcggaacagtgacactgacatgtggcagctctacaggcgccgtgaccaccagcaattacgccaactgggtgcaagagaagcccggccaggctttcagaggactgatcggcggcaccaacaagagagcccctggaacaccagccagattcagcggatctctgctcggaggaaaggccgctctgacactgtctggtgcccagcctgaagatgaggccgagtactattgcgccctgtggtacagcaacctgtgggttttcggcggaggcaccaagctgaccgtgctgagaacagtggctgcccctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaacagaggcgaatgtggcggcggaggatctggcggaggtggaagcggaggcggaggaagcggcggaggcggttctggtggtggtggttctggcggtggcggcagtggtggtggcggctcaggtggcggtggatctgaagttcagctcctggaaagtggcggaggcctggttcaacctggtggcagtctgagactgagctgtgccgccagcggcttcacctttagcacctacgccatgaactgggtccgacaggctcctggcaaaggcctggaatgggtgtcccggatcagaagcaagtacaacaactacgccacctactacgccgacagcgtgaagggcagattcaccatcagccgggacgacagcaagaacaccctgtacctgcagatgaactccctgagagccgaggacaccgccgtgtattattgtgtgcggcacggcaacttcggcaacagctatgtgtcttggtttgcctactggggccagggcacactcgtgactgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 232 - DNA, 3465 bp, synthetic construct
  source 1..3465: mol_type = other DNA; note = ScFab_CD3_SAR_Hcc_hOx40L LALAPA
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcggacagtggccgctcctagcgtgttcatctttccacctagcgacgagcagctgaagtccggcacagcctctgttgtgtgcctgctgaacaacttctaccccagagaagccaaggtgcagtggaaggtggacaacgccctgcagagcggcaatagccaagagtctgtgaccgagcaggacagcaaggactccacctacagcctgagcagcaccctgacactgagcaaggccgactacgagaagcacaaagtgtacgcctgcgaagtgacccaccagggcctttctagccctgtgaccaagagcttcaacaggggcgaatgtggcggcggaggatctggcggaggcggtagcggtggcggaggaagcggaggcggaggttcaggtggtggtggttcaggcggtggcggcagcggaggtggtggcagcggcggaggcggatctcaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctgtga

SEQ ID NO: 233 - DNA, 3858 bp, synthetic construct
  source 1..3858: mol_type = other DNA; note = ScFab_CD3_SAR_Hcc_h4-1BBL LALAPA
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcggacagtggccgctcctagcgtgttcatctttccacctagcgacgagcagctgaagtccggcacagcctctgttgtgtgcctgctgaacaacttctaccccagagaagccaaggtgcagtggaaggtggacaacgccctgcagagcggcaatagccaagagtctgtgaccgagcaggacagcaaggactccacctacagcctgagcagcaccctgacactgagcaaggccgactacgagaagcacaaagtgtacgcctgcgaagtgacccaccagggcctttctagccctgtgaccaagagcttcaacaggggcgaatgtggcggcggaggatctggcggaggcggtagcggtggcggaggaagcggaggcggaggttcaggtggtggtggttcaggcggtggcggcagcggaggtggtggcagcggcggaggcggatctcaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 234 - DNA, 2133 bp, synthetic construct
  source 1..2133: mol_type = other DNA; note = ahTrop2_h20xCD3_hOKT_Kcc LALAPA
gacatccagatgacacagagccctagcagcctgtctgccagcgtgggagacagagtgaccattacctgtcaggccagccaggacatcaacaagtacctggcctggtatcagaagaagcccggcaaggcccctaagctgctgatccactacaccagcaatctgcagaccggcgtgcccagcagattttctggctctggcagcggcaccgacttcaccttcaccatatctagcctgcagcctgaggatatcgccacctactactgcctgcagtacgacgacctgttcacctttggccagggcaccaagctggaaatcaaaggcggcagctctggcgacattcagctgacccagtctccaagctctctgagcgcctctgtgggcgatagagtgacaatcacatgccgcgccagccagagcatcagctacctgaattggtatcagcaaaaacccgggaagtctcccaagcggtggatctacgataccagcaagctggcctctggcgtgccctcaagattcagcggatctggctccggcaccgattacaccctgaccatcagcagcatgcagcccgaagatgccgccacatattactgtcagcagtggtccagcaatcccctgacctttggcggcggaacaaaggtcgagatcaaaggtggcggaggctctggcggttctggaagcggaggcggaggatctgcttctggatctggacaggtgcagctggtgcagtctggcgccgaagttgctaaacctggcgcctccgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccacactgaccaccgacaagagcaccagcaccgcctacatggaactgtccagcctgagaagcgaggacaccgccgtgtactactgtgcccggtactacgacgatcactacagcctggattactggggccagggaacaaccctgacagtgtctagcggcggatctagtggccaggtccagcttgttcagagcggagccgaagtgaaaaagccaggcgccagcgtgaaagtgtcttgcaaggcctccggcagcagcttcaccggctataccatgaattgggttaagcaagccccaggccaaggcctggaatggatcggaatcatcaacccttaccagggcgacaccacctacaaccagaaatttcagggaaaagtgaccatcaccgtggacaagtccacctccaccgtgtatatggaactcagcagcctgcgctccgaggatacagctgtgtactattgcgctagaggcgccggactgtactacttcgactattggggacagggcactaccgtgaccgtgtcctctgcatctggtggcagcggagacaagacccacacctgtcctccatgtcctgctccagaagctgcaggcggcccttccgtgtttctgttccctccaaagcctaaggacaccctgatgatctccagaacacccgaagtgacctgcgtggtggtggatgtgtcccacgaagatcctgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 235 - DNA, 3846 bp, synthetic construct
  source 1..3846: mol_type = other DNA; note = ahTrop2_h20xCD3_hOKT_Kcc_41BBLrw8 LALAPA
gacatccagatgacacagagccctagcagcctgtctgccagcgtgggagacagagtgaccattacctgtcaggccagccaggacatcaacaagtacctggcctggtatcagaagaagcccggcaaggcccctaagctgctgatccactacaccagcaatctgcagaccggcgtgcccagcagattttctggctctggcagcggcaccgacttcaccttcaccatatctagcctgcagcctgaggatatcgccacctactactgcctgcagtacgacgacctgttcacctttggccagggcaccaagctggaaatcaaaggcggcagctctggcgacattcagctgacccagtctccaagctctctgagcgcctctgtgggcgatagagtgacaatcacatgccgcgccagccagagcatcagctacctgaattggtatcagcaaaaacccgggaagtctcccaagcggtggatctacgataccagcaagctggcctctggcgtgccctcaagattcagcggatctggctccggcaccgattacaccctgaccatcagcagcatgcagcccgaagatgccgccacatattactgtcagcagtggtccagcaatcccctgacctttggcggcggaacaaaggtcgagatcaaaggtggcggaggctctggcggttctggaagcggaggcggaggatctgcttctggatctggacaggtgcagctggtgcagtctggcgccgaagttgctaaacctggcgcctccgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccacactgaccaccgacaagagcaccagcaccgcctacatggaactgtccagcctgagaagcgaggacaccgccgtgtactactgtgcccggtactacgacgatcactacagcctggattactggggccagggaacaaccctgacagtgtctagcggcggatctagtggccaggtccagcttgttcagagcggagccgaagtgaaaaagccaggcgccagcgtgaaagtgtcttgcaaggcctccggcagcagcttcaccggctataccatgaattgggttaagcaagccccaggccaaggcctggaatggatcggaatcatcaacccttaccagggcgacaccacctacaaccagaaatttcagggaaaagtgaccatcaccgtggacaagtccacctccaccgtgtatatggaactcagcagcctgcgctccgaggatacagctgtgtactattgcgctagaggcgccggactgtactacttcgactattggggacagggcactaccgtgaccgtgtcctctgcatctggtggcagcggagacaagacccacacctgtcctccatgtcctgctccagaagctgcaggcggcccttccgtgtttctgttccctccaaagcctaaggacaccctgatgatctccagaacacccgaagtgacctgcgtggtggtggatgtgtcccacgaagatcctgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 236 - DNA, 2157 bp, synthetic construct
  source 1..2157: mol_type = other DNA; note = ahcMetxahCD3_hOKT_ Kcc_LALAPA
gacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaaaggcggcagcagcggcgacatccagctgacacaaagcccatctagcctgagcgccagcgtgggcgatagagtgaccatcacatgtagagccagccagtccatcagctacctgaattggtatcaacaaaagcctggcaagagccccaagcggtggatctacgatacaagcaagctggcctctggcgtgcccagcagattcagcggatctggatctggcaccgattacacactgaccatcagcagcatgcagcccgaagatgccgccacctactattgtcagcagtggtccagcaatcccctgaccttcggaggcggaacaaaggtcgagatcaaaggtggcggaggatctggcggttctggaagtggcggtggtggatctgcttctggatcaggacaggtgcagctggtgcagtctggcgccgaagttgctaaacctggcgcctctgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccactctgaccaccgacaagagcaccagcaccgcctacatggaactgagcagcctgagaagcgaggacaccgccgtgtattattgcgcccggtactacgacgaccactacagcctggattattggggccagggcacaaccctgaccgtgtctagcggcggatcttctggtcaagtccagctggttcagagcggagccgaggttgtgaaaccaggcgctagcgtgaagctgagctgcaaggcctccggctacacctttaccagctactggctgcattgggtccgacaggcacctggccaaggactggaatgggtcggaatgatcgaccccagcaacagcgacacccggttcaaccccaacttcaaggaccgcgtgacaatcaccgccgacaccagcacatccacagcttatatggaactgtcctctctgcggagcgaagataccgctgtctactactgtgccacctacaagtcctacgtgtcccctctggactactggggacagggaacatctgtgacagtgtcctctgccagcggcggctctggcgataagacacacacatgccctccatgtccagctccagaagctgctggcggcccttccgtgtttctgttccctccaaagcctaaggacaccctgatgatcagcaggacccctgaagtgacctgcgtggtggtcgatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggc

SEQ ID NO: 237 - DNA, 3870 bp, synthetic construct
  source 1..3870: mol_type = other DNA; note = ahcMetxahCD3_hOKTKcc_LALAPA_4-1BBL
gacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaaaggcggcagcagcggcgacatccagctgacacaaagcccatctagcctgagcgccagcgtgggcgatagagtgaccatcacatgtagagccagccagtccatcagctacctgaattggtatcaacaaaagcctggcaagagccccaagcggtggatctacgatacaagcaagctggcctctggcgtgcccagcagattcagcggatctggatctggcaccgattacacactgaccatcagcagcatgcagcccgaagatgccgccacctactattgtcagcagtggtccagcaatcccctgaccttcggaggcggaacaaaggtcgagatcaaaggtggcggaggatctggcggttctggaagtggcggtggtggatctgcttctggatcaggacaggtgcagctggtgcagtctggcgccgaagttgctaaacctggcgcctctgtgaaggtgtcctgtaaagccagcggctacacattcacccggtacaccatgcactgggtcaagcaggctccaggacagggactcgagtggatcggctacatcaaccccagcagaggctacaccaactacgcccagaagttccagggcaaagccactctgaccaccgacaagagcaccagcaccgcctacatggaactgagcagcctgagaagcgaggacaccgccgtgtattattgcgcccggtactacgacgaccactacagcctggattattggggccagggcacaaccctgaccgtgtctagcggcggatcttctggtcaagtccagctggttcagagcggagccgaggttgtgaaaccaggcgctagcgtgaagctgagctgcaaggcctccggctacacctttaccagctactggctgcattgggtccgacaggcacctggccaaggactggaatgggtcggaatgatcgaccccagcaacagcgacacccggttcaaccccaacttcaaggaccgcgtgacaatcaccgccgacaccagcacatccacagcttatatggaactgtcctctctgcggagcgaagataccgctgtctactactgtgccacctacaagtcctacgtgtcccctctggactactggggacagggaacatctgtgacagtgtcctctgccagcggcggctctggcgataagacacacacatgccctccatgtccagctccagaagctgctggcggcccttccgtgtttctgttccctccaaagcctaaggacaccctgatgatcagcaggacccctgaagtgacctgcgtggtggtcgatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 238 - DNA, 3867 bp, synthetic construct
  source 1..3867: mol_type = other DNA; note = ahcMetxahTrop2_h20_Kcc_41BBLrw8 LALAPA
gacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaagggcggatcttctggagacatccagatgacacagagccctagcagcctgtctgccagcgtgggagacagagtgaccattacctgtcaggccagccaggacatcaacaagtacctggcctggtatcagaagaagcccggcaaggctcctaagctgctgatccactacaccagcaacctgcagacaggcgtgcccagcagattttctggcagcggctctggcaccgacttcaccttcaccataagcagcctgcagcctgaggatatcgccacctactactgcctgcagtacgacgacctgttcacctttggccagggcaccaagctggaaatcaaaggaggcggaggatctggcggatctggaagtggcggcggtggatctgcttctggctctggccaggtgcagctggtgcagtctggcgccgaagttaagaaacctggcgcctccgtgaaggtgtcctgtaaagccagcggcagcagcttcaccggctacaccatgaactgggtcaagcaggctcctggacagggactcgagtggatcggcatcatcaacccctaccagggcgacaccacctacaaccagaagttccagggcaaagtcaccatcaccgtggacaagagcaccagcaccgtgtacatggaactgtccagcctgagaagcgaggacaccgccgtgtactactgtgctagaggcgccggactgtactacttcgactattggggccagggaaccaccgtgaccgttagctctggaggcagctctggccaggttcagctggttcagtctggcgccgaggttgtgaaacctggcgcctctgtgaagctgagctgtaaagccagcggctacaccttcaccagctactggctgcactgggtccgacaggctccaggacaaggactggaatgggtcggaatgatcgaccccagcaacagcgacacccggttcaaccccaacttcaaggacagagtgaccatcaccgccgacaccagcacaagcaccgcctacatggaactgagcagcctgagaagcgaggacaccgccgtgtactactgcgccacctacaagagctacgtgtcccctctggattactggggccagggcacaagcgtgacagtgtcatctgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 239 - DNA, 3867 bp, synthetic construct
  source 1..3867: mol_type = other DNA; note = ahTrop2_h20xahcMet_Kcc_41BBLrw8 LALAPA
gacatccagatgacacagagccctagcagcctgtctgccagcgtgggagacagagtgaccattacctgtcaggccagccaggacatcaacaagtacctggcctggtatcagaagaagcccggcaaggctcctaagctgctgatccactacaccagcaacctgcagacaggcgtgcccagcagattttctggcagcggctctggcaccgacttcaccttcaccataagcagcctgcagcctgaggatatcgccacctactactgcctgcagtacgacgacctgttcacctttggccagggcaccaagctggaaatcaaaggcggatcttctggagacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaagggaggcggaggatctggcggatctggaagtggcggcggtggatctgcttctggctctggccaggttcagctggttcagtctggcgccgaggttgtgaaacctggcgcctctgtgaagctgagctgtaaagccagcggctacaccttcaccagctactggctgcactgggtccgacaggctccaggacaaggactggaatgggtcggaatgatcgaccccagcaacagcgacacccggttcaaccccaacttcaaggacagagtgaccatcaccgccgacaccagcacaagcaccgcctacatggaactgagcagcctgagaagcgaggacaccgccgtgtactactgcgccacctacaagagctacgtgtcccctctggattactggggccagggcacaagcgtgacagtgtcatctggaggcagctctggccaggtgcagctggtgcagtctggcgccgaagttaagaaacctggcgcctccgtgaaggtgtcctgtaaagccagcggcagcagcttcaccggctacaccatgaactgggtcaagcaggctcctggacagggactcgagtggatcggcatcatcaacccctaccagggcgacaccacctacaaccagaagttccagggcaaagtcaccatcaccgtggacaagagcaccagcaccgtgtacatggaactgtccagcctgagaagcgaggacaccgccgtgtactactgtgctagaggcgccggactgtactacttcgactattggggccagggaaccaccgtgaccgttagctctgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 240 - DNA, 3072 bp, synthetic construct
  source 1..3072: mol_type = other DNA; note = ahTrop2_h20Hrw3_Hcc_41BBLrw8 LALAPA
caggtgcagctggtgcagtctggcgccgaagttaagaaacctggcgcctccgtgaaggtgtcctgtaaagccagcggcagcagcttcaccggctacaccatgaactgggtcaagcaggctcctggacagggactcgagtggatcggcatcatcaacccctaccagggcgacaccacctacaaccagaagttccagggcaaagtcaccatcaccgtggacaagagcaccagcaccgtgtacatggaactgtccagcctgagaagcgaggacaccgccgtgtactactgtgctagaggcgccggactgtactacttcgactattggggccagggaaccaccgtgaccgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 241 - DNA, 3075 bp, synthetic construct
  source 1..3075: mol_type = other DNA; note = ahcMet_Hrw5_Hcc_41BBLrw8 LALAPA
caggttcagctggttcagtctggcgccgaggttgtgaaacctggcgcctctgtgaagctgagctgtaaagccagcggctacaccttcaccagctactggctgcactgggtccgacaggctccaggacaaggactggaatgggtcggaatgatcgaccccagcaacagcgacacccggttcaaccccaacttcaaggacagagtgaccatcaccgccgacaccagcacaagcaccgcctacatggaactgagcagcctgagaagcgaggacaccgccgtgtactactgcgccacctacaagagctacgtgtcccctctggattactggggccagggcacaagcgtgacagtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 242 - DNA, 3825 bp, synthetic construct
  source 1..3825: mol_type = other DNA; note = VRC01.23scFab_Kcc_4-1BBL LALAPA
ctgactcagtctcccggaacactgtcactgagccctggcgagacagccatcatcagctgtcgtacaagccagtacggaagcctggcctggtatcagcaaagacctggacaggcccctcggctggtcatctactctggctctacaagagccgctggcatccccgatagattcagcggaagtagatggggacccgactacaatctgaccatcagcaatctggaaagcggcgacttcggcgtgtactactgccagcagtacgagttctttggccagggcaccaaggtccaggtggacatcaaacgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggcggaggtggaagtggcggaggcggttctggtggtggtggctctggtggcggcggttcaggcggcggaggaagcggaggcggcggaagtggtggcggtggatctcaggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 243 - DNA, 3084 bp, synthetic construct
  source 1..3084: mol_type = other DNA; note = VRC01.23H_Kcc_4-1BBL LALAPA
caggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 244 - DNA, 2757 bp, synthetic construct
  source 1..2757: mol_type = other DNA; note = VRC01.23scFv_Kcc_hOx40L LALAPA
ctgactcagtctcccggaacactgtcactgagccctggcgagacagccatcatcagctgtcgtacaagccagtacggaagcctggcctggtatcagcaaagacctggacaggcccctcggctggtcatctactctggctctacaagagccgctggcatccccgatagattcagcggaagtagatggggacccgactacaatctgaccatcagcaatctggaaagcggcgacttcggcgtgtactactgccagcagtacgagttctttggccagggcaccaaggtccaggtggacatcaaaggaggcggaggatctggcggatctggaagtggcggcggtggatctgcttctggctctggccaggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttctggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 245 - DNA, 3153 bp, synthetic construct
  source 1..3153: mol_type = other DNA; note = VRC01.23scFv_Kcc_4-1BBL LALAPA
ctgactcagtctcccggaacactgtcactgagccctggcgagacagccatcatcagctgtcgtacaagccagtacggaagcctggcctggtatcagcaaagacctggacaggcccctcggctggtcatctactctggctctacaagagccgctggcatccccgatagattcagcggaagtagatggggacccgactacaatctgaccatcagcaatctggaaagcggcgacttcggcgtgtactactgccagcagtacgagttctttggccagggcaccaaggtccaggtggacatcaaaggaggcggaggatctggcggatctggaagtggcggcggtggatctgcttctggctctggccaggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttctggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 246 - DNA, 3885 bp, synthetic construct
  source 1..3885: mol_type = other DNA; note = ahCD20_hRitxahCD19_Kcc_41BBL LALAPA
gagatccagctgacacagagcccttctagcctgtctgccagcgtgggcgacagagtgaccatcacatgtagagccagcagcagcgtgtcctatatccactggttccagcagaagcctggaagcgcccctaagtccctgatctatgccacaagcaacctggcctctggcgtgcccagcagattttctggcagcggctctggcaccgactacaccctgacaatcagctccctgcagcctgaggacttcgccacctactactgccagcagtggacaagcaaccctcctacatttggcggaggcaccaagctggaaatcaagggcggatcttctggagacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggaggcggaggatctggcggatctggaagtggcggcggtggatctgcttctggctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctggaggcagctctggccaggttcagctggttcagcctggcgccgaagtgaaaaaacctggcgcctccgtgaagatgagctgcaaggccagcggctacaccttcaccagctacaacatgcactgggtccgacaggcccctggcagaggacttgaatggatgggcgccatctatcccggcaacggcgatacaagctacaaccagaaattccagggcagagtgaccctgaccgccgacaagagcacaagcaccgcctacatggaactgagcagcctggaaagcgaggacaccgccgtgtactactgtgccagaagcacctactacggcggcgactggtacttcaatgtgtggggagccggcaccaccgtgacagtttctgctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 247 - DNA, 3885 bp, synthetic construct
  source 1..3885: mol_type = other DNA; note = ahCD19xahCD20_hRit_Kcc_41BBL LALAPA
gacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggcggatcttctggagagatccagctgacacagagcccttctagcctgtctgccagcgtgggcgacagagtgaccatcacatgtagagccagcagcagcgtgtcctatatccactggttccagcagaagcctggaagcgcccctaagtccctgatctatgccacaagcaacctggcctctggcgtgcccagcagattttctggcagcggctctggcaccgactacaccctgacaatcagctccctgcagcctgaggacttcgccacctactactgccagcagtggacaagcaaccctcctacatttggcggaggcaccaagctggaaatcaagggaggcggaggatctggcggatctggaagtggcggcggtggatctgcttctggctctggccaggttcagctggttcagcctggcgccgaagtgaaaaaacctggcgcctccgtgaagatgagctgcaaggccagcggctacaccttcaccagctacaacatgcactgggtccgacaggcccctggcagaggacttgaatggatgggcgccatctatcccggcaacggcgatacaagctacaaccagaaattccagggcagagtgaccctgaccgccgacaagagcacaagcaccgcctacatggaactgagcagcctggaaagcgaggacaccgccgtgtactactgtgccagaagcacctactacggcggcgactggtacttcaatgtgtggggagccggcaccaccgtgacagtttctgctggaggcagctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 248 - DNA, 2127 bp, synthetic construct
  source 1..2127: mol_type = other DNA; note = ScFab_CD28_TGNcs_LALAPA_Kcc
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaacagaggcgaatgtggcggaggcggatcaggcggcggaggtagcggtggtggtggtagcggaggtggcggaagtggtggcggaggaagcggaggcggaggatctggtggtggcggatctggcggaggtggaagtcaagttcagctggtgcagtctggcgccgaagtgaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctgga

SEQ ID NO: 249 - DNA, 2145 bp, synthetic construct
  source 1..2145: mol_type = other DNA; note = ScFab_CD3_SAR_Hcc_LALAPA
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaagcggacagtggccgctcctagcgtgttcatctttccacctagcgacgagcagctgaagtccggcacagcctctgttgtgtgcctgctgaacaacttctaccccagagaagccaaggtgcagtggaaggtggacaacgccctgcagagcggcaatagccaagagtctgtgaccgagcaggacagcaaggactccacctacagcctgagcagcaccctgacactgagcaaggccgactacgagaagcacaaagtgtacgcctgcgaagtgacccaccagggcctttctagccctgtgaccaagagcttcaacaggggcgaatgtggcggcggaggatctggcggaggcggtagcggtggcggaggaagcggaggcggaggttcaggtggtggtggttcaggcggtggcggcagcggaggtggtggcagcggcggaggcggatctcaagttcagcttgtggaatccggcggaggggttgtgcaacctggaagaagcctgagactgagctgtgccgccagcggcttcacctttacaaaggcctggatgcactgggtccgacaggcccctggaaaacagctggaatgggtcgcccagatcaaggacaagagcaacagctacgccacctactacgccgacagcgtgaagggcagattcaccatcagccgggacgacagcaagaacaccctgtacctgcagatgaactccctgagagccgaggataccgccgtgtactactgccggggagtgtactatgctctgagccccttcgattactggggccagggaacactggtcaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 250 - DNA, 3483 bp, synthetic construct
  source 1..3483: mol_type = other DNA; note = CD3_SAR x VRC01.23_Hcc_hOx40L LALAPA
gacatcgtgatgacccagacacctctgagcctgagcgtgacacctggacagcctgccagcatcagctgcaagtctagccagagcctggtgcacaacaacgccaacacctacctgagctggtatctgcagaagcccggccagtctcctcagagcctgatctacaaggtgtccaacagattcagcggcgtgcccgacagattttctggcagcggctctggcaccgacttcaccctgaagatcagcagagtggaagccgaggacgtgggcgtgtactattgtggccagggcacacagtaccccttcacatttggaagcggcaccaaggtggaaatcaaaggcggcagctctggcctgacacagagccctggaacactgtcactgagccctggcgagacagccatcatctcttgcagaaccagccagtacggaagcctggcctggtatcagcaaaggcctggacaggcccctcggctggttatctactctggctctacaagagccgcaggcatccccgatagattctccggtagtagatggggacccgactacaacctgaccatcagcaacctggaaagcggcgacttcggagtgtactactgccagcagtacgagttcttcggacagggcacaaaggtgcaggtcgacatcaaaggtggcggcggaagcggaggatctggatctggcggaggcggatctgctagtggatctggacaggtgcagctggtgcagtctggcggccagatgaagaaaccaggcgagagcatgcggatcagctgtagagcctccggctacgagttcatcgattgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttctgcaccagaggcaagaactgcgattacaactgggacttcgagcactggggcagaggcacacctgtgatcgttagcagcggcggatctagccaggtccagctcgttgaatcaggtggcggagttgtgcagcctggcagatccctgagactgtcttgtgccgccagcggcttcacctttaccaaagcctggatgcactgggtccgacaggctccaggcaaacagctggaatgggtcgcccagatcaaggacaagagcaacagctacgccacctactacgccgacagcgtgaagggcagattcaccatctccagagatgacagcaagaacaccctgtacctgcagatgaacagcctgagagccgaggatacagccgtctactactgtagaggggtgtactacgctctgagccccttcgattattggggacagggaaccctggtcaccgtgtctagtgcttctggcggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 251 - DNA, 3468 bp, synthetic construct
  source 1..3468: mol_type = other DNA; note = CD28 x VRC01.23_Kcc_hOx40L LALAPA
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaaaggcggcagcagcggcctgactcagtctcctggaacactgtcactgagccctggcgagacagccatcatcagctgtagaaccagccagtacggcagcctggcctggtatcaacaaagacctggacaggcccctcggctggtcatctactctggctctacaagagccgctggcatccccgatagattcagcggaagtagatggggacccgactacaatctgaccatcagcaacctggaaagcggcgacttcggcgtgtactactgccagcagtacgagttctttggccagggcaccaaggtgcaggtcgacatcaaaggtggcggaggctctggcggatctggaagcggaggcggaggatctgcttctggatctggacaggtgcagctggtgcagtctggcggccagatgaagaaaccaggcgagagcatgcggatcagctgcagagcctctggctacgagttcatcgattgcaccctgaattggatcagactggcccctggcaagaggcctgaatggatgggatggctgaagcccagatggggagccgtgaattacgccagacctctgcagggcagagtgacaatgacccggcagctgtctcaggaccccgacgatcctgattggggcaccgcttttctggaactgagaagcctgaccgtggacgataccgccgtgtacttctgcaccagaggcaagaactgcgactacaactgggacttcgagcactggggcagaggcacacctgtgatcgtttctagcggcggctctagtggccaggtccagcttgttcaatctggcgccgaagtgaaaaagccaggcgcctctgtgaaggtgtcctgcaaggcctccggctacacattcaccagctactacatccactgggtccgacaggctccaggacagggacttgagtggatcggcagcatctaccccggcaacgtgaacaccaactacaacgagaagttcaaggaccgggccacactgacagtggacaccagcatcagcaccgcctacatggaactgtcccggctgagatccgatgacactgctgtgtatttctgtacacggtcccactacggcctggactggaactttgatgtgtggggccagggaaccaccgtgaccgtttcttctgcttctggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 252 - DNA, 2688 bp, synthetic construct
  source 1..2688: mol_type = other DNA; note = VRC01.23H_Kcc_Ox40L LALAPA
caggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 253 - DNA, 3429 bp, synthetic construct
  source 1..3429: mol_type = other DNA; note = VRC01.23scFab_Kcc_Ox40L LALAPA
ctgactcagtctcccggaacactgtcactgagccctggcgagacagccatcatcagctgtcgtacaagccagtacggaagcctggcctggtatcagcaaagacctggacaggcccctcggctggtcatctactctggctctacaagagccgctggcatccccgatagattcagcggaagtagatggggacccgactacaatctgaccatcagcaatctggaaagcggcgacttcggcgtgtactactgccagcagtacgagttctttggccagggcaccaaggtccaggtggacatcaaacgtacggtggccgctcccagcgtgttcatcttcccacctagcgacgagcagctgaagtccggcacagcctctgtcgtgtgcctgctgaacaacttctacccccgcgaggccaaagtgcagtggaaggtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgtggaggcggaggatctggcggaggtggaagtggcggaggcggttctggtggtggtggctctggtggcggcggttcaggcggcggaggaagcggaggcggcggaagtggtggcggtggatctcaggttcagctggttcagtctggcggtcagatgaagaaacccggcgagagcatgcggatcagctgtagagccagcggctacgagttcatcgactgcaccctgaactggatcagactggcccctggcaaaagacccgagtggatgggatggctgaagcccagatggggagccgtgaattacgccagacctctgcagggcagagtgaccatgacacggcagctgtctcaggaccccgacgatcctgattggggcaccgcttttctggaactgagaagcctgaccgtggacgataccgccgtgtacttttgcacgagaggcaagaactgcgactacaactgggacttcgagcactggggcagaggcacacctgtgatcgttagctctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatcctctggatctggaagcggcggctctggctctagcggagtgtctcacagatacagcagaatccagagcatcaaggtgcagttcaccgagtacaagaaagagaagggcttcatcctgaccagccagaaagaggacgagatcatgaaggtgcagaacaacagcgtgatcatcaactgcgacggcttctacctgatcagcctgaagggctacttcagccaagaagtgaacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccatgtgaatggcggcgagctgatcctgatccatcagaaccctggcgagttctgcgtgctcggaggatctggtggatctggcggtggaagttctggtagcggcgtgtcccatcggtacagccggattcagtccattaaggtccagtttacagagtataagaaagaaaaaggattcattctcacctctcagaaagaagatgagattatgaaggtccaaaacaattccgtcatcatcaattgtgacgggttttatctcatctccctcaagggctatttctctcaagaagtcaatatctcactccactatcagaaagatgaagaaccgctctttcagctcaaaaaagttcgctccgtgaactctctgatggtcgcctctctgacgtataaggacaaagtctatctcaatgtcacgacggacaacacatccctcgacgatttccacgtcaacggcggagaactgattctcattcaccagaatcctggggagttttgtgtcctcggcggaagtggtggaagcggaggcggaagttcaggctctggcgtcagccaccgctactccaggattcaatccatcaaagttcaattcacagagtacaaaaaagaaaaaggttttatcctgacgtcccaaaaagaggacgaaattatgaaggttcaaaacaactctgtgatcattaactgtgacggtttttacttgatctctctgaagggatacttttcccaagaagtcaacatttccctgcattatcaaaaggatgaggaaccacttttccaattgaaaaaagttcggagcgtcaactcactcatggtggcttccctgacgtacaaagataaggtttacttgaatgtcaccacagataatacgagcctcgatgactttcatgttaacggtggcgaactcatactgatccaccaaaatccaggcgaattctgtgtgctg

SEQ ID NO: 254 - DNA, 3888 bp, synthetic construct
  source 1..3888: mol_type = other DNA; note = ahCD19axahCD20PS_Kcc_41BBL LALAPA
gacatccagctgacacagagccctgcttctctggccgtgtctctgggacagagagccaccatcagctgcaaggccagccagagcgttgactacgacggcgacagctacctgaactggtatcagcagatccccggccagcctcctaagctgctgatctacgatgccagcaacctggtgtctggcatccctcctagattttccggcagcggctctggcaccgacttcaccctgaatatccatcctgtggaaaaggtggacgccgccacctaccactgtcagcagagcacagaagatccctggacctttggcggaggcaccaagctggaaatcaaaggcggaagcagcggcgacatcgtgctgactcagtctccagccatcctgtctgctagccctggcgagaaagtgaccatgacctgtagagccagcagcagcgtgaactacatggattggtatcagaagaagcccggcagcagccccaagccttggatctacgccacaagcaatctggccagcggagtgcctgccagattttctggctctggaagcggcaccagctacagcctgacaatcagcagagtggaagccgaggatgccgccacttactattgccagcagtggtccttcaatcctcctaccttcggaggcggaacaaagcttgagatcaaaggtggcggaggaagcggcggatctggatctggcggcggaggatctgctagtggaagcggagaagttcagctgcagcagtctggcgccgagcttgtgaaacctggcgcctctgtgaagatgtcttgcaaggcctccggctacaccttcaccagctataacatgcactgggtcaagcagacccctggacagggcctcgaatggatcggagccatctatcccggcaacggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagttagttctggcggaagctccggccaggtgcagcttcaacaaagcggagccgaacttgtgcggcctggctctagcgtgaagatcagctgtaaagccagtggctacgcctttagcagctactggatgaattgggtcaaacagcggccaggccagggacttgagtggatcggacaaatttggcctggcgacggcgataccaactacaatggcaagtttaaaggcaaggctaccctgacagccgacgagtctagctccaccgcttatatgcagctgtctagcctggcctccgaggactccgccgtgtacttttgcgctcggagagagacaaccaccgtcggcagatattactacgccatggactactggggccagggcacaaccgtgacagtgtctagtgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 255 - DNA, 3888 bp, synthetic construct
  source 1..3888: mol_type = other DNA; note = ahCD20_PSxahCD19_Kcc_41BBL LALAPA
gacatcgtgctgacacagagccctgccatcctgagtgctagccctggcgagaaagtgaccatgacctgtagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaaaggcggaagcagcggcgacatccagctgactcaatctccagcctctctggccgtgtcactgggacagagagccaccatcagctgcaaggccagccagtccgtggactacgatggcgacagctacctgaattggtatcagcagatccccggccagcctcctaagctgctgatctacgatgccagcaacctggtgtctggcatccctcctagattttccggcagcggaagcggcaccgacttcaccctgaatatccatcctgtggaaaaggtggacgccgccacatatcactgccagcagagcaccgaagatccctggacatttggaggcggaacaaagcttgagatcaaaggtggcggaggctccggcggatctggatctggcggcggaggatctgctagtggatctggacaggttcagctgcagcagtctggcgccgaactcgttagacctggcagctccgtgaagatcagctgtaaagcctccggctacgccttcagcagctactggatgaactgggtcaagcagaggccaggccagggcctcgaatggatcggacaaatttggcctggcgacggcgacaccaactacaacggcaagttcaagggcaaagccacactgaccgccgacgagtctagcagcacagcctacatgcagctgtctagcctggctagcgaggacagcgccgtgtacttctgcgctagaagagagacaaccaccgtcggccggtactactacgccatggattattggggccagggcaccaccgtgacagtgtcaagtggtggatcttccggcgaggtgcagcttcagcaaagcggagccgaactggtcaaacctggcgcaagcgtgaagatgtcttgcaaggcctctggctacacctttacgagctacaacatgcattgggttaagcagacccctggccaaggacttgagtggatcggcgccatctatcccggcaatggcgacacctcctacaatcagaagtttaaaggcaaggctaccctcaccgccgacaagagcagctctaccgcttatatgcagctgagcagcctgacctccgaggactccgccgattactactgtgccagaagcaactactacggctcctcctactggttcttcgacgtgtggggagccggcacaacagtgactgtgtctagcgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 256 - DNA, 2121 bp, synthetic construct
  source 1..2121: mol_type = other DNA; note = ScFab_CD3_hOKT_v23_Hcc_LALAPA
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcacactgaccctgagcaaggccgactacgagaagcacaaagtgtacgcctgcgaagtgacccaccagggcctttctagccctgtgaccaagagcttcaacagaggcgaatgtggcggcggaggaagcggaggcggaggttctggtggtggtggctctggtggcggaggatctggcggaggtggaagtggcggtggcggttcaggcggaggcggtagcggcggaggcggatctcaagttcagctggttcagtctggcgccgaggtggcaaaacctggcgcctctgtgaaagtgtcctgcaaggccagcggctacaccttcaccagatacaccatgcactgggtcaagcaggcccctggacaaggcttggagtggatcggctacatcaaccccagccggggctacaccaattacgcccagaagttccagggcaaagccactctgaccaccgacaagagcaccagcaccgcctacatggaactgtccagcctgagaagcgaggacaccgccgtgtactactgtgcccggtactacgacgaccactactccctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggc

SEQ ID NO: 257 - DNA, 3888 bp, synthetic construct
  source 1..3888: mol_type = other DNA; note = CD20_PSxCD19 LALAPA_41BBL
gacatcgtgctgacacagagccctgccatcctgagtgctagccctggcgagaaagtgaccatgacctgtagagccagcagcagcgtgaactacatggactggtatcagaagaagcccggcagcagccccaagccttggatctacgccacaagcaatctggccagcggagtgcctgccagattttctggctctggcagcggcacaagctacagcctgacaatcagcagagtggaagccgaggatgccgccacctactactgtcagcagtggtccttcaatcctcctaccttcggcggaggcaccaagctggaaatcaaaggcggaagcagcggcgacatccagctgactcaatctccagcctctctggccgtgtcactgggacagagagccaccatcagctgcaaggccagccagtccgtggactacgatggcgacagctacctgaattggtatcagcagatccccggccagcctcctaagctgctgatctacgatgccagcaacctggtgtctggcatccctcctagattttccggcagcggaagcggcaccgacttcaccctgaatatccatcctgtggaaaaggtggacgccgccacatatcactgccagcagagcaccgaagatccctggacatttggaggcggaacaaagcttgagatcaaaggtggcggaggctccggcggatctggatctggcggcggaggatctgctagtggatctggacaggttcagctgcagcagtctggcgccgaactcgttagacctggcagctccgtgaagatcagctgtaaagcctccggctacgccttcagcagctactggatgaactgggtcaagcagaggccaggccagggcctcgaatggatcggacaaatttggcctggcgacggcgacaccaactacaacggcaagttcaagggcaaagccacactgaccgccgacgagtctagcagcacagcctacatgcagctgtctagcctggctagcgaggacagcgccgtgtacttctgcgctagaagagagacaaccaccgtcggccggtactactacgccatggattattggggccagggcaccaccgtgacagtgtcaagtggtggatcttccggcgaggtgcagcttcagcaaagcggagccgaactggtcaaacctggcgcaagcgtgaagatgtcttgcaaggcctctggctacacctttacgagctacaacatgcattgggttaagcagacccctggccaaggacttgagtggatcggcgccatctatcccggcaatggcgacacctcctacaatcagaagtttaaaggcaaggctaccctcaccgccgacaagagcagctctaccgcttatatgcagctgagcagcctgacctccgaggactccgccgattactactgtgccagaagcaactactacggctcctcctactggttcttcgacgtgtggggagccggcacaacagtgactgtgtctagcgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 258 - DNA, 3888 bp, synthetic construct
  source 1..3888: mol_type = other DNA; note = CD19 x CD20_PS LALAPA_41BBL
gacatccagctgacacagagccctgcttctctggccgtgtctctgggacagagagccaccatcagctgcaaggccagccagagcgttgactacgacggcgacagctacctgaactggtatcagcagatccccggccagcctcctaagctgctgatctacgatgccagcaacctggtgtctggcatccctcctagattttccggcagcggctctggcaccgacttcaccctgaatatccatcctgtggaaaaggtggacgccgccacctaccactgtcagcagagcacagaagatccctggacctttggcggaggcaccaagctggaaatcaaaggcggaagcagcggcgacatcgtgctgactcagtctccagccatcctgtctgctagccctggcgagaaagtgaccatgacctgtagagccagcagcagcgtgaactacatggattggtatcagaagaagcccggcagcagccccaagccttggatctacgccacaagcaatctggccagcggagtgcctgccagattttctggctctggaagcggcaccagctacagcctgacaatcagcagagtggaagccgaggatgccgccacttactattgccagcagtggtccttcaatcctcctaccttcggaggcggaacaaagcttgagatcaaaggtggcggaggaagcggcggatctggatctggcggcggaggatctgctagtggaagcggagaagttcagctgcagcagtctggcgccgagcttgtgaaacctggcgcctctgtgaagatgtcttgcaaggcctccggctacaccttcaccagctataacatgcactgggtcaagcagacccctggacagggcctcgaatggatcggagccatctatcccggcaacggcgacacctcctacaaccagaagttcaagggcaaagccacactgaccgccgacaagagcagcagcacagcctacatgcagctgagcagcctgaccagcgaggacagcgccgattactactgcgccagaagcaactactacggcagctcctactggttcttcgacgtgtggggagccggcaccacagtgacagttagttctggcggaagctccggccaggtgcagcttcaacaaagcggagccgaacttgtgcggcctggctctagcgtgaagatcagctgtaaagccagtggctacgcctttagcagctactggatgaattgggtcaaacagcggccaggccagggacttgagtggatcggacaaatttggcctggcgacggcgataccaactacaatggcaagtttaaaggcaaggctaccctgacagccgacgagtctagctccaccgcttatatgcagctgtctagcctggcctccgaggactccgccgtgtacttttgcgctcggagagagacaaccaccgtcggcagatattactacgccatggactactggggccagggcacaaccgtgacagtgtctagtgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 259 - DNA, 3867 bp, synthetic construct
  source 1..3867: mol_type = other DNA; note = cMetxTrop2_h20_41BBL
gacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaagggcggatcttctggagacatccagatgacacagagccctagcagcctgtctgccagcgtgggagacagagtgaccattacctgtcaggccagccaggacatcaacaagtacctggcctggtatcagaagaagcccggcaaggctcctaagctgctgatccactacaccagcaacctgcagacaggcgtgcccagcagattttctggcagcggctctggcaccgacttcaccttcaccataagcagcctgcagcctgaggatatcgccacctactactgcctgcagtacgacgacctgttcacctttggccagggcaccaagctggaaatcaaaggaggcggaggatctggcggatctggaagtggcggcggtggatctgcttctggctctggccaggtgcagctggtgcagtctggcgccgaagttaagaaacctggcgcctccgtgaaggtgtcctgtaaagccagcggcagcagcttcaccggctacaccatgaactgggtcaagcaggctcctggacagggactcgagtggatcggcatcatcaacccctaccagggcgacaccacctacaaccagaagttccagggcaaagtcaccatcaccgtggacaagagcaccagcaccgtgtacatggaactgtccagcctgagaagcgaggacaccgccgtgtactactgtgctagaggcgccggactgtactacttcgactattggggccagggaaccaccgtgaccgttagctctggaggcagctctggccaggttcagctggttcagtctggcgccgaggttgtgaaacctggcgcctctgtgaagctgagctgtaaagccagcggctacaccttcaccagctactggctgcactgggtccgacaggctccaggacaaggactggaatgggtcggaatgatcgaccccagcaacagcgacacccggttcaaccccaacttcaaggacagagtgaccatcaccgccgacaccagcacaagcaccgcctacatggaactgagcagcctgagaagcgaggacaccgccgtgtactactgcgccacctacaagagctacgtgtcccctctggattactggggccagggcacaagcgtgacagtgtcatctgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 260 - DNA, 3867 bp, synthetic construct
  source 1..3867: mol_type = other DNA; note = ahTrop2_h20xahcMet 41BBL
gacatccagatgacacagagccctagcagcctgtctgccagcgtgggagacagagtgaccattacctgtcaggccagccaggacatcaacaagtacctggcctggtatcagaagaagcccggcaaggctcctaagctgctgatccactacaccagcaacctgcagacaggcgtgcccagcagattttctggcagcggctctggcaccgacttcaccttcaccataagcagcctgcagcctgaggatatcgccacctactactgcctgcagtacgacgacctgttcacctttggccagggcaccaagctggaaatcaaaggcggatcttctggagacatcgtgatgacacagagccctgatagcctggccgtgtctctgggagagagagccaccatcaactgcaagagcagccagagcctgctgtacacctccagccagaagaactacctggcctggtatcagcagaagcccggccagcctcctaagctgctgatctactgggccagcaccagagaaagcggcgtgcccgatagattttctggctctggcagcggcaccgacttcaccctgacaatttctagcctgcaagccgaggacgtggccgtgtactactgccagcagtactacgcttacccctggacctttggcggaggcaccaagctggaaatcaagggaggcggaggatctggcggatctggaagtggcggcggtggatctgcttctggctctggccaggttcagctggttcagtctggcgccgaggttgtgaaacctggcgcctctgtgaagctgagctgtaaagccagcggctacaccttcaccagctactggctgcactgggtccgacaggctccaggacaaggactggaatgggtcggaatgatcgaccccagcaacagcgacacccggttcaaccccaacttcaaggacagagtgaccatcaccgccgacaccagcacaagcaccgcctacatggaactgagcagcctgagaagcgaggacaccgccgtgtactactgcgccacctacaagagctacgtgtcccctctggattactggggccagggcacaagcgtgacagtgtcatctggaggcagctctggccaggtgcagctggtgcagtctggcgccgaagttaagaaacctggcgcctccgtgaaggtgtcctgtaaagccagcggcagcagcttcaccggctacaccatgaactgggtcaagcaggctcctggacagggactcgagtggatcggcatcatcaacccctaccagggcgacaccacctacaaccagaagttccagggcaaagtcaccatcaccgtggacaagagcaccagcaccgtgtacatggaactgtccagcctgagaagcgaggacaccgccgtgtactactgtgctagaggcgccggactgtactacttcgactattggggccagggaaccaccgtgaccgttagctctgctagtggcggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 261 - DNA, 2712 bp, synthetic construct
  source 1..2712: mol_type = other DNA; note = ScFab_CD3_Hcc_mono4-1BBL8
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcacactgaccctgagcaaggccgactacgagaagcacaaagtgtacgcctgcgaagtgacccaccagggcctttctagccctgtgaccaagagcttcaacagaggcgaatgtggcggcggaggaagcggaggcggaggttctggtggtggtggctctggtggcggaggatctggcggaggtggaagtggcggtggcggttcaggcggaggcggtagcggcggaggcggatctcaagttcagctggttcagtctggcgccgaggtggcaaaacctggcgcctctgtgaaagtgtcctgcaaggccagcggctacaccttcaccagatacaccatgcactgggtcaagcaggcccctggacaaggcttggagtggatcggctacatcaaccccagccggggctacaccaattacgcccagaagttccagggcaaagccactctgaccaccgacaagagcaccagcaccgcctacatggaactgtccagcctgagaagcgaggacaccgccgtgtactactgtgcccggtactacgacgaccactactccctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggctctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatct

SEQ ID NO: 262 - DNA, 2718 bp, synthetic construct
  source 1..2718: mol_type = other DNA; note = ScFab_CD28_Kcc_mono4-1BBL8
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaacagaggcgaatgtggcggaggcggatcaggcggcggaggtagcggtggtggtggtagcggaggtggcggaagtggtggcggaggaagcggaggcggaggatctggtggtggcggatctggcggaggtggaagtcaagttcagctggtgcagtctggcgccgaagtgaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggctctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatct

SEQ ID NO: 263 - DNA, 2610 bp, synthetic construct
  source 1..2610: mol_type = other DNA; note = ScFab_CD3_Hcc_mono4-1BBL7
gacatccagctgacacagagcccttctagcctgtctgccagcgtgggcgacagagtgaccatcacatgtagagccagccagagcatcagctacctgaactggtatcagcagaagcccggcaagagccccaagcggtggatctacgatacaagcaagctggcctctggcgtgcccagcagattttctggctctggcagcggcaccgactacaccctgacaatcagcagcatgcagcctgaggatgccgccacctactactgccagcagtggtccagcaatcccctgacatttggcggaggcaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcacactgaccctgagcaaggccgactacgagaagcacaaagtgtacgcctgcgaagtgacccaccagggcctttctagccctgtgaccaagagcttcaacagaggcgaatgtggcggcggaggaagcggaggcggaggttctggtggtggtggctctggtggcggaggatctggcggaggtggaagtggcggtggcggttcaggcggaggcggtagcggcggaggcggatctcaagttcagctggttcagtctggcgccgaggtggcaaaacctggcgcctctgtgaaagtgtcctgcaaggccagcggctacaccttcaccagatacaccatgcactgggtcaagcaggcccctggacaaggcttggagtggatcggctacatcaaccccagccggggctacaccaattacgcccagaagttccagggcaaagccactctgaccaccgacaagagcaccagcaccgcctacatggaactgtccagcctgagaagcgaggacaccgccgtgtactactgtgcccggtactacgacgaccactactccctggattattggggccagggcacaaccctgaccgtgtcatctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaggctgctggtggcccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctcgcgaacctcaagtctgtacactgcctcctagccgggacgagctgaccaagaatcaggtgtcactgagctgcgccgtgaagggcttttaccccagcgacattgccgtggaatgggagagcaatggccagccagagaacaactacaagacaacccctcctgtgctggacagcgacggctcatttttcctggtgtccaagctgacagtggacaagagcagatggcagcagggcaacgtgttcagctgcagcgtgatgcacgaggccctgcacaaccactacacccagaaaagcctgtctctgagccctggcggaggatccggctctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacaccc

SEQ ID NO: 264 - DNA, 2616 bp, synthetic construct
  source 1..2616: mol_type = other DNA; note = ScFab_CD28_Kcc_mono4-1BBL7
gacatccagatgacacagagccctagcagcctgtctgccagcgtgggagacagagtgaccatcacatgtcacgccagccagaacatctacgtgtggctgaactggtatcagcagaagcccggcaaggcccctaagctgctgatctacaaggccagcaatctgcacaccggcgtgcccagcagattttctggctctggcagcggcaccgacttcaccctgaccatatctagcctgcagcctgaggacttcgccacctactattgtcagcagggccagacatacccctacacctttggcggcggaacaaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctactctctgagcagcaccctgacactgagcaaggccgactacgagaagcacaaagtgtacgcctgcgaagtgacccaccagggcctttctagccctgtgaccaagagcttcaacagaggcgaatgtggcggaggcggatcaggcggcggaggtagcggtggtggtggtagcggaggtggcggaagtggtggcggaggaagcggaggcggaggatctggtggtggcggatctggcggaggtggaagtcaagttcagctggtgcagtctggcgccgaagtgaagaaacctggcgcctccgtgaaggtgtcctgtaaagccagcggctacacattcaccagctactacatccactgggtccgacaggcccctggacaaggacttgagtggatcggcagcatctaccccggcaacgtgaacaccaactacaacgagaagttcaaggaccgggccacactgaccgtggacacctctatcagcaccgcctacatggaactgagccggctgagatccgatgataccgccgtgtacttctgcaccagaagccactacggcctggactggaacttcgatgtgtggggccagggcaccaccgtgacagtttcttctgcttcgaccaagggcccatccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgctagcggaggctccggagataagacccacacatgtcctccatgtcctgctccagaagctgccggcggaccttccgtgtttctgttccctccaaagcctaaggacaccctgatgatcagcaggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggctctggcggtggaagcggaggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggctctgcatctgcagcctctgagatcagctgctggtgctgctgctctggccctgacagttgatctgcctcctgcctctagcgaggccagaaacagcgcctttggcttccaaggcagactgctgcatctgtctgccggccagagactgggagtccatctgcacacagaggccagagctagacatgcctggcagctgacacagggcgctacagtgctgggcctgtttagagtgacaccc

SEQ ID NO: 265 - DNA, 3891 bp, synthetic construct
  source 1..3891: mol_type = other DNA; note = ahCD20_ofxahCD19_Kcc_41BBL LALAPA
gagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagggcggatcttctggagacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggaggcggaggatctggcggatctggaagtggcggcggtggatctgcttctggctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctggaggcagctctggcgaggtgcagctggttgaatctggcggaggactggtgcagcctggcagaagtctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 266 - DNA, 3891 bp, synthetic construct
  source 1..3891: mol_type = other DNA; note = ahCD20_ofx ahCD19_41BBLrw8
gagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagggcggatcttctggagacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggaggcggaggatctggcggatctggaagtggcggcggtggatctgcttctggctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctggaggcagctctggcgaggtgcagctggttgaatctggcggaggactggtgcagcctggcagaagtctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 267 - DNA, 3891 bp, synthetic construct
  source 1..3891: mol_type = other DNA; note = ahCD19xahCD20_of_Kcc_41BBL LALAPA
gacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggcggatcttctggagagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagggaggcggaggatctggcggatctggaagtggcggcggtggatctgcttctggctctggcgaggtgcagctggttgaatctggcggaggactggtgcagcctggcagaagtctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctggaggcagctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctggctgcccctatcgagaaaaccatcagcaaggccaagggccagcctagggaaccccaggtttacacccttcctccatgccgggatgagctgaccaagaaccaggtgtccctgtggtgtctggtcaagggcttctaccccagcgacattgccgtggaatgggagagcaatggccagcctgagaacaactacaagaccacacctcctgtgctggactccgacggatcattcttcctgtacagcaagctgactgtggacaagagcagatggcagcagggcaacgtgttcagctgcagcgtgatgcacgaggccctgcacaaccactacacacagaagtccctgtctctgagccctggcggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 268 - DNA, 3891 bp, synthetic construct
  source 1..3891: mol_type = other DNA; note = ahCD19xahCD20_of_41BBLrw8
gacatccagctgacccagtctccaagctttctgagcgccagcgtgggcgacagagtgaccatcacatgtaaagccagccagagcgtggactacagcggcgacagctacctgaactggtatcagcagaagcccggcaaggcccctaagctgctgatctacgacgcctccaatctggtgtccggcgtgcccagcagattttctggctctggaagcggcaccgagttcaccctgaccatatctagcctgcagcctgaggacttcgccacctactactgccagcagagcacagagaacccctggacatttggcggcggaacaaagctggaaatcaagggcggatcttctggagagatcgtgctgacacagtctcccgccacactgtcactgtctccaggcgaaagagccacactgagctgtagagccagccagagcgtgtcctcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcccatcacctttggccagggcaccagactggaaatcaagggaggcggaggatctggcggatctggaagtggcggcggtggatctgcttctggctctggcgaggtgcagctggttgaatctggcggaggactggtgcagcctggcagaagtctgagactgtcttgtgccgccagcggcttcaccttcaacgactatgccatgcactgggtccgacaggcccctggcaaaggacttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacaacagtgacagtctcttctggaggcagctctggccaggttcagctggttcagtctggcgccgaagtgaagaaacctggcagcagcgtgaaggtgtcctgcaaggcctctggctacgccttcagcagctactggatgaactgggtccgacaggcccctggacaaggcttggaatggatgggccaaatctggcctggcgacagcgacaccaattacgcccagaaattccagggcagagtgaccatcaccgccgacgagtctacaagcaccgcctacatggaactgagcagcctgagaagcgaggacaccgccgtgtactactgtgccagacgggaaaccaccaccgtgggcagatattactacgccatggactactggggccagggcaccacagtgacagtctcttctgcttctggcggctccggagataagacccacacatgtcctccatgtcctgctccagaagctgctggcggcccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctggccgctcctatcgagaaaaccatcagcaaggccaagggccagcctagggaaccccaggtttacacactgcctccaagcagggacgagctgaccaagaatcaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggaggaggatccggatctggcggaggatctggactgagagagggacctgagctgagccctgatgatcctgctggactgctggatctgcggcagggaatgtttgctcagctggtggcccagaacgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggttctggatctgtgtctctggctctgcatctgcagcctctgagatcagctgctggtgctgctgctctggccctgacagttgatctgcctcctgcctctagcgaggccagaaacagcgcctttggcttccaaggcagactgctgcacctgtctgctggacagagactgggagtgcacctccacacagaggccagagctagacatgcctggcagctgacacagggcgctacagtgctgggcctgtttagagtgacccctgagattccagccggcctgccttctccaagaagcgagggatctctgagagaaggcccagagctgtccccagatgatccagccggactccttgacctgagacagggcatgttcgcccagctcgtcgctcagaatgtcctgctgatcgacggacccctgtcctggtattctgatccaggactggcaggcgtgtcactgacaggcggactgtcttacaaagaagatacgaaagagcttgtcgtcgccaaagctggggtctactatgtgtttttccagcttgagctgaggcgcgtggtcgctggcgaaggcagtggttctgtttcactggccctgcaccttcagccactcagaagtgctgcaggcgcagctgcactggctctcactgtggatttgcctccagccagctctgaggcccggaatagcgccttcggatttcaaggacgcctgctgcatctgagcgcaggccaaagactcggagtccatctgcacaccgaagctagagccagacacgcttggcaactcacccaaggcgcaactgtcctgggactgttccgcgtgacacctgaaatccctgccggacttccatctcctagatccgagggcagcctcagagaaggacccgaactgtctcctgacgacccagctggcctgctcgatcttaggcaagggatgttcgcacaactggtggcacaaaatgtgctcctcatcgacgggccacttagctggtactcagatcccggtcttgcaggggtgtcccttactggtggcctctcttacaaagaggatacaaaagaactcgttgtcgcaaaggcaggggtctactacgtgttcttccaactcgaactccggcgcgtcgtggcaggcgaaggctcaggttcagttagcctggcactccacttgcagccccttagaagcgcagctggcgcagcagctcttgcactgactgtcgatcttccaccagccagcagcgaggctaggaactccgcttttggttttcaaggcaggctcctccatctctctgccggccaaaggcttggcgtccaccttcatactgaggctagggctcgtcacgcatggcagttgactcaaggtgccacagtcctcgggctgttcagagtcactccagaaatccccgctggcttgcccagtcctagaagtgaaggatct

SEQ ID NO: 269 - protein, 119 aa, synthetic construct
  source 1..119: note = cMet VH
QVQLVQSGAEVVKPGASVKLSCKASGYTFTSYWLHWVRQAPGQGLEWVGMIDPSNSDTRFNPNFKDRVTITADTSTSTAYMELSSLRSEDTAVYYCATYKSYVSPLDYWGQGTSVTVSS

SEQ ID NO: 270 - protein, 8 aa, synthetic construct
  source 1..8: note = cMet HCDR1
GYTFTSYW

SEQ ID NO: 271 - protein, 18 aa, synthetic construct
  source 1..18: note = cMet HCDR2
GMIDPSNSDTRFNPNFKD

SEQ ID NO: 272 - protein, 12 aa, synthetic construct
  source 1..12: note = cMet HCDR3
ATYKSYVSPLDY

SEQ ID NO: 273 - protein, 113 aa, synthetic construct
  source 1..113: note = cMet VL
DIVMTQSPDSLAVSLGERATINCKSSQSLLYTSSQKNYLAWYQQKPGQPPKLLIYWASTRESGVPDRFSGSGSGTDFTLTISSLQAEDVAVYYCQQYYAYPWTFGGGTKLEIK

SEQ ID NO: 274 - protein, 18 aa, synthetic construct
  source 1..18: note = cMet LCDR1
KSSQSLLYTSSQKNYLAW

SEQ ID NO: 275 - protein, 7 aa, synthetic construct
  source 1..7: note = cMet LCDR2
WASTRES

SEQ ID NO: 276 - protein, 9 aa, synthetic construct
  source 1..9: note = cMet LCDR3
QQYYAYPWT

SEQ ID NO: 277 - protein, 118 aa, synthetic construct
  source 1..118: note = Trop2_h20 VH
QVQLVQSGAEVKKPGASVKVSCKASGSSFTGYTMNWVKQAPGQGLEWIGIINPYQGDTTYNQKFQGKVTITVDKSTSTVYMELSSLRSEDTAVYYCARGAGLYYFDYWGQGTTVTVSS

SEQ ID NO: 278 - protein, 8 aa, synthetic construct
  source 1..8: note = Trop2_h20 HCDR1
GSSFTGYT

SEQ ID NO: 279 - protein, 7 aa, synthetic construct
  source 1..7: note = Trop2_h20 HCDR2
NPYQGDT

SEQ ID NO: 280 - protein, 11 aa, synthetic construct
  source 1..11: note = Trop2_h20 HCDR3
ARGAGLYYFDY

SEQ ID NO: 281 - protein, 106 aa, synthetic construct
  source 1..106: note = Trop2_h20 VL
DIQMTQSPSSLSASVGDRVTITCQASQDINKYLAWYQKKPGKAPKLLIHYTSNLQTGVPSRFSGSGSGTDFTFTISSLQPEDIATYYCLQYDDLFTFGQGTKLEIK

SEQ ID NO: 282 - protein, 6 aa, synthetic construct
  source 1..6: note = Trop2_h20 LCDR1
QDINKY

SEQ ID NO: 283 - skipped

SEQ ID NO: 284 - protein, 8 aa, synthetic construct
  source 1..8: note = Trop2_h20 LCDR3
LQYDDLFT

SEQ ID NO: 285 - protein, 124 aa, synthetic construct
  source 1..124: note = hrCD19 VH
QVQLVQSGAEVKKPGSSVKVSCKASGYAFSSYWMNWVRQAPGQGLEWMGQIWPGDSDTNYAQKFQGRVTITADESTSTAYMELSSLRSEDTAVYYCARRETTTVGRYYYAMDYWGQGTTVTVSS

SEQ ID NO: 286 - protein, 8 aa, synthetic construct
  source 1..8: note = hrCD19 HCDR1
GYAFSSYW

SEQ ID NO: 287 - protein, 8 aa, synthetic construct
  source 1..8: note = hrCD19 HCDR2
IWPGDSDT

SEQ ID NO: 288 - protein, 17 aa, synthetic construct
  source 1..17: note = hrCD19 HCDR3
ARRETTTVGRYYYAMDY

SEQ ID NO: 289 - protein, 111 aa, synthetic construct
  source 1..111: note = hrCD19 VL
DIQLTQSPSFLSASVGDRVTITCKASQSVDYSGDSYLNWYQQKPGKAPKLLIYDASNLVSGVPSRFSGSGSGTEFTLTISSLQPEDFATYYCQQSTENPWTFGGGTKLEIK

SEQ ID NO: 290 - protein, 10 aa, synthetic construct
  source 1..10: note = hrCD19 LCDR1
QSVDYSGDSY

SEQ ID NO: 291 - protein, 7 aa, synthetic construct
  source 1..7: note = hrCD19 LCDR2
DASNLVS

SEQ ID NO: 292 - protein, 9 aa, synthetic construct
  source 1..9: note = hrCD19 LCDR3
QQSTENPWT

SEQ ID NO: 293 - protein, 125 aa, synthetic construct
  source 1..125: note = ahCD3_Glo VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 294 - protein, 5 aa, synthetic construct
  source 1..5: note = ahCD3_Glo HCDR1
TYAMN

SEQ ID NO: 295 - protein, 19 aa, synthetic construct
  source 1..19: note = ahCD3_Glo HCDR2
RIRSKYNNYATYYADSVKG

SEQ ID NO: 296 - protein, 14 aa, synthetic construct
  source 1..14: note = ahCD3_Glo HCDR3
HGNFGNSYVSWFAY

SEQ ID NO: 297 - protein, 109 aa, synthetic construct
  source 1..109: note = ahCD3_Glo VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 298 - protein, 5 aa, synthetic construct
  source 1..5: note = ahCD3_Glo LCDR1
NYANW

SEQ ID NO: 299 - protein, 7 aa, synthetic construct
  source 1..7: note = ahCD3_Glo LCDR2
GTNKRAP

SEQ ID NO: 300 - protein, 9 aa, synthetic construct
  source 1..9: note = ahCD3_Glo LCDR3
ALWYSNLWV

SEQ ID NO: 301 - protein, 121 aa, synthetic construct
  source 1..121: note = ahCD3_SAR VH
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 302 - protein, 5 aa, synthetic construct
  source 1..5: note = ahCD3_SAR HCDR1
KAWMH

SEQ ID NO: 303 - protein, 19 aa, synthetic construct
  source 1..19: note = ahCD3_SAR HCDR2
QIKDKSNSYATYYADSVKG

SEQ ID NO: 304 - protein, 10 aa, synthetic construct
  source 1..10: note = ahCD3_SAR HCDR3
VYYALSPFDY

SEQ ID NO: 305 - protein, 112 aa, synthetic construct
  source 1..112: note = ahCD3_SAR VL
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 306 - protein, 16 aa, synthetic construct
  source 1..16: note = ahCD3_SAR LCDR1
KSSQSLVHNNANTYLS

SEQ ID NO: 307 - protein, 7 aa, synthetic construct
  source 1..7: note = ahCD3_SAR LCDR2
KVSNRFS

SEQ ID NO: 308 - protein, 9 aa, synthetic construct
  source 1..9: note = ahCD3_SAR LCDR3
GQGTQYPFT

SEQ ID NO: 309 - protein, 122 aa, synthetic construct
  source 1..122: note = ahCD20_PS VH
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 310 - protein, 5 aa, synthetic construct
  source 1..5: note = ahCD20_PS HCDR1
SYNMH

SEQ ID NO: 311 - protein, 17 aa, synthetic construct
  source 1..17: note = ahCD20_PS HCDR2
AIYPGNGDTSYNQKFKG

SEQ ID NO: 312 - protein, 13 aa, synthetic construct
  source 1..13: note = ahCD20_PS HCDR3
SNYYGSSYWFFDV

SEQ ID NO: 313 - protein, 106 aa, synthetic construct
  source 1..106: note = ahCD20_PS VL
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 314 - protein, 10 aa, synthetic construct
  source 1..10: note = ahCD20_PS LCDR1
RASSSVNYMD

SEQ ID NO: 315 - protein, 7 aa, synthetic construct
  source 1..7: note = ahCD20_PS LCDR2
ATSNLAS

SEQ ID NO: 316 - protein, 9 aa, synthetic construct
  source 1..9: note = ahCD20_PS LCDR3
QQWSFNPPT

SEQ ID NO: 317 - protein, 122 aa, synthetic construct
  source 1..122: note = ahCD20_of VH
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 318 - protein, 5 aa, synthetic construct
  source 1..5: note = ahCD20_of HCDR1
DYAMH

SEQ ID NO: 319 - protein, 17 aa, synthetic construct
  source 1..17: note = ahCD20_of HCDR2
TISWNSGSIGYADSVKG

SEQ ID NO: 320 - protein, 13 aa, synthetic construct
  source 1..13: note = ahCD20_of HCDR3
DIQYGNYYYGMDV

SEQ ID NO: 321 - protein, 107 aa, synthetic construct
  source 1..107: note = ahCD20_of VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 322 - protein, 11 aa, synthetic construct
  source 1..11: note = ahCD20_of LCDR1
RASQSVSSYLA

SEQ ID NO: 323 - protein, 7 aa, synthetic construct
  source 1..7: note = ahCD20_of LCDR2
DASNRAT

SEQ ID NO: 324 - protein, 9 aa, synthetic construct
  source 1..9: note = ahCD20_of LCDR3
QQRSNWPIT

(19) United States
(12) Patent Application Publication
(10) Pub. No.: US 2024/0034808 A1
(43) Pub. Date: Feb. 1, 2024

(54) ANTIGEN BINDING POLYPEPTIDE COMPLEXES CONTAINING EXTRACELLULAR DOMAINS OF TNFSF LIGANDS

(71) Applicant: ModeX Therapeutics, Inc., NATICK, MA (US)

(72) Inventors: HEATHER DAWN KAMP, WESTWOOD, MA (US); EDWARD SEUNG, MEDFORD, MA (US); ZHI-YONG YANG, NEWTON CENTER, MA (US); GARY J. NABEL, DELRAY BEACH, FL (US); RONNIE R. WEI, WESTON, MA (US); ELIAS ZERHOUNI, FORT LAUDERDALE, FL (US)

(21) Appl. No.: 18/067,230

(22) Filed: Dec. 16, 2022

Related U.S. Application Data
(60) Provisional application No. 63/291,305, filed on Dec. 17, 2021.

(51) Int. Cl. C07K 16/32; C07K 14/525; C07K 16/28; A61P 35/00

(52) U.S. Cl. CPC C07K 16/32; C07K 14/525; C07K 16/2818; C07K 16/2809; A61P 35/00; C07K 2317/31; A61K 38/00

(57) ABSTRACT

Disclosed are antigen binding polypeptide complexes (e.g., antibodies and antigen binding fragments thereof) having certain structural features. Also disclosed are polynucleotides and vectors encoding such polypeptide complexes; cells, pharmaceutical compositions, and kits containing such polypeptide complexes; and methods of using such polypeptide complexes.

### CROSS REFERENCE TO RELATED APPLICATIONS

This application claims the priority benefit of U.S. Provisional Application No. 63/291,305, filed Dec. 17, 2021, which is incorporated herein by reference in its entirety.

### REFERENCE TO SEQUENCE LISTING SUBMITTED ELECTRONICALLY

The content of the electronically submitted sequence listing (Name: 4850_0080001_Seqlisting_ST26; Size: 699,814 bytes; Date of Creation: Dec. 13, 2022) is herein incorporated by reference in its entirety.

### FIELD

The present disclosure relates to antigen binding polypeptide complexes (e.g., antibodies and antigen binding fragments thereof) containing extracellular domains of tumor necrosis factor superfamily (TNFSF) ligands. The present disclosure also relates to polynucleotides and vectors encoding such polypeptide complexes, cells, pharmaceutical compositions, and kits containing such polypeptide complexes; and methods of using such polypeptide complexes.

### BACKGROUND

T cells are a subtype of white blood cells that play a key role in the immune system and fighting cancer. Cancer cell antigens can be presented by antigen presenting cells (APCs), which in turn can activate T cells to recognize and kill cancer cells. T cell activation requires two signaling events: (i) primary signaling through peptide-loaded major histocompatibility complexes (MHCs) on APCs and T cell receptor (TCR) complexes on T cells, and (ii) co-stimulatory signaling by CD28 family or tumor necrosis factor receptor superfamily (TNFRSF) members. The co-stimulatory signaling pathways are complementary to each other, as CD28 is the primary co-stimulatory pathway on naive T cells, while TNFRSF play a more important role in antigen-experienced or memory T cells.

Treatment of T cells with anti-CD3 antibodies and anti-CD28 antibodies provides a co-stimulatory signal that engages TCRs and can be used for antigen-induced T cell activation. The primary T cell activation signal is often provided by the anti-CD3 antibodies, as CD3 is a conserved member of the TCR complex. A second signal is then often provided by the anti-CD28 antibodies or CD28 ligand (B7.1, B7.2, etc.), and anti-TNF receptor (TNFR) members or their ligands TNFRSF members such as OX40 and 4-1BB have been well studied for biotherapeutic development for immunomodulation and immunotherapy of cancers either using antagonistic or agonistic approaches.

Except for CD27, which is constitutively expressed on naive T cells, other TNFRSFs are expressed only upon T cell activation. In addition, memory T cells and regulatory T cells (Tregs constitutively express certain family members. These expression patterns have suggested that the TNFRSF/TNFSF axis may be important in controlling effector and memory responses. Co-signaling receptors, and particularly TNFRSF co-stimulatory receptors, have a substantial role in regulating effector T cell responses. CD27-, OX40- and DR3-mediated co-stimulation promotes proliferation and survival of both CD4+ and CD8+ effector T cells, whereas 4-1BB- and GITR-mediated co-stimulation preferentially enhances the expansion and survival of CD8+ effector T cells.

OX40 is activated through binding by its ligand OX40L, and 4-1BB signals by engaging its ligand 4-1BBL. OX40L and 4-1BBL are trimeric molecules that can form homotrimer complexes with trimeric OX40 or 4-1BB on T cells.

Co-stimulation via TNFSF has emerged as a promising strategy to support antitumor immune responses. However, there is still a need for bispecific and multispecific antibodies that can bind specific target molecules or combinations of target molecules and more effectively activate T cells in antitumor immune responses. There is a further need for bispecific and multispecific antibodies that yield high efficacy and, at the same time, provide a wider therapeutic window and better tolerability.

### BRIEF SUMMARY

Provided herein is an antigen binding polypeptide complex comprising a first polypeptide, a second polypeptide, and a third polypeptide; wherein the first polypeptide has a structure represented by VL1-L1-CL; VL1-L1-CH1; VH1-L1-CL; or VH1-L1-CH1; wherein (i) the second polypeptide has a structure represented by VH1-L2-CH1-L3-Fc-L4-TNF1-L5-TNF2-L6-TNF3; VH1-L2-CL-L3-Fc-L4-TNF1-L5-TNF2-L6-TNF3; VH1-L7-CH1-L8-Fc; VH1-L7-CL-L8-Fc; VL1-L2-CH1-L3-Fc-L4-TNF1-L5-TNF2-L6-TNF3; VL1-L2-CL-L3-Fc-L4-TNF1-L5-TNF2-L6-TNF3; VL1-L7-CH1-L8-Fc; or VL1-L7-CL-L8-Fc; and the third polypeptide has a structure represented by VL2-L9-VH2-L10-Fc-L11-TNF1-L12-TNF2-L13-TNF3; or VH2-L14-VL2-L15-Fc-L16-TNF1-L17-TNF2-L18-TNF3; or (ii) the second polypeptide has a structure represented by VH1-L19-CH1-L20-Fc-L21-TNF1-L22-TNF2-L23-TNF3; VH1-L19-CL-L20-Fc-L21-TNF1-L22-TNF2-L23-TNF3; VL1-L19-CH1-L20-Fc-L21-TNF1-L22-TNF2-L23-TNF3; or VL1-L19-CL-L20-Fc-L21-TNF1-L22-TNF2-L23-TNF3; and the third polypeptide has a structure represented by VL2-L24-VH2-L25-Fc; or VH2-L26-VL2-L27-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L27 are amino acid linkers.

Provided herein is an antigen binding polypeptide complex comprising a first polypeptide, a second polypeptide, and a third polypeptide; wherein the first polypeptide has a structure represented by VL1-L1-CL; VL1-L1-CH1; VH1-L1-CL; or VH1-L1-CH1; wherein (i) the second polypeptide has a structure represented by VH1-CH1-L2-Fc-L3-TNF1-L4-TNF2-L5-TNF3; VH1-CL-L2-Fc-L3-TNF1-L4-TNF2-L5-TNF3; VH1-CH1-L6-Fc; VH1-CL-L7-Fc; VL1-CH1-L2-Fc-L3-TNF1-L4-TNF2-L5-TNF3; VL1-CL-L2-Fc-L3-TNF1-L4-TNF2-L5-TNF3; VL1-CH1-L6-Fc; or VL1-CL-L7-Fc; and the third polypeptide has a structure represented by VL2-L8-VH2-L9-Fc-L10-TNF1-L11-TNF2-L12-TNF3; or VH2-L13-VL2-L14-Fc-L15-TNF1-L16-TNF2-L17-TNF3; or (ii) the second polypeptide has a structure represented by VH1-CH1-L18-Fc-L19-TNF1-L20-TNF2-L21-TNF3; VH1-CL-L18-Fc-L19-TNF1-L20-TNF2-L21-TNF3; VL1-CH1-L18-Fc-L19-TNF1-L20-TNF2-L21-TNF3; or VL1-CL-L18-Fc-L19-TNF1-L20-TNF2-L21-TNF3; and the third polypeptide has a structure represented by VL2-L22-VH2-L23-Fc or VH2-L24-VL2-L25-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L25 are amino acid linkers.

Provided herein is an antigen binding polypeptide complex comprising a first polypeptide and a second polypeptide; wherein (i) the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L37-VH2-L38-Fc-L39-TNF1-L40-TNF2-L41-TNF3; VH2-L42-VL2-L43-Fc-L44-TNF1-L45-TNF2-L46-TNF3; VL2-L47-VH2-L48-CL-L49-CH1-L50-Fc-L51-TNF1-L52-TNF2-L53-TNF3; VL2-L54-CL-L55-VH2-L56-CH1-L57-Fc-L58-TNF1-L59-TNF2-L60-TNF3; VL2-L47-VH2-L48-CH1-L49-CL-L50-Fc-L51-TNF1-L52-TNF2-L53-TNF3; VL2-L54-CH1-L55-VH2-L56-CL-L57-Fc-L58-TNF1-L59-TNF2-L60-TNF3; VL2-VH2-L38-Fc-L39-TNF1-L40-TNF2-L41-TNF3; VH2-VL2-L43-Fc-L44-TNF1-L45-TNF2-L46-TNF3; VL2-VH2-L48-CL-L49-CH1-L50-Fc-L51-TNF1-L52-TNF2-L53-TNF3; VL2-CL-L55-VH2-L56-CH1-L57-Fc-L58-TNF1-L59-TNF2-L60-TNF3; VL2-VH2-L48-CH1-L49-CL-L50-Fc-L51-TNF1-L52-TNF2-L53-TNF3; or VL2-CH1-L55-VH2-L56-CL-L57-Fc-L58-TNF1-L59-TNF2-L60-TNF3; or (ii) the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L85-VH2-L86-Fc; VH2-L87-VL2-L88-Fc; VL2-L89-VH2-L90-CL-L91-CH1-L92-Fc; VL2-L89-VH2-L90-CH1-L91-CL-L92-Fc; VL2-L93-CL-L94-VH2-L95-CH1-L96-Fc; or VL2-L93-CH1-L94-VH2-L95-CL-L96-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a TNFSF ligand; TNF2 is a second extracellular domain of a TNFSF ligand; TNF3 is a third extracellular domain of a TNFSF ligand; and L1-L96 are amino acid linkers.

Provided herein is an antigen binding polypeptide complex comprising a first polypeptide and a second polypeptide; wherein (i) the first polypeptide has a structure represented by Fc; VL1-L1-VL2-L2-VH2-L3-VH1-L4-Fc; VH1-L5-VH2-L6-VL2-L7-VL1-L8-Fc; Fc-L9-TNF1-L10-TNF2-L11-TNF3; VL1-L12-VL2-L13-VH2-L14-VH1-L15-Fc-L16-TNF1-L17-TNF2-L18-TNF3; or VH1-L19-VH2-L20-VL2-L21-VL1-L22-Fc-L23-TNF1-L24-TNF2-L25-TNF3; and the second polypeptide has a structure represented by VL3-L26-VL4-L27-VH4-L28-VH3-L29-Fc-L30-TNF1-L31-TNF2-L32-TNF3; or VH3-L33-VH4-L34-VL4-L35-VL3-L36-Fc-L37-TNF1-L38-TNF2-L39-TNF3; or (ii) the first polypeptide has a structure represented by Fc-L40-TNF1-L41-TNF2-L42-TNF3; VL1-L43-VL2-L44-VH2-L45-VH1-L46-Fc-L47-TNF1-L48-TNF2-L49-TNF3; or VH1-L50-VH2-L51-VL2-L52-VL1-L53-Fc-L54-TNF1-L55-TNF2-L56-TNF3; and the second polypeptide has a structure represented by Fc; VL3-L57-VL4-L58-VH4-L59-VH3-L60-Fc; or VH3-L61-VH4-L62-VL4-L63-VL3-L64-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; TNF1 is a first extracellular domain of a TNFSF ligand; TNF2 is a second extracellular domain of a TNFSF ligand; TNF3 is a third extracellular domain of a TNFSF ligand; and L1-L64 are amino acid linkers.

Provided herein is an antibody or antigen binding fragment thereof comprising the antigen binding polypeptide complex described herein.

Provided herein is a pharmaceutical composition comprising an antigen binding polypeptide complex or antibody or antigen binding fragment described herein, and a pharmaceutically acceptable carrier.

Provided herein is a method for inducing or enhancing an immune response, comprising administering to a subject in need thereof an antigen binding polypeptide complex or the antibody or antigen binding fragment, or pharmaceutical composition described herein.

Provided herein is a method for overcoming cancer-mediated immune suppression, comprising administering to a subject in need thereof an antigen binding polypeptide complex, antibody or antigen binding polypeptide complex, or pharmaceutical composition described herein.

### BRIEF DESCRIPTION OF THE DRAWINGS

FIG. 1A-1F illustrates exemplary antigen binding polypeptide complexes of the disclosure. Fv1 and Fv2 represent regions that bind to immune activating receptors or tumor associated antigens (TAAs). In some aspects, Fv1 and Fv2 bind to human CD3 or CD28. In some aspects, Fv1 and Fv2 are in the form of a single-chain variable fragment (scFv). In some aspects, Fv1 and Fv2 are in the form of a Fab or single chain Fab (scFab), optionally with CH1 and CL regions. TNFSF represents a trimer of TNF superfamily member extracellular domains fused to the Fc. In some aspects, the TNFSF is in the form of a fusion homotrimer. In some aspects, the TNFSF is a dimer of a fusion homotrimer.

FIG. 2A shows ELISA binding results of three exemplary antigen binding polypeptide complexes containing OX40L trimers (MX169, MX368 and MX369) to human CD3.

FIG. 2B shows ELISA binding results of three exemplary antigen binding polypeptide complexes containing OX40L trimers (MX169, MX368 and MX369) to human CD28.

FIG. 2C shows ELISA binding results of three exemplary antigen binding polypeptide complexes containing OX40L trimers (MX169, MX368 and MX369) to human OX40.

FIG. 3A shows the fold change in T cell activation tested in Jurkat cell lines expressing luciferase under the control of the NF-κB (NFkb) promoter after overnight stimulation with different concentrations of three exemplary antigen binding polypeptide complexes containing OX40L trimers (MX169, MX368 and MX369). Results from a control IgG1 isotype antibody are also shown (IgG1 isotype).

FIG. 3B shows the fold change in T cell activation tested in Jurkat cell lines expressing luciferase under the control of the NFAT promoter after overnight stimulation with different concentrations of three exemplary antigen binding polypeptide complexes containing OX40L trimers (MX169, MX368 and MX369). Results from a control IgG1 isotype antibody are also shown (IgG1 isotype).

FIG. 4A-4F shows the fold change in proliferation of primary human CD4+ T cells (FIGS. 4A-4C) and CD8+ T cells (FIGS. 4D-4F) from three different donors upon treatment with one of three exemplary antigen binding polypeptide complexes containing OX40L trimers (MX169, MX368 and MX369). Results from a control IgG1 isotype antibody are also shown (IgG1 isotype). Human peripheral blood peripheral blood mononuclear cells (PBMCs) were incubated with the antigen binding polypeptide complexes for 7 days and stained for flow cytometry. CD4+ and CD8+ T cells were identified and their concentrations were determined using Precision Count Beads™. Fold change was calculated by dividing cell concentrations from Day 7 and Day 0.

FIG. 5A shows ELISA binding results of three exemplary antigen binding polypeptide complexes containing 4-1BBL trimers (MX306, MX424 and MX425) to human CD3.

FIG. 5B shows ELISA binding results of three exemplary antigen binding polypeptide complexes containing 4-1BBL trimers (MX306, MX424 and MX425) to human CD28.

FIG. 5C shows ELISA binding results of three exemplary antigen binding polypeptide complexes containing 4-1BBL trimers (MX306, MX424 and MX425) to human 4-1BBL.

FIG. 6A-6F shows the fold change in proliferation of primary human CD4+ T cells (FIGS. 6A-6C) and CD8+ T cells (FIGS. 6D-6F) from three different donors upon treatment with three exemplary antigen binding polypeptide complexes containing 4-1BBL trimers (MX306, MX424 and MX425). Results from a control IgG1 isotype antibody are also shown (IgG1 isotype). Human PBMCs were incubated with the antigen binding polypeptide complexes for 7 days and stained for flow cytometry. CD4+ and CD8+ T cells were identified and their concentrations were determined using Precision Count Beads™. Fold change was calculated by dividing cell concentrations from Day 7 and Day 0.

FIG. 7A-7B shows T cell proliferation, measured as the fold change of CD4 Tcm and Tem from two donors (FIG. 7A and FIG. 7B, respectively) in peripheral blood mononuclear cells (PBMCs), caused by MX169 and MX240. Results from a control antibody (hIgG1 isotype) are also shown.

FIG. 8 shows T cell proliferation, measured as the fold change of CD4 Tcm and Tem in PBMCs, caused by MX169, MX368 and MX369 from three donors. Results from a control antibody (IgG1 isotype) are also shown.

FIG. 9 shows T cell proliferation, measured as the fold change of CD8 Tcm and Tem in PBMCs, caused by MX169 and MX240 from two donors. Results from a control antibody (IgG1 isotype) are also shown.

FIG. 10 shows T cell proliferation, measured as the fold change of CD8 Tcm and Tem in PBMCs, caused by MX169, MX368 and MX369 from three donors. Results from a control antibody (IgG1 isotype) are also shown.

FIG. 11 shows T cell proliferation, measured as the fold change of CD4 Tcm and Tem or CD8 Tcm and Tem in PBMCs, caused by MX306 and MX321 from two donors. Results from a control antibody (IgG1 isotype) are also shown.

FIG. 12 shows T cell proliferation, measured as the fold change of CD4 Tcm and Tem in PBMCs, caused by MX306, MX424 and MX425 from three donors. Results from a control antibody (IgG1 isotype) are also shown.

FIG. 13 shows T cell proliferation, measured as the fold change of CD8 Tcm and Tem in PBMCs, caused by MX306, MX424 and MX425 from three donors. Results from a control antibody (IgG1 isotype) are also shown.

FIG. 14A shows IFNgamma, IL-2, IL-6 and TNFa release from primary human T cells, caused by MX169, MX170, MX250, MX368 and MX369. Results from a control antibody (IgG iso) are also shown. FIG. 14B shows the structures of MX169, MX170, MX250, MX368 and MX369.

FIG. 15 shows IL-4, IL-5 and IL-10 release from primary human T cells, caused by MX169, MX170, MX250, MX368 and MX369. Results from a control antibody (IgG iso) are also shown.

FIG. 16 shows IFNgamma, IL-2, IL-6 and TNFa release from primary human T cells, caused by MX169, MX170, MX250, MX368 and MX369. Results from a control antibody (IgG iso) are also shown.

FIG. 17 shows IL-4, IL-5 and IL-10 release from primary human T cells, caused by MX169, MX170, MX250, MX368 and MX369. Results from a control antibody (IgG iso) are also shown.

FIG. 18A shows IFNgamma, IL-2, IL-6 and TNFa release from primary human T cells, caused by MX306, MX170, MX318, MX424 and MX425. Results from a control antibody (IgG iso) are also shown. FIG. 18B shows the structures of MX306, MX170, MX318, MX424 and MX425.

FIG. 19 shows IL-4, IL-5 and IL-10 release from primary human T cells, caused by MX306, MX170, MX318, MX424 and MX425. Results from a control antibody (IgG iso) are also shown.

FIG. 20 shows IFNgamma, IL-2, IL-6 and TNFa release from primary human T cells, caused by MX306, MX170, MX318, MX424 and MX425. Results from a control antibody (IgG iso) are also shown.

FIG. 21 shows IL-4, IL-5 and IL10 release from primary human T cells, caused by MX306, MX170, MX318, MX424 and MX425. Results from a control antibody (IgG iso) are also shown.

FIG. 22A shows activation of non-human primate (NHP) CD4 and CD8 T cells, caused by MX424, MX485, MX487, MX620 and MX622. Results from a control antibody (IgG1 isotype) are also shown. FIG. 22B shows the structures of MX424, MX485, MX487, MX620 and MX622.

FIG. 23 shows fold change of CD4 and CD8 cells, caused by MX424, MX485, MX487, MX620 and MX622. Results from a control antibody (IgG1 isotype) are also shown.

FIG. 24 shows release of IFNgamma, IL-6, IL-2 and TNFa, caused by MX424, MX485, MX487, MX620 and MX622. Results from a control antibody (IgG1 isotype) are also shown.

FIG. 25A shows activation of CD4 and CD8 NHP T cells caused by MX368 and MX489. Results from a control antibody (IgG1 isotype) are also shown. FIG. 25B shows the structures of MX368 and MX489.

FIG. 26 shows proliferation of CD4 and CD8 NHP T cells caused by MX368 and MX489. Results from a control antibody (IgG1 isotype) are also shown.

FIG. 27 shows release of IFNgamma, IL-6, IL-2 and TNFa from NHP T cells caused by MX368 and MX489. Results from a control antibody (IgG1 isotype) are also shown.

FIG. 28 shows T cell count and percent T cell activation in NHPs, following treatment with MX487 in two donors.

FIG. 29 shows the percentage of naïve, Tcm, Teff and Tem populations of CD4 and CD8 cells from two different NHPs, following treatment with MX487.

FIG. 30 shows the number of T cells and percent CD4 and CD8 T cell activation from two NHPs treated with MX620. Arrows indicate antibody dosing.

FIG. 31 shows the percentage of naïve, Tcm, Teff and Tem populations of CD4 and CD8 T cells from two donors, following treatment with MX620.

FIG. 32 shows the fold change in T cell number and CD4 and CD8 T cell activation from two NHPs, following treatment with MX620. Arrows indicate antibody dosing.

FIG. 33 shows the fold change in naïve, Tcm, Teff and Tem populations of CD8 T cells from two donor NHPs. Arrows indicate antibody dosing.

FIG. 34 shows percent lysis of Z138 cells following treatment with increasing concentrations of MX582 and MX583. Treatment with a control antibody (hIgG1LALAPA) is also shown.

FIG. 35 shows percent lysis of Z138 cells following treatment with increasing concentrations of MX751, MX777 and MX778. Treatment with a control antibody (hIgG1LALAPA) is also shown.

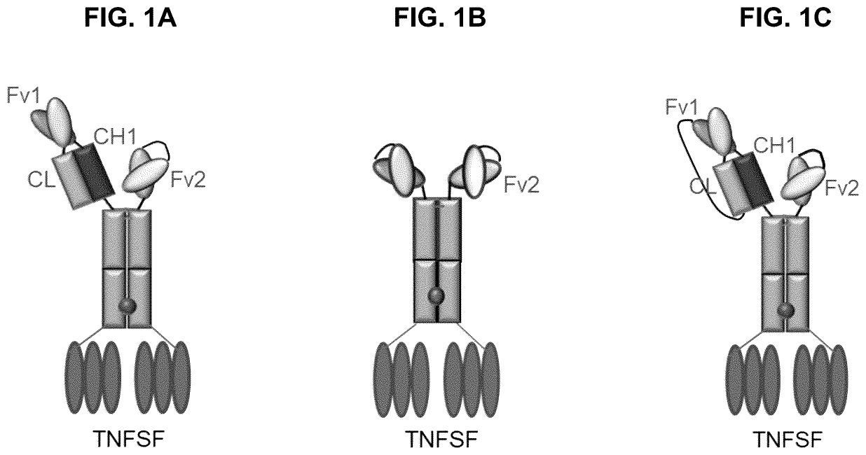

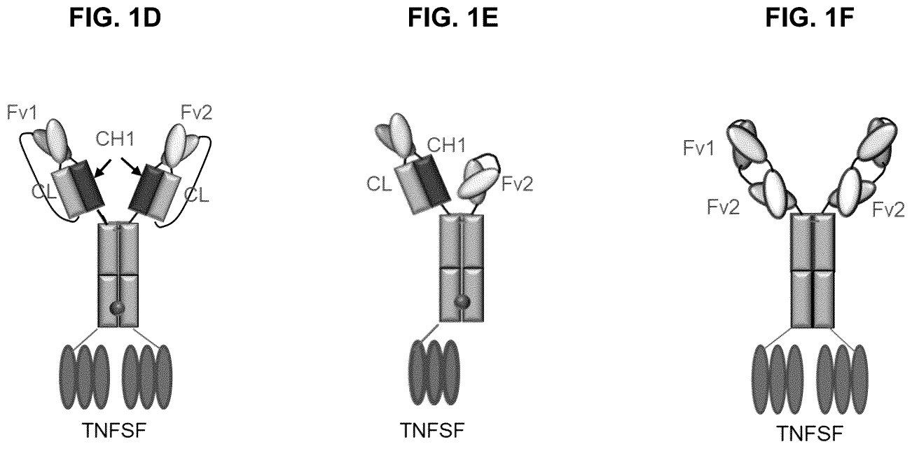

### DETAILED DESCRIPTION

In view of the roles of anti-CD3 antibodies, OX40 and 4-1BB in T cell activation and antitumor immune responses, combining anti-CD3 antibodies with OX40 or 4-1BB co-stimulatory signals may broaden the activation of various T cell populations, and provide sustained stimulus for T cell survival and long-term expansion. Therefore, antigen binding polypeptide complexes (e.g., antibodies or antigen binding fragments thereof) integrating one or more anti-CD3 regions (e.g., a complementarity determining region (CDR), heavy chain variable region (VH), light chain variable region (VL), single-chain variable fragment (scFv), Fab, single-chain Fab (scFab), heavy chain, or light chain) and one or more trimers of an extracellular domain of a tumor necrosis factor superfamily (TNFSF) ligand (e.g., OX40L or 4-1BBL) were developed. In some aspects, one or more anti-tumor associated antigen (TAA) regions such as one or more anti-HER2 binding regions (e.g., CDR, VH, VL, scFv, Fab, scFab, heavy chain or light chain) were further integrated into the antigen binding polypeptide complexes of the disclosure. In some aspects, one or more anti-immune stimulatory receptor regions such as one or more anti-CD28 binding regions (e.g., CDR, VH, VL, scFv, Fab, scFab, heavy chain or light chain) were further integrated into the antigen binding complexes of the invention.

Accordingly, the invention is directed to antigen binding polypeptide complexes (e.g., antibodies or antigen binding fragments thereof) having improved features. In some aspects, the invention enables the generation of multispecific and multifunctional antigen binding polypeptide complexes through the expression of complementary self-assembling heavy and light chains expressed with a single polypeptide per arm and, optionally, with the addition of specific amino acid linkers. Because of this multifunctionality, antigen binding polypeptide complexes of the invention can bind to specific combinations of target molecules for selectivity or breadth/neutralization, bring together two or more cell types, bring together targets and deliver activation signals, modify the disease microenvironment, and enhance avidity of binding for improved potency.

Various terms relating to aspects of disclosure are used throughout the specification and claims Such terms are to be given their ordinary meaning in the art, unless otherwise indicated. Other specifically defined terms are to be construed in a manner consistent with the definition provided herein.

### I. Definitions

As used herein, the term “antigen binding polypeptide complex” refers to a group of two, three, or four associated polypeptides, wherein at least one polypeptide has the ability to specifically bind to one or more antigens. An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each L chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art, which affect, e.g., half-life, effector function, and/or immune responses to the antibody in a patient. The term antibody also includes artificial polypeptide constructs, which comprise at least one antibody-derived antigen binding site.

An “antigen binding fragment” refers to one or more fragments or portions of an antibody that retain the ability to bind specifically to the antigen bound by the whole antibody. It has been shown that the antigen binding function of an antibody can be performed by fragments or portions of a full-length antibody. An antigen binding fragment can contain the antigenic determining regions of an intact antibody (e.g., the complementarity determining regions (CDRs)). Examples of antigen binding fragments of antibodies include, but are not limited to, Fab, Fab′, F(ab′)₂, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments are screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human FRs. In some aspects, the variable region is a primate (e.g., non-human primate) variable region. In some aspects, the variable region comprises rodent or murine CDRs and primate (e.g., non-human primate) FRs.

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and so on. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide complex, antibody or antigen binding fragment thereof. In some aspects, a VH region is referred to herein as VH1 to denote a first heavy chain variable region, VH2 to denote a second heavy chain variable region, VH3 to denote a third heavy chain variable region, and so on. An enumerated VH region (e.g., VH1) can have the same or different antigen binding properties and/or the same or different sequence as another enumerated VH region (e.g., VH2).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Arm NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (a), delta (6), epsilon (E), gamma (γ), and mu 40, based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide complex, antibody, antigen binding fragment thereof, polynucleotide, vector, or cell which is “isolated” is a polypeptide complex, antibody, antigen binding fragment thereof, polynucleotide, vector, or cell which is in a form not found in nature. Isolated polypeptide complexes, antibodies, antigen binding fragments thereof, polynucleotides, vectors, or cells include those which have been purified to a degree that they are no longer in a form in which they are found in nature. In some aspects, a polypeptide complex, antibody, antigen binding fragment thereof, polynucleotide, vector, or cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” “having” and the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### II. Antigen Binding Polypeptide Complexes

In some aspects, the invention is directed to antigen binding polypeptide complexes having certain structural features described further herein. In some aspects, an antigen binding polypeptide complex of the invention (e.g., antibody or antigen binding fragment thereof) comprises an anti-CD3 region (e.g., CDR, VH, VL, scFv, Fab, scFab, heavy chain or light chain) and one or more trimers of an extracellular domain of a tumor necrosis factor superfamily (TNFSF) ligand. In some aspects, an antigen binding polypeptide complex of the invention further comprises one or more anti-tumor associated antigen (TAA) regions such as one or more anti-HER2 binding regions (e.g., CDR, VH, VL, scFv, Fab, scFab, heavy chain or light chain). In some aspects, an antigen binding polypeptide complex of the invention further comprises one or more anti-immune stimulatory receptor regions such as one or more anti-CD28 binding regions (e.g., CDR, VH, VL, scFv, Fab, scFab, heavy chain or light chain). In some aspects, one or more constant regions (e.g., CH1 and/or CL) can also be incorporated into the polypeptides of the antigen binding polypeptide complexes.

A. Extracellular Domain of a TNFSF Ligand and Linkers

As used herein, an “extracellular domain of a tumor necrosis factor superfamily ligand” or “extracellular domain of a TNFSF ligand” refer to a peptide comprising a portion of a ligand of the tumor necrosis superfamily that forms trimers (also referred to as the TNF homology domain or ectodomain) As such, the extracellular domain of a TNFSF ligand can also include the full-length TNFSF ligand sequence. In some aspects, a structure of an antigen binding polypeptide complex described herein can refer to an extracellular domain of a TNFSF ligand by the terms TNF1, TNF2 and/or TNF3, representing a first, second and/or third extracellular domain of a TNFSF ligand, respectively.

Examples of an extracellular domain of a TNFSF ligand include, but are not limited to, OX40L (OX40 ligand, TNFSF4), 4-1BBL (4-1BB ligand, TNFSF9), TNF, TNF-related apoptosis inducing ligand (TRAIL), CD40L (TNFSF5), CD27L (TNFSF7), CD30L (TNFSF8), FasL (TNFSF6), EDAM, LTA (TNFSF1), LTB (TNFSF3), CD153 (TNFSF8), RANKL (TNFSF11), TWEAK (TNFSF12), APRIL (TNFSF13), BAFF (TNFSF13B), LIGHT (TNFSF14), VEGI (TNFSF15), and GITRL (TNFSF18). In some aspects, the extracellular domain of a TNFSF ligand is OX40L or 4-1BBL. In some aspects, the extracellular domain of a TNFSF ligand is OX40L. In some aspects, the OX40L comprises an amino acid sequence of SEQ ID NO:1 or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:1. For example, the OX40L may comprise the amino acid sequence of SEQ ID NO:1 In some aspects, the extracellular domain of a TNFSF ligand is 4-1BBL. In some aspects, the 4-1BBL comprises an amino acid sequence of SEQ ID NO:2 or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:2. For example, the 4-1BBL may comprise the amino acid sequence of SEQ ID NO:2.

In some aspects, an antigen binding polypeptide complex of the disclosure comprises a trimer of three extracellular domains of a TNFSF ligand. In some aspects, the trimer comprises or consists of the same type of extracellular domain of a TNFSF ligand (e.g., three OX40L or three 4-1BBL (a homotrimer)), or the trimer can comprise or consist of a mixture of two or three different extracellular domains of a TNFSF ligand (e.g., one OX40L and two 4-1BBL, or two OX40L and one 4-1BBL, in any order). In some aspects, an antigen binding polypeptide complex comprises or consists of one trimer of extracellular domains of a TNFSF ligand (e.g., a homotrimer). For example, the antigen binding polypeptide complex may comprise a trimer of three OX40L domains, wherein each OX40L comprises or consists of an amino acid sequence of SEQ ID NO:1 or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:1. For example, each OX40L in the trimer may comprise or consist of the sequence of SEQ ID NO:1. For example, the antigen binding polypeptide complex may comprise a trimer of three 4-1BBL domains, wherein each 4-1BBL comprises or consists of an amino acid sequence of SEQ ID NO:2 or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:2. For example, each 4-1BBL in the trimer may comprise or consist of the sequence of SEQ ID NO:2. In some aspects, an antigen binding polypeptide complex of the disclosure comprises two trimers of extracellular domains of a TNFSF ligand (e.g., a dimer of trimers such as a dimer of homotrimers). For example, the antigen binding polypeptide complex may comprise a dimer of the OX40L trimers or the 4-1BBL trimers described herein.

In some aspects, an extracellular domain of a TNFSF ligand comprises or consists of the amino acid sequence of SEQ ID NO:1 or 2, or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:1 or 2. For example, the extracellular domain of a TNFSF ligand may comprise or consist of the sequence of SEQ ID NO:1 or 2. Other sequences of extracellular domains of a TNFSF ligand are known and include, for example, Accession Numbers XP_016857719.1, XP_016857718.1, XP_016857717.1, XP_011508266.2, NP_001284491.1, NP_003317.1, NP_003802.1, P41273.1, 6A3V_X, 6A3V_W, 6A3V_V, 6A3V_U, 6A3V_S, 6A3V_R, 6A3V_Q, 6A3V_P, 6A3V_O, 6A3V_N, 6A3V M, 6A3V_L, and 6A3V_K.

In some aspects, a trimer of extracellular domains of a TNFSF ligand contains an amino acid linker between one or more of the extracellular domains of a TNFSF ligand (e.g., having a structure represented by TNF1-L1-TNF2-L2-TNF3, where L1 and L2 are amino acid linkers). In some aspects, the amino acid linker comprises or consists of the amino acid sequence of any one of SEQ ID NOs:3-10 and 148-175 or a sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3-10 and 148-175. For example, the amino acid linker may comprise or consist of the amino acid sequence of any one of SEQ ID NOs:3-10 and 148-175. In some aspects, the amino acid linker comprises or consists of the amino acid sequence of any one of SEQ ID NOs:3-10 or a sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3-10. For example, the amino acid linker may comprise or consists of the amino acid sequence of any one of SEQ ID NOs:3-10. In some aspects, the amino acid linker comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 3. For example, the amino acid linker may comprise or consist of the amino acid sequence of SEQ ID NO:3. In some aspects, the amino acid linker comprises or consists of an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 10. For example, the amino acid linker may comprise or consist of the amino acid sequence of SEQ ID NO:10. For example, the amino acid linker may comprise or consists of the amino acid sequence of any one of SEQ ID NOs:4-10. In some aspects, the extracellular domains of a TNFSF ligand are OX40L (e.g., comprising or consisting of an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:1) and the amino acid linkers comprise or consist of the amino acid sequence of SEQ ID NO:3 or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:3. For example, the extracellular domains of a TNFSF ligand in the trimer may comprise or consist of the amino acid sequence of SEQ ID NO:1 and the amino acid linkers may comprise or consist of the amino acid sequence of SEQ ID NO:3. In some aspects, the extracellular domains of a TNFSF ligand are 4-1BBL (e.g., comprising or consisting of an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:2) and the amino acid linkers comprise or consist of any one of SEQ ID NOs:4-10 or a sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID Nos:4-10. For example, the extracellular domains of a TNFSF ligand in the trimer may comprise or consist of the amino acid sequence of SEQ ID NO:2 and the amino acid linkers may comprise or consist of the amino acid sequence of any one of SEQ ID NOs:4-10. In some aspects, the extracellular domains of a TNFSF ligand are 4-1BBL (e.g., comprising or consisting of an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:2) and the amino acid linkers comprise or consist of SEQ ID NO:10 or a sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:10. For example, the extracellular domains of a TNFSF ligand in the trimer may comprise or consist of the amino acid sequence of SEQ ID NO:2 and the amino acid linkers may comprise or consist of the amino acid sequence of SEQ ID NO:10.

In some aspects, a trimer of extracellular domains of a TNFSF ligand comprises or consists of the sequence of any one of SEQ ID NOs:11-18, or a sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:11-18. For example, a trimer of extracellular domains of a TNFSF ligand may comprise or consist of the sequence of any one of SEQ ID NOs:11-18. For example, the trimer of extracellular domains of a TNFSF ligand may comprise or consist of the sequence of SEQ ID NO:11, or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO: 11. For example, the trimer of extracellular domains of a TNFSF ligand may comprise or consist of the sequence of SEQ ID NO:18, or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO: 18. In some aspects, a dimer of trimers of extracellular domains of a TNFSF ligand comprises or consists of two trimers of extracellular domains of a TNFSF ligand, each comprising the sequence of any one of SEQ ID NOs:11-18, or a sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:11-18. In some aspects, the two trimers of the dimer are the same. In some aspects, one trimer is different than the other trimer of the dimer. For example, a dimer of trimers of extracellular domains of a TNFSF ligand may comprise or consist of two trimers of extracellular domains of a TNFSF ligand, each comprising the sequence of any one of SEQ ID NOs:11-18. For example, a dimer of trimers of extracellular domains of a TNFSF ligand may comprise or consist of two trimers of extracellular domains of a TNFSF ligand, each comprising the sequence of SEQ ID NO:11, or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO: 11. For example, a dimer of trimers of extracellular domains of a TNFSF ligand may comprise or consist of two trimers of extracellular domains of a TNFSF ligand, each comprising the sequence of SEQ ID NO:18, or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO: 18.

B. CD3

In some aspects, an antigen binding polypeptide complex of the invention (e.g., antibody or antigen binding fragment thereof) contains at least one region (e.g., CDR, VH, VL, scFv, Fab, scFab, heavy chain or light chain) that specifically binds to CD3.

In some aspects, the antigen binding polypeptide complex comprises a VL and/or VH that specifically bind to CD3. In some aspects, the VL comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH comprises a CDR1 comprising an amino acid sequence having at least 90% identity, at least 95% identity, or 100% identity to any one of SEQ ID NOs:19, 25, 182, 294 and 312; a CDR2 comprising an amino acid sequence having at least 90% identity, at least 95% identity, or 100% identity to any one of SEQ ID NOs:20, 26, 183, 295 and 313; and/or a CDR3 comprising an amino acid sequence having at least 90% identity, at least 95% identity, or 100% identity to any one of SEQ ID NOs:21, 27, 184, 296 and 314. In some aspects, the VL comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH comprises a CDR1 comprising an amino acid sequence having at least 90% identity to SEQ ID NO:182; a CDR2 comprising an amino acid sequence having at least 90% identity to SEQ ID No:183; and/or a CDR3 comprising an amino acid sequence having at least 90% identity to SEQ ID NO:184. In some aspects, the VL comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304. As used herein, “at least 90% identity” includes at least 91%, 92%, 93%, 94%, 95%, 96%, 97%, 98%, 99% and 100% identity to the recited reference sequence. In some aspects, the VL comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH comprises a CDR1 comprising the amino acid sequence of SEQ ID NO:182; a CDR2 comprising the amino acid sequence of SEQ ID No:183; and/or a CDR3 comprising the amino acid sequence of SEQ ID NO:184. In some aspects, the VL comprises a CDR1 comprising the amino acid sequence of SEQ ID NO:298; a CDR2 comprising the amino acid sequence of SEQ ID NO:299; and/or a CDR3 comprising the amino acid sequence of SEQ ID NO:300; and/or the VH comprises a CDR1 comprising the amino acid sequence of SEQ ID NO:294; a CDR2 comprising the amino acid sequence of SEQ ID No:295; and/or a CDR3 comprising the amino acid sequence of SEQ ID NO:296. In some aspects, the VL comprises a CDR1 comprising the amino acid sequence of SEQ ID NO:306; a CDR2 comprising the amino acid sequence of SEQ ID NO:307; and/or a CDR3 comprising the amino acid sequence of SEQ ID NO:308; and/or the VH comprises a CDR1 comprising the amino acid sequence of SEQ ID NO:312; a CDR2 comprising the amino acid sequence of SEQ ID No:313; and/or a CDR3 comprising the amino acid sequence of SEQ ID NO:314. In some aspects, the VL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45, and/or the VH comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43 or 44. In some aspects, the VL comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45; and/or the VH comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. For example, the VL may comprise the amino acid sequence of SEQ ID NO:45; and/or the VH may comprise the amino acid sequence of SEQ ID NO:43. In some aspects, the VL comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45; and/or the VH comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. For example, the VL may comprise the amino acid sequence of SEQ ID NO:45; and/or the VH may comprise the amino acid sequence of SEQ ID NO:44.

In some aspects, VH comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188. In some aspects, the VL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:297 or 305, and/or the VH comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188, 293 or 301. In some aspects, the VL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:297, and/or the VH comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293. In some aspects, the VL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:305, and/or the VH comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:301.

In some aspects, the antigen binding polypeptide complex comprises a light chain that specifically binds to CD3. In some aspects, the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54 or 176. For example, the light chain may comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO: 54. For example, the light chain may comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO: 176. For example, the light chain may comprise the amino acid sequence of SEQ ID NO:54. For example, the light chain may comprise the amino acid sequence of SEQ ID NO: 176. In some aspects, the light chain comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:55 or 177. For example, the light chain may comprise an amino acid sequence encoded by a polynucleotide having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO: 55. For example, the light chain may comprise an amino acid sequence encoded by a polynucleotide having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO: 177. For example, the light chain may comprise an amino acid sequence encoded by the polynucleotide sequence of SEQ ID NO: 55. For example, the light chain may comprise an amino acid sequence encoded by the polynucleotide sequence of SEQ ID NO: 177.

In some aspects, the antigen binding polypeptide complex comprises a heavy chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178. In some aspects, the heavy chain comprises the amino acid sequence of SEQ ID NO:178.

In some aspects, the antigen binding polypeptide complex comprises a heavy chain that specifically binds to CD3 and a light chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176. In some aspects, the heavy chain comprises the amino acid sequence of SEQ ID NO:178; and the light chain comprises the amino acid sequence of SEQ ID NO:176.

In some aspects, the antigen binding polypeptide complex comprises a heavy chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:98. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:104. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:120. In some aspects, the heavy chain comprises the amino acid sequence of SEQ ID NO:98. In some aspects, the heavy chain comprises the amino acid sequence of SEQ ID NO:104. In some aspects, the heavy chain comprises the amino acid sequence of SEQ ID NO:120.

In some aspects, the antigen binding polypeptide complex comprises a heavy chain that specifically binds to CD3 and a light chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:98; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:104; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:120; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises the amino acid sequence of SEQ ID NO:98; and the light chain comprises the amino acid sequence of SEQ ID NO:54. In some aspects, the heavy chain comprises the amino acid sequence of SEQ ID NO:104; and the light chain comprises the amino acid sequence of SEQ ID NO:54. In some aspects, the heavy chain comprises the amino acid sequence of SEQ ID NO:120; and the light chain comprises the amino acid sequence of SEQ ID NO:54.

In some aspects, the antigen binding polypeptide complex comprises a light chain that specifically binds to CD3. In some aspects, the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:56 (ahCD3_h34L chain). For example, the light chain may comprise the amino acid sequence of SEQ ID NO:56. In some aspects, the light chain comprises an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99% or 100% identity to SEQ ID NO:57 (ahCD3_h34L chain). For example, the light chain may comprise an amino acid sequence encoded by the polynucleotide sequence of SEQ ID NO:57. In some aspects, the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. For example, the light chain may comprise the amino acid sequence of SEQ ID NO:54.

In some aspects, the antigen binding polypeptide complex comprises a heavy chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:98. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:104. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:120. In some aspects, the heavy chain comprises the amino acid sequence of SEQ ID NO:98. In some aspects, the heavy chain comprises the amino acid sequence of SEQ ID NO:104. In some aspects, the heavy chain comprises the amino acid sequence of SEQ ID NO:120.

In some aspects, the antigen binding polypeptide complex comprises a heavy chain that specifically binds to CD3 and a light chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:98; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:104; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:120; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:54. In some aspects, the heavy chain comprises the amino acid sequence of SEQ ID NO:98; and the light chain comprises the amino acid sequence of SEQ ID NO:54. In some aspects, the heavy chain comprises the amino acid sequence of SEQ ID NO:104; and the light chain comprises the amino acid sequence of SEQ ID NO:54. In some aspects, the heavy chain comprises the amino acid sequence of SEQ ID NO:120; and the light chain comprises the amino acid sequence of SEQ ID NO:54.

In some aspects, the antigen binding polypeptide complex comprises a heavy chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:188. For example, the heavy chain may comprise the amino acid sequence of SEQ ID NO:188.

Other examples of sequences that specifically bind to CD3 are well known and described, for example, in U.S. Pat. Nos. 11,186,650; 11,155,621; 11,098,120; 11,072,656; 11,007,267; 10,968,276; 10,961,315; 10,906,978; 10,865,251; 10,759,858; 10,690,678; 10,688,186; 10,669,33; 10,640,572; 10,174,124; 9,850,304; 9,657,102; 8,551,478; 7,994,289; and 7,993,641.

C. Tumor-Associated Antigens and Immune-Activating Receptors

In some aspects, an antigen binding polypeptide complex of the invention (e.g., antibody or antigen binding fragment thereof) contains one or more region (e.g., CDR, VH, VL, scFv, Fab, scFab, heavy chain or light chain) that specifically binds to an immune activating receptor or tumor-associated antigen (TAA).

As used herein a “tumor-associated antigen” or TAA is a protein or molecule that is more prevalent on cancer cells compared to normal cells. Examples of a TAA include, but are not limited to, tyrosine-protein kinase Met (cMet), trophoblast cell surface antigen 2 (Trop2), CD20, CD19, receptor tyrosine-protein kinase erbB-2 (HER2), receptor tyrosine-protein kinase erbB-3 (HER3), adenosine A2A receptor (A2AR), a proliferation-inducing ligand (APRIL), epidermal growth factor receptor (EGFR), fibroblast growth factor receptor (FGFR), B cell activating factor (BAFF), BAFF receptor (BAFFR), B cell maturation antigen (BCMA), Bruton's tyrosine kinase (BTK), B and T lymphocyte attenuator (BTLA), B7DC (programmed death ligand 2), B7 homolog 1 (B7H1), B7 homolog 4 (B7H4), delta-like ligand 3 (DLL3), ectonucleoside triphosphate diphosphohydrolase 1 (ENTPD1), Fc fragment of IgE receptor 1a (FCER1A), Fc fragment of IgE receptor 1 (FCER1), arachidonate 5-lipoxygenase-activating protein (FLAP), folate hydrolase 1 (FOLH1), mucin 1 (MUC-1), CD133, mucin 16 (MUC-16), lysosomal-associated membrane protein 1 (LAMP1), CD38, programmed death ligand 1 (PD-L1), CEA cell adhesion molecule 5 (CEACAM5), six-transmembrane epithelial antigen of prostate 1 (STEAP1), and epithelial cellular adhesion molecule (EpCAM). In some aspects, the TAA is HER2.

As used herein, an “immune stimulatory receptor” is a heterogeneous group of cell surface molecules that act to amplify or counteract the initial activating signals provided to T cells (e.g., from the T cell receptor (TCR) following its interaction with an antigen/major histocompatibility complex (MHC)), thereby influencing T cell differentiation, activation and/or proliferation. Examples of immune stimulatory receptors are well-known and include, but are not limited to, CD3 and CD28.

In some aspects, the antigen binding polypeptide complex comprises a VH and/or VL that specifically bind to a TAA or an immune stimulatory receptor (e.g., CD28). For example, the antigen binding polypeptide complex may comprise a VH and/or VL that specifically bind to CD28. In some aspects, the VL comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL comprises a CDR1 comprising an amino acid sequence having at least 90% to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL comprises the amino acid sequence of SEQ ID NO:47 or 49 or a sequence having at least 80%, at least 85%, at least 90%, or at least 95% identity to SEQ ID NO:47 or 49. In some aspects, the VL comprises a sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47. In some aspects, the VL comprises a sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49. For example, the VL may comprise the amino acid sequence of SEQ ID NO:47. For example, the VL may comprise the amino acid sequence of SEQ ID NO:49. In some aspects, the VH comprises the amino acid sequence of SEQ ID NO:46 or 48 or a sequence having at least 80%, at least 85%, at least 90%, or at least 95% identity to SEQ ID NO:46 or 48. In some aspects, the VH comprises a sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VH comprises a sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. For example, the VH may comprise the amino acid sequence of SEQ ID NO:46. For example, the VH may comprise the amino acid sequence of SEQ ID NO:48. In some aspects, the antigen binding polypeptide complex comprises a VL comprising the amino acid sequence of SEQ ID NO:47 (or a sequence having at least 80%, at least 85%, at least 90%, or at least 95% identity to SEQ ID NO:47); and a VH comprising the amino acid sequence of SEQ ID NO:46 (or a sequence having at least 80%, at least 85%, at least 90%, or at least 95% identity to SEQ ID NO:46). In some aspects, the antigen binding polypeptide complex comprises a VL comprising the amino acid sequence of SEQ ID NO:49 (or a sequence having at least 80%, at least 85%, at least 90%, or at least 95% identity to SEQ ID NO:49); and a VH comprising the amino acid sequence of SEQ ID NO:48 (or a sequence having at least 80%, at least 85%, at least 90%, or at least 95% identity to SEQ ID NO:48).

In some aspects, the antigen binding polypeptide complex comprises a light chain that specifically binds to a TAA or an immune stimulatory receptor. In some aspects, the light chain comprises an amino acid sequence having at least 80%, at least 85%, at least 90%, at least 95%, or 100% identity to SEQ ID NO:50 or 52. In some aspects, the light chain comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:50. In some aspects, the light chain comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:52. For example, the light chain may comprise the amino acid sequence of SEQ ID NO:50. For example, the light chain may comprise the amino acid sequence of SEQ ID NO:52. In some aspects, the light chain comprises an amino acid sequence encoded by a polynucleotide having at least 80%, at least 85%, at least 90%, at least 95%, or 100% identity to SEQ ID NO:51 or 53. In some aspects, the light chain comprises an amino acid sequence encoded by a polynucleotide having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:51. In some aspects, the light chain comprises an amino acid sequence encoded by a polynucleotide having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:53. For example, the light chain may comprise an amino acid sequence encoded by the polynucleotide sequence of SEQ ID NO:51. For example, the light chain may comprise an amino acid sequence encoded by the polynucleotide sequence of SEQ ID NO:53.

In some aspects, the antigen binding polypeptide complex comprises a VH and/or VL that specifically binds to CD20. In some aspects, the VL comprises a CDR1 comprising an amino acid sequence having at least 90% identity, at least 95% identity, or 100% identity to SEQ ID NO:314 or 322; a CDR2 comprising an amino acid sequence having at least 90% identity, at least 95% identity, or 100% identity to SEQ ID NO:315 or 323; and/or a CDR3 comprising an amino acid sequence having at least 90% identity, at least 95% identity, or 100% identity to SEQ ID NO:316 or 324; and/or the VH comprises a CDR1 comprising an amino acid sequence having at least 90% identity, at least 95% identity, or 100% identity to SEQ ID NO:310 or 318; a CDR2 comprising an amino acid sequence having at least 90% identity, at least 95% identity, or 100% identity to SEQ ID NO:311 or 319; and/or a CDR3 comprising an amino acid sequence having at least 90% identity, at least 95% identity, or 100% identity to SEQ ID NO:312 or 320. In some aspects, the VL comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320. In some aspects, the VL comprises an amino acid sequence having at least 90% identity, at least 95% identity, or 100% identity to SEQ ID NO:313 or 321; and/or the VH comprises an amino acid sequence having at least 90% identity, at least 95% identity, or 100% identity to SEQ ID NO:309 or 317. In some aspects, the VL comprises an amino acid sequence having at least 90% identity, at least 95% identity, or 100% identity to SEQ ID NO:313; and/or the VH comprises an amino acid sequence having at least 90% identity, at least 95% identity, or 100% identity to SEQ ID NO:309. In some aspects, the VL comprises an amino acid sequence having at least 90% identity, at least 95% identity, or 100% identity to SEQ ID NO:321; and/or the VH comprises an amino acid sequence having at least 90% identity, at least 95% identity, or 100% identity to SEQ ID NO:317. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to cMet. In some aspects, the VL comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL comprises an amino acid sequence having at least 90% identity, at least 95% identity, or 100% identity to SEQ ID NO:273; and/or the VH comprises an amino acid sequence having at least 90% identity, at least 95% identity, or 100% identity to SEQ ID NO:269. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to Trop2. In some aspects, the VL comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL comprises an amino acid sequence having at least 90% identity, at least 95% identity, or 100% identity to SEQ ID NO:281; and/or the VH comprises an amino acid sequence having at least 90% identity, at least 95% identity, or 100% identity to SEQ ID NO:277. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to CD19. In some aspects, the VL comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL comprises an amino acid sequence having at least 90% identity, at least 95% identity, or 100% identity to SEQ ID NO:289; and/or the VH comprises an amino acid sequence having at least 90% identity, at least 95% identity, or 100% identity to SEQ ID NO:285. As used herein, “at least 90% identity” includes at least 91%, 92%, 93%, 94%, 95%, 96%, 97%, 98%, 99% and 100% identity to the recited reference sequence.

Other examples of sequences that specifically bind to a TAA are well known and include, but are not limited to, GenBank Accession Nos. AAA39272.1, AAA39159.1, ABN79462.1, AVW80143.1, AVW80142.1, AVW80141.1, AAB34430.1, AAB34429.1, CAD45042.1, 4CMH_C and 4CMH_B. Such sequences are also described, for example, in Wernly et al., Cells, 9(2):295, 2020; Arakawa et al., Journal of Biochemistry, 120(3):657-662, 1996; Cole et al., Transplantation, 68(4):563-571, 1999; Li et al., International Immunopharmacology, 62:299-308, 2018; Castella et al., Methods & Clinical Development, 12:134-144, 2019; Sun et al., Molecular Immunology, 41(9):929-938, 2004; Iwaszkiewicz-Grzes et al., Cytotherapy, 22(11):629-641, 2020, Rosinski et al., Transplant Direct, 1(2):e7, 2015; Ellis et al., J Immunology, 155(2):925-937, 1995; Stevenson et al., Blood, 77(5):1071-1079, 1991; Chillemi et al., Molecular Medicine, 19:99-108, 2013, and Int'l Pub. No. WO 2020/076853.

D. Antigen Binding Polypeptide Complex Structures

In some aspects, an antigen binding polypeptide complex of the invention comprises a first polypeptide, a second polypeptide, and a third polypeptide; wherein the first polypeptide has a structure represented by VL1-L1-CL; VL1-L1-CH1; VH1-L1-CL; or VH1-L1-CH1; wherein (i) the second polypeptide has a structure represented by VH1-L2-CH1-L3-Fc-L4-TNF1-L5-TNF2-L6-TNF3; VH1-L2-CL-L3-Fc-L4-TNF1-L5-TNF2-L6-TNF3; VH1-L7-CH1-L8-Fc; VH1-L7-CL-L8-Fc; VL1-L2-CH1-L3-Fc-L4-TNF1-L5-TNF2-L6-TNF3; VL1-L2-CL-L3-Fc-L4-TNF1-L5-TNF2-L6-TNF3; VL1-L7-CH1-L8-Fc; or VL1-L7-CL-L8-Fc; and the third polypeptide has a structure represented by VL2-L9-VH2-L10-Fc-L11-TNF1-L12-TNF2-L13-TNF3; or VH2-L14-VL2-L15-Fc-L16-TNF1-L17-TNF2-L18-TNF3; or (ii) the second polypeptide has a structure represented by VH1-L19-CH1-L20-Fc-L21-TNF1-L22-TNF2-L23-TNF3; VH1-L19-CL-L20-Fc-L21-TNF1-L22-TNF2-L23-TNF3; VL1-L19-CH1-L20-Fc-L21-TNF1-L22-TNF2-L23-TNF3; or VL1-L19-CL-L20-Fc-L21-TNF1-L22-TNF2-L23-TNF3; and the third polypeptide has a structure represented by VL2-L24-VH2-L25-Fc; or VH2-L26-VL2-L27-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L27 are amino acid linkers. In some aspects, the first polypeptide has a structure represented by VL1-L1-CL; VL1-L1-CH1; VH1-L1-CL; or VH1-L1-CH1; the second polypeptide has a structure represented by VH1-L2-CH1-L3-Fc-L4-TNF1-L5-TNF2-L6-TNF3; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VH1-L2-CH1-L3-Fc-L4-TNF1-L5-TNF2-L6-TNF3; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VH1-L2-CL-L3-Fc-L4-TNF1-L5-TNF2-L6-TNF3; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VH1-L2-CL-L3-Fc-L4-TNF1-L5-TNF2-L6-TNF3; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VH1-L7-CH1-L8-Fc; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VH1-L7-CH1-L8-Fc; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VH1-L7-CL-L8-Fc; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VH1-L7-CL-L8-Fc; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VL1-L2-CH1-L3-Fc-L4-TNF1-L5-TNF2-L6-TNF3; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VL1-L2-CH1-L3-Fc-L4-TNF1-L5-TNF2-L6-TNF3; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VL1-L2-CL-L3-Fc-L4-TNF1-L5-TNF2-L6-TNF3; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VL1-L2-CL-L3-Fc-L4-TNF1-L5-TNF2-L6-TNF3; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VL1-L7-CH1-L8-Fc; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VL1-L7-CH1-L8-Fc; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VL1-L7-CL-L8-Fc; and the third polypeptide has a structure represented by: VL2-L9-VH2-L10-Fc-L11-TNF1-L12-TNF2-L13-TNF3. In some aspects, the first polypeptide has a structure represented by VL1-L1-CL; VL1-L1-CH1; VH1-L1-CL; or VH1-L1-CH1; the second polypeptide has a structure represented by VL1-L7-CL-L8-Fc; and the third polypeptide has a structure represented by: VH2-L14-VL2-L15-Fc-L16-TNF1-L17-TNF2-L18-TNF3. In some aspects, the first polypeptide has a structure represented by VL1-L1-CL; VL1-L1-CH1; VH1-L1-CL; or VH1-L1-CH1; the second polypeptide has a structure represented by VH1-L19-CH1-L20-Fc-L21-TNF1-L22-TNF2-L23-TNF3; and the third polypeptide has a structure represented by: VL2-L24-VH2-L25-Fc. In some aspects, the first polypeptide has a structure represented by VL1-L1-CL; VL1-L1-CH1; VH1-L1-CL; or VH1-L1-CH1; the second polypeptide has a structure represented by VH1-L19-CH1-L20-Fc-L21-TNF1-L22-TNF2-L23-TNF3; and the third polypeptide has a structure represented by: VH2-L26-VL2-L27-Fc. In some aspects, the first polypeptide has a structure represented by VL1-L1-CL; VL1-L1-CH1; VH1-L1-CL; or VH1-L1-CH1; the second polypeptide has a structure represented by VH1-L19-CL-L20-Fc-L21-TNF1-L22-TNF2-L23-TNF3; and the third polypeptide has a structure represented by: VL2-L24-VH2-L25-Fc. In some aspects, the first polypeptide has a structure represented by VL1-L1-CL; VL1-L1-CH1; VH1-L1-CL; or VH1-L1-CH1; the second polypeptide has a structure represented by VH1-L19-CL-L20-Fc-L21-TNF1-L22-TNF2-L23-TNF3; and the third polypeptide has a structure represented by: VH2-L26-VL2-L27-Fc. In some aspects, the first polypeptide has a structure represented by VL1-L1-CL; VL1-L1-CH1; VH1-L1-CL; or VH1-L1-CH1; the second polypeptide has a structure represented by VL1-L19-CH1-L20-Fc-L21-TNF1-L22-TNF2-L23-TNF3; and the third polypeptide has a structure represented by: VL2-L24-VH2-L25-Fc. In some aspects, the first polypeptide has a structure represented by VL1-L1-CL; VL1-L1-CH1; VH1-L1-CL; or VH1-L1-CH1; the second polypeptide has a structure represented by VL1-L19-CH1-L20-Fc-L21-TNF1-L22-TNF2-L23-TNF3; and the third polypeptide has a structure represented by: VH2-L26-VL2-L27-Fc. In some aspects, the first polypeptide has a structure represented by VL1-L1-CL; VL1-L1-CH1; VH1-L1-CL; or VH1-L1-CH1; the second polypeptide has a structure represented by VL1-L19-CL-L20-Fc-L21-TNF1-L22-TNF2-L23-TNF3; and the third polypeptide has a structure represented by: VL2-L24-VH2-L25-Fc. In some aspects, the first polypeptide has a structure represented by VL1-L1-CL; VL1-L1-CH1; VH1-L1-CL; or VH1-L1-CH1; the second polypeptide has a structure represented by VL1-L19-CL-L20-Fc-L21-TNF1-L22-TNF2-L23-TNF3; and the third polypeptide has a structure represented by: VH2-L26-VL2-L27-Fc. In some aspects, an antigen binding polypeptide complex of the invention comprises a first polypeptide, a second polypeptide, and a third polypeptide; wherein the first polypeptide has a structure represented by VL1-L1-CL; VL1-L1-CH1; VH1-L1-CL; or VH1-L1-CH1; wherein (i) the second polypeptide has a structure represented by VH1-CH1-L2-Fc-L3-TNF1-L4-TNF2-L5-TNF3; VH1-CL-L2-Fc-L3-TNF1-L4-TNF2-L5-TNF3; VH1-CH1-L6-Fc; VH1-CL-L7-Fc; VL1-CH1-L2-Fc-L3-TNF1-L4-TNF2-L5-TNF3; VL1-CL-L2-Fc-L3-TNF1-L4-TNF2-L5-TNF3; VL1-CH1-L6-Fc; or VL1-CL-L7-Fc; and the third polypeptide has a structure represented by VL2-L8-VH2-L9-Fc-L10-TNF1-L11-TNF2-L12-TNF3; or VH2-L13-VL2-L14-Fc-L15-TNF1-L16-TNF2-L17-TNF3; or (ii) the second polypeptide has a structure represented by VH1-CH1-L18-Fc-L19-TNF1-L20-TNF2-L21-TNF3; VH1-CL-L18-Fc-L19-TNF1-L20-TNF2-L21-TNF3; VL1-CH1-L18-Fc-L19-TNF1-L20-TNF2-L21-TNF3; or VL1-CL-L18-Fc-L19-TNF1-L20-TNF2-L21-TNF3; and the third polypeptide has a structure represented by VL2-L22-VH2-L23-Fc; or VH2-L24-VL2-L25-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L26 are amino acid linkers. In some aspects, the first polypeptide has a structure represented by VL1-L1-CL; VL1-L1-CH1; VH1-L1-CL; or VH1-L1-CH1; the second polypeptide has a structure represented by VH1-CH1-L2-Fc-L3-TNF1-L4-TNF2-L5-TNF3; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VH1-CH1-L2-Fc-L3-TNF1-L4-TNF2-L5-TNF3; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VH1-CL-L2-Fc-L3-TNF1-L4-TNF2-L5-TNF3; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VH1-CL-L2-Fc-L3-TNF1-L4-TNF2-L5-TNF3; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VH1-CH1-L6-Fc; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VH1-CH1-L6-Fc; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VH1-CL-L7-Fc; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VH1-CL-L7-Fc; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VL1-CH1-L2-Fc-L3-TNF1-L4-TNF2-L5-TNF3; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VL1-CH1-L2-Fc-L3-TNF1-L4-TNF2-L5-TNF3; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VL1-CL-L2-Fc-L3-TNF1-L4-TNF2-L5-TNF3; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VL1-CL-L2-Fc-L3-TNF1-L4-TNF2-L5-TNF3; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VL1-CH1-L6-Fc; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VL1-CH1-L6-Fc; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VL1-CL-L7-Fc; and the third polypeptide has a structure represented by: VL2-L8-VH2-L9-Fc-L10-TNF1-L11-TNF2-L12-TNF3. In some aspects, the first polypeptide has a structure represented by VL1-L1-CL; VL1-L1-CH1; VH1-L1-CL; or VH1-L1-CH1; the second polypeptide has a structure represented by VL1-CL-L7-Fc; and the third polypeptide has a structure represented by: VH2-L13-VL2-L14-Fc-L15-TNF1-L16-TNF2-L17-TNF. In some aspects, the first polypeptide has a structure represented by VL1-L1-CL; VL1-L1-CH1; VH1-L1-CL; or VH1-L1-CH1; the second polypeptide has a structure represented by VH1-CH1-L18-Fc-L19-TNF1-L20-TNF2-L21-TNF3; and the third polypeptide has a structure represented by: VL2-L22-VH2-L23-Fc. In some aspects, the first polypeptide has a structure represented by VL1-L1-CL; VL1-L1-CH1; VH1-L1-CL; or VH1-L1-CH1; the second polypeptide has a structure represented by VH1-CH1-L18-Fc-L19-TNF1-L20-TNF2-L21-TNF3; and the third polypeptide has a structure represented by: VH2-L24-VL2-L25-Fc. In some aspects, the first polypeptide has a structure represented by VL1-L1-CL; VL1-L1-CH1; VH1-L1-CL; or VH1-L1-CH1; the second polypeptide has a structure represented by VH1-CL-L18-Fc-L19-TNF1-L20-TNF2-L21-TNF3; and the third polypeptide has a structure represented by: VL2-L22-VH2-L23-Fc. In some aspects, the first polypeptide has a structure represented by VL1-L1-CL; VL1-L1-CH1; VH1-L1-CL; or VH1-L1-CH1; the second polypeptide has a structure represented by VH1-CL-L18-Fc-L19-TNF1-L20-TNF2-L21-TNF3; and the third polypeptide has a structure represented by: VH2-L24-VL2-L25-Fc. In some aspects, the first polypeptide has a structure represented by VL1-L1-CL; VL1-L1-CH1; VH1-L1-CL; or VH1-L1-CH1; the second polypeptide has a structure represented by VL1-CH1-L18-Fc-L19-TNF1-L20-TNF2-L21-TNF3; and the third polypeptide has a structure represented by: VL2-L22-VH2-L23-Fc. In some aspects, the first polypeptide has a structure represented by VL1-L1-CL; VL1-L1-CH1; VH1-L1-CL; or VH1-L1-CH1; the second polypeptide has a structure represented by VL1-CH1-L18-Fc-L19-TNF1-L20-TNF2-L21-TNF3; and the third polypeptide has a structure represented by: VH2-L24-VL2-L25-Fc. In some aspects, the first polypeptide has a structure represented by VL1-L1-CL; VL1-L1-CH1; VH1-L1-CL; or VH1-L1-CH1; the second polypeptide has a structure represented by VL1-CL-L18-Fc-L19-TNF1-L20-TNF2-L21-TNF3; and the third polypeptide has a structure represented by: VL2-L22-VH2-L23-Fc. In some aspects, the first polypeptide has a structure represented by VL1-L1-CL; VL1-L1-CH1; VH1-L1-CL; or VH1-L1-CH1; the second polypeptide has a structure represented by VL1-CL-L18-Fc-L19-TNF1-L20-TNF2-L21-TNF3; and the third polypeptide has a structure represented by: VH2-L24-VL2-L25-Fc.

In some aspects, the first polypeptide has a structure represented by VL1-L1-CL; the second polypeptide has a structure represented by VH1-L2-CH1-L3-Fc-L4-TNF1-L5-TNF2-L6-TNF3; and the third polypeptide has a structure represented by VL2-L9-VH2-L10-Fc-L11-TNF1-L12-TNF2-L13-TNF3. In some aspects, the first polypeptide has a structure represented by VL1-L1-CL; the second polypeptide has a structure represented by VH1-CH1-L2-Fc-L3-TNF1-L4-TNF2-L5-TNF3; and the third polypeptide has a structure represented by VL2-L8-VH2-L9-Fc-L10-TNF1-L11-TNF2-L12-TNF3.

In some aspects, the first polypeptide has a structure represented by VL1-L1-CL; the second polypeptide has a structure represented by VH1-L2-CH1-L3-Fc-L4-TNF1-L5-TNF2-L6-TNF3; and the third polypeptide has a structure represented by VH2-L14-VL2-L15-Fc-L16-TNF1-L17-TNF2-L18-TNF3. In some aspects, the first polypeptide has a structure represented by VL1-L1-CL; the second polypeptide has a structure represented by VH1-L7-CH1-L8-Fc; and the third polypeptide has a structure represented by VL2-L9-VH2-L10-Fc-L11-TNF1-L12-TNF2-L13-TNF3. In some aspects, the first polypeptide has a structure represented by VL1-L1-CL; the second polypeptide has a structure represented by VH1-L7-CH1-L8-Fc; and the third polypeptide has a structure represented by VH2-L14-VL2-L15-Fc-L16-TNF1-L17-TNF2-L18-TNF3. In some aspects, the first polypeptide has a structure represented by VL1-L1-CL; the second polypeptide has a structure represented by VH1-L19-CH1-L20-Fc-L21-TNF1-L22-TNF2-L23-TNF3; and the third polypeptide has a structure represented by VL2-L24-VH2-L25-Fc. In some aspects, the first polypeptide has a structure represented by VL1-L1-CL; the second polypeptide has a structure represented by VH1-L19-CH1-L20-Fc-L21-TNF1-L22-TNF2-L23-TNF3; and the third polypeptide has a structure represented by VH2-L26-VL2-L27-Fc. In some aspects, the first polypeptide has a structure represented by VL1-L1-CL; the second polypeptide has a structure represented by VH1-CH1-L2-Fc-L3-TNF1-L4-TNF2-L5-TNF3; and the third polypeptide has a structure represented by VH2-L13-VL2-L14-Fc-L15-TNF1-L16-TNF2-L17-TNF3. In some aspects, the first polypeptide has a structure represented by VL1-L1-CL; the second polypeptide has a structure represented by VH1-L6-CH1-L7-Fc; and the third polypeptide has a structure represented by VL2-L8-VH2-L9-Fc-L10-TNF1-L11-TNF2-L12-TNF3. In some aspects, the first polypeptide has a structure represented by VL1-L1-CL; the second polypeptide has a structure represented by VH1-L6-CH1-L7-Fc; and the third polypeptide has a structure represented by VH2-L13-VL2-L14-Fc-L15-TNF1-L16-TNF2-L17-TNF3. In some aspects, the first polypeptide has a structure represented by VL1-L1-CL; the second polypeptide has a structure represented by VH1-L18-CH1-L19-Fc-L20-TNF1-L21-TNF2-L22-TNF3; and the third polypeptide has a structure represented by VL2-L23-VH2-L24-Fc. In some aspects, the first polypeptide has a structure represented by VL1-L1-CL; the second polypeptide has a structure represented by VH1-L18-CH1-L19-Fc-L20-TNF1-L21-TNF2-L22-TNF3; and the third polypeptide has a structure represented by VH2-L25-VL2-L26-Fc.

In some aspects, the VL1 and VH1 of the antigen binding polypeptide complex specifically bind to CD3.

In some aspects, the VL1 of the antigen binding polypeptide complex comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH1 of the antigen binding polypeptide complex comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26. 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL1 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. In some aspects, the VL1 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH1 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL1 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH1 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304. As used herein, “at least 90% identity” includes at least 91%, 92%, 93%, 94%, 95%, 96%, 97%, 98%, 99% and 100% identity to the recited reference sequence. In some aspects, the VL1 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 of the antigen binding polypeptide complex comprises the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 of the antigen binding polypeptide complex comprises a CDR1 comprising the amino acid sequence of SEQ ID NO:298; a CDR2 comprising the amino acid sequence of SEQ ID NO:299; and/or a CDR3 comprising the amino acid sequence of SEQ ID NO:300; and/or the VH1 of the antigen binding polypeptide complex comprises a CDR1 comprising the amino acid sequence of SEQ ID NO:294; a CDR2 comprising the amino acid sequence of SEQ ID NO:295; and/or a CDR3 comprising the amino acid sequence of SEQ ID NO:296. In some aspects, the VL1 of the antigen binding polypeptide complex comprises a CDR1 comprising the amino acid sequence of SEQ ID NO:306; a CDR2 comprising the amino acid sequence of SEQ ID NO:307; and/or a CDR3 comprising the amino acid sequence of SEQ ID NO:308; and/or the VH1 of the antigen binding polypeptide complex comprises a CDR1 comprising the amino acid sequence of SEQ ID NO:302; a CDR2 comprising the amino acid sequence of SEQ ID NO:303; and/or a CDR3 comprising the amino acid sequence of SEQ ID NO:304.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 of the antigen binding polypeptide complex comprises the amino acid sequence of SEQ ID NO:45, and/or the VH1 of the antigen binding polypeptide complex comprises the amino acid sequence of SEQ ID NO:43. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 of the antigen binding polypeptide complex comprises the amino acid sequence of SEQ ID NO:45, and/or the VH1 of the antigen binding polypeptide complex comprises the amino acid sequence of SEQ ID NO:44.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297 or 305, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:188, 293 or 301. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL2 and VH2 of the antigen binding polypeptide complex specifically bind to a tumor-associated antigen (TAA) or an immune stimulatory receptor. In some aspects, the immune stimulatory receptor is CD28. In some aspects, the TAA is tyrosine-protein kinase Met (cMet), trophoblast cell surface antigen 2 (Trop2), CD20, CD19, receptor tyrosine-protein kinase erbB-2 (HER2), receptor tyrosine-protein kinase erbB-3 (HER3), adenosine A2A receptor (A2AR), a proliferation-inducing ligand (APRIL), epidermal growth factor receptor (EGFR), fibroblast growth factor receptor (FGFR), B cell activating factor (BAFF), BAFF receptor (BAFFR), B cell maturation antigen (BCMA), Bruton's tyrosine kinase (BTK), B and T lymphocyte attenuator (BTLA), B7DC (programmed death ligand 2), B7 homolog 1 (B7H1), B7 homolog 4 (B7H4), delta-like ligand 3 (DLL3), ectonucleoside triphosphate diphosphohydrolase 1 (ENTPD1), Fc fragment of IgE receptor 1a (FCER1A), Fc fragment of IgE receptor 1 (FCER1), arachidonate 5-lipoxygenase-activating protein (FLAP), folate hydrolase 1 (FOLH1), mucin 1 (MUC-1), CD133, mucin 16 (MUC-16), lysosomal-associated membrane protein 1 (LAMP1), CD38, programmed death ligand 1 (PD-L1), CEA cell adhesion molecule 5 (CEACAM5), six-transmembrane epithelial antigen of prostate 1 (STEAP1), or epithelial cellular adhesion molecule (EpCAM). In some aspects, the TAA is HER2.

In some aspects, the VL2 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH2 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL2 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH2 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL2 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH2 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL2 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH2 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL2 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH2 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:47 or 49, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46 or 48. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL2 of the antigen binding polypeptide complex comprises the amino acid sequence of SEQ ID NO:47, and/or the VH2 of the antigen binding polypeptide complex comprises the amino acid sequence of SEQ ID NO:46. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL2 of the antigen binding polypeptide complex comprises the amino acid sequence of SEQ ID NO:49, and/or the VH2 of the antigen binding polypeptide complex comprises the amino acid sequence of SEQ ID NO:48.

In some aspects, the VL2 and VH2 of the antigen binding polypeptide complex specifically bind to CD3.

In some aspects, the VL2 of the antigen binding polypeptide complex comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH2 of the antigen binding polypeptide complex comprises a CDR1 comprising an amino acid sequence having at least 90% identity, at least 95% identity, or 100% identity to any one of SEQ ID NOs:19, 25, 182, 294 and 312; a CDR2 comprising an amino acid sequence having at least 90% identity, at least 95% identity, or 100% identity to any one of SEQ ID NOs:20, 26, 183, 295 and 313; and/or a CDR3 comprising an amino acid sequence having at least 90% identity, at least 95% identity, or 100% identity to any one of SEQ ID NOs:21, 27, 184, 296 and 314. In some aspects, the VL2 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH2 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL2 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH2 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL2 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH2 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL2 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH2 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL2 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH2 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL2 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH2 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL2 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH2 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL2 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH2 of the antigen binding polypeptide complex comprises a CDR1 comprising an amino acid sequence having at least 90% identity to SEQ ID NO:312; a CDR2 comprising an amino acid sequence having at least 90% identity to SEQ ID NO:313; and/or a CDR3 comprising an amino acid sequence having at least 90% identity to SEQ ID NO:314.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:45, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43 or 44. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL2 of the antigen binding polypeptide complex comprises the amino acid sequence of SEQ ID NO:45, and/or the VH2 of the antigen binding polypeptide complex comprises the amino acid sequence of SEQ ID NO:43. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL2 of the antigen binding polypeptide complex comprises the amino acid sequence of SEQ ID NO:45, and/or the VH2 of the antigen binding polypeptide complex comprises the amino acid sequence of SEQ ID NO:44.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:297, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:305, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:301.

In some aspects, the VL1 and VH1 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL1 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH1 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL1 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH1 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL1 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH1 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL1 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH1 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL1 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH1 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL1 of the antigen binding polypeptide complex comprises the amino acid sequence of SEQ ID NO:47, and/or the VH1 of the antigen binding polypeptide complex comprises the amino acid sequence of SEQ ID NO:46. In some aspects, the VL1 of the antigen binding polypeptide complex comprises the amino acid sequence of SEQ ID NO:49, and/or the VH1 of the antigen binding polypeptide complex comprises the amino acid sequence of SEQ ID NO:48.

In some aspects, the antigen binding polypeptide complex comprises a VH and/or VL that specifically binds to CD20. In some aspects, the VL comprises a CDR1 comprising an amino acid sequence having at least 90% identity, at least 95% identity, or 100% identity to SEQ ID NO:314 or 322; a CDR2 comprising an amino acid sequence having at least 90% identity, at least 95% identity, or 100% identity to SEQ ID NO:315 or 323; and/or a CDR3 comprising an amino acid sequence having at least 90% identity, at least 95% identity, or 100% identity to SEQ ID NO:316 or 324; and/or the VH comprises a CDR1 comprising an amino acid sequence having at least 90% identity, at least 95% identity, or 100% identity to SEQ ID NO:310 or 318; a CDR2 comprising an amino acid sequence having at least 90% identity, at least 95% identity, or 100% identity to SEQ ID NO:311 or 319; and/or a CDR3 comprising an amino acid sequence having at least 90% identity, at least 95% identity, or 100% identity to SEQ ID NO:312 or 320. In some aspects, the VL comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320. In some aspects, the VL comprises an amino acid sequence having at least 90% identity, at least 95% identity, or 100% identity to SEQ ID NO:313 or 321; and/or the VH comprises an amino acid sequence having at least 90% identity, at least 95% identity, or 100% identity to SEQ ID NO:309 or 317. In some aspects, the VL comprises an amino acid sequence having at least 90% identity, at least 95% identity, or 100% identity to SEQ ID NO:313; and/or the VH comprises an amino acid sequence having at least 90% identity, at least 95% identity, or 100% identity to SEQ ID NO:309. In some aspects, the VL comprises an amino acid sequence having at least 90% identity, at least 95% identity, or 100% identity to SEQ ID NO:321; and/or the VH comprises an amino acid sequence having at least 90% identity, at least 95% identity, or 100% identity to SEQ ID NO:317. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to cMet. In some aspects, the VL comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL comprises an amino acid sequence having at least 90% identity, at least 95% identity, or 100% identity to SEQ ID NO:273; and/or the VH comprises an amino acid sequence having at least 90% identity, at least 95% identity, or 100% identity to SEQ ID NO:269. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to Trop2. In some aspects, the VL comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL comprises an amino acid sequence having at least 90% identity, at least 95% identity, or 100% identity to SEQ ID NO:281; and/or the VH comprises an amino acid sequence having at least 90% identity, at least 95% identity, or 100% identity to SEQ ID NO:277. As used herein, “at least 90% identity” includes at least 91%, 92%, 93%, 94%, 95%, 96%, 97%, 98%, 99% and 100% identity to the recited reference sequence.

In some aspects, the antigen binding polypeptide complex comprises a VH and/or VL that specifically binds to CD19. In some aspects, the VL comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL comprises an amino acid sequence having at least 90% identity, at least 95% identity, or 100% identity to SEQ ID NO:289; and/or the VH comprises an amino acid sequence having at least 90% identity, at least 95% identity, or 100% identity to SEQ ID NO:285. As used herein, “at least 90% identity” includes at least 91%, 92%, 93%, 94%, 95%, 96%, 97%, 98%, 99% and 100% identity to the recited reference sequence.

In some aspects, an antigen binding polypeptide complex of the invention comprises a first polypeptide and a second polypeptide; wherein (i) the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L37-VH2-L38-Fc-L39-TNF1-L40-TNF2-L41-TNF3; VH2-L42-VL2-L43-Fc-L44-TNF1-L45-TNF2-L46-TNF3; VL2-L47-VH2-L48-CL-L49-CH1-L50-Fc-L51-TNF1-L52-TNF2-L53-TNF3; or VL2-L54-CL-L55-VH2-L56-CH1-L57-Fc-L58-TNF1-L59-TNF2-L60-TNF3; VL2-L47-VH2-L48-CH1-L49-CL-L50-Fc-L51-TNF1-L52-TNF2-L53-TNF3; VL2-L54-CH1-L55-VH2-L56-CL-L57-Fc-L58-TNF1-L59-TNF2-L60-TNF3; VL2-VH2-L38-Fc-L39-TNF1-L40-TNF2-L41-TNF3; VH2-VL2-L43-Fc-L44-TNF1-L45-TNF2-L46-TNF3; VL2-VH2-L48-CL-L49-CH1-L50-Fc-L51-TNF1-L52-TNF2-L53-TNF3; VL2-CL-L55-VH2-L56-CH1-L57-Fc-L58-TNF1-L59-TNF2-L60-TNF3; VL2-VH2-L48-CH1-L49-CL-L50-Fc-L51-TNF1-L52-TNF2-L53-TNF3; or VL2-CH1-L55-VH2-L56-CL-L57-Fc-L58-TNF1-L59-TNF2-L60-TNF3; or (ii) the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L85-VH2-L86-Fc; VH2-L87-VL2-L88-Fc; VL2-L89-VH2-L90-CL-L91-CH1-L92-Fc; VL2-L89-VH2-L90-CH1-L91-CL-L92-Fc; VL2-L93-CL-L94-VH2-L95-CH1-L96-Fc; or VL2-L93-CH1-L94-VH2-L95-CL-L96-Fc; wherein VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a TNFSF ligand; TNF2 is a second extracellular domain of a TNFSF ligand; TNF3 is a third extracellular domain of a TNFSF ligand; and L1-L96 are amino acid linkers. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L37-VH2-L38-Fc-L39-TNF1-L40-TNF2-L41-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VH2-L42-VL2-L43-Fc-L44-TNF1-L45-TNF2-L46-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L47-VH2-L48-CL-L49-CH1-L50-Fc-L51-TNF1-L52-TNF2-L53-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L54-CL-L55-VH2-L56-CH1-L57-Fc-L58-TNF1-L59-TNF2-L60-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L47-VH2-L48-CH1-L49-CL-L50-Fc-L51-TNF1-L52-TNF2-L53-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-L54-CH1-L55-VH2-L56-CL-L57-Fc-L58-TNF1-L59-TNF2-L60-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-VH2-L38-Fc-L39-TNF1-L40-TNF2-L41-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VH2-VL2-L43-Fc-L44-TNF1-L45-TNF2-L46-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-VH2-L48-CL-L49-CH1-L50-Fc-L51-TNF1-L52-TNF2-L53-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-CL-L55-VH2-L56-CH1-L57-Fc-L58-TNF1-L59-TNF2-L60-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-VH2-L48-CH1-L49-CL-L50-Fc-L51-TNF1-L52-TNF2-L53-TNF3. In some aspects, the first polypeptide has a structure represented by VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by VL2-CH1-L55-VH2-L56-CL-L57-Fc-L58-TNF1-L59-TNF2-L60-TNF3. For example, the first polypeptide may have a structure represented by VL1-L13-VH1-L14-Fc-L15-TNF1-L16-TNF2-L17-TNF3 and the second polypeptide may have a structure represented by VL2-L37-VH2-L38-Fc-L39-TNF1-L40-TNF2-L41-TNF3. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L85-VH2-L86-Fc. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VH2-L87-VL2-L88-Fc. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L89-VH2-L90-CL-L91-CH1-L92-Fc. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L93-CL-L94-VH2-L95-CH1-L96-Fc. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; or VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L89-VH2-L90-CH1-L91-CL-L92-Fc. In some aspects, the first polypeptide has a structure represented by VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; or VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by VL2-L93-CH1-L94-VH2-L95-CL-L96-Fc.

In some aspects, the VL1 and VH1 of the antigen binding polypeptide complex specifically bind to CD3.

In some aspects, the VL1 of the antigen binding polypeptide complex comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH1 of the antigen binding polypeptide complex comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL1 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL1 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH1 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL1 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH1 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 of the antigen binding polypeptide complex comprises the amino acid sequence of SEQ ID NO:45, and/or the VH1 of the antigen binding polypeptide complex comprises the amino acid sequence of SEQ ID NO:43. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 of the antigen binding polypeptide complex comprises the amino acid sequence of SEQ ID NO:45, and/or the VH1 of the antigen binding polypeptide complex comprises the amino acid sequence of SEQ ID NO:44.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL2 and VH2 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL2 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH2 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL2 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH2 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL2 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH2 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL2 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH2 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL2 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH2 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL2 of the antigen binding polypeptide complex comprises the amino acid sequence of SEQ ID NO:47, and/or the VH2 of the antigen binding polypeptide complex comprises the amino acid sequence of SEQ ID NO:46. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL2 of the antigen binding polypeptide complex comprises the amino acid sequence of SEQ ID NO:49, and/or the VH2 of the antigen binding polypeptide complex comprises the amino acid sequence of SEQ ID NO:48.

In some aspects, the VL2 comprises a CDR1 comprising an amino acid sequence having at least 90% identity, at least 95% identity, or 100% identity to SEQ ID NO:314 or 322; a CDR2 comprising an amino acid sequence having at least 90% identity, at least 95% identity, or 100% identity to SEQ ID NO:315 or 323; and/or a CDR3 comprising an amino acid sequence having at least 90% identity, at least 95% identity, or 100% identity to SEQ ID NO:316 or 324; and/or the VH2 comprises a CDR1 comprising an amino acid sequence having at least 90% identity, at least 95% identity, or 100% identity to SEQ ID NO:310 or 318; a CDR2 comprising an amino acid sequence having at least 90% identity, at least 95% identity, or 100% identity to SEQ ID NO:311 or 319; and/or a CDR3 comprising an amino acid sequence having at least 90% identity, at least 95% identity, or 100% identity to SEQ ID NO:312 or 320. In some aspects, the VL2 comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH2 comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL2 comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH2 comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320. In some aspects, the VL2 comprises an amino acid sequence having at least 90% identity, at least 95% identity, or 100% identity to SEQ ID NO:313 or 321; and/or the VH2 comprises an amino acid sequence having at least 90% identity, at least 95% identity, or 100% identity to SEQ ID NO:309 or 317. In some aspects, the VL2 comprises an amino acid sequence having at least 90% identity, at least 95% identity, or 100% identity to SEQ ID NO:313; and/or the VH2 comprises an amino acid sequence having at least 90% identity, at least 95% identity, or 100% identity to SEQ ID NO:309. In some aspects, the VL2 comprises an amino acid sequence having at least 90% identity, at least 95% identity, or 100% identity to SEQ ID NO:321; and/or the VH2 comprises an amino acid sequence having at least 90% identity, at least 95% identity, or 100% identity to SEQ ID NO:317. As used herein, “at least 90% identity” includes at least 91%, 92%, 93%, 94%, 95%, 96%, 97%, 98%, 99% and 100% identity to the recited reference sequence.

In some aspects, the VL2 comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH2 comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL2 comprises an amino acid sequence having at least 90% identity, at least 95% identity, or 100% identity to SEQ ID NO:273; and/or the VH2 comprises an amino acid sequence having at least 90% identity, at least 95% identity, or 100% identity to SEQ ID NO:269. As used herein, “at least 90% identity” includes at least 91%, 92%, 93%, 94%, 95%, 96%, 97%, 98%, 99% and 100% identity to the recited reference sequence.

In some aspects, the VL2 comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH2 comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL2 comprises an amino acid sequence having at least 90% identity, at least 95% identity, or 100% identity to SEQ ID NO:281; and/or the VH2 comprises an amino acid sequence having at least 90% identity, at least 95% identity, or 100% identity to SEQ ID NO:277. As used herein, “at least 90% identity” includes at least 91%, 92%, 93%, 94%, 95%, 96%, 97%, 98%, 99% and 100% identity to the recited reference sequence.

In some aspects, the VL2 comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH2 comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL2 comprises an amino acid sequence having at least 90% identity, at least 95% identity, or 100% identity to SEQ ID NO:289; and/or the VH2 comprises an amino acid sequence having at least 90% identity, at least 95% identity, or 100% identity to SEQ ID NO:285. As used herein, “at least 90% identity” includes at least 91%, 92%, 93%, 94%, 95%, 96%, 97%, 98%, 99% and 100% identity to the recited reference sequence.

In some aspects, the VL2 and VH2 of the antigen binding polypeptide complex specifically bind to CD3.

In some aspects, the VL2 of the antigen binding polypeptide complex comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH2 of the antigen binding polypeptide complex comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL2 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH2 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL2 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH2 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL2 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH2 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL2 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH2 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL2 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH2 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL2 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH2 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL2 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH2 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL2 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH2 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL2 of the antigen binding polypeptide complex comprises the amino acid sequence of SEQ ID NO:45, and/or the VH2 of the antigen binding polypeptide complex comprises the amino acid sequence of SEQ ID NO:43. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL2 of the antigen binding polypeptide complex comprises the amino acid sequence of SEQ ID NO:45, and/or the VH2 of the antigen binding polypeptide complex comprises the amino acid sequence of SEQ ID NO:44. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL1 and VH1 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL1 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH1 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL1 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH1 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL1 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH1 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL1 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH1 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL1 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH1 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL1 of the antigen binding polypeptide complex comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH1 of the antigen binding polypeptide complex comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL1 of the antigen binding polypeptide complex comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH1 of the antigen binding polypeptide complex comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL1 of the antigen binding polypeptide complex comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH1 of the antigen binding polypeptide complex comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL1 of the antigen binding polypeptide complex comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH1 of the antigen binding polypeptide complex comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL1 of the antigen binding polypeptide complex comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH1 of the antigen binding polypeptide complex comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL1 of the antigen binding polypeptide complex comprises the amino acid sequence of SEQ ID NO:47, and/or the VH1 of the antigen binding polypeptide complex comprises the amino acid sequence of SEQ ID NO:46. In some aspects, the VL1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH1 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL1 of the antigen binding polypeptide complex comprises the amino acid sequence of SEQ ID NO:49, and/or the VH1 of the antigen binding polypeptide complex comprises the amino acid sequence of SEQ ID NO:48. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH1 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, the VL1 and VH1 of the antigen binding polypeptide specifically bind to CD3 and the VL2 and VH2 specifically bind to a TAA (e.g., HER2) or an immune stimulatory receptor (e.g., CD28). In some aspects, the VL1 and VH1 of the antigen binding polypeptide specifically bind to CD3 and the VL2 and VH2 specifically bind to HER2. In some aspects, the VL1 and VH1 of the antigen binding polypeptide specifically bind to CD3 and the VL2 and VH2 specifically bind to CD28. In some aspects, the VL2 and VH2 of the antigen binding polypeptide specifically bind to CD3 and the VL1 and VH1 specifically bind to a TAA (e.g., HER2) or an immune stimulatory receptor (e.g., CD28). In some aspects, the VL2 and VH2 of the antigen binding polypeptide specifically bind to CD3 and the VL1 and VH1 specifically bind to HER2. In some aspects, the VL2 and VH2 of the antigen binding polypeptide specifically bind to CD3 and the VL1 and VH1 specifically bind to CD28.

In some aspects, an antigen binding polypeptide complex of the invention comprises a first polypeptide and a second polypeptide; wherein (i) the first polypeptide has a structure represented by Fc; VL1-L1-VL2-L2-VH2-L3-VH1-L4-Fc; VH1-L5-VH2-L6-VL2-L7-VL1-L8-Fc; Fc-L9-TNF1-L10-TNF2-L11-TNF3; VL1-L12-VL2-L13-VH2-L14-VH1-L15-Fc-L16-TNF1-L17-TNF2-L18-TNF3; or VH1-L19-VH2-L20-VL2-L21-VL1-L22-Fc-L23-TNF1-L24-TNF2-L25-TNF3; and the second polypeptide has a structure represented by VL3-L26-VL4-L27-VH4-L28-VH3-L29-Fc-L30-TNF1-L31-TNF2-L32-TNF3; or VH3-L33-VH4-L34-VL4-L35-VL3-L36-Fc-L37-TNF1-L38-TNF2-L39-TNF3; or (ii) the first polypeptide has a structure represented by Fc-L40-TNF1-L41-TNF2-L42-TNF3; VL1-L43-VL2-L44-VH2-L45-VH1-L46-Fc-L47-TNF1-L48-TNF2-L49-TNF3; or VH1-L50-VH2-L51-VL2-L52-VL1-L53-Fc-L54-TNF1-L55-TNF2-L56-TNF3; and the second polypeptide has a structure represented by Fc; VL3-L57-VL4-L58-VH4-L59-VH3-L60-Fc; or VH3-L61-VH4-L62-VL4-L63-VL3-L64-Fc; wherein VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; TNF1 is a first extracellular domain of a TNFSF ligand; TNF2 is a second extracellular domain of a TNFSF ligand; TNF3 is a third extracellular domain of a TNFSF ligand; and L1-L64 are amino acid linkers. In some aspects, the first polypeptide has a structure represented by Fc; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by Fc; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by VL1-L1-VL2-L2-VH2-L3-VH1-L4-Fc; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by VL1-L1-VL2-L2-VH2-L3-VH1-L4-Fc; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by VH1-L5-VH2-L6-VL2-L7-VL1-L8-Fc; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by VH1-L5-VH2-L6-VL2-L7-VL1-L8-Fc; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by Fc-L9-TNF1-L10-TNF2-L11-TNF3; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by Fc-L9-TNF1-L10-TNF2-L11-TNF3; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by VL1-L12-VL2-L13-VH2-L14-VH1-L15-Fc-L16-TNF1-L17-TNF2-L18-TNF3; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by VL1-L12-VL2-L13-VH2-L14-VH1-L15-Fc-L16-TNF1-L17-TNF2-L18-TNF3; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by VH1-L19-VH2-L20-VL2-L21-VL1-L22-Fc-L23-TNF1-L24-TNF2-L25-TNF3; and the second polypeptide has a structure represented by VL3-L26-VL4-L27-VH4-L28-VH3-L29-Fc-L30-TNF1-L31-TNF2-L32-TNF3. In some aspects, the first polypeptide has a structure represented by VH1-L19-VH2-L20-VL2-L21-VL1-L22-Fc-L23-TNF1-L24-TNF2-L25-TNF3; and the second polypeptide has a structure represented by VH3-L33-VH4-L34-VL4-L35-VL3-L36-Fc-L37-TNF1-L38-TNF2-L39-TNF3. In some aspects, the first polypeptide has a structure represented by Fc-L40-TNF1-L41-TNF2-L42-TNF3; and the second polypeptide has a structure represented by Fc. In some aspects, the first polypeptide has a structure represented by Fc-L40-TNF1-L41-TNF2-L42-TNF3; and the second polypeptide has a structure represented by VL3-L57-VL4-L58-VH4-L59-VH3-L60-Fc. In some aspects, the first polypeptide has a structure represented by Fc-L40-TNF1-L41-TNF2-L42-TNF3; and the second polypeptide has a structure represented by VH3-L61-VH4-L62-VL4-L63-VL3-L64-Fc. In some aspects, the first polypeptide has a structure represented by VL1-L43-VL2-L44-VH2-L45-VH1-L46-Fc-L47-TNF1-L48-TNF2-L49-TNF3; and the second polypeptide has a structure represented by Fc. In some aspects, the first polypeptide has a structure represented by VL1-L43-VL2-L44-VH2-L45-VH1-L46-Fc-L47-TNF1-L48-TNF2-L49-TNF3; and the second polypeptide has a structure represented by VL3-L57-VL4-L58-VH4-L59-VH3-L60-Fc. In some aspects, the first polypeptide has a structure represented by VL1-L43-VL2-L44-VH2-L45-VH1-L46-Fc-L47-TNF1-L48-TNF2-L49-TNF3; and the second polypeptide has a structure represented by VH3-L61-VH4-L62-VL4-L63-VL3-L64-Fc. In some aspects, the first polypeptide has a structure represented by VH1-L50-VH2-L51-VL2-L52-VL1-L53-Fc-L54-TNF1-L55-TNF2-L56-TNF3; and the second polypeptide has a structure represented by Fc. In some aspects, the first polypeptide has a structure represented by VH1-L50-VH2-L51-VL2-L52-VL1-L53-Fc-L54-TNF1-L55-TNF2-L56-TNF3; and the second polypeptide has a structure represented by VL3-L57-VL4-L58-VH4-L59-VH3-L60-Fc. In some aspects, the first polypeptide has a structure represented by VH1-L50-VH2-L51-VL2-L52-VL1-L53-Fc-L54-TNF1-L55-TNF2-L56-TNF3; and the second polypeptide has a structure represented by VH3-L61-VH4-L62-VL4-L63-VL3-L64-Fc.

In some aspects, the VL1, VH1, VL3 and VH3 of the antigen binding polypeptide complex specifically bind to CD3.

In some aspects, the VL1 and VL3 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH1 and VH3 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 394. In some aspects, the VL1 and VL3 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 and VH3 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 and VL3 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 and VH3 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 and VL3 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 and VH3 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL1 and VL3 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 and VH3 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 and VL3 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 and VH3 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 and VL3 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 and VH3 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 and VL3 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH1 and VH3 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL1 and VL3 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH1 and VH3 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 and VL3 of the antigen binding polypeptide complex comprises the amino acid sequence of SEQ ID NO:45, and/or the VH1 and VH3 of the antigen binding polypeptide complex comprises the amino acid sequence of SEQ ID NO:43. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 and VL3 of the antigen binding polypeptide complex comprises the amino acid sequence of SEQ ID NO:45, and/or the VH1 and VH3 of the antigen binding polypeptide complex comprises the amino acid sequence of SEQ ID NO:44. In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL2, VH2, VL4 and VH4 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL2 and VL4 of the antigen binding polypeptide complex comprise a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH2 and VH4 of the antigen binding polypeptide complex comprise a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL2 and VL4 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH2 and VH4 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL2 and VL4 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH2 and VH4 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL2 and VL4 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH2 and VH4 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL2 and VL4 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH2 and VH4 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL2 and VL4 of the antigen binding polypeptide complex comprise a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH2 and VH4 of the antigen binding polypeptide complex comprise a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL2 and VL4 of the antigen binding polypeptide complex comprise a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH2 and VH4 of the antigen binding polypeptide complex comprise a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL2 and VL4 of the antigen binding polypeptide complex comprise a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH2 and VH4 of the antigen binding polypeptide complex comprise a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL2 and VL4 of the antigen binding polypeptide complex comprise a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH2 and VH4 of the antigen binding polypeptide complex comprise a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL2 and VL4 of the antigen binding polypeptide complex comprise a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH2 and VH4 of the antigen binding polypeptide complex comprise a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL2 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH2 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL2 and VL4 of the antigen binding polypeptide complex comprises the amino acid sequence of SEQ ID NO:47, and/or the VH2 and VH4 of the antigen binding polypeptide complex comprises the amino acid sequence of SEQ ID NO:46. In some aspects, the VL2 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH2 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL2 and VL4 of the antigen binding polypeptide complex comprises the amino acid sequence of SEQ ID NO:49, and/or the VH2 and VH4 of the antigen binding polypeptide complex comprises the amino acid sequence of SEQ ID NO:48. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, the VL1, VH1, VL4 and VH4 of the antigen binding polypeptide complex specifically bind to CD3.

In some aspects, the VL1 and VL4 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH1 and VH4 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL1 and VL4 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 and VH4 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 and VL4 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 and VH4 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 and VL4 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 and VH4 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL1 and VL4 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 and VH4 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 and VL4 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 and VH4 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 and VL4 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 and VH4 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 and VL4 of the antigen binding polypeptide complex comprise a CDR1 comprising an amino acid sequence having at least 90% identity, at least 95% identity, or 100% identity to any one of SEQ ID NO:298; a CDR2 comprising an amino acid sequence having at least 90% identity, at least 95% identity, or 100% identity to any one of SEQ ID NO:299; and/or a CDR3 comprising an amino acid sequence having at least 90% identity, at least 95% identity, or 100% identity to any one of SEQ ID NO:300; and/or the VH1 and VH4 of the antigen binding polypeptide complex comprise a CDR1 comprising an amino acid sequence having at least 90% identity, at least 95% identity, or 100% identity to any one of SEQ ID NOs:294; a CDR2 comprising an amino acid sequence having at least 90% identity, at least 95% identity, or 100% identity to any one of SEQ ID NO:295; and/or a CDR3 comprising an amino acid sequence having at least 90% identity, at least 95% identity, or 100% identity to any one of SEQ ID NO:296. In some aspects, the VL1 and VL4 of the antigen binding polypeptide complex comprise a CDR1 comprising an amino acid sequence having at least 90% identity, at least 95% identity, or 100% identity to any one of SEQ ID NO:306; a CDR2 comprising an amino acid sequence having at least 90% identity, at least 95% identity, or 100% identity to any one of SEQ ID NO:307; and/or a CDR3 comprising an amino acid sequence having at least 90% identity, at least 95% identity, or 100% identity to any one of SEQ ID NO:308; and/or the VH1 and VH4 of the antigen binding polypeptide complex comprise a CDR1 comprising an amino acid sequence having at least 90% identity, at least 95% identity, or 100% identity to any one of SEQ ID NOs:302; a CDR2 comprising an amino acid sequence having at least 90% identity, at least 95% identity, or 100% identity to any one of SEQ ID NO:303; and/or a CDR3 comprising an amino acid sequence having at least 90% identity, at least 95% identity, or 100% identity to any one of SEQ ID NO:304.

In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 and VL4 of the antigen binding polypeptide complex comprises the amino acid sequence of SEQ ID NO:45, and/or the VH1 and VH4 of the antigen binding polypeptide complex comprises the amino acid sequence of SEQ ID NO:43. In some aspects, the VL1 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 and VL4 of the antigen binding polypeptide complex comprises the amino acid sequence of SEQ ID NO:45, and/or the VH1 and VH4 of the antigen binding polypeptide complex comprises the amino acid sequence of SEQ ID NO:44. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL2, VH2, VL3 and VH3 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL2 and VL3 of the antigen binding polypeptide complex comprise a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH2 and VH3 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL2 and VL3 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH2 and VH3 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL2 and VL3 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH2 and VH3 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL2 and VL3 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH2 and VH3 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL2 and VL3 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH2 and VH3 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL2 and VL3 of the antigen binding polypeptide complex comprise a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH2 and VH3 of the antigen binding polypeptide complex comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL2 and VL3 of the antigen binding polypeptide complex comprise a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH2 and VH3 of the antigen binding polypeptide complex comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL2 and VL3 of the antigen binding polypeptide complex comprise a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH2 and VH3 of the antigen binding polypeptide complex comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL2 and VL3 of the antigen binding polypeptide complex comprise a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH2 and VH3 of the antigen binding polypeptide complex comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL2 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH2 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL2 and VL3 of the antigen binding polypeptide complex comprises the amino acid sequence of SEQ ID NO:47, and/or the VH2 and VH3 of the antigen binding polypeptide complex comprises the amino acid sequence of SEQ ID NO:46. In some aspects, the VL2 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH2 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL2 and VL3 of the antigen binding polypeptide complex comprises the amino acid sequence of SEQ ID NO:49, and/or the VH2 and VH3 of the antigen binding polypeptide complex comprises the amino acid sequence of SEQ ID NO:48. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, the VL2, VH2, VL4 and VH4 of the antigen binding polypeptide complex specifically bind to CD3.

In some aspects, the VL2 and VL4 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH2 and VH4 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL2 and VL4 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH2 and VH4 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL2 and VL4 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH2 and VH4 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL2 and VL4 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH2 and VH4 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL2 and VL4 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH2 and VH4 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL2 and VL4 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH2 and VH4 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL2 and VL4 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH2 and VH4 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL2 and VL4 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH2 and VH4 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL2 and VL4 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH2 and VH4 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL2 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL2 and VL4 of the antigen binding polypeptide complex comprises the amino acid sequence of SEQ ID NO:45, and/or the VH2 and VH4 of the antigen binding polypeptide complex comprises the amino acid sequence of SEQ ID NO:43. In some aspects, the VL2 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL2 and VL4 of the antigen binding polypeptide complex comprises the amino acid sequence of SEQ ID NO:45, and/or the VH2 and VH4 of the antigen binding polypeptide complex comprises the amino acid sequence of SEQ ID NO:44. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL2 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH2 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL1, VH1, VL3 and VH3 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL1 and VL3 of the antigen binding polypeptide complex comprise a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH1 and VH3 of the antigen binding polypeptide complex comprise a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL1 and VL3 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH1 and VH3 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL1 and VL3 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH1 and VH3 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL1 and VL3 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH1 and VH3 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL1 and VL3 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH1 and VH3 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL1 and VL3 of the antigen binding polypeptide complex comprises a CDR1 comprising an amino acid sequence having at least 90% identity to SEQ ID NO:274; a CDR2 comprising an amino acid sequence having at least 90% identity to SEQ ID NO:275; and/or a CDR3 comprising an amino acid sequence having at least 90% identity to SEQ ID NO:276; and/or the VH1 and VH3 of the antigen binding polypeptide complex comprises a CDR1 comprising an amino acid sequence having at least 90% identity to SEQ ID NO:270; a CDR2 comprising an amino acid sequence having at least 90% identity to SEQ ID NO:271; and/or a CDR3 comprising an amino acid sequence having at least 90% identity to SEQ ID NO:272. In some aspects, the VL1 and VL3 of the antigen binding polypeptide complex comprises a CDR1 comprising an amino acid sequence having at least 90% identity to SEQ ID NO:282; a CDR2 comprising an amino acid sequence having at least 90% identity to SEQ ID NO:283; and/or a CDR3 comprising an amino acid sequence having at least 90% identity to SEQ ID NO:284; and/or the VH1 and VH3 of the antigen binding polypeptide complex comprises a CDR1 comprising an amino acid sequence having at least 90% identity to SEQ ID NO:278; a CDR2 comprising an amino acid sequence having at least 90% identity to SEQ ID NO:279; and/or a CDR3 comprising an amino acid sequence having at least 90% identity to SEQ ID NO:280. In some aspects, the VL1 and VL3 of the antigen binding polypeptide complex comprises a CDR1 comprising an amino acid sequence having at least 90% identity to SEQ ID NO:290; a CDR2 comprising an amino acid sequence having at least 90% identity to SEQ ID NO:291; and/or a CDR3 comprising an amino acid sequence having at least 90% identity to SEQ ID NO:292; and/or the VH1 and VH3 of the antigen binding polypeptide complex comprises a CDR1 comprising an amino acid sequence having at least 90% identity to SEQ ID NO:286; a CDR2 comprising an amino acid sequence having at least 90% identity to SEQ ID NO:287; and/or a CDR3 comprising an amino acid sequence having at least 90% identity to SEQ ID NO:288. In some aspects, the VL1 and VL3 of the antigen binding polypeptide complex comprises a CDR1 comprising an amino acid sequence having at least 90% identity to SEQ ID NO:314; a CDR2 comprising an amino acid sequence having at least 90% identity to SEQ ID NO:315; and/or a CDR3 comprising an amino acid sequence having at least 90% identity to SEQ ID NO:316; and/or the VH1 and VH3 of the antigen binding polypeptide complex comprises a CDR1 comprising an amino acid sequence having at least 90% identity to SEQ ID NO:310; a CDR2 comprising an amino acid sequence having at least 90% identity to SEQ ID NO:311; and/or a CDR3 comprising an amino acid sequence having at least 90% identity to SEQ ID NO:312. In some aspects, the VL1 and VL3 of the antigen binding polypeptide complex comprises a CDR1 comprising an amino acid sequence having at least 90% identity to SEQ ID NO:322; a CDR2 comprising an amino acid sequence having at least 90% identity to SEQ ID NO:323; and/or a CDR3 comprising an amino acid sequence having at least 90% identity to SEQ ID NO:324; and/or the VH1 and VH3 of the antigen binding polypeptide complex comprises a CDR1 comprising an amino acid sequence having at least 90% identity to SEQ ID NO:318; a CDR2 comprising an amino acid sequence having at least 90% identity to SEQ ID NO:319; and/or a CDR3 comprising an amino acid sequence having at least 90% identity to SEQ ID NO:320.

In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL1 and VL3 of the antigen binding polypeptide complex comprises the amino acid sequence of SEQ ID NO:47, and/or the VH1 and VH3 of the antigen binding polypeptide complex comprises the amino acid sequence of SEQ ID NO:46. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL1 and VL3 of the antigen binding polypeptide complex comprises the amino acid sequence of SEQ ID NO:49, and/or the VH1 and VH3 of the antigen binding polypeptide complex comprises the amino acid sequence of SEQ ID NO:48. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:273, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:269. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:281, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:277. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:289, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:285. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:313, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:309. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:321, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:317.

In some aspects, the VL2, VH2, VL3 and VH3 of the antigen binding polypeptide complex specifically bind to CD3.

In some aspects, the VL2 and VL3 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH2 and VH3 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL2 and VL3 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH2 and VH3 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL2 and VL3 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH2 and VH3 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL2 and VL3 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH2 and VH3 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL2 and VL3 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH2 and VH3 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL2 and VL3 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH2 and VH3 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL2 and VL3 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH2 and VH3 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL2 and VL3 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH2 and VH3 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL2 and VL3 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH2 and VH3 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL2 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL2 and VL3 of the antigen binding polypeptide complex comprises the amino acid sequence of SEQ ID NO:45, and/or the VH2 and VH3 of the antigen binding polypeptide complex comprises the amino acid sequence of SEQ ID NO:43. In some aspects, the VL2 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH2 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL2 and VL3 of the antigen binding polypeptide complex comprises the amino acid sequence of SEQ ID NO:45, and/or the VH2 and VH3 of the antigen binding polypeptide complex comprises the amino acid sequence of SEQ ID NO:44. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL2 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH2 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL1, VH1, VL4 and VH4 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL1 and VL4 of the antigen binding polypeptide complex comprise a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH1 and VH4 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL1 and VL4 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH1 and VH4 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL1 and VL4 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH1 and VH4 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL1 and VL4 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH1 and VH4 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL1 and VL4 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH1 and VH4 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL1 and VL4 of the antigen binding polypeptide complex comprise a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH1 and VH4 of the antigen binding polypeptide complex comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL1 and VL4 of the antigen binding polypeptide complex comprise a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH1 and VH4 of the antigen binding polypeptide complex comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL1 and VL4 of the antigen binding polypeptide complex comprise a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH1 and VH4 of the antigen binding polypeptide complex comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL1 and VL4 of the antigen binding polypeptide complex comprise a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH1 and VH4 of the antigen binding polypeptide complex comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL1 and VL4 of the antigen binding polypeptide complex comprise a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH1 and VH4 of the antigen binding polypeptide complex comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL1 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH1 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL1 and VL4 of the antigen binding polypeptide complex comprises the amino acid sequence of SEQ ID NO:47, and/or the VH1 and VH4 of the antigen binding polypeptide complex comprises the amino acid sequence of SEQ ID NO:46. In some aspects, the VL1 and VL4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH1 and VH4 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL1 and VL4 of the antigen binding polypeptide complex comprises the amino acid sequence of SEQ ID NO:49, and/or the VH1 and VH4 of the antigen binding polypeptide complex comprises the amino acid sequence of SEQ ID NO:48. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL1 and VL4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH1 and VH4 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, an antigen binding polypeptide complex of the invention comprises a first polypeptide and a second polypeptide; wherein (i) the first polypeptide has a structure represented by VL1-L1-VH1-L2-Fc; VH1-L3-VL1-L4-Fc; VL1-L5-VH1-L6-Fc-L7-TNF1-L8-TNF2-L9-TNF3; or VH1-L10-VL1-L11-Fc-L12-TNF1-L13-TNF2-L14-TNF3; and the second polypeptide has a structure represented by VL2-L15-VL3-L16-VH3-L17-VH2-L18-Fc-L19-TNF1-L20-TNF2-L21-TNF3; or VH2-L22-VH3-L23-VL3-L24-VL2-L25-Fc-L26-TNF1-L27-TNF2-L28-TNF3; or (ii) the first polypeptide has a structure represented by VL1-L29-VH1-L30-Fc-L31-TNF1-L32-TNF2-L33-TNF3; or VH1-L34-VL1-L35-Fc-L36-TNF1-L37-TNF2-L38-TNF3; and the second polypeptide has a structure represented by VL2-L39-VL3-L40-VH3-L41-VH2-L42-Fc; or VH2-L43-VH3-L44-VL3-L45-VL2-L46-Fc; wherein VL1 is a first immunoglobulin light chain variable region; VL2 is a second immunoglobulin light chain variable region; VL3 is a third immunoglobulin light chain variable region; VH1 is a first immunoglobulin heavy chain variable region; VH2 is a second immunoglobulin heavy chain variable region; VH3 is a third immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; TNF1 is a first extracellular domain of a TNFSF ligand; TNF2 is a second extracellular domain of a TNFSF ligand; TNF3 is a third extracellular domain of a TNFSF ligand; and L1-L46 are amino acid linkers. In some aspects, the first polypeptide has a structure represented by VL1-L1-VH1-L2-Fc and the second polypeptide has a structure represented by VL2-L15-VL3-L16-VH3-L17-VH2-L18-Fc-L19-TNF1-L20-TNF2-L21-TNF3. In some aspects, the first polypeptide has a structure represented by VL1-L1-VH1-L2-Fc and the second polypeptide has a structure represented by VH2-L22-VH3-L23-VL3-L24-VL2-L25-Fc-L26-TNF1-L27-TNF2-L28-TNF3. In some aspects, the first polypeptide has a structure represented by VH1-L3-VL1-L4-Fc and the second polypeptide has a structure represented by VL2-L15-VL3-L16-VH3-L17-VH2-L18-Fc-L19-TNF1-L20-TNF2-L21-TNF3. In some aspects, the first polypeptide has a structure represented by VH1-L3-VL1-L4-Fc and the second polypeptide has a structure represented by VH2-L22-VH3-L23-VL3-L24-VL2-L25-Fc-L26-TNF1-L27-TNF2-L28-TNF3. In some aspects, the first polypeptide has a structure represented by VL1-L5-VH1-L6-Fc-L7-TNF1-L8-TNF2-L9-TNF3 and the second polypeptide has a structure represented by VL2-L15-VL3-L16-VH3-L17-VH2-L18-Fc-L19-TNF1-L20-TNF2-L21-TNF3. In some aspects, the first polypeptide has a structure represented by VL1-L5-VH1-L6-Fc-L7-TNF1-L8-TNF2-L9-TNF3 and the second polypeptide has a structure represented by VH2-L22-VH3-L23-VL3-L24-VL2-L25-Fc-L26-TNF1-L27-TNF2-L28-TNF3. In some aspects, the first polypeptide has a structure represented by VH1-L10-VL1-L11-Fc-L12-TNF1-L13-TNF2-L14-TNF3 and the second polypeptide has a structure represented by VL2-L15-VL3-L16-VH3-L17-VH2-L18-Fc-L19-TNF1-L20-TNF2-L21-TNF3. In some aspects, the first polypeptide has a structure represented by VH1-L10-VL1-L11-Fc-L12-TNF1-L13-TNF2-L14-TNF3 and the second polypeptide has a structure represented by VH2-L22-VH3-L23-VL3-L24-VL2-L25-Fc-L26-TNF1-L27-TNF2-L28-TNF3. In some aspects, the first polypeptide has a structure represented by VL1-L29-VH1-L30-Fc-L31-TNF1-L32-TNF2-L33-TNF3 and the second polypeptide has a structure represented by VL2-L39-VL3-L40-VH3-L41-VH2-L42-Fc. In some aspects, the first polypeptide has a structure represented by VL1-L29-VH1-L30-Fc-L31-TNF1-L32-TNF2-L33-TNF3 and the second polypeptide has a structure represented by VH2-L43-VH3-L44-VL3-L45-VL2-L46-Fc. In some aspects, the first polypeptide has a structure represented by VH1-L34-VL1-L35-Fc-L36-TNF1-L37-TNF2-L38-TNF3 and the second polypeptide has a structure represented by VL2-L39-VL3-L40-VH3-L41-VH2-L42-Fc. In some aspects, the first polypeptide has a structure represented by VH1-L34-VL1-L35-Fc-L36-TNF1-L37-TNF2-L38-TNF3 and the second polypeptide has a structure represented by VH2-L43-VH3-L44-VL3-L45-VL2-L46-Fc.

In some aspects, the VL1, VH1, VL2 and VH2 of the antigen binding polypeptide complex specifically bind to CD3.

In some aspects, the VL1 and VL2 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308; and/or the VH1 and VH2 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL1 and VL2 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 and VH2 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 and VL2 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 and VH2 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 and VL2 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 and VH2 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL1 and VL2 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 and VH2 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 and VL2 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 and VH2 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 and VL2 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 and VH2 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 and VL2 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH1 and VH2 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL1 and VL2 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH1 and VH2 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL1 and VL2 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 and VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 and VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 and VL2 of the antigen binding polypeptide complex comprises the amino acid sequence of SEQ ID NO:45, and/or the VH1 and VH2 of the antigen binding polypeptide complex comprises the amino acid sequence of SEQ ID NO:43. In some aspects, the VL1 and VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 and VL2 of the antigen binding polypeptide complex comprises the amino acid sequence of SEQ ID NO:45, and/or the VH1 and VH2 of the antigen binding polypeptide complex comprises the amino acid sequence of SEQ ID NO:44. In some aspects, the VL1 and VL2 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 and VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL1 and VL2 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 and VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL3 and VH3 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL3 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH3 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL3 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH3 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL3 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH3 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL3 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH3 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL3 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH3 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL3 of the antigen binding polypeptide complex comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH3 of the antigen binding polypeptide complex comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL3 of the antigen binding polypeptide complex comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH3 of the antigen binding polypeptide complex comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL3 of the antigen binding polypeptide complex comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH3 of the antigen binding polypeptide complex comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL3 of the antigen binding polypeptide complex comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH3 of the antigen binding polypeptide complex comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL3 of the antigen binding polypeptide complex comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH3 of the antigen binding polypeptide complex comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL3 of the antigen binding polypeptide complex comprises the amino acid sequence of SEQ ID NO:47, and/or the VH3 of the antigen binding polypeptide complex comprises the amino acid sequence of SEQ ID NO:46. In some aspects, the VL3 of the antigen binding polypeptide complex comprises the amino acid sequence of SEQ ID NO:49, and/or the VH3 of the antigen binding polypeptide complex comprises the amino acid sequence of SEQ ID NO:48. In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH3 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, the VL1, VH1, VL3 and VH3 of the antigen binding polypeptide complex specifically bind to CD3.

In some aspects, the VL1 and VL3 of the antigen binding polypeptide complex comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308 and/or the VH1 and VH3 of the antigen binding polypeptide complex comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VL1 and VL3 of the antigen binding polypeptide complex comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or the VH1 and VH3 of the antigen binding polypeptide complex comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VL1 and VL3 of the antigen binding polypeptide complex comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or the VH1 and VH3 of the antigen binding polypeptide complex comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VL1 and VL3 of the antigen binding polypeptide complex comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or the VH1 and VH3 of the antigen binding polypeptide complex comprises a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VL1 and VL3 of the antigen binding polypeptide complex comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or the VH1 and VH3 of the antigen binding polypeptide complex comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VL1 and VL3 of the antigen binding polypeptide complex comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or the VH1 and VH3 of the antigen binding polypeptide complex comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VL1 and VL3 of the antigen binding polypeptide complex comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 and VH3 of the antigen binding polypeptide complex comprises a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VL1 and VL3 of the antigen binding polypeptide complex comprises a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300; and/or the VH1 and VH3 of the antigen binding polypeptide complex comprises a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VL1 and VL3 of the antigen binding polypeptide complex comprises a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308; and/or the VH1 and VH3 of the antigen binding polypeptide complex comprises a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VL1 and VL3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VH1 and VH3 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VL1 and VL3 of the antigen binding polypeptide complex comprises the amino acid sequence of SEQ ID NO:45, and/or the VH1 and VH3 of the antigen binding polypeptide complex comprises the amino acid sequence of SEQ ID NO:43. In some aspects, the VL1 and VL3 of the antigen binding polypeptide complex comprises the amino acid sequence of SEQ ID NO:45, and/or the VH1 and VH3 of the antigen binding polypeptide complex comprises the amino acid sequence of SEQ ID NO:44. In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VL1 and VL3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VH1 and VH3 of the antigen binding polypeptide complex comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VL2 and VH2 of the antigen binding polypeptide complex specifically bind to a TAA or an immune stimulatory receptor. In some aspects, the immune stimulatory receptor is CD28. In some aspects, the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM. In some aspects, the TAA is HER2.

In some aspects, the VL2 of the antigen binding polypeptide complex comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42; and/or the VH2 of the antigen binding polypeptide complex comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VL2 of the antigen binding polypeptide complex comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or the VH2 of the antigen binding polypeptide complex comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VL2 of the antigen binding polypeptide complex comprises a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or the VH2 of the antigen binding polypeptide complex comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VL2 of the antigen binding polypeptide complex comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or the VH2 of the antigen binding polypeptide complex comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VL2 of the antigen binding polypeptide complex comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or the VH2 of the antigen binding polypeptide complex comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VL2 of the antigen binding polypeptide complex comprises a CDR1 comprising an amino acid sequence having at least 90% identity, at least 95% identity, or 100% identity to SEQ ID NO:274; a CDR2 comprising an amino acid sequence having at least 90% identity, at least 95% identity, or 100% identity to SEQ ID NO:275; and/or a CDR3 comprising an amino acid sequence having at least 90% identity, at least 95% identity, or 100% identity to SEQ ID NO:276; and/or the VH2 of the antigen binding polypeptide complex comprises a CDR1 comprising an amino acid sequence having at least 90% identity, at least 95% identity, or 100% identity to SEQ ID NO:270; a CDR2 comprising an amino acid sequence having at least 90% identity, at least 95% identity, or 100% identity to SEQ ID NO:271; and/or a CDR3 comprising an amino acid sequence having at least 90% identity, at least 95% identity, or 100% identity to SEQ ID NO:272. In some aspects, the VL2 of the antigen binding polypeptide complex comprises a CDR1 comprising an amino acid sequence having at least 90% identity, at least 95% identity, or 100% identity to SEQ ID NO:282; a CDR2 comprising an amino acid sequence having at least 90% identity, at least 95% identity, or 100% identity to SEQ ID NO:283; and/or a CDR3 comprising an amino acid sequence having at least 90% identity, at least 95% identity, or 100% identity to SEQ ID NO:284; and/or the VH2 of the antigen binding polypeptide complex comprises a CDR1 comprising an amino acid sequence having at least 90% identity, at least 95% identity, or 100% identity to SEQ ID NO:278; a CDR2 comprising an amino acid sequence having at least 90% identity, at least 95% identity, or 100% identity to SEQ ID NO:279; and/or a CDR3 comprising an amino acid sequence having at least 90% identity, at least 95% identity, or 100% identity to SEQ ID NO:280. In some aspects, the VL2 of the antigen binding polypeptide complex comprises a CDR1 comprising an amino acid sequence having at least 90% identity, at least 95% identity, or 100% identity to SEQ ID NO:290; a CDR2 comprising an amino acid sequence having at least 90% identity, at least 95% identity, or 100% identity to SEQ ID NO:291; and/or a CDR3 comprising an amino acid sequence having at least 90% identity, at least 95% identity, or 100% identity to SEQ ID NO:292; and/or the VH2 of the antigen binding polypeptide complex comprises a CDR1 comprising an amino acid sequence having at least 90% identity, at least 95% identity, or 100% identity to SEQ ID NO:286; a CDR2 comprising an amino acid sequence having at least 90% identity, at least 95% identity, or 100% identity to SEQ ID NO:287; and/or a CDR3 comprising an amino acid sequence having at least 90% identity, at least 95% identity, or 100% identity to SEQ ID NO:288. In some aspects, the VL2 of the antigen binding polypeptide complex comprises a CDR1 comprising an amino acid sequence having at least 90% identity, at least 95% identity, or 100% identity to SEQ ID NO:314; a CDR2 comprising an amino acid sequence having at least 90% identity, at least 95% identity, or 100% identity to SEQ ID NO:315; and/or a CDR3 comprising an amino acid sequence having at least 90% identity, at least 95% identity, or 100% identity to SEQ ID NO:316; and/or the VH2 of the antigen binding polypeptide complex comprises a CDR1 comprising an amino acid sequence having at least 90% identity, at least 95% identity, or 100% identity to SEQ ID NO:310; a CDR2 comprising an amino acid sequence having at least 90% identity, at least 95% identity, or 100% identity to SEQ ID NO:311; and/or a CDR3 comprising an amino acid sequence having at least 90% identity, at least 95% identity, or 100% identity to SEQ ID NO:312. In some aspects, the VL2 of the antigen binding polypeptide complex comprises a CDR1 comprising an amino acid sequence having at least 90% identity, at least 95% identity, or 100% identity to SEQ ID NO:322; a CDR2 comprising an amino acid sequence having at least 90% identity, at least 95% identity, or 100% identity to SEQ ID NO:323; and/or a CDR3 comprising an amino acid sequence having at least 90% identity, at least 95% identity, or 100% identity to SEQ ID NO:324; and/or the VH2 of the antigen binding polypeptide complex comprises a CDR1 comprising an amino acid sequence having at least 90% identity, at least 95% identity, or 100% identity to SEQ ID NO:318; a CDR2 comprising an amino acid sequence having at least 90% identity, at least 95% identity, or 100% identity to SEQ ID NO:319; and/or a CDR3 comprising an amino acid sequence having at least 90% identity, at least 95% identity, or 100% identity to SEQ ID NO:320.

In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VL2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VH2 of the antigen binding polypeptide complex comprises an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VL2 of the antigen binding polypeptide complex comprises the amino acid sequence of SEQ ID NO:47, and/or the VH2 of the antigen binding polypeptide complex comprises the amino acid sequence of SEQ ID NO:46. In some aspects, the VL2 of the antigen binding polypeptide complex comprises the amino acid sequence of SEQ ID NO:49, and/or the VH2 of the antigen binding polypeptide complex comprises the amino acid sequence of SEQ ID NO:48. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VL2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VH2 of the antigen binding polypeptide complex comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

In some aspects, the VLs of the antigen binding polypeptide complex specifically binding to CD3 comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and/or a CDR3 comprising an amino acid sequence having at least 90% identity, at least 95% identity, or 100% identity to any one of SEQ ID NOs:24, 30, 187, 300 and 308. In some aspects, the VLs of the antigen binding polypeptide complex specifically binding to CD3 comprise a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24. In some aspects, the VLs of the antigen binding polypeptide complex specifically binding to CD3 comprise a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30. In some aspects, the VLs of the antigen binding polypeptide complex specifically binding to CD3 comprise a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187. As used herein, “at least 90% identity” includes at least 91%, 92%, 93%, 94%, 95%, 96%, 97%, 98%, 99% and 100% identity to the recited reference sequence. In some aspects, the VLs of the antigen binding polypeptide complex specifically binding to CD3 comprise a CDR1 comprising the amino acid sequence of SEQ ID NO:22; a CDR2 comprising the amino acid sequence of SEQ ID NO:23; and/or a CDR3 comprising the amino acid sequence of SEQ ID NO:24. In some aspects, the VLs of the antigen binding polypeptide complex specifically binding to CD3 comprise a CDR1 comprising the amino acid sequence of SEQ ID NO:28; a CDR2 comprising the amino acid sequence of SEQ ID NO:29; and/or a CDR3 comprising the amino acid sequence of SEQ ID NO:30. In some aspects, the VLs of the antigen binding polypeptide complex specifically binding to CD3 comprise a CDR1 comprising the amino acid sequence of SEQ ID NO:185; a CDR2 comprising the amino acid sequence of SEQ ID NO:186; and/or a CDR3 comprising the amino acid sequence of SEQ ID NO:187. In some aspects, the VLs of the antigen binding polypeptide complex specifically binding to CD3 comprise a CDR1 comprising an amino acid sequence having at least 90% identity to SEQ ID NO:298; a CDR2 comprising an amino acid sequence having at least 90% identity to SEQ ID NO:299; and/or a CDR3 comprising an amino acid sequence having at least 90% identity to SEQ ID NO:300. In some aspects, the VLs of the antigen binding polypeptide complex specifically binding to CD3 comprise a CDR1 comprising an amino acid sequence having at least 90% identity to SEQ ID NO:306; a CDR2 comprising an amino acid sequence having at least 90% identity to SEQ ID NO:307; and/or a CDR3 comprising an amino acid sequence having at least 90% identity to SEQ ID NO:308.

In some aspects, the VHs of the antigen binding polypeptide complex specifically binding to CD3 comprise a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and/or a CDR3 comprising an amino acid sequence having at least 90% identity, at least 95% identity, or 100% identity to any one of SEQ ID NOs:21, 27, 184, 296 and 304. In some aspects, the VHs of the antigen binding polypeptide complex specifically binding to CD3 comprise a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21. In some aspects, the VHs of the antigen binding polypeptide complex specifically binding to CD3 comprise a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27. In some aspects, the VHs of the antigen binding polypeptide complex specifically binding to CD3 comprise a CDR1 comprising an amino acid sequence having at least 90% identity to SEQ ID NO:182; a CDR2 comprising an amino acid sequence having at least 90% identity to SEQ ID NO:183; and/or a CDR3 comprising an amino acid sequence having at least 90% identity to SEQ ID NO:184. As used herein, “at least 90% identity” includes at least 91%, 92%, 93%, 94%, 95%, 96%, 97%, 98%, 99% and 100% identity to the recited reference sequence. In some aspects, the VHs of the antigen binding polypeptide complex specifically binding to CD3 comprise a CDR1 comprising the amino acid sequence of SEQ ID NO:19; a CDR2 comprising the amino acid sequence of SEQ ID NO:20; and/or a CDR3 comprising the amino acid sequence of SEQ ID NO:21. In some aspects, the VHs of the antigen binding polypeptide complex specifically binding to CD3 comprise a CDR1 comprising the amino acid sequence of SEQ ID NO:25; a CDR2 comprising the amino acid sequence of SEQ ID NO:26; and/or a CDR3 comprising the amino acid sequence of SEQ ID NO:27. In some aspects, the VHs of the antigen binding polypeptide complex specifically binding to CD3 comprise a CDR1 comprising the amino acid sequence of SEQ ID NO:182; a CDR2 comprising the amino acid sequence of SEQ ID NO:183; and/or a CDR3 comprising the amino acid sequence of SEQ ID NO:184. In some aspects, the VHs of the antigen binding polypeptide complex specifically binding to CD3 comprise a CDR1 comprising an amino acid sequence having at least 90% identity to SEQ ID NO:294; a CDR2 comprising an amino acid sequence having at least 90% identity to SEQ ID NO:295; and/or a CDR3 comprising an amino acid sequence having at least 90% identity to SEQ ID NO:296. In some aspects, the VHs of the antigen binding polypeptide complex specifically binding to CD3 comprise a CDR1 comprising an amino acid sequence having at least 90% identity to SEQ ID NO:302; a CDR2 comprising an amino acid sequence having at least 90% identity to SEQ ID NO:303; and/or a CDR3 comprising an amino acid sequence having at least 90% identity to SEQ ID NO:304.

In some aspects, the VLs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:45, and/or the VHs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:43 or 44. In some aspects, the VLs of the antigen binding polypeptide complex specifically binding to CD3 comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VHs of the antigen binding polypeptide complex specifically binding to CD3 comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:43. In some aspects, the VLs of the antigen binding polypeptide complex specifically binding to CD3 comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:45, and/or the VHs of the antigen binding polypeptide complex specifically binding to CD3 comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:44. In some aspects, the VLs of the antigen binding polypeptide complex specifically binding to CD3 comprise the amino acid sequence of SEQ ID NO:45, and/or the VHs of the antigen binding polypeptide complex specifically binding to CD3 comprise the amino acid sequence of SEQ ID NO:43. In some aspects, the VLs of the antigen binding polypeptide complex specifically binding to CD3 comprise the amino acid sequence of SEQ ID NO:45, and/or the VHs of the antigen binding polypeptide complex specifically binding to CD3 comprise the amino acid sequence of SEQ ID NO:44. In some aspects, the VLs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:297, and/or the VHs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:293. In some aspects, the VLs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:305, and/or the VHs of the antigen binding polypeptide complex specifically binding to CD3 comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:301.

In some aspects, the VLs of the antigen binding polypeptide complex specifically binding to a TAA or an immune stimulatory receptor comprise a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and/or a CDR3 comprising an amino acid sequence having at least 90% identity, at least 95% identity, or 100% identity to SEQ ID NO:36 or 42. In some aspects, the VLs of the antigen binding polypeptide complex specifically binding to a TAA or an immune stimulatory receptor comprise a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36. In some aspects, the VLs of the antigen binding polypeptide complex specifically binding to a TAA or an immune stimulatory receptor comprise a CDR1 comprising an amino acid sequence having at least 90% identity to SEQ ID NO:40; a CDR2 comprising an amino acid sequence having at least 90% identity to SEQ ID NO:41; and/or a CDR3 comprising an amino acid sequence having at least 90% identity to SEQ ID NO:42. As used herein, “at least 90% identity” includes at least 91%, 92%, 93%, 94%, 95%, 96%, 97%, 98%, 99% and 100% identity to the recited reference sequence. In some aspects, the VLs of the antigen binding polypeptide complex specifically binding to a TAA or an immune stimulatory receptor comprise a CDR1 comprising the amino acid sequence of SEQ ID NO:34; a CDR2 comprising the amino acid sequence of SEQ ID NO:35; and/or a CDR3 comprising the amino acid sequence of SEQ ID NO:36. In some aspects, the VLs of the antigen binding polypeptide complex specifically binding to a TAA or an immune stimulatory receptor comprise a CDR1 comprising the amino acid sequence of SEQ ID NO:40; a CDR2 comprising the amino acid sequence of SEQ ID NO:41; and/or a CDR3 comprising the amino acid sequence of SEQ ID NO:42. In some aspects, the VLs of the antigen binding polypeptide complex specifically binding to a TAA or an immune stimulatory receptor comprise a CDR1 comprising an amino acid sequence having at least 90% identity to SEQ ID NO:274; a CDR2 comprising an amino acid sequence having at least 90% identity to SEQ ID NO:275; and/or a CDR3 comprising an amino acid sequence having at least 90% identity to SEQ ID NO:276. In some aspects, the VLs of the antigen binding polypeptide complex specifically binding to a TAA or an immune stimulatory receptor comprise a CDR1 comprising an amino acid sequence having at least 90% identity to SEQ ID NO:282; a CDR2 comprising an amino acid sequence having at least 90% identity to SEQ ID NO:283; and/or a CDR3 comprising an amino acid sequence having at least 90% identity to SEQ ID NO:284. In some aspects, the VLs of the antigen binding polypeptide complex specifically binding to a TAA or an immune stimulatory receptor comprise a CDR1 comprising an amino acid sequence having at least 90% identity to SEQ ID NO:290; a CDR2 comprising an amino acid sequence having at least 90% identity to SEQ ID NO:291; and/or a CDR3 comprising an amino acid sequence having at least 90% identity to SEQ ID NO:292. In some aspects, the VLs of the antigen binding polypeptide complex specifically binding to a TAA or an immune stimulatory receptor comprise a CDR1 comprising an amino acid sequence having at least 90% identity to SEQ ID NO:314; a CDR2 comprising an amino acid sequence having at least 90% identity to SEQ ID NO:315; and/or a CDR3 comprising an amino acid sequence having at least 90% identity to SEQ ID NO:316. In some aspects, the VLs of the antigen binding polypeptide complex specifically binding to a TAA or an immune stimulatory receptor comprise a CDR1 comprising an amino acid sequence having at least 90% identity to SEQ ID NO:322; a CDR2 comprising an amino acid sequence having at least 90% identity to SEQ ID NO:323; and/or a CDR3 comprising an amino acid sequence having at least 90% identity to SEQ ID NO:324.

In some aspects, the VHs of the antigen binding polypeptide complex specifically binding to a TAA or an immune stimulatory receptor comprise a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and/or a CDR3 comprising an amino acid sequence having at least 90% identity, at least 95% identity, or 100% identity to SEQ ID NO:33 or 39. In some aspects, the VHs of the antigen binding polypeptide complex specifically binding to a TAA or an immune stimulatory receptor comprise a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33. In some aspects, the VHs of the antigen binding polypeptide complex specifically binding to a TAA or an immune stimulatory receptor comprise a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39. As used herein, “at least 90% identity” includes at least 91%, 92%, 93%, 94%, 95%, 96%, 97%, 98%, 99% and 100% identity to the recited reference sequence. In some aspects, the VHs of the antigen binding polypeptide complex specifically binding to a TAA or an immune stimulatory receptor comprise a CDR1 comprising the amino acid sequence of SEQ ID NO:31; a CDR2 comprising the amino acid sequence of SEQ ID NO:32; and/or a CDR3 comprising the amino acid sequence of SEQ ID NO:33. In some aspects, the VHs of the antigen binding polypeptide complex specifically binding to a TAA or an immune stimulatory receptor comprise a CDR1 comprising the amino acid sequence of SEQ ID NO:37; a CDR2 comprising the amino acid sequence of SEQ ID NO:38; and/or a CDR3 comprising the amino acid sequence of SEQ ID NO:39. In some aspects, the VHs of the antigen binding polypeptide complex specifically binding to a TAA or an immune stimulatory receptor comprise a CDR1 comprising an amino acid sequence having at least 90% identity to SEQ ID NO:270; a CDR2 comprising an amino acid sequence having at least 90% identity to SEQ ID NO:271; and/or a CDR3 comprising an amino acid sequence having at least 90% identity to SEQ ID NO:272. In some aspects, the VHs of the antigen binding polypeptide complex specifically binding to a TAA or an immune stimulatory receptor comprise a CDR1 comprising an amino acid sequence having at least 90% identity to SEQ ID NO:278; a CDR2 comprising an amino acid sequence having at least 90% identity to SEQ ID NO:279; and/or a CDR3 comprising an amino acid sequence having at least 90% identity to SEQ ID NO:280. In some aspects, the VHs of the antigen binding polypeptide complex specifically binding to a TAA or an immune stimulatory receptor comprise a CDR1 comprising an amino acid sequence having at least 90% identity to SEQ ID NO:286; a CDR2 comprising an amino acid sequence having at least 90% identity to SEQ ID NO:287; and/or a CDR3 comprising an amino acid sequence having at least 90% identity to SEQ ID NO:288. In some aspects, the VHs of the antigen binding polypeptide complex specifically binding to a TAA or an immune stimulatory receptor comprise a CDR1 comprising an amino acid sequence having at least 90% identity to SEQ ID NO:310; a CDR2 comprising an amino acid sequence having at least 90% identity to SEQ ID NO:312; and/or a CDR3 comprising an amino acid sequence having at least 90% identity to SEQ ID NO:313. In some aspects, the VHs of the antigen binding polypeptide complex specifically binding to a TAA or an immune stimulatory receptor comprise a CDR1 comprising an amino acid sequence having at least 90% identity to SEQ ID NO:318; a CDR2 comprising an amino acid sequence having at least 90% identity to SEQ ID NO:319; and/or a CDR3 comprising an amino acid sequence having at least 90% identity to SEQ ID NO:320.

In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:47 or 49, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:46 or 48. In some aspects, the VLs of the antigen binding polypeptide complex specifically binding to a TAA or an immune stimulatory receptor comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:47, and/or the VHs specifically binding to a TAA or an immune stimulatory receptor comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:46. In some aspects, the VLs of the antigen binding polypeptide complex specifically binding to a TAA or an immune stimulatory receptor comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:49, and/or the VHs specifically binding to a TAA or an immune stimulatory receptor comprise an amino acid sequence having at least 80% identity (such as at least 85%, at least 90%, at least 91%, at least 92%, at least 93%, at least 94%, at least 95%, at least 96%, at least 97%, at least 98%, at least 99% or 100% identity) to SEQ ID NO:48. In some aspects, the VLs of the antigen binding polypeptide complex specifically binding to a TAA or an immune stimulatory receptor comprise the amino acid sequence of SEQ ID NO:47, and/or the VHs specifically binding to a TAA or an immune stimulatory receptor comprise the amino acid sequence of SEQ ID NO:46. In some aspects, the VLs of the antigen binding polypeptide complex specifically binding to a TAA or an immune stimulatory receptor comprise the amino acid sequence of SEQ ID NO:49, and/or the VHs specifically binding to a TAA or an immune stimulatory receptor comprise the amino acid sequence of SEQ ID NO:48. In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:273, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:269. In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:281, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:277. In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:289, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:285. In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:313, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:309. In some aspects, the VLs of the antigen binding polypeptide complex specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:321, and/or the VHs specifically binding to a TAA or an immune stimulatory receptor comprise an amino acid sequence having at least 80%, at least 85%, at least 90%, at least 91%, at least 92%, at least 93%, at least 94%, at least 95%, at least 96%, at least 97%, at least 98%, at least 99%, or 100% identity to SEQ ID NO:317.

For the avoidance of doubt, all the antigen binding polypeptide complex structures described herein can be combined with any one or more of the targets described herein. Any and all disclosure herein in relation to targets for antigen binding polypeptide complexes of the invention is generally applicable, and applies equally and without reservation to each and every antigen binding polypeptide complex described herein. For the avoidance of doubt, the VL1, VL2, VL3, VL4, VH1, VH2, VH3, and/or VH4 of each and every antigen binding polypeptide complex described herein may independently bind to any one of said particularly preferred targets.

In some aspects, the TNF1, TNF2 and TNF3 of an antigen binding polypeptide complex structure described herein are each an extracellular domain of a TNFSF ligand. In some aspects, TNF1, TNF2 and TNF3 are selected from the group consisting of OX40L (TNFSF4), 4-1BBL (TNFSF9), TNF, TNF-related apoptosis inducing ligand (TRAIL), CD40L (TNFSF5), CD27L (TNFSF7), CD30L (TNFSF8), FasL (TNFSF6), EDAM, LTA (TNFSF1), LTB (TNFSF3), CD153 (TNFSF8), RANKL (TNFSF11), TWEAK (TNFSF12), APRIL (TNFSF13), BAFF (TNFSF13B), LIGHT (TNFSF14), VEGI (TNFSF15), and GITRL (TNFSF18). In some aspects, TNF1, TNF2 and TNF3 are each OX40L or 4-1BBL. In some aspects, TNF1, TNF2 and TNF3 are each OX40L. In some aspects, TNF1, TNF2 and TNF3 are each 4-1BBL. In some aspects, TNF1, TNF2 and TNF3 are OX40L, OX40L and 4-1BBL, respectively. In some aspects, TNF1, TNF2 and TNF3 are OX40L, 4-1BBL, and 4-1BBL respectively. In some aspects, TNF1, TNF2 and TNF3 are OX40L, 4-1BBL and OX40L, respectively. In some aspects, TNF1, TNF2 and TNF3 are 4-1BBL, OX40L and OX40L, respectively. In some aspects, TNF1, TNF2 and TNF3 are 4-1BBL, OX40L and 4-1BBL, respectively. In some aspects, TNF1, TNF2 and TNF3 are 4-1BBL, 4-1BBL and OX40L respectively. Exemplary OX40L and 4-1BBL sequences that can be used are known and described further herein.

In some aspects, an antigen binding polypeptide complex described herein comprises a polypeptide comprising an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 178, 180, and 188-228. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:58. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:64. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:118. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:120. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:176. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:178. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:180. In some aspects, the antigen binding complex comprises a polypeptide comprising the amino acid sequence of SEQ ID NO:58. In some aspects, the antigen binding complex comprises a polypeptide comprising the amino acid sequence of SEQ ID NO:64. In some aspects, the antigen binding complex comprises a polypeptide comprising the amino acid sequence of SEQ ID NO:118. In some aspects, the antigen binding complex comprises a polypeptide comprising the amino acid sequence of SEQ ID NO:120. In some aspects, the antigen binding complex comprises a polypeptide comprising the amino acid sequence of SEQ ID NO:176. In some aspects, the antigen binding complex comprises a polypeptide comprising the amino acid sequence of SEQ ID NO:178. In some aspects, the antigen binding complex comprises a polypeptide comprising the amino acid sequence of SEQ ID NO:180.

In some aspects, the antigen binding polypeptide complex comprises a heavy chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178. In some aspects, the heavy chain comprises the amino acid sequence of SEQ ID NO:178.

In some aspects, the antigen binding polypeptide complex comprises a heavy chain that specifically binds to CD3 and a light chain that specifically binds to CD3. In some aspects, the heavy chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:178; and the light chain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:176. In some aspects, the heavy chain comprises the amino acid sequence of SEQ ID NO:178; and the light chain comprises the amino acid sequence of SEQ ID NO:176.

In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:58, and a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:64. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:58, and a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:178. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:118, and a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:120. In some aspects, the antigen binding complex comprises a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:118, and a polypeptide comprising an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:180.

In some aspects, an antigen binding polypeptide complex described herein comprises a polypeptide comprising an amino acid sequence encoded by a polynucleotide having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 179, 181 and 229-268. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:59. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:65. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:119. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:121. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:177. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:179. In some aspects, the antigen binding complex comprises a polypeptide comprising an amino acid sequence encoded by a polynucleotide having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO:181. In some aspects, the antigen binding complex comprises a polypeptide comprising an amino acid sequence encoded by the polynucleotide of SEQ ID NO:59. In some aspects, the antigen binding complex comprises a polypeptide comprising an amino acid sequence encoded by the polynucleotide of SEQ ID NO:65. In some aspects, the antigen binding complex comprises a polypeptide comprising an amino acid sequence encoded by the polynucleotide of SEQ ID NO:119. In some aspects, the antigen binding complex comprises a polypeptide comprising an amino acid sequence encoded by the polynucleotide of SEQ ID NO:121. In some aspects, the antigen binding complex comprises a polypeptide comprising an amino acid sequence encoded by the polynucleotide of SEQ ID NO:177. In some aspects, the antigen binding complex comprises a polypeptide comprising an amino acid sequence encoded by the polynucleotide of SEQ ID NO:179. In some aspects, the antigen binding complex comprises a polypeptide comprising an amino acid sequence encoded by the polynucleotide of SEQ ID NO:181.

Molecular biology and recombinant DNA methods for making, screening and engineering antigen binding complexes and antibodies containing such sequences are well known and described, for example, in Adair et al. Human Antibodies, 5(1-2):41-47, 1994; Kostelny et al., J Immunol, 148(5):1547-1553 (1992), Shiraiwa et al., Methods, 154:10-20, 2019; and Zola, “Monoclonal Antibodies: A Manual of Techniques,” 1987, 1^{st} Ed., CRC Press; and Steinitz, Human Antibodies, 18(1-2):1-10, 2009.

In some aspects, an antigen binding polypeptide complex (e.g., an antibody or antigen binding fragment thereof) of the disclosure comprises an immunoglobulin hinge. In some aspects, the immunoglobulin hinge comprises an upper hinge region, a middle hinge region, a lower hinge region, or a combination thereof.

As used herein, an antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49(Pt 1): 37-60, Bricogne G (1997) Meth Enzymol 276A: 361-423, ed Carter C W, and Roversi et al., (2000) Acta Crystallogr D Biol Crystallogr 56(Pt 10): 1316-1323 (X-ray diffraction crystallography studies); and Champe et al., (1995) J Biol Chem 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet BLI, BIAcore® or KinExA.

Accordingly, in some aspects, an antigen binding polypeptide complex of the invention is an antibody or antigen binding fragment thereof.

In some aspects, the antibody or antigen binding fragment thereof specifically binds to an antigen with an equilibrium dissociation constant (K D) of from about 10 μM to about 1 pM. In some aspects, the antibody is IgG, IgM, IgE, IgA or IgD. For example, the antibody may be IgG. For example, the antibody may be IgM. For example, the antibody may be IgE. For example, the antibody may be IgA. For example, the antibody may be IgD. In some aspects, the IgG is IgG1, IgG2, IgG3 or IgG4. For example, the antibody may be IgG1. For example, the antibody may be IgG2. For example, the antibody may be IgG3. For example, the antibody may be IgG4. In some aspects, the antigen binding fragment is a Fab, scFab, Fab′, F(ab′)₂, Fv or scFv. For example, the antigen binding fragment may be a Fab. For example, the antigen binding fragment may be a scFab. For example, the antigen binding fragment may be a Fab′. For example, the antigen binding fragment may be a F(ab′)2. For example, the antigen binding fragment may be a Fv. For example, the antigen binding fragment may be a scFv. In some aspect, the antibody is human or humanized. For example, the antibody may be human. For the example, the antibody may be humanized.

In some aspects, an antigen binding polypeptide complex of the invention (e.g., an antibody or antigen binding fragment thereof) is bivalent, trivalent, tetravalent, pentavalent or hexavalent.

### III. Amino Acid Linkers

In some aspects, an antigen binding polypeptide complex (e.g., an antibody or antigen binding fragment thereof) of the invention comprises one or more amino acid linkers between one or more regions of the antigen binding polypeptide complex.

As used herein, an “amino acid linker” refers to a single amino acid or short amino acid sequence that is capable of joining two polypeptide regions of the invention described herein in a stable manner that maintains or promotes a function associated with the polypeptide regions. In some aspects, an amino acid linker is represented herein in a structure of an antigen binding polypeptide complex by the abbreviation “1” or “L” and a number (e.g., L1 to denote a first linker, L2 to denote a second linker, L3 to denote a third linker, L4 to denote a fourth linker, L5 to denote a fifth linker, L6 to denote a sixth linker, L7 to denote a seventh linker, L8 to denote an eighth linker, and so on). In some aspects, such enumerated amino acid linkers (e.g., L1) can have the same or different sequence as any other enumerated amino acid linker (e.g., L2, etc.). Furthermore, in some aspects, an enumerated amino acid linker present in one polypeptide (e.g., L1 on a first polypeptide of an antigen binding polypeptide complex structure described herein) can have the same or different sequence as the same enumerated amino acid linker present in another polypeptide (e.g., L1 on a second polypeptide, third polypeptide, etc. of an antigen binding polypeptide complex structure described herein).

In some aspects, an amino acid linker has a length of from 0 amino acids (i.e., an amino acid linker is not present) to about 50 amino acids (e.g., one or more of L1, L2, L3, L4, L5, L6, L7, L8, L9, L10, L11, L12, L13, L14, L15, L16, L17, L18, L19, L20, etc. to L96 or more of a first, second, third, fourth, etc. polypeptide of an antigen binding polypeptide complex structure described herein). In some aspects, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids.

In some aspects, the amino acid linker has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, L6, L7, L8, L9, L10, L11, L12, L13, L14, L15, L16, L17, L18, L19, L20, etc. to L96 or more of a first, second, third, fourth, etc. polypeptide of an antigen binding polypeptide complex structure described herein).

In some aspects, the amino acid linker consists of one or more amino acid residues (e.g., one or more of L1, L2, L3, L4, L5, L6, L7, L8, L9, L10, L11, L12, L13, L14, L15, L16, L17, L18, L19, L20, etc. to L96 or more of a first, second, third, fourth, etc. polypeptide of an antigen binding polypeptide complex structure described herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker of the invention is non-immunogenic. In some aspects, the non-immunogenic linker consists of serine, glycine and/or alanine residues, or consists of serine and/or glycine residues. In some aspects, an amino acid linker of the invention does not contain a T cell epitope or consensus T cell epitope.

In some aspects, the amino acid linker consists of one or more residues of alanine, cysteine, glycine, isoleucine, leucine, methionine, phenylalanine, proline, tryptophan, tyrosine, valine (e.g., one or more of L1, L2, L3, L4, L5, L6, L7, L8, L9, L10, L11, L12, L13, L14, L15, L16, L17, L18, L19, L20, etc. to L96 or more of a first, second, third, fourth, etc. polypeptide of an antigen binding polypeptide complex structure described herein).

Amino acid linker sequences that can be used with the antigen binding polypeptide complexes (e.g., an antibody or antigen binding fragment thereof) of the invention are well known and can be incorporated into antigen binding polypeptide complexes of the invention using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In some aspects, the amino acid linker (e.g., one or more of L1, L2, L3, L4, L5, L6, L7, L8, L9, L10, L11, L12, L13, L14, L15, L16, L17, L18, L19, L20, etc. to L96 or more of a first, second, third, fourth, etc. polypeptide of an antigen binding polypeptide complex structure described herein) has the sequence of any one of SEQ ID NOs:3-10 and 148-175 or a sequence having at least 50%, at least 60%, at least 70%, at least 80%, at least 90%, at least 91%, at least 92%, at least 93%, at least 94%, at least 95%, at least 96%, at least 97%, at least 98%, at least 99% or 100% identity to any one of SEQ ID NOs:3-10 and 148-175. In some aspects, the amino acid linker comprises or consists of the sequence of any one of SEQ ID NOs: 3-10 and 148-175. In some aspects, the amino acid linker comprises or consists of an amino acid sequence that is at least 80% (such as at least 85%, 90%, 91%, 92%, 93%, 94%, 95%, 96%, 97%, 98%, 99% or 100%) identical to SEQ ID NO: 10. In some aspects, the amino acid linker comprises or consists of the sequence of SEQ ID NO:10. In some aspects, the amino acid linker comprises or consists of an amino acid sequence that is at least 80% (such as at least 85%, 90%, 91%, 92%, 93%, 94%, 95%, 96%, 97%, 98%, 99% or 100%) identical to SEQ ID NO: 3. In some aspects, the amino acid linker comprises or consists of the sequence of SEQ ID NO:3.

In some aspects, the amino acid linker between a VH and VL in a polypeptide structure of an antigen binding polypeptide complex described herein is GGGGSGGSGSGGGGSASGSG (SEQ ID NO:168) or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:168. In some aspects, the amino acid linker between a VH and VL in a polypeptide structure of an antigen binding polypeptide complex comprises or consists of the sequence of SEQ ID NO:168.

In some aspects, the amino acid linker between a Fc and first extracellular domain of a TNFSF ligand in a polypeptide structure of an antigen binding polypeptide complex described herein is GGSGSGGGSGG (SEQ ID NO:149), GGSSGSGSGGSGSSG (SEQ ID NO:150), or GGSGSGGGSGLREGPELSPDDPAGLLDLRQG (SEQ ID NO:151) or a sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:149-151. In some aspects, the amino acid linker comprises or consists of an amino acid sequence that is at least 80% (such as at least 85%, 90%, 91%, 92%, 93%, 94%, 95%, 96%, 97%, 98%, 99% or 100%) identical to SEQ ID NO: 149. In some aspects, the amino acid linker comprises or consists of an amino acid sequence that is at least 80% (such as at least 85%, 90%, 91%, 92%, 93%, 94%, 95%, 96%, 97%, 98%, 99% or 100%) identical to SEQ ID NO: 150. In some aspects, the amino acid linker comprises or consists of an amino acid sequence that is at least 80% (such as at least 85%, 90%, 91%, 92%, 93%, 94%, 95%, 96%, 97%, 98%, 99% or 100%) identical to SEQ ID NO: 151. In some aspects, the amino acid linker comprises or consists of the sequence of SEQ ID NO:149. In some aspects, the amino acid linker comprises or consists of the sequence of SEQ ID NO:150. In some aspects, the amino acid linker comprises or consists of the sequence of SEQ ID NO:151.

In some aspects, the linker between a VH and Fc or between a CH1 and Fc in a polypeptide structure of an antigen binding polypeptide complex described herein is ASGGSG (SEQ ID NO:161) or a sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:161. In some aspects, the amino acid linker comprises or consists of the sequence of SEQ ID NO:161.

### IV. Detectable Labels and Drug Conjugates

In some aspects, an antigen binding polypeptide complex (e.g., an antibody or antigen binding fragment thereof) of the invention comprises one or more detectable labels. An antigen binding polypeptide complex (e.g., an antibody or antigen binding fragment thereof) containing a detectable label is useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof.

In some aspects, the detectable label is a peptide tag. In some aspects, the peptide tag is located at the N-terminus of the polypeptide or polypeptide complex. In some aspects, the peptide tag is located at the C-terminus of the polypeptide or polypeptide complex. In some aspects, the peptide tag is an affinity tag or fusion tag.

In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73:Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

In some aspects, an antigen binding polypeptide complex (e.g., an antibody or antigen binding fragment thereof) of the invention is conjugated to an agent as an antibody-drug conjugate (ADC). An ADC of the invention is useful in therapeutic, diagnostic, imaging (e.g., radioimaging), or basic research applications.

In some aspects, an antigen binding polypeptide complex (e.g., an antibody or antigen binding fragment thereof) of the invention is conjugated to a cytotoxic agent, immunomodulating agent, imaging agent, or therapeutic protein, typically via a linker. The linker can comprise a cleavable unit or can be non-cleavable. Cleavable units include, for example, disulfide containing linkers that are cleavable through disulfide exchange, acid-labile linkers that are cleavable at acidic pH, and linkers that are cleavable by hydrolases, esterases, peptidases, and glucoronidases (e.g., peptide linkers and glucoronide linkers). Non-cleavable linkers are believed to release drug via a proteolytic antibody degradation mechanism.

Methods for making an ADC are known and include, but are not limited to, conjugation via thiols, amides, aldehydes, or azides, as well as other routine chemical, molecular biology and recombinant DNA techniques. See, e.g., Yao et al., Int. J. Mol. Sci., 17(2):194, 2016; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1):DOI: 10/1080/23312025.2019.1665406.

### V. Modifications

In some aspects, the invention is directed to an antigen binding polypeptide complex (e.g., an antibody or antigen binding fragment thereof) comprising an effector function mutation or half-life extension mutation.

Effector functions are an important part of the humoral immune response and form an link between innate and adaptive immunity. Most effector functions are induced via the Fc region of an antibody, which can interact with complement proteins and specialized Fc receptors. As used herein, an “effector function mutation” refers to a change in the amino acid sequence, typically in the Fc region, which increases or decreases effector function, for example, increasing binding affinity of Fc for specific Fc receptors, or increasing antibody-dependent cellular cytotoxicity (ADCC) activity.

“Half-life” of a pharmaceutically active substance is the time it takes for the amount of the substance, once administered to the body, to reduce by half. A “half-life extension mutation” of an antigen binding polypeptide complex of the invention refers to a change in the amino acid sequence, typically in the Fc region, which increases the half-life of the antigen binding polypeptide complex (e.g., by increasing Fc receptor binding affinity, slowing off-rate for Fc and Fc receptors, and/or increased sialylation).

Examples of effector function mutations that increase function include, but are not limited to, the following substitutions in the Fc region, based on the EU numbering scheme: S298A/E333A/K334A, S239D/I332E, S239D/A330L/I332E, and G236A/S239D/I332E. Examples of effector function mutations that decrease function include, but are not limited to, the following substitutions in the Fc region, based on the EU numbering scheme: N297A and L234A/L235A. Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019.

In some aspects, the invention is directed to an antigen binding polypeptide complex (e.g., an antibody or antigen binding fragment thereof) comprising one or more knob-into-hole modifications.

The term “knob-into-hole modification” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In some aspects, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Protein Science 6:781-788, 1987.

In some aspects, the antigen binding polypeptide complex comprises one, two, three, four, five, six, seven, eight, nine, ten, or more knob-into-hole modifications.

Knob-into-hole modifications are well known and can be incorporated into the antigen binding polypeptide complexes of the invention using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998.

In some aspects, the knob-into-hole modification is an amino acid substitution. As used herein, such a substitution is described based on the EU numbering scheme of Kabat, which corresponds to the numbering in the Protein Data Bank (PDB).

In some aspects, the knob-into-hole modification is a knob substitution of S354C and/or T366W, based on the EU numbering scheme.

In some aspects, the knob-into-hole modification is a hole substitution of Y349C, T366S, L368A, Y407V, L234A, L235A, P329A, M428L, N433S, M252Y, S254T, T256E, or any combination thereof, based on the EU numbering scheme.

In some aspects, the knob-into-hole modifications are hole substitutions of Y349C, T366S, L368A and Y407V, based on the EU numbering scheme. In some aspects, the knob-into-hole modifications are a hole substitutions of L234A, L235A and P329A, based on the EU numbering scheme. In some aspects, the knob-into-hole modifications are hole substitutions of L234A and L235A, based on the EU numbering scheme. In some aspects, the knob-into-hole modifications are hole substitutions of M428L and N433S, based on the EU numbering scheme. In some aspects, the knob-into-hole modifications are hole substitutions of M252Y, S254T and T256E, based on the EU numbering scheme.

In some aspects, an antigen binding polypeptide complex is an IgG1 or IgG4 antibody and the knob-into-hole modifications are knob substitutions of S354C and T366W and hole substitutions of Y349C, T366S, L368A and Y407V.

In some aspects, the antigen binding polypeptide complex is an IgG1 or IgG4 antibody and the knob-into-hole modifications are hole substitutions of L234A, L235A and P329A.

In some aspects, the antigen binding polypeptide complex is an IgG1 or IgG4 antibody and the knob-into-hole modifications are hole substitutions of L234A and L235A.

In some aspects, the antigen binding polypeptide complex is an IgG1 or IgG4 antibody and the knob-into-hole modifications are hole substitutions of M428L and N433S.

In some aspects, the antigen binding polypeptide complex is an IgG1 or IgG4 antibody and the knob-into-hole modifications are hole substitutions of M252Y, S254T and T256E.

### VI. Polypeptides, Polynucleotides, Vectors, Cells, and Protein Production Methods

In some aspects, the invention is directed to a polypeptide encoding a portion of an antigen binding polypeptide complex (e.g., an antibody or antigen binding fragment thereof) described herein.

In some aspects, the invention is directed to a polypeptide comprising or consisting of an amino acid sequence of one or more of SEQ ID NOs:1-228 and 269-324 or an amino acid sequence having at least 80%, at least 85%, at least 90%, at least 91%, at least 92%, at least 93%, at least 94%, at least 95%, at least 96%, at least 97%, at least 98%, or at least 99% identity to one or more of SEQ ID NOs:1-228 and 269-324. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 58. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 64. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 118. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 120. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 176. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 178. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 180. In some aspects, the polypeptide comprises or consists of the amino acid sequence of one or more of SEQ ID NOs:1-228 and 269-324. In some aspects, the polypeptide comprises or consists of the amino acid sequence of SEQ ID NO: 58. In some aspects, the polypeptide comprises or consists of the amino acid sequence of SEQ ID NO:64. In some aspects, the polypeptide comprises or consists of the amino acid sequence of SEQ ID NO:118. In some aspects, the polypeptide comprises or consists of the amino acid sequence of SEQ ID NO:120. In some aspects, the polypeptide comprises or consists of the amino acid sequence of SEQ ID NO:176. In some aspects, the polypeptide comprises or consists of the amino acid sequence of SEQ ID NO:178. In some aspects, the polypeptide comprises or consists of the amino acid sequence of SEQ ID NO:180.

In some aspects, the invention is directed to a polypeptide comprising or consisting of an amino acid sequence of one or more of SEQ ID NOs:12-18, 58-147, 178, 180, 188-228 and 269-324 or an amino acid sequence having at least 80%, at least 85%, at least 90%, at least 91%, at least 92%, at least 93%, at least 94%, at least 95%, at least 96%, at least 97%, at least 98%, or at least 99% identity to one or more of SEQ ID NOs:12-18, 58-147, 178 and 180. In some aspects, the polypeptide comprises or consists of the amino acid sequence of one or more of SEQ ID NOs:12-18, 58-147, 178, 180, 188-228 and 269-324.

In some aspects, the invention is directed to a polypeptide comprising or consisting of an amino acid sequence of one or more of SEQ ID NOs:58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 178, 180, 188-228 and 269-324 or an amino acid sequence having at least 80%, at least 85%, at least 90%, at least 91%, at least 92%, at least 93%, at least 94%, at least 95%, at least 96%, at least 97%, at least 98% or at least 99% identity to one or more of SEQ ID NOs:58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 178, 180, 188-228 and 269-324. In some aspects, the polypeptide comprises or consists of the amino acid sequence of one or more of SEQ ID NOs:58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 178, 180, 188-228 and 269-324.

In some aspects, the invention is directed to a polynucleotide encoding a portion of an antigen binding polypeptide complex (e.g., an antibody or antigen binding fragment thereof) described herein. As used herein, a “polynucleotide” includes DNA and RNA (e.g., mRNA).

In some aspects, the invention is directed to a polynucleotide comprising or consisting of a polynucleotide sequence of one or more of SEQ ID NOs:1-175 and 229-268, or a polynucleotide having at least 90%, at least 91%, at least 92%, at least 93%, at least 94%, at least 95%, at least 96%, at least 97%, at least 98% or at least 99% identity to one or more of SEQ ID NOs:1-175 and 229-268. In some aspects, the invention is directed to a polynucleotide comprising the polynucleotide sequence of one or more of SEQ ID NOs:1-175 and 229-268. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 59. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 65. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 119. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 121. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 177. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 179. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 181. In some aspects, the polynucleotide comprises or consists of the polynucleotide sequence of SEQ ID NO: 59. In some aspects, the polynucleotide comprises or consists of the polynucleotide sequence of SEQ ID NO: 65. In some aspects, the polynucleotide comprises or consists of the polynucleotide sequence of SEQ ID NO: 119. In some aspects, the polynucleotide comprises or consists of the polynucleotide sequence of SEQ ID NO: 121. In some aspects, the polynucleotide comprises or consists of the polynucleotide sequence of SEQ ID NO: 177. In some aspects, the polynucleotide comprises or consists of the polynucleotide sequence of SEQ ID NO: 179. In some aspects, the polynucleotide comprises or consists of the polynucleotide sequence of SEQ ID NO: 181.

In some aspects, the invention is directed to a polynucleotide comprising or consisting of a polynucleotide sequence of one or more of SEQ ID NOs:59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 179, 181, and 229-268, or a polynucleotide having at least 90%, at least 91%, at least 92%, at least 93%, at least 94%, at least 95%, at least 96%, at least 97%, at least 98% or at least 99% identity to one or more of SEQ ID NOs:59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 179, 181 and 229-268. In some aspects, the invention is directed to a polynucleotide comprising or consisting of the polynucleotide sequence of one or more of SEQ ID NOs:59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 179, 181 nad 229-268.

In some aspects, the invention is directed to a vector comprising one or more polynucleotides described herein.

In yet some aspects, the invention is directed to a host cell comprising one or more polynucleotides and/or vectors described herein.

As used herein, the term “host cell” can be any type of cell, e.g., a primary cell, a cell in culture, or a cell from a cell line. In some aspects, the term “host cell” refers to a cell containing a foreign gene [e.g., a cell subjected to gene delivery or transfected with a polynucleotide (e.g., DNA or mRNA) encoding the gene] and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

Methods which are well known to those skilled in the art can be used to construct vectors encoding antigen binding polypeptide complexes (e.g., CDR, VH, VL, scFv, Fab, scFab, heavy chain or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cells can then be cultured by conventional techniques to produce an antigen binding polypeptide complex comprising, e.g., six CDRs, VH, VL, scFv, Fab, scFab, heavy chain or light chain, or a domain thereof. Thus, provided herein are host cells containing a polynucleotide encoding an antigen binding polypeptide complex comprising, e.g., comprising six CDRs, VH, VL, scFv, Fab, scFab, heavy chain or light chain, or a domain thereof, operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes described herein. Such host-vector systems represent vehicles by which the coding sequences of interest can be produced and subsequently purified, but also represent cells which can, when transformed or transfected with the appropriate nucleotide coding sequences, express a polypeptide or polypeptide complex described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes described herein are CHO cells, for example CHO cells from the CHO GS System™ (Lonza). In some aspects, cells for expressing polypeptides or polypeptide complexes of the invention are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, polypeptides or polypeptide complexes described herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex described herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes described herein can be fused to heterologous polypeptide sequences described herein (e.g., tags) or otherwise known in the art to facilitate purification.

In some aspects, a polypeptide or polypeptide complex described herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex described herein is substantially free of cellular material and/or chemical precursors.

### VII. Pharmaceutical Compositions and Kits

In some aspects, the invention is directed to a pharmaceutical composition comprising an antigen binding polypeptide complex (e.g., antibody or antigen binding fragment thereof), polypeptide(s), polynucleotide(s), vector(s), or cell(s) described herein.

In some aspects, the invention is directed to a pharmaceutical composition comprising (1) an antigen binding polypeptide complex (e.g., antibody or antigen binding fragment thereof), polynucleotide, vector, or cell described herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions of the invention. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

In some aspects, the invention is directed to a kit comprising an antigen binding polypeptide complex (e.g., antibody or antigen binding fragment thereof), polypeptide(s), polynucleotide(s), vector(s), cell(s), or pharmaceutical composition described herein, or a combination thereof, optionally with instructions for use. In some aspects, the invention provides kits for producing a single-dose administration unit. In some aspects, the kits of the invention can contain both a first container having a dried protein and a second container having an aqueous formulation. In some aspects, kits containing single and multi-chambered pre-filled syringes (e.g., liquid syringes and lyosyringes) are also provided. In some aspects, the kit contains components for intravenous or subcutaneous administration.

### VIII. Methods of Use

In some aspects, the invention is directed to certain methods of use of an antigen binding polypeptide complex (e.g., an antibody or antigen binding fragment thereof), polypeptide(s), polynucleotide(s), vector(s), cell(s), or pharmaceutical composition described herein, or a combination thereof. Any of the antigen binding polypeptide complex structures described herein targeting one or more of the targets described herein may be used in any of the methods and uses of the invention.

In some aspects, the invention is directed to a method of treating or preventing a disease or condition, comprising administering to a subject in need thereof an antigen binding polypeptide complex (e.g., antibody or antigen binding fragment thereof), polypeptide, polynucleotide, vector, cell, or pharmaceutical composition described herein, or a combination thereof. In some aspects, the invention is directed to a method of treating or preventing a disease or condition, comprising administering to a subject in need thereof a therapeutically effective amount of an antigen binding polypeptide complex (e.g., antibody or antigen binding fragment thereof), polypeptide, polynucleotide, vector, cell, or pharmaceutical composition described herein, or a combination thereof. The present invention further provides an antigen binding polypeptide complex (e.g., antibody or antigen binding fragment thereof), polypeptide, polynucleotide, vector, cell, or pharmaceutical composition described herein, or a combination thereof, for use in treating or preventing a disease or condition in a subject. The present invention further provides the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a disease or condition in a subject. Said treatment may comprise or consist of inducing or enhancing an immune response in the subject. Thus, the present invention provides an antigen binding polypeptide complex (e.g., antibody or antigen binding fragment thereof), polypeptide, polynucleotide, vector, cell, or pharmaceutical composition described herein, or a combination thereof, for use in inducing or enhancing an immune response in a subject to treat or prevent a disease or condition in said subject. The present invention further provides the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the induction or enhancement of an immune response in a subject to treat or prevent a disease or condition in said subject.

In some aspects, the invention is directed to a method for inducing or enhancing an immune response comprising administering to a subject in need thereof an antigen binding polypeptide complex, antibody or antigen binding fragment thereof, polypeptide, polynucleotide, vector, cell, or pharmaceutical composition described herein. The present invention further provides an antigen binding polypeptide complex (e.g., antibody or antigen binding fragment thereof), polypeptide, polynucleotide, vector, cell, or pharmaceutical composition described herein, or a combination thereof, for use in inducing or enhancing an immune response in a subject. The present invention further provides the use of an antigen binding polypeptide complex (e.g., antibody or antigen binding fragment thereof), polypeptide, polynucleotide, vector, cell, or pharmaceutical composition described herein, or a combination thereof in the manufacture of a medicament for inducing or enhancing an immune response in a subject.

As used herein, “inducing or enhancing an immune response” includes, but is not limited to increasing activation, proliferation, differentiation, and/or maturation of immune cells (e.g., lymphocytes (T cells, B cells and/or NK cells), neutrophils, and/or monocytes/macrophages). Thus, “inducing or enhancing an immune response” may be useful in the treatment or prevention of diseases where an induced or enhanced immune response has potential clinical benefit, such as in the treatment of cancer or other diseases or disorders as described herein. Antigen binding polypeptide complexes of the disclosure can be used to modulate the magnitude, duration, and/or quality of an immune response to a tumor-associated antigen (TAA) or cancer. Enhanced immune cell activation, proliferation, differentiation, and/or maturation of immune cells can be determined by routine assays, such as T cell proliferation and binding assays described herein.

In some aspects, the method enhances the production of antibodies that recognize a TAA or cancer. Enhanced antibody production can be determined by routine assays such as detecting increased antibody levels in a subject treated with an antigen binding polypeptide complex of the disclosure as compared to antibody levels in a subject not receiving the antigen binding polypeptide complex.

The methods of the invention can be used to modulate or enhance the immune response both prophylactically and therapeutically.

Accordingly, in some aspects, the invention is directed to a method for overcoming cancer-mediated immune suppression, comprising administering to a subject in need thereof an antigen binding polypeptide complex, antibody or antigen binding fragment thereof, polypeptide, polynucleotide, vector, cell, or pharmaceutical composition described herein.

In some aspects, the invention is directed to a method of treating cancer comprising administering to a subject in need thereof an antigen binding polypeptide complex (e.g., antibody or antigen binding fragment thereof), polypeptide, polynucleotide, vector, cell, or pharmaceutical composition described herein, or a combination thereof. The present invention further provides an antigen binding polypeptide complex (e.g., antibody or antigen binding fragment thereof), polypeptide, polynucleotide, vector, cell, or pharmaceutical composition described herein, or a combination thereof, for use in treating or preventing a cancer in a subject. The present invention further provides the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a cancer in a subject. Said treatment may comprise or consist of “inducing or enhancing an immune response” in a subject, as described herein. As used herein, the term “subject” means a human or a non-human mammal, e.g., a dog, a cat, a mouse, a rat, a cow, a sheep, a pig, a goat, a non-human primate or a bird, e.g., a chicken, as well as any other vertebrate or invertebrate. In some aspects, the subject is a human. In some aspects, the subject is a veterinary animal. In some aspects, the subject is a mammal

TABLE 1
Antibody Sequences

| Antibody | Peptide SEQ ID NOS (DNA SEQ ID NOS) |
|---|---|
| MX169 | 54 (55), 58 (59), 104 (105) |
| MX170 | 54 (55), 72 (73), 98 (99) |
| MX306 | 54 (55), 118 (119), 120 (121) |
| MX318 | 54 (55), 120 (121) |
| MX368 | 132 (133), 134 (135) |
| MX369 | 54 (55), 132 (133) |
| MX424 | 140 (141), 142 (143) |
| MX425 | 118 (119), 140 (141) |
| MX485 | 193, 189 |
| MX487 | 193 |
| MX620 | 193, 208 |
| MX622 | 208, 209 |

TABLE 2
Plasmid Sequences

| Plasmid | Description | Protein SEQ ID NO | DNA SEQ ID NO |
|---|---|---|---|
| MD668 | ScFab_CD28_Kcc_4-1BBL LALAPA | 189 | 229 |
| MD703 | ScFab_CD3_Glo_Hco_hOx40L LALAPA | 190 | 230 |
| MD704 | ScFab_CD3_Glo_Hcc_h4-1BBL LALAPA | 191 | 231 |
| MD705 | ScFab_CD3_SAR_Hoc_hOx40L LALAPA | 192 | 232 |
| MD706 | ScFab_CD3_SAR_Hcc_h4-1BBL LALAPA | 193 | 233 |
| MD747 | ahTrop2_h20xCD3_hOKT_Kcc LALAPA | 194 | 234 |
| MD748 | ahTrop2_h20xCD3_hOKT_Kcc_41BBLrw8 LALAPA | 195 | 235 |
| MD749 | ahcMetxahCD3_hoKT_Kcc_LALAPA | 196 | 236 |
| MD750 | ahcMetxahCD3_hOKTKcc_LALAPA_4-1BBL | 197 | 237 |
| MD751 | ahcMetxahTrop2_h20_Kcc_41BBLrw8 LALAPA | 198 | 238 |
| MD752 | ahTrop2_h20xahcMet_Kcc_41BBLrw8 LALAPA | 199 | 239 |
| MD753 | ahTrop2_h20Hrw3_Hcc_41BBLrw8 LALAPA | 200 | 240 |
| MD754 | ahcMet_Hrw5_Hcc_41BBLrw8 LALAPA | 201 | 241 |
| MD778 | VRC01.23scFab_Kcc_4-1BBL LALAPA | 202 | 242 |
| MD779 | VRC01.23H_Kcc_4-1BBL LALAPA | 203 | 243 |
| MD780 | VRC01.23scFv_Kcc_hOx40L LALAPA | 204 | 244 |
| MD781 | VRC01.23scFy_Kcc_4-1BBL LALAPA | 205 | 245 |
| MD797 | ahCD20_hRitxahCD19_Kcc_41BBL LALAPA | 206 | 246 |
| MD798 | ahCD19xahCD20_hRit_Kcc_41BBL LALAPA | 207 | 247 |
| MD799 | ScFab_CD28_TGNcs_LALAPA_KCC | 208 | 248 |
| MD800 | ScFab_CD3_SAR_Hcc_LALAPA | 209 | 249 |
| MD885 | CD3_SAR x VRC01.23_Hcc_hOx40L LALAPA | 210 | 250 |
| MD886 | CD28 x VRC01.23_Kcc_hOx40L LALAPA | 211 | 251 |
| MD900 | VRC01.23H_Kcc_Ox40L LALAPA | 212 | 252 |
| MD901 | VRC01.23scFab_Kcc_Ox40L LALAPA | 213 | 253 |
| MD902 | ahCD19axahCD20PS_Kcc_41BBL LALAPA | 214 | 254 |
| MD903 | ahCD20_PSxahCD19_Kcc_41BBL LALAPA | 215 | 255 |
| MD918 | ScFab_CD3_hOKT_v23_Hcc_LALAPA | 216 | 256 |
| MD956 | CD20_PSxCD19 LALAPA_418BL | 217 | 257 |
| MD957 | CD19 x CD20_PS LALAPA_41BBL | 218 | 258 |
| MD958 | cMetxTrop2_h20_41BBL | 219 | 259 |
| MD959 | ahTrop2_h20xahcMet 41BBL | 220 | 260 |
| MD962 | ScFab_CD3_Hcc_mono4-1BBL8 | 221 | 261 |
| MD963 | ScFab_CD28_Kcc_mono4-1BBL8 | 222 | 262 |
| MD964 | Sofab_CD3_Hcc_mono4-18BL7 | 223 | 263 |
| MD965 | ScFab_CD28_Kcc_mono4-1BBL7 | 224 | 264 |
| MD966 | ahCD20_ofxahCD19_Kcc_41BBL LALAPA | 225 | 265 |
| MD967 | ahCD20_ofx ahCD19_41BBLrw8 | 226 | 266 |
| MD968 | ahCD19xahCD20_of_Kcc_41BBL LALAPA | 227 | 267 |
| MD969 | ahCD19xahCD20_of_41BBLrw8 | 228 | 268 |

Clauses Relating to Aspects of the Disclosure

1. An antigen binding polypeptide complex comprising a first polypeptide, a second polypeptide, and a third polypeptide;
wherein the first polypeptide has a structure represented by: VL1-L1-CL; VL1-L1-CH1; VH1-L1-CL; or VH1-L1-CH1;
wherein:
(i) the second polypeptide has a structure represented by: VH1-L2-CH1-L3-Fc-L4-TNF1-L5-TNF2-L6-TNF3; VH1-L2-CL-L3-Fc-L4-TNF1-L5-TNF2-L6-TNF3; VH1-L7-CH1-L8-Fe; VH1-L7-CL-L8-Fe; VL1-L2-CH1-L3-Fc-L4-TNF1-L5-TNF2-L6-TNF3; VL1-L2-CL-L3-Fe-L4-TNF1-L5-TNF2-L6-TNF3; VL1-L7-CH1-L8-Fc; or VL1-L7-CL-L8-Fc; and the third polypeptide has a structure represented by: VL2-L9-VH2-L10-Fc-L11-TNF1-L12-TNF2-L13-TNF3; or VH2-L14-VL2-L15-Fe-L16-TNF1-L17-TNF2-L18-TNF3; or
(ii) the second polypeptide has a structure represented by: VH1-L19-CH1-L20-Fc-L21-TNF1-L22-TNF2-L23-TNF3; VH1-L19-CL-L20-Fc-L21-TNF1-L22-TNF2-L23-TNF3; VL1-L19-CH1-L20-Fc-L21-TNF1-L22-TNF2-L23-TNF3; or VL1-L19-CL-L20-Fc-L21-TNF1-L22-TNF2-L23-TNF3; and the third polypeptide has a structure represented by: VL2-L24-VH2-L25-Fc; or VH2-L26-VL2-L27-Fc;
wherein: VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L27 are amino acid linkers.

2. An antigen binding polypeptide complex comprising a first polypeptide, a second polypeptide, and a third polypeptide;
wherein the first polypeptide has a structure represented by: VL1-L1-CL; VL1-L1-CH1; VH1-L1-CL; or VH1-L1-CH1;
wherein:
(i) the second polypeptide has a structure represented by: VH1-CH1-L2-Fc-L3-TNF1-L4-TNF2-L5-TNF3; VH1-CL-L2-Fc-L3-TNF1-L4-TNF2-L5-TNF3; VH1-CH1-L6-Fc; VH1-CL-L7-Fc; VL1-CH1-L2-Fc-L3-TNF1-L4-TNF2-L5-TNF3; VL1-CL-L2-Fc-L3-TNF1-L4-TNF2-L5-TNF3; VL1-CH1-L6-Fc; or VL1-CL-L7-Fc; and the third polypeptide has a structure represented by: VL2-L8-VH2-L9-Fc-L10-TNF1-L11-TNF2-L12-TNF3; or VH2-L13-VL2-L14-Fe-L15-TNF1-L16-TNF2-L17-TNF3; or
(ii) the second polypeptide has a structure represented by: VH1-CH1-L18-Fc-L19-TNF1-L20-TNF2-L21-TNF3; VH1-CL-L18-Fc-L19-TNF1-L20-TNF2-L21-TNF3; VL1-CH1-L18-Fc-L19-TNF1-L20-TNF2-L21-TNF3; or VL1-CL-L18-Fc-L19-TNF1-L20-TNF2-L21-TNF3; and the third polypeptide has a structure represented by: VL2-L22-VH2-L23-Fc; or VH2-L24-VL2-L25-Fc;
wherein: VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a tumor necrosis factor superfamily (TNFSF) ligand; TNF2 is a second extracellular domain of a TNFSF ligand; TNF3 is a third extracellular domain of a TNFSF ligand; and L1-L25 are amino acid linkers.

3. The antigen binding polypeptide complex of clause 1, wherein the first polypeptide has a structure represented by VL1-L1-CL; the second polypeptide has a structure represented by VH1-L2-CH1-L3-Fc-L4-TNF1-L5-TNF2-L6-TNF3; and the third polypeptide has a structure represented by VL2-L9-VH2-L10-Fc-L11-TNF1-L12-TNF2-L13-TNF3.

4. The antigen binding polypeptide complex of clause 2, wherein the first polypeptide has a structure represented by VL1-L1-CL; the second polypeptide has a structure represented by VH1-CH1-L2-Fc-L3-TNF1-L4-TNF2-L5-TNF3; and the third polypeptide has a structure represented by VL2-L8-VH2-L9-Fc-L10-TNF1-L11-TNF2-L12-TNF3.

5. An antigen binding polypeptide complex comprising a first polypeptide and a second polypeptide; wherein:
(i) the first polypeptide has a structure represented by: VL1-L1-VH1-L2-Fc; VH1-L3-VL1-L4-Fc; VL1-L5-VH1-L6-CL-L7-CH1-L8-Fc; VL1-L5-VH1-L6-CH1-L7-CL-L8-Fc; VH1-L5-VL1-L6-CL-L7-CH1-L8-Fc; VH1-L5-VL1-L6-CH1-L7-CL-L8-Fc; VL1-L9-CL-L10-VH1-L11-CH1-L12-Fc; VL1-L9-CH1-L10-VH1-L11-CL-L12-Fc; VH1-L9-CL-L10-VL1-L11-CH1-L12-Fc; VH1-L9-CH1-L10-VL1-L11-CL-L12-Fc; VL1-L13-VH1-L14-Fc-L15-TNF1-L16-TNF2-L17-TNF3; VH1-L18-VL1-L19-Fc-L20-TNF1-L21-TNF2-L22-TNF3; VL1-L23-VH1-L24-CL-L25-CH1-L26-Fc-L27-TNF1-L28-TNF2-L29-TNF3; VL1-L23-VH1-L24-CH1-L25-CL-L26-Fc-L27-TNF1-L28-TNF2-L29-TNF3; VH1-L23-VL1-L24-CL-L25-CH1-L26-Fc-L27-TNF1-L28-TNF2-L29-TNF3; VH1-L23-VL1-L24-CH1-L25-CL-L26-Fc-L27-TNF1-L28-TNF2-L29-TNF3; VL1-VH1-L14-Fc-L15-TNF1-L16-TNF2-L17-TNF3; VH1-VL1-L19-Fc-L20-TNF1-L21-TNF2-L22-TNF3; VL1-VH1-L24-CL-L25-CH1-L26-Fc-L27-TNF1-L28-TNF2-L29-TNF3; VL1-VH1-L24-CH1-L25-CL-L26-Fc-L27-TNF1-L28-TNF2-L29-TNF3; VH1-VL1-L24-CL-L25-CH1-L26-Fc-L27-TNF1-L28-TNF2-L29-TNF3; VH1-VL1-L24-CH1-L25-CL-L26-Fc-L27-TNF1-L28-TNF2-L29-TNF3; VL1-L30-CL-L31-VH1-L32-CH1-L33-Fc-L34-TNF1-L35-TNF2-L36-TNF3; VL1-L30-CH1-L31-VH1-L32-CL-L33-Fc-L34-TNF1-L35-TNF2-L36-TNF3; VH1-L30-CL-L31-VL1-L32-CH1-L33-Fc-L34-TNF1-L35-TNF2-L36-TNF3; or VH1-L30-CH1-L31-VL1-L32-CL-L33-Fc-L34-TNF1-L35-TNF2-L36-TNF3; and the second polypeptide has a structure represented by: VL2-L37-VH2-L38-Fc-L39-TNF1-L40-TNF2-L41-TNF3; VH2-L42-VL2-L43-Fc-L44-TNF1-L45-TNF2-L46-TNF3; VL2-L47-VH2-L48-CL-L49-CH1-L50-Fc-L51-TNF1-L52-TNF2-L53-TNF3; VL2-L54-CL-L55-VH2-L56-CH1-L57-Fc-L58-TNF1-L59-TNF2-L60-TNF3; VL2-L47-VH2-L48-CH1-L49-CL-L50-Fc-L51-TNF1-L52-TNF2-L53-TNF3; VL2-L54-CH1-L55-VH2-L56-CL-L57-Fc-L58-TNF1-L59-TNF2-L60-TNF3; VL2-VH2-L38-Fc-L39-TNF1-L40-TNF2-L41-TNF3; VH2-VL2-L43-Fc-L44-TNF1-L45-TNF2-L46-TNF3; VL2-VH2-L48-CL-L49-CH1-L50-Fc-L51-TNF1-L52-TNF2-L53-TNF3; VL2-CL-L55-VH2-L56-CH1-L57-Fc-L58-TNF1-L59-TNF2-L60-TNF3; VL2-VH2-L48-CH1-L49-CL-L50-Fc-L51-TNF1-L52-TNF2-L53-TNF3; or VL2-CH1-L55-VH2-L56-CL-L57-Fc-L58-TNF1-L59-TNF2-L60-TNF3; or
(ii) the first polypeptide has a structure represented by: VL1-L61-VH1-L62-Fc-L63-TNF1-L64-TNF2-L65-TNF3; VH1-L66-VL1-L67-Fc-L68-TNF1-L69-TNF2-L70-TNF3; VL1-L71-VH1-L72-CL-L73-CH1-L74-Fc-L75-TNF1-L76-TNF2-L77-TNF3; VL1-L71-VH1-L72-CH1-L73-CL-L74-Fc-L75-TNF1-L76-TNF2-L77-TNF3; VL1-L78-CL-L79-VH1-L80-CH1-L81-Fc-L82-TNF1-L83-TNF2-L84-TNF3; or VL1-L78-CH1-L79-VH1-L80-CL-L81-Fc-L82-TNF1-L83-TNF2-L84-TNF3; and the second polypeptide has a structure represented by: VL2-L85-VH2-L86-Fc; VH2-L87-VL2-L88-Fc; VL2-L89-VH2-L90-CL-L91-CH1-L92-Fc; VL2-L89-VH2-L90-CH1-L91-CL-L92-Fc; VL2-L93-CL-L94-VH2-L95-CH1-L96-Fc; or VL2-L93-CH1-L94-VH2-L95-CL-L96-Fc;
wherein: VL1 is a first immunoglobulin light chain variable region; VL2 is a second immunoglobulin light chain variable region; VH1 is a first immunoglobulin heavy chain variable region; VH2 is a second immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; TNF1 is a first extracellular domain of a TNFSF ligand; TNF2 is a second extracellular domain of a TNFSF ligand; TNF3 is a third extracellular domain of a TNFSF ligand; and L1-L96 are amino acid linkers.

6. The antigen binding polypeptide complex of clause 5, wherein the first polypeptide has a structure represented by VL1-L13-VH1-L14-Fc-L15-TNF1-L16-TNF2-L17-TNF3; and the second polypeptide has a structure represented by VL2-L37-VH2-L38-Fc-L39-TNF1-L40-TNF2-L41-TNF3.

7. An antigen binding polypeptide complex comprising a first polypeptide and a second polypeptide; wherein:
(i) the first polypeptide has a structure represented by: Fc; VL1-L1-VL2-L2-VH2-L3-VH1-L4-Fc; VH1-L5-VH2-L6-VL2-L7-VL1-L8-Fc; Fc-L9-TNF1-L10-TNF2-L11-TNF3; VL1-L12-VL2-L13-VH2-L14-VH1-L15-Fc-L16-TNF1-L17-TNF2-L18-TNF3; or VH1-L19-VH2-L20-VL2-L21-VL1-L22-Fc-L23-TNF1-L24-TNF2-L25-TNF3; and the second polypeptide has a structure represented by: VL3-L26-VL4-L27-VH4-L28-VH3-L29-Fc-L30-TNF1-L31-TNF2-L32-TNF3; or VH3-L33-VH4-L34-VL4-L35-VL3-L36-Fc-L37-TNF1-L38-TNF2-L39-TNF3; or
(ii) the first polypeptide has a structure represented by: Fc-L40-TNF1-L41-TNF2-L42-TNF3; VL1-L43-VL2-L44-VH2-L45-VH1-L46-Fc-L47-TNF1-L48-TNF2-L49-TNF3; or VH1-L50-VH2-L51-VL2-L52-VL1-L53-Fc-L54-TNF1-L55-TNF2-L56-TNF3; and the second polypeptide has a structure represented by: Fc; VL3-L57-VL4-L58-VH4-L59-VH3-L60-Fc; or VH3-L61-VH4-L62-VL4-L63-VL3-L64-Fc;
wherein: VL1 is a first immunoglobulin light chain variable region; VL2 is a second immunoglobulin light chain variable region; VL3 is a third immunoglobulin light chain variable region; VL4 is a fourth immunoglobulin light chain variable region; VH1 is a first immunoglobulin heavy chain variable region; VH2 is a second immunoglobulin heavy chain variable region; VH3 is a third immunoglobulin heavy chain variable region; VH4 is a fourth immunoglobulin heavy chain variable region; Fc is a region comprising an immunoglobulin heavy chain constant region 2 (CH2), an immunoglobulin heavy chain constant region 3 (CH3), and optionally, an immunoglobulin hinge; TNF1 is a first extracellular domain of a TNFSF ligand; TNF2 is a second extracellular domain of a TNFSF ligand; TNF3 is a third extracellular domain of a TNFSF ligand; and L1-L64 are amino acid linkers.

8. The antigen binding polypeptide complex of any one of clauses 1 to 7, wherein one or more of linkers L1-L96 have a length of from about 0 amino acids to about 50 amino acids.

9. The antigen binding polypeptide complex of any one of clauses 1 to 8, wherein one or more of linkers L1-L96 are non-immunogenic.

10. The antigen binding polypeptide complex of any one of clauses 1 to 9, wherein one or more of linkers L1-L96 do not contain a consensus T cell epitope.

11. The antigen binding polypeptide complex of any one of clauses 1 to 10, wherein one or more of linkers L1-L96 comprise the amino acid sequence of any one of SEQ ID NOs:3-10 and 148-175 or a sequence having at least 50%, at least 60%, at least 70%, at least 80%, at least 90%, or at least 95% identity to any one of SEQ ID NOs:3-10 and 148-175.

12. The antigen binding polypeptide of clause 11, wherein:
(i) one or more of linkers L1-L96 comprise an amino acid sequence having at least 80% identity to SEQ ID NO:3;
(ii) one or more of linkers L1-L96 comprise an amino acid sequence having at least 80% identity to SEQ ID NO:10;
(iii) one or more of linkers L1-L96 comprise an amino acid sequence having at least 80% identity to SEQ ID NO:150;
(iv) one or more of linkers L1-L96 comprise an amino acid sequence having at least 80% identity to SEQ ID NO:151;
(v) one or more of linkers L1-L96 comprise an amino acid sequence having at least 80% identity to SEQ ID NO:161; and/or
(vi) one or more of linkers L1-L96 comprise an amino acid sequence having at least 80% identity to SEQ ID NO168.

13. The antigen binding polypeptide complex of any one of clauses 1 to 6 and 8 to 12, wherein VL1 and VH1 specifically bind to CD3.

14. The antigen binding polypeptide complex of clause 13, wherein VL1 comprises a CDR1 comprising an amino acid sequence having at least 90% identity, at least 95% identity, or 100% identity to any one of SEQ ID NOs:22, 28 and 185; a CDR2 comprising an amino acid sequence having at least 90% identity, at least 95% identity, or 100% identity to any one of SEQ ID NOs:23, 29 and 186; and a CDR3 comprising an amino acid sequence having at least 90% identity, at least 95% identity, or 100% identity to any one of SEQ ID NOs:24, 30 and 187; and wherein VH1 comprises a CDR1 comprising an amino acid sequence having at least 90% identity, at least 95% identity, or 100% identity to any one of SEQ ID NOs:19, 25 and 182; a CDR2 comprising an amino acid sequence having at least 90% identity, at least 95% identity, or 100% identity to any one of SEQ ID NOs:20, 26 and 183; and a CDR3 comprising an amino acid sequence having at least 90% identity, at least 95% identity, or 100% identity to any one of SEQ ID NOs:21, 27 and 184.

15. The antigen binding polypeptide complex of clause 14, wherein VL1 comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or wherein VH1 comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21; optionally wherein VL1 comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or wherein VH1 comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21.

16. The antigen binding polypeptide complex of clause 14, wherein VL1 comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or wherein VH1 comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27; optionally wherein VL1 comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or wherein VH1 comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27.

17. The antigen binding polypeptide complex of clause 14, wherein VL1 comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or wherein VH1 comprises a CDR1 comprising an amino acid sequence having at least 90% identity to SEQ ID NO:182; a CDR2 comprising an amino acid sequence having at least 90% identity to SEQ ID No:183; and/or a CDR3 comprising an amino acid sequence having at least 90% identity to SEQ ID NO:184; optionally wherein VL1 comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH1 comprises a CDR1 comprising the amino acid sequence of SEQ ID NO:182; a CDR2 comprising the amino acid sequence of SEQ ID No:183; and/or a CDR3 comprising the amino acid sequence of SEQ ID NO:184.

18. The antigen binding polypeptide complex of clause 14, wherein VL1 comprises an amino acid sequence having at least 80%, at least 85%, at least 90%, at least 95%, or 100% identity to SEQ ID NO:45, and VH1 comprises an amino acid sequence having at least 80%, at least 85%, at least 90%, at least 95%, or 100% identity to SEQ ID NO:43 or 44; optionally wherein VL1 comprises an amino acid sequence having at least 80%, at least 85%, at least 90%, at least 95%, or 100% identity to SEQ ID NO:45, and VH1 comprises an amino acid sequence having at least 80%, at least 85%, at least 90%, at least 95%, or 100% identity to SEQ ID NO:43; or wherein VL1 comprises an amino acid sequence having at least 80%, at least 85%, at least 90%, at least 95%, or 100% identity to SEQ ID NO:45, and VH1 comprises an amino acid sequence having at least 80%, at least 85%, at least 90%, at least 95%, or 100% identity to SEQ ID NO:44.

19. The antigen binding polypeptide complex of any one of clauses 1 to 6 and 8 to 18, wherein VL2 and VH2 specifically bind to a tumor-associated antigen (TAA) or an immune stimulatory receptor.

20. The antigen binding polypeptide complex of clause 19, wherein the tumor-associated antigen is tyrosine-protein kinase Met (cMet), trophoblast cell surface antigen 2 (Trop2), CD20, CD19, receptor tyrosine-protein kinase erbB-2 (HER2), receptor tyrosine-protein kinase erbB-3 (HER3), adenosine A2A receptor (A2AR), a proliferation-inducing ligand (APRIL), epidermal growth factor receptor (EGFR), fibroblast growth factor receptor (FGFR), B cell activating factor (BAFF), BAFF receptor (BAFFR), B cell maturation antigen (BCMA), Bruton's tyrosine kinase (BTK), B and T lymphocyte attenuator (BTLA), B7DC (programmed death ligand 2), B7 homolog 1 (B7H1), B7 homolog 4 (B7H4), delta-like ligand 3 (DLL3), ectonucleoside triphosphate diphosphohydrolase 1 (ENTPD1), Fc fragment of IgE receptor 1a (FCER1A), Fc fragment of IgE receptor 1 (FCER1), arachidonate 5-lipoxygenase-activating protein (FLAP), folate hydrolase 1 (FOLH1), mucin 1 (MUC-1), CD133, mucin 16 (MUC-16), lysosomal-associated membrane protein 1 (LAMP1), CD38, programmed death ligand 1 (PD-L1), CEA cell adhesion molecule 5 (CEACAM5), six-transmembrane epithelial antigen of prostate 1 (STEAP1), or epithelial cellular adhesion molecule (EpCAM); or wherein the immune stimulatory receptor is CD28.

21. The antigen binding polypeptide complex of clause 19 or clause 20, wherein the VL2 and VH2 specifically bind to HER2.

22. The antigen binding polypeptide complex of clause 19, wherein the VL2 and VH2 specifically bind to the immune stimulatory receptor CD28.

23. The antigen binding polypeptide complex of any one of clauses 19 to 21, wherein VL2 comprises a CDR1 comprising an amino acid sequence having at least 90% identity, at least 95% identity, or 100% identity to SEQ ID NO:34 or 40; a CDR2 comprising an amino acid sequence having at least 90% identity, at least 95% identity, or 100% identity to SEQ ID NO:35 or 41; and a CDR3 comprising an amino acid sequence having at least 90% identity, at least 95% identity, or 100% identity to SEQ ID NO:36 or 42; and wherein VH2 comprises a CDR1 comprising an amino acid sequence having at least 90% identity, at least 95% identity, or 100% identity to SEQ ID NO:31 or 37; a CDR2 comprising an amino acid sequence having at least 90% identity, at least 95% identity, or 100% identity to SEQ ID NO:32 or 38; and a CDR3 comprising an amino acid sequence having at least 90% identity, at least 95% identity, or 100% identity to SEQ ID NO:33 or 39.

24. The antigen binding polypeptide complex of clause 23, wherein VL2 comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or wherein VH2 comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33; optionally wherein VL2 comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or wherein VH2 comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33.

25. The antigen binding polypeptide complex of clause 23, wherein VL2 comprises a CDR1 comprising an amino acid sequence having at least 90% to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or wherein VH2 comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39; optionally wherein VL2 comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or wherein VH2 comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

26. The antigen binding polypeptide complex of clause 23, wherein VL2 comprises an amino acid sequence having at least 80%, at least 85%, at least 90%, at least 95%, or 100% identity to SEQ ID NO:47 or 49, and VH2 comprises an amino acid sequence having at least 80%, at least 85%, at least 90%, at least 95%, or 100% identity to SEQ ID NO:46 or 48.

27. The antigen binding polypeptide complex of clause 26, wherein VL2 comprises a sequence having at least 80% identity to SEQ ID NO:47 and/or VH2 comprises a sequence having at least 80% identity to SEQ ID NO:46; optionally wherein VL2 comprises the amino acid sequence of SEQ ID NO:47 and/or VH2 comprises the amino acid sequence of SEQ ID NO: 46.

28. The antigen binding polypeptide complex of clause 26, wherein VL2 comprises a sequence having at least 80% identity to SEQ ID NO:49 and/or VH2 comprises a sequence having at least 80% identity to SEQ ID NO:48; optionally wherein VL2 comprises the amino acid sequence of SEQ ID NO:49 and/or VH2 comprises the amino acid sequence of SEQ ID NO: 48.

29. The antigen binding polypeptide complex of any one of clauses 1 to 6 and 8 to 12, wherein VL2 and VH2 specifically bind to CD3.

30. The antigen binding polypeptide complex of clause 29, wherein VL2 comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and a CDR3 comprising an amino acid sequence having at least 90% identity, at least 95% identity, or 100% identity to any one of SEQ ID NOs:24, 30, 187, 300 and 308; and wherein VH2 comprises a CDR1 comprising an amino acid sequence having at least 90% identity, at least 95% identity, or 100% identity to any one of SEQ ID Nos:19, 25, 182, 294 and 302; a CDR2 comprising an amino acid sequence having at least 90% identity, at least 95% identity, or 100% identity to any one of SEQ ID NOs:20, 26, 183, 295 and 303; and a CDR3 comprising an amino acid sequence having at least 90% identity, at least 95% identity, or 100% identity to any one of SEQ ID NOs:21, 27, 184, 296 and 304.

31. The antigen binding polypeptide complex of clause 30, wherein VL2 comprises a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or wherein VH2 comprises a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21; optionally wherein VL2 comprises a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or wherein VH2 comprises a CDR1 comprising the amino acid sequence of SEQ ID NO:19; a CDR2 comprising the amino acid sequence of SEQ ID NO:20; and/or a CDR3 comprising the amino acid sequence of SEQ ID NO:21.

32. The antigen binding polypeptide complex of clause 30, wherein VL2 comprises a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or wherein VH2 comprises a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27; optionally wherein VL2 comprises a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or wherein VH2 comprises a CDR1 comprising the amino acid sequence of SEQ ID NO:25; a CDR2 comprising the amino acid sequence of SEQ ID NO:26; and/or a CDR3 comprising the amino acid sequence of SEQ ID NO:27.

33. The antigen binding polypeptide complex of clause 30, wherein VL2 comprises a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or wherein VH2 comprises a CDR1 comprising an amino acid sequence having at least 90% identity to SEQ ID NO:182; a CDR2 comprising an amino acid sequence having at least 90% identity to SEQ ID No:183; and/or a CDR3 comprising an amino acid sequence having at least 90% identity to SEQ ID NO:184; optionally wherein VL2 comprises a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VH2 comprises a CDR1 comprising the amino acid sequence of SEQ ID NO:182; a CDR2 comprising the amino acid sequence of SEQ ID No:183; and/or a CDR3 comprising the amino acid sequence of SEQ ID NO:184.

34. The antigen binding polypeptide complex of clause 30, wherein VL2 comprises an amino acid sequence having at least 80%, at least 85%, at least 90%, at least 95%, or 100% identity to SEQ ID NO:45, 297 and 305, and VH2 comprises an amino acid sequence having at least 80%, at least 85%, at least 90%, at least 95%, or 100% identity to SEQ ID NO:43, 44, 188, 293 and 301; optionally wherein VL2 comprises an amino acid sequence having at least 80%, at least 85%, at least 90%, at least 95%, or 100% identity to SEQ ID NO:45, and VH2 comprises an amino acid sequence having at least 80%, at least 85%, at least 90%, at least 95%, or 100% identity to SEQ ID NO:43; wherein VL2 comprises an amino acid sequence having at least 80%, at least 85%, at least 90%, at least 95%, or 100% identity to SEQ ID NO:45, and VH2 comprises an amino acid sequence having at least 80%, at least 85%, at least 90%, at least 95%, or 100% identity to SEQ ID NO:44; wherein VL2 comprises an amino acid sequence having at least 80%, at least 85%, at least 90%, at least 95%, or 100% identity to SEQ ID NO:297, and VH2 comprises an amino acid sequence having at least 80%, at least 85%, at least 90%, at least 95%, or 100% identity to SEQ ID NO:293; or wherein VL2 comprises an amino acid sequence having at least 80%, at least 85%, at least 90%, at least 95%, or 100% identity to SEQ ID NO:305, and VH2 comprises an amino acid sequence having at least 80%, at least 85%, at least 90%, at least 95%, or 100% identity to SEQ ID NO:301.

35. The antigen binding polypeptide complex of any one of clauses 1 to 6, 8 to 12 and 29 to 34, wherein VL1 and VH1 specifically bind to a TAA or an immune stimulatory receptor.

36. The antigen binding polypeptide complex of clause 35, wherein the VL1 and VH1 specifically bind to a TAA selected from: cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, and EpCAM.

37. The antigen binding polypeptide complex of clause 35 or clause 36, wherein the TAA is HER2.

38. The antigen binding polypeptide complex of clause 35, wherein the VL1 and VH1 specifically bind to the immune stimulatory receptor CD28.

39. The antigen binding polypeptide complex of any one of clauses 35 to 38, wherein VL1 comprises a CDR1 comprising an amino acid sequence having at least 90% identity, at least 95% identity, or 100% identity to SEQ ID NO:34, 40, 274, 282, 290, 314 or 322; a CDR2 comprising an amino acid sequence having at least 90% identity, at least 95% identity, or 100% identity to any one of SEQ ID NO:35, 41, 275, 283, 291, 315 or 323; and a CDR3 comprising an amino acid sequence having at least 90% identity, at least 95% identity, or 100% identity to SEQ ID NO:36, 42, 275, 284, 292, 316 or 324; and wherein VH1 comprises a CDR1 comprising an amino acid sequence having at least 90% identity, at least 95% identity, or 100% identity to SEQ ID NO:31, 37, 270, 278, 286, 310 or 318; a CDR2 comprising an amino acid sequence having at least 90% identity, at least 95% identity, or 100% identity to SEQ ID NO:32, 38, 271, 279, 287, 311 or 319; and a CDR3 comprising an amino acid sequence having at least 90% identity, at least 95% identity, or 100% identity to SEQ ID NO:33, 39, 272, 280, 288, 312 or 320.

40. The antigen binding polypeptide complex of clause 39, wherein VL1 comprises a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or wherein VH1 comprises a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33; optionally wherein VL1 comprises a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or wherein VH1 comprises a CDR1 comprising the amino acid sequence of SEQ ID NO:31; a CDR2 comprising the amino acid sequence of SEQ ID NO:32; and/or a CDR3 comprising the amino acid sequence of SEQ ID NO:33.

41. The antigen binding polypeptide complex of clause 39, wherein VL1 comprises a CDR1 comprising an amino acid sequence having at least 90% to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or wherein VH1 comprises a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39; optionally wherein VL1 comprises a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or wherein VH1 comprises a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

42. The antigen binding polypeptide complex of clause 39, wherein VL1 comprises an amino acid sequence having at least 80%, at least 85%, at least 90%, at least 95%, or 100% identity to SEQ ID NO:47 or 49, and VH1 comprises an amino acid sequence having at least 80%, at least 85%, at least 90%, at least 95%, or 100% identity to SEQ ID NO:46 or 48.

43. The antigen binding polypeptide complex of clause 42, wherein VL1 comprises a sequence having at least 80% identity to SEQ ID NO:47 and/or VH1 comprises a sequence having at least 80% identity to SEQ ID NO:46; optionally wherein VL1 comprises the amino acid sequence of SEQ ID NO:47 and/or VH1 comprises the amino acid sequence of SEQ ID NO: 46.

44. The antigen binding polypeptide complex of clause 42, wherein VL1 comprises a sequence having at least 80% identity to SEQ ID NO:49 and/or VH1 comprises a sequence having at least 80% identity to SEQ ID NO:48; optionally wherein VL1 comprises the amino acid sequence of SEQ ID NO:49 and/or VH1 comprises the amino acid sequence of SEQ ID NO: 48.

45. The antigen binding polypeptide complex of any one of clauses 1 to 6 and 8 to 28, wherein the VL1 and VH1 of the antigen binding polypeptide specifically bind to CD3 and the VL2 and VH2 specifically bind to a TAA or an immune stimulatory receptor.

46. The antigen binding polypeptide complex of any one of clauses 1 to 6, 8 to 12 and 29 to 44, wherein the VL2 and VH2 of the antigen binding polypeptide specifically bind to CD3 and the VL1 and VH1 specifically bind to a TAA or an immune stimulatory receptor.

47. The antigen binding polypeptide complex of any one of clauses 7 to 12, wherein VL1, VH1, VL3 and VH3 specifically bind to CD3.

48. The antigen binding polypeptide complex of any one of clauses 7 to 12 and 47, wherein VL2, VH2, VL4 and VH4 specifically bind to a TAA or an immune stimulatory receptor.

49. The antigen binding polypeptide complex of clause 48, wherein the TAA is HER2 or the immune stimulatory receptor is CD28.

50. The antigen binding polypeptide complex of any one of clauses 7 to 12, wherein VL1, VH1, VL4 and VH4 specifically bind to CD3.

51. The antigen binding polypeptide complex of any one of clauses 7 to 12 and 50, wherein VL2, VH2, VL3 and VH3 specifically bind to a TAA or an immune stimulatory receptor. 52. The antigen binding polypeptide complex of clause 51, wherein the TAA is HER2 or the immune stimulatory receptor is CD28.

53. The antigen binding polypeptide complex of any one of clauses 7 to 12, wherein VL2, VH2, VL4 and VH4 specifically bind to CD3.

54. The antigen binding polypeptide complex of any one of clauses 7 to 12 and 53, wherein VL1, VH1, VL3 and VH3 specifically bind to a TAA or an immune stimulatory receptor.

55. The antigen binding polypeptide complex of clause 54, wherein the TAA is HER2 or the immune stimulatory receptor is CD28.

56. The antigen binding polypeptide complex of any one of clauses 7 to 12, wherein VL2, VH2, VL3 and VH3 specifically bind to CD3.

57. The antigen binding polypeptide complex of any one of clauses 7 to 12 and 56, wherein VL1, VH1, VL4 and VH4 specifically bind to a TAA or an immune stimulatory receptor.

58. The antigen binding polypeptide complex of clause 57, wherein the TAA is HER2 or the immune stimulatory receptor is CD28.

59. The antigen binding polypeptide complex of any one of clauses 47 to 58, wherein the VLs specifically binding to CD3 comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and a CDR3 comprising an amino acid sequence having at least 90% identity, at least 95% identity, or 100% identity to any one of SEQ ID NOs:24, 30, 187, 300 and 308; and wherein the VHs specifically binding to CD3 comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and a CDR3 comprising an amino acid sequence having at least 90% identity, at least 95% identity, or 100% identity to any one of SEQ ID NOs:21, 27, 184, 296 and 304.

60. The antigen binding polypeptide complex of clause 59, wherein the VLs specifically binding to CD3 comprise a CDR1 comprising an amino acid sequence having at least 90% identity to SEQ ID NO:22; a CDR2 comprising an amino acid sequence having at least 90% identity to SEQ ID NO:23; and/or a CDR3 comprising an amino acid sequence having at least 90% identity to SEQ ID NO:24; and/or wherein the VHs specifically binding to CD3 comprise a CDR1 comprising an amino acid sequence having at least 90% identity to SEQ ID NO:19; a CDR2 comprising an amino acid sequence having at least 90% identity to SEQ ID NO:20; and/or a CDR3 comprising an amino acid sequence having at least 90% identity to SEQ ID NO:21; optionally wherein the VLs comprise a CDR1 comprising the amino acid sequence of SEQ ID NO:22; a CDR2 comprising the amino acid sequence of SEQ ID NO:23; and/or a CDR3 comprising the amino acid sequence of SEQ ID NO:24; and/or wherein the VHs comprise a CDR1 comprising the amino acid sequence of SEQ ID NO:19; a CDR2 comprising the amino acid sequence of SEQ ID NO:20; and/or a CDR3 comprising the amino acid sequence of SEQ ID NO:21.

61. The antigen binding polypeptide complex of clause 59, wherein the VLs specifically binding to CD3 comprise a CDR1 comprising an amino acid sequence having at least 90% identity to SEQ ID NO:28; a CDR2 comprising an amino acid sequence having at least 90% identity to SEQ ID NO:29; and/or a CDR3 comprising an amino acid sequence having at least 90% identity to SEQ ID NO:30; and/or wherein the VHs specifically binding to CD3 comprise a CDR1 comprising an amino acid sequence having at least 90% identity to SEQ ID NO:25; a CDR2 comprising an amino acid sequence having at least 90% identity to SEQ ID NO:26; and/or a CDR3 comprising an amino acid sequence having at least 90% identity to SEQ ID NO:27; optionally wherein the VLs comprise a CDR1 comprising the amino acid sequence of SEQ ID NO:28; a CDR2 comprising the amino acid sequence of SEQ ID NO:29; and/or a CDR3 comprising the amino acid sequence of SEQ ID NO:30; and/or wherein the VHs comprise a CDR1 comprising the amino acid sequence of SEQ ID NO:25; a CDR2 comprising the amino acid sequence of SEQ ID NO:26; and/or a CDR3 comprising the amino acid sequence of SEQ ID NO:27.

62. The antigen binding polypeptide complex of clause 59, wherein the VLs specifically binding to CD3 comprise a CDR1 comprising an amino acid sequence having at least 90% identity to SEQ ID NO:185; a CDR2 comprising an amino acid sequence having at least 90% identity to SEQ ID NO:186; and/or a CDR3 comprising an amino acid sequence having at least 90% identity to SEQ ID NO:187; and/or wherein the VHs specifically binding CD3 comprise a CDR1 comprising an amino acid sequence having at least 90% identity to SEQ ID NO:182; a CDR2 comprising an amino acid sequence having at least 90% identity to SEQ ID No:183; and/or a CDR3 comprising an amino acid sequence having at least 90% identity to SEQ ID NO:184; optionally wherein the VLs comprise a CDR1 comprising the amino acid sequence of SEQ ID NO:185; a CDR2 comprising the amino acid sequence of SEQ ID NO:186; and/or a CDR3 comprising the amino acid sequence of SEQ ID NO:187; and/or the VHs comprise a CDR1 comprising the amino acid sequence of SEQ ID NO:182; a CDR2 comprising the amino acid sequence of SEQ ID No:183; and/or a CDR3 comprising the amino acid sequence of SEQ ID NO:184.

63. The antigen binding polypeptide complex of clause 59, wherein the VLs specifically binding to CD3 comprise an amino acid sequence having at least 80%, at least 85%, at least 90%, at least 95%, or 100% identity to SEQ ID NO:45, 297 or 305, and the VHs specifically binding to CD3 comprise an amino acid sequence having at least 80%, at least 85%, at least 90%, at least 95%, or 100% identity to SEQ ID NO:43, 44, 188, 293 or 301.

64. The antigen binding polypeptide complex of clause 63, wherein the VLs specifically binding to CD3 comprise an amino acid sequence having at least 80%, at least 85%, at least 90%, at least 95%, or 100% identity to SEQ ID NO:45, and the VHs specifically binding to CD3 comprise an amino acid sequence having at least 80%, at least 85%, at least 90%, at least 95%, or 100% identity to SEQ ID NO:43; or wherein the VLs specifically binding to CD3 comprise an amino acid sequence having at least 80%, at least 85%, at least 90%, at least 95%, or 100% identity to SEQ ID NO:45, and the VHs specifically binding to CD3 comprise an amino acid sequence having at least 80%, at least 85%, at least 90%, at least 95%, or 100% identity to SEQ ID NO:44.

65. The antigen binding polypeptide complex of any one of clauses 47 to 64, wherein the VLs specifically binding to a TAA or immune stimulatory receptor comprise a CDR1 comprising an amino acid sequence having at least 90% identity, at least 95% identity, or 100% identity to SEQ ID NO:34, 40, 274, 282, 289, 314 or 322; a CDR2 comprising an amino acid sequence having at least 90% identity, at least 95% identity, or 100% identity to SEQ ID NO:35, 41, 275, 283, 290, 315 or 323; and a CDR3 comprising an amino acid sequence having at least 90% identity, at least 95% identity, or 100% identity to SEQ ID NO:36, 42, 276, 284, 291, 316 or 324; and wherein the VHs specifically binding to a TAA or immune stimulatory receptor comprises a CDR1 comprising an amino acid sequence having at least 90% identity, at least 95% identity, or 100% identity to SEQ ID NO:31, 37, 270, 278, 286, 310 or 318; a CDR2 comprising an amino acid sequence having at least 90% identity, at least 95% identity, or 100% identity to SEQ ID NO:32, 38, 271, 279, 287, 311 or 319; and a CDR3 comprising an amino acid sequence having at least 90% identity, at least 95% identity, or 100% identity to SEQ ID NO:33, 39, 272, 280, 288, 312 or 320.

66. The antigen binding polypeptide complex of clause 65, wherein the VLs that specifically bind an immune stimulatory receptor comprise a CDR1 comprising an amino acid sequence having at least 90% identity to SEQ ID NO:34; a CDR2 comprising an amino acid sequence having at least 90% identity to SEQ ID NO:35; and/or a CDR3 comprising an amino acid sequence having at least 90% identity to SEQ ID NO:36; and/or wherein the VHs that specifically bind an immune stimulatory receptor comprise a CDR1 comprising an amino acid sequence having at least 90% identity to SEQ ID NO:31; a CDR2 comprising an amino acid sequence having at least 90% identity to SEQ ID NO:32; and/or a CDR3 comprising an amino acid sequence having at least 90% identity to SEQ ID NO:33; optionally wherein the VLs comprise a CDR1 comprising the amino acid sequence of SEQ ID NO:34; a CDR2 comprising the amino acid sequence of SEQ ID NO:35; and/or a CDR3 comprising the amino acid sequence of SEQ ID NO:36; and/or wherein the VHs comprise a CDR1 comprising the amino acid sequence of SEQ ID NO:31; a CDR2 comprising the amino acid sequence of SEQ ID NO:32; and/or a CDR3 comprising the amino acid sequence of SEQ ID NO:33.

67. The antigen binding polypeptide complex of clause 65, wherein the VLs that specifically bind to a TAA comprise a CDR1 comprising an amino acid sequence having at least 90% to SEQ ID NO:40; a CDR2 comprising an amino acid sequence having at least 90% identity to SEQ ID NO:41; and/or a CDR3 comprising an amino acid sequence having at least 90% identity to SEQ ID NO:42; and/or wherein the VHs that specifically bind to a TAA comprise a CDR1 comprising an amino acid sequence having at least 90% identity to SEQ ID NO:37; a CDR2 comprising an amino acid sequence having at least 90% identity to SEQ ID NO:38; and/or a CDR3 comprising an amino acid sequence having at least 90% identity to SEQ ID NO:39; optionally wherein the VLs comprise a CDR1 comprising the amino acid sequence of SEQ ID NO:40; a CDR2 comprising the amino acid sequence of SEQ ID NO:41; and/or a CDR3 comprising the amino acid sequence of SEQ ID NO:42; and/or wherein the VHs comprise a CDR1 comprising the amino acid sequence of SEQ ID NO:37; a CDR2 comprising the amino acid sequence of SEQ ID NO:38; and/or a CDR3 comprising the amino acid sequence of SEQ ID NO:39.

68. The antigen binding polypeptide complex of clause 65, wherein the VLs specifically binding to a TAA or immune stimulatory receptor comprise an amino acid sequence having at least 80%, at least 85%, at least 90%, at least 95%, or 100% identity to SEQ ID NO:47 or 49, and the VHs specifically binding to a TAA or an immune stimulatory receptor comprise an amino acid sequence having at least 80%, at least 85%, at least 90%, at least 95%, or 100% identity to SEQ ID NO:46 or 48.

69. The antigen binding polypeptide complex of clause 68, wherein the VLs that specifically binding to an immune stimulatory receptor comprise a sequence having at least 80% identity to SEQ ID NO:47 and/or the VHs that specifically binding to an immune stimulatory receptor comprise a sequence having at least 80% identity to SEQ ID NO:46; optionally wherein the VLs comprise the amino acid sequence of SEQ ID NO:47 and/or the VHs comprise the amino acid sequence of SEQ ID NO: 46.

70. The antigen binding polypeptide complex of clause 68, wherein the VLs specifically binding to a TAA comprise a sequence having at least 80% identity to SEQ ID NO:49 and/or the VHs that specifically binding to a TAA comprise a sequence having at least 80% identity to SEQ ID NO:48; optionally wherein the VLs comprise the amino acid sequence of SEQ ID NO:49 and/or the VHs comprise the amino acid sequence of SEQ ID NO: 48.

71. The antigen binding polypeptide complex of any one of clauses 1 to 70, wherein TNF1, TNF2 and TNF3 are each selected from the group consisting of OX40L (TNFSF4), 4-1BBL (TNFSF9), TNF, TNF-related apoptosis inducing ligand (TRAIL), CD40L (TNFSF5), CD27L (TNFSF7), CD30L (TNFSF8), FasL (TNFSF6), EDAM, LTA (TNFSF1), LTB (TNFSF3), CD153 (TNFSF8), RANKL (TNFSF11), TWEAK (TNFSF12), APRIL (TNFSF13), BAFF (TNFSF13B), LIGHT (TNFSF14), VEGI (TNFSF15), and GITRL (TNFSF18).

72. The antigen binding polypeptide complex of any one of clauses 1 to 71, wherein TNF1, TNF2 and TNF3 are each OX40L.

73. The antigen binding polypeptide complex of any one of clauses 1 to 72, wherein TNF1, TNF2, and TNF3 are each 4-1BBL.

74. The antigen binding polypeptide complex of any one of clauses 1 to 73, wherein the antigen binding polypeptide complex is an antibody or antigen binding fragment thereof.

75. The antigen binding polypeptide complex of any one of clauses 1 to 74, wherein the immunoglobulin hinge comprises an upper hinge region, a middle hinge region, a lower hinge region, or a combination thereof.

76. The antigen binding polypeptide complex of any one of clauses 1 to 75, wherein the Fc region comprises at least one knob-into-hole modification.

77. The antigen binding polypeptide complex of clause 76, wherein the antigen binding polypeptide complex is an IgG1 or IgG4 antibody and the knob-into-hole modification comprises: (i) knob substitutions of S354C and T366W and hole substitutions of Y349C, T366S, L368A and Y407V; (ii) hole substitutions of L234A, L235A and P329A; (iii) hole substitutions of L234A and L235A; (iv) hole substitutions of M428L and N433S; (v) hole substitutions of M252Y, S254T and T256E; or (vi) a combination thereof; based on the EU numbering scheme.

78. An antibody or antigen binding fragment thereof comprising the antigen binding polypeptide complex of any one of clauses 1 to 77.

79. A pharmaceutical composition comprising the antigen binding polypeptide complex of any one of clauses 1 to 77 or the antibody or antigen binding fragment thereof of clause 78, and a pharmaceutically acceptable carrier.

80. An antigen binding polypeptide complex according to any one of clauses 1 to 77, an antibody or antigen binding fragment thereof according to clause 78, or a pharmaceutical composition according to clause 79 for use in treating a disease or condition in a patient.

81. An antigen binding polypeptide complex according to any one of clauses 1 to 77, an antibody or antigen binding polypeptide complex according to clause 78, or a pharmaceutical composition according to clause 79 for use in treating cancer in a patient.

### EXAMPLES

The following examples are provided to further illustrate aspects of the disclosure, and are not meant to constrain the disclosure to any particular application or theory of operation.

### Example 1

### Multispecific TNFSF Fusion Antibody Expression and Purification

Multispecific tumor necrosis factor superfamily (TNFSF) fusion antibodies of the disclosure were produced by transient transfection of expression plasmids into Expi293F cells at a density of 2.5-3.0×10⁶/mL using polyethylenimine (PEI)(Polyscience). Plasmid DNA and PEI were diluted in OPTi-MEM (LifeTech) separately and mixed later. The plasmid/PEI mixture, at a ratio of 1:3 (w:w), was added to the cell culture 10 minutes after mixing. Valproic acid and sodium propionate were added to final concentrations of 0.5 mM and 5 mM, respectively, 16-20 hours post-transfection. Supernatant was harvested 5 days post-transfection, and filtered through a 0.45 μm filter.

Multispecific antibodies were then purified first by affinity chromatography using Protein A resins in batch mode according to manufacturer's standard procedures. After antibodies were eluted using 3M Magnesium Chloride from protein A, they were dialyzed into 10 mM Histidine (pH 6.0)+150 mM NaCl overnight with two changes of buffer. Antibodies were further purified by size exclusion chromatography using Hiload 16/600 Superdex 200 PG or Superdex 200 Increase 10/300 GL (Cytiva Lifesciences). Fractions with the correct elusion profile were collected and concentrated for further characterization.

### Example 2

### Multispecific OX40L Fusion Antibody ELISA Binding Analysis

Three multispecific antibodies that bind to CD3 and CD28 and contain a dimer of OX40L trimers were prepared and purified as explained in Example 1 (MX169, MX368 and MX369). To assess binding of the antibodies to their target proteins, an enzyme-linked immunosorbent assay (ELISA) was performed.

Target protein for each binding site of the multispecific antibodies was coated in the wells of 96-well Immuno Plates (Thermo Fisher Scientific) overnight at 4° C. Coated plates were blocked using 5% skim milk+2% bovine serum albumin (BSA) in phosphate buffered saline (PBS)+0.25% Tween for one hour at room temperature, then washed with PBS+0.25% Tween 20 three times. Serial diluted antibodies and control molecules were added to the plates and incubated at room temperature for 1 hour. Plates were washed three times with PBS+0.25% Tween 20, incubated with horseradish peroxidase (HRP)-conjugated detection antibody for one hour at room temperature, washed again, and then developed with Peroxidase Substrate (KPL, Gaithersburg, MD, USA). After the reaction was terminated by adding 100 μl of KPL TMB BlueSTOP solution, the plates were read at OD650 using a plate reader and data were analyzed in GraphPad Prism.

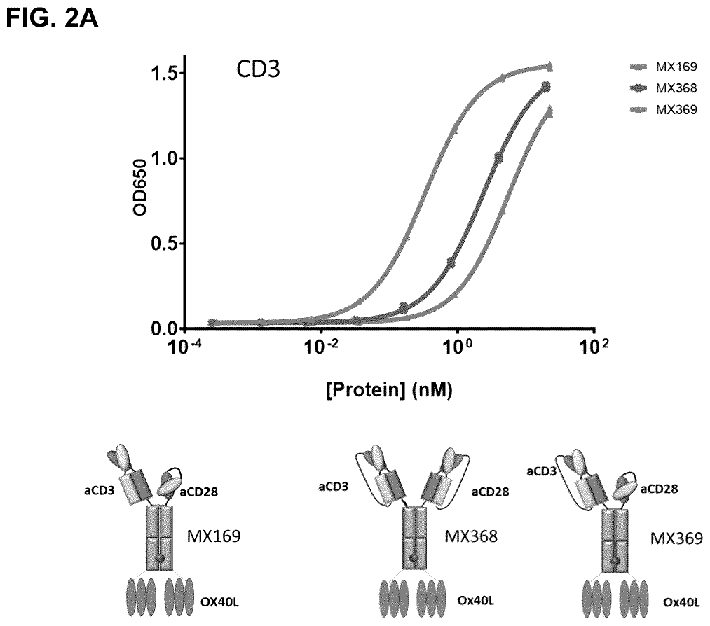

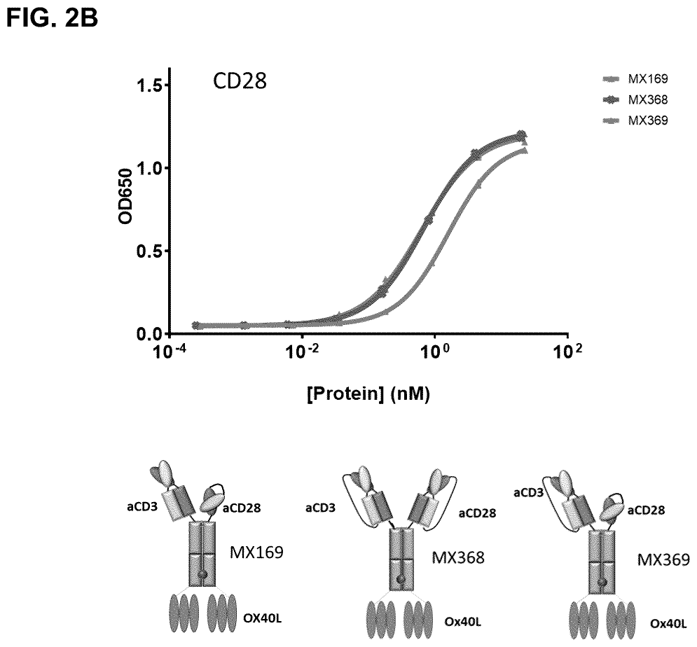

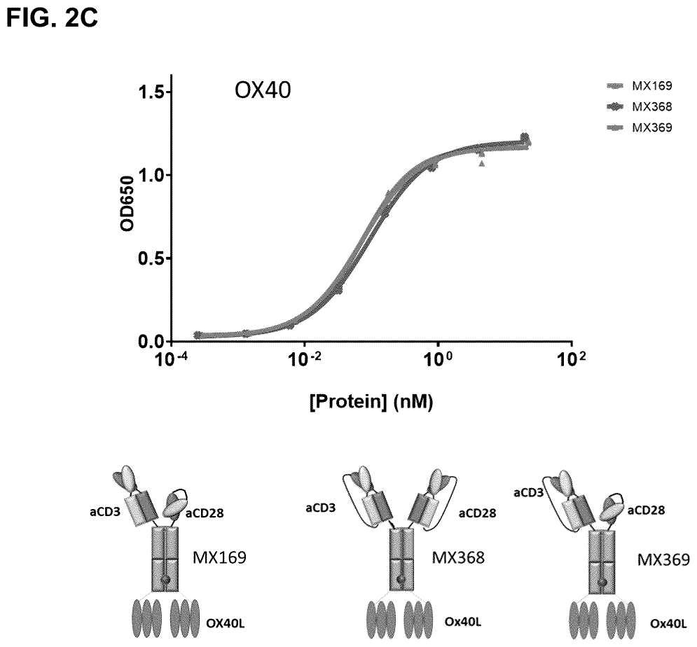

FIG. 2A shows ELISA binding results of MX169, MX368 and MX369 to human CD3. FIG. 2B shows ELISA binding results of MX169, MX368 and MX369 to human CD28. FIG. 2C shows ELISA binding results of MX169, MX368 and MX369 to human OX40. These results show that all three multispecific antibodies were functionally active and bound to CD3, CD28 and OX40 target proteins.

### Example 3

### Luciferase Reporter Assay—MX169, MX368 and MX369

To assess the effect of multispecific antibodies MX169, MX368 and MX369 on T cell activation, an in vitro luciferase reporter assay was performed. NF-kB Luciferase Reporter Jurkat Stable Cell Line (Signosis, CA, USA) and Jurkat-Lucia™ NFAT Cells (InvivoGen, CA, USA) were prepared according to the manufacturer's protocols. Briefly, cells were thawed for 2 minutes in a 37° C. water bath and gently transferred to a 15 mL conical centrifuge tube containing 10 mL pre-warmed R10 media. Cells were pelleted at 300 g for 5 minutes at room temperature. After removing the supernatant, cells were resuspended in 20 mL of pre-warmed culture media and transferred to a 75 cm² culture flask, followed by incubation in a mammalian tissue culture incubator until cells were growing and stable (^{˜}3-4 days). Cells were maintained in culture media+selective antibiotics and used approximately 7 days after thawing.

For antibody stimulation, multispecific or control antibodies were serially diluted in PBS and coated onto 96-well flat-bottom culture plates by incubating 2-4 hours in a 37° C. tissue culture incubator. NF-kB Luciferase Reporter Jurkat Stable Cells were resuspended to 2×10⁶ cells/mL, with 100 μL of cells added to each well containing the antibodies, and incubated in a mammalian incubator for 24 hours. The assay plates were then taken out and allowed to equilibrate to ambient temperature (10-15 minutes). Bio-Glo™ Reagent (Promega Cat #G7941) (ambient temperature) was added at 50 μL for each well of the assay plate. After 5 minutes, luminescence activity was measured using Varioskan microplate reader (Thermo Fisher). Data were plotted using GraphPad Prism software.

Jurkat-Lucia™ NFAT Cells were resuspended to 7.5×10⁵ cells/mL, with 200 μL of cells added to each well containing the antibodies and incubated in a mammalian incubator for 24 hours. 20 uL of the cell culture supernatant was pipetted into a new 96-well white-walled microtiter plate. 50 uL of Quanti-Luc solution (InvivoGen) was then added to each well before luminescence activity was measured using Varioskan microplate reader (Thermo Fisher). Data were plotted using GraphPad Prism software.

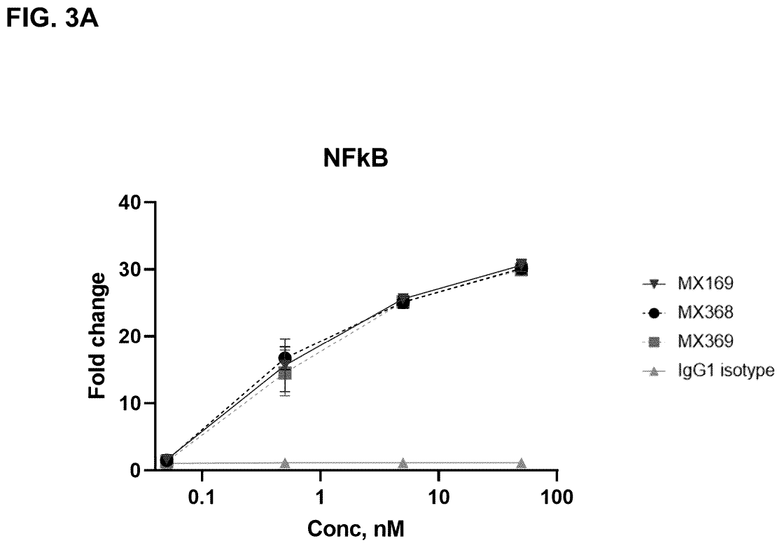

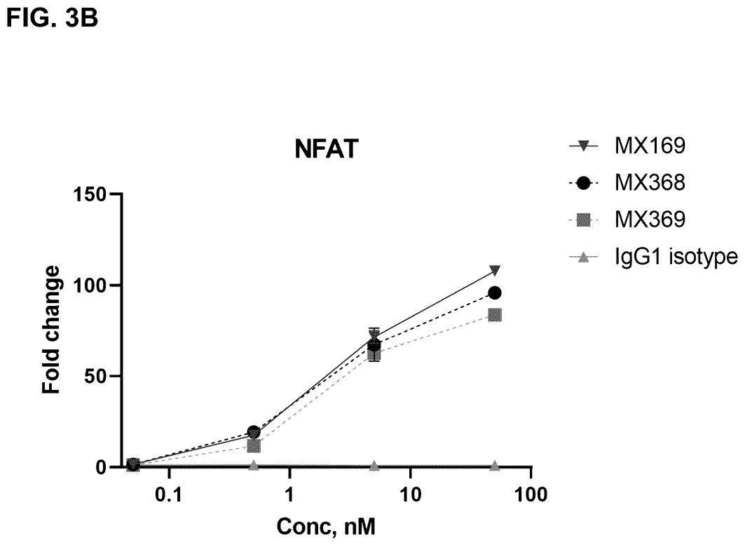

FIG. 3A shows the fold change in T cell activation tested in Jurkat cell lines expressing luciferase under the control of the NF-κB (NFkB) promoter for MX169, MX368 and MX369). FIG. 3B shows the fold change in T cell activation tested in Jurkat cell lines expressing luciferase under the control of the NFAT promoter for MX169, MX368 and MX369. Results from a control IgG1 isotype antibody are also shown (IgG1 isotype). These results show that all three multispecific antibodies were functionally active and resulted in a dose-dependent activation of T cells using either promoter.

### Example 4

### T Cell Proliferation Assay—MX169, MX368 and MX369

To assess the effect of multispecific antibodies MX169, MX368 and MX369 on T cells, and in vitro T cell proliferation assay and flow cytometry were performed. Purified human peripheral blood mononuclear cells (PBMCs) (Blood Research Component, Brookline, MA, USA) were resuspended in culture medium (RPMI1640 with 10% fetal bovine serum (FBS) and supplemented with Penicillin Streptomycin)(Gibco) (2.5×10⁵ cells/ml). Serial diluted multispecific and control antibodies were first coated onto 96-well flat-bottom culture plates by incubating 2-4 hours in a 37° C. tissue culture incubator. PBMCs (200 μL) were then added to each well containing the antibodies and incubated for 7 days in a 37° C. tissue culture incubator.

Cell culture media was replaced after 4 days during incubation. The cells were centrifuged, washed, and stained with fluorescent-labeled antibodies for T cell markers, such as CD2, CD4 and CD8, along with viability dye and Precision Count Beads™ (Biolegend), before being acquired on an Attune flow cytometer (Thermo Fisher Scientific, USA). Data were analyzed using FlowJo software. Fold change in CD4+ and CD8+ T cell proliferation with treatment was calculated by dividing cell concentrations from Day 7 and Day 0.

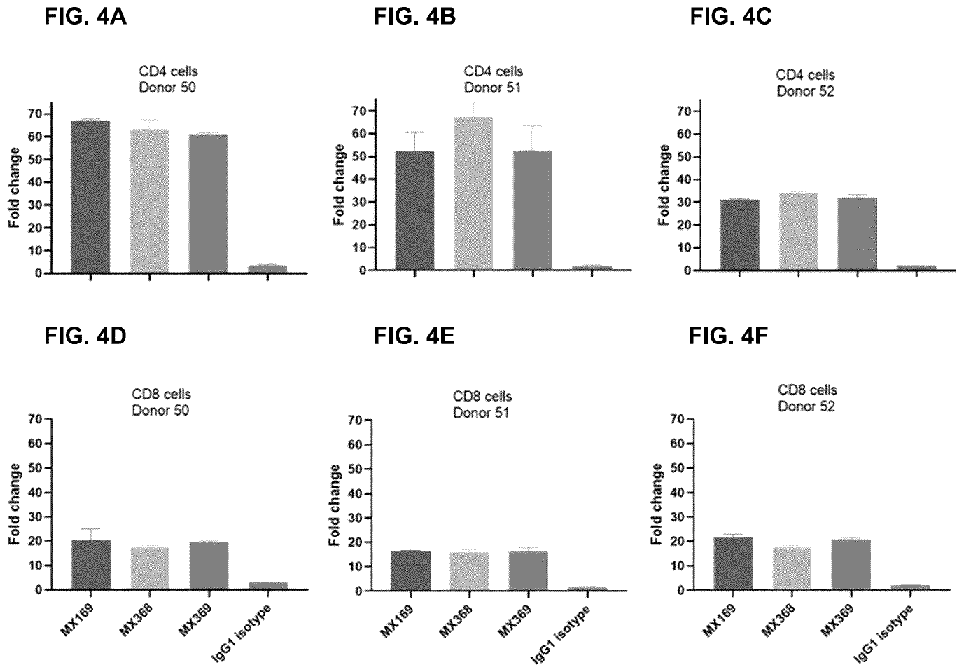

FIGS. 4A-4F shows the fold change in proliferation of primary human CD4+ T cells (FIGS. 4A-4C) and CD8+ T cells (FIGS. 4D-4F) from three different donors upon treatment with MX169, MX368 and MX369. Results from a control IgG1 isotype antibody are also shown (IgG1 isotype). These results show that all three multispecific antibodies induced CD4+ and CD8+ T cell proliferation.

### Example 5

### Multispecific 4-1BBL Fusion Antibody ELISA Binding Analysis

Three multispecific antibodies that bind to CD3 and CD28 and contain 4-1BBL trimers were prepared and purified as explained in Example 1 (MX306, MX424 and MX425). To assess binding of the antibodies to their target proteins, an ELISA binding assay was performed.

Target protein for each binding site of the multispecific antibodies was coated in the wells of 96-well Immuno Plates (Thermo Fisher Scientific) overnight at 4° C. Coated plates were blocked using 5% skim milk+2% bovine serum albumin (BSA) in phosphate buffered saline (PBS)+0.25% Tween for one hour at room temperature, then washed with PBS+0.25% Tween 20 for three times. Serial diluted antibodies and control molecules were added to the plates and incubated at room temperature for 1 hour. Plates were washed three times with PBS+0.25% Tween 20, incubated with horseradish peroxidase (HRP)-conjugated detection antibody for one hour at room temperature, washed again, and then developed with Peroxidase Substrate (KPL, Gaithersburg, MD, USA). After the reaction was terminated by adding 100 μl of KPL TMB BlueSTOP solution, the plates were read at OD650 using a plate reader and data were analyzed in GraphPad Prism.

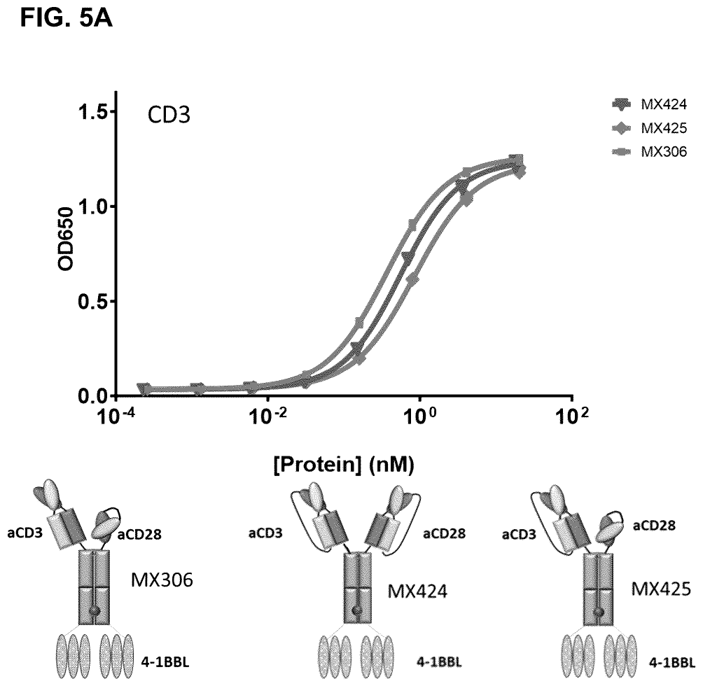

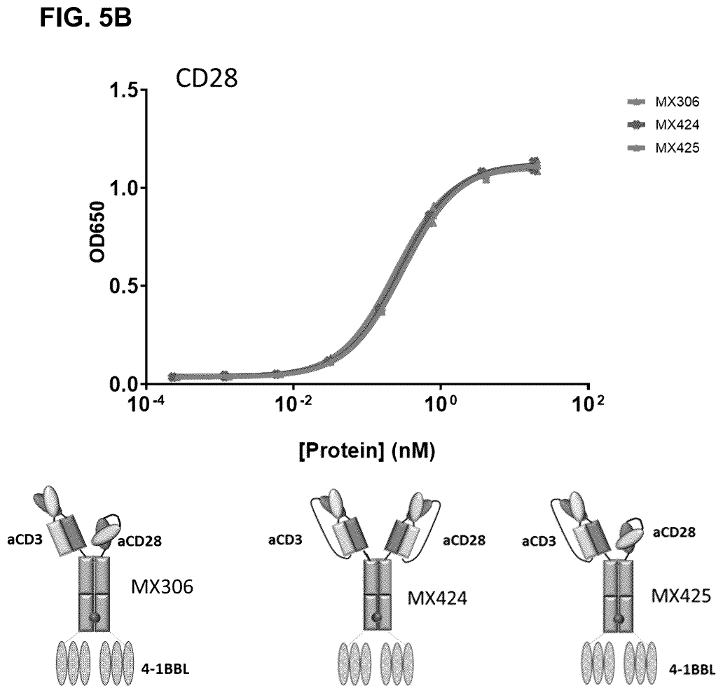

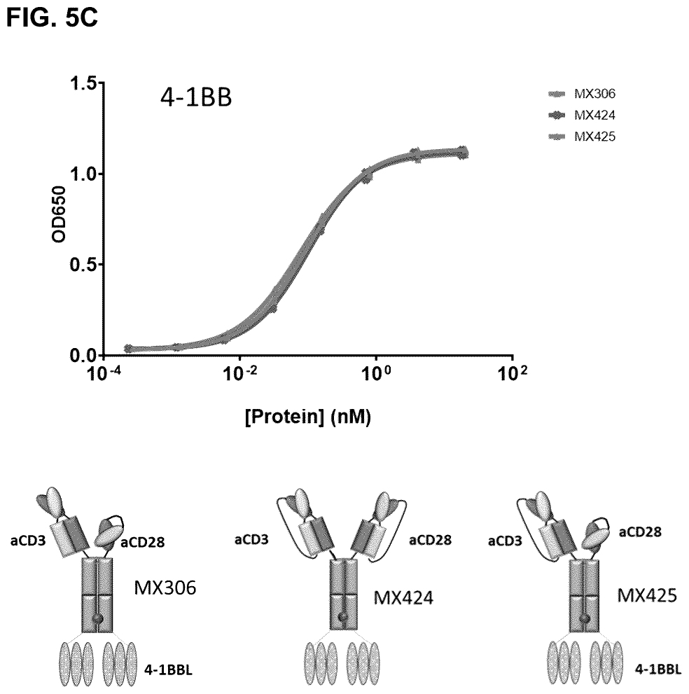

FIG. 5A shows ELISA binding results for MX306, MX424 and MX425 to human CD3. FIG. 5B shows ELISA binding results for MX306, MX424 and MX425 to human CD28. FIG. 5C shows ELISA binding results for MX306, MX424 and MX425 to human 4-1BBL. These results show that all three multispecific antibodies were functionally active and bound to CD3, CD28 and 4-1BBL target proteins.

### Example 6

### T Cell Proliferation Assay—MX306, MX424 and MX425

To assess the effect of multispecific antibodies MX306, MX424 and MX425 on T cells, and in vitro T cell proliferation assay and flow cytometry were performed. PBMCs (Blood Research Component, Brookline, MA, USA) were resuspended in culture medium (RPMI1640 with 10% fetal bovine serum (FBS) and supplemented with Penicillin Streptomycin)(Gibco) (2.5×10⁵ cells/ml). Serial diluted multispecific and control antibodies were first coated onto 96-well flat-bottom culture plates by incubating 2-4 hours in a 37° C. tissue culture incubator. PBMCs (200 μL) were then added to each well containing the antibodies and incubated for 7 days in a 37° C. tissue culture incubator.

Cell culture media was replaced after 4 days during incubation. The cells were centrifuged, washed, and stained with fluorescent-labeled antibodies for T cell markers, such as CD2, CD4 and CD8, along with viability dye and Precision Count Beads™ (Biolegend), before being acquired on an Attune flow cytometer (Thermo Fisher Scientific, USA). Data were analyzed using FlowJo software. Fold change in CD4+ and CD8+ T cell proliferation with treatment was calculated by dividing cell concentrations from Day 7 and Day 0.

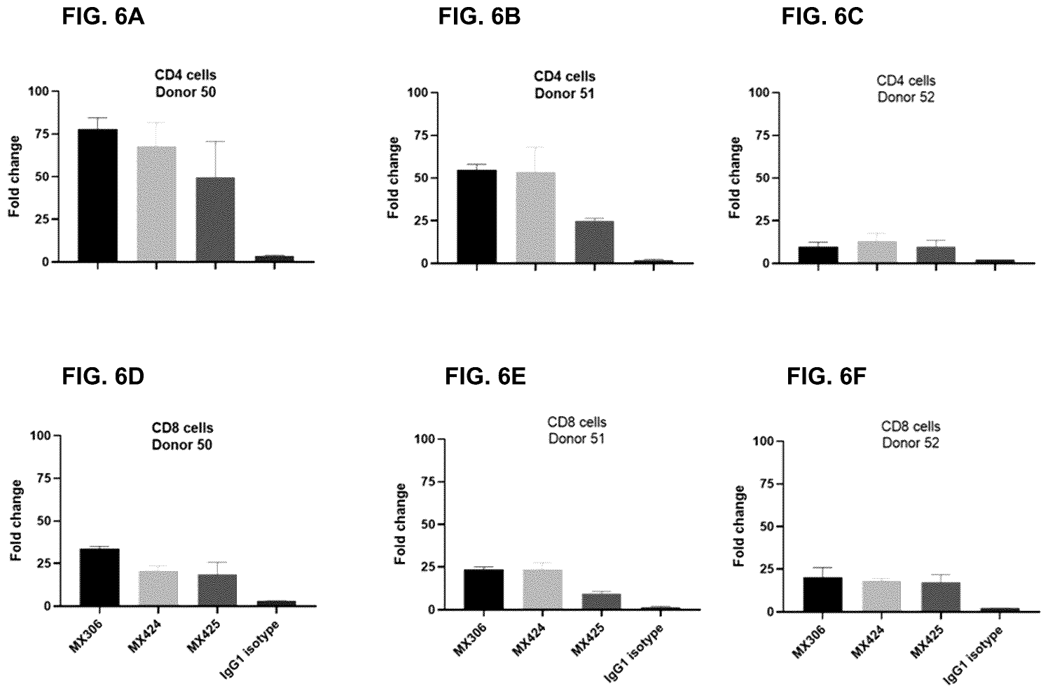

FIGS. 6A-6F shows the fold change in proliferation of primary human CD4+ T cells (FIGS. 6A-6C) and CD8+ T cells (FIGS. 6D-6F) from three different donors upon treatment with MX306, MX424 and MX425. Results from a control IgG1 isotype antibody are also shown (IgG1 isotype). These results show that all three multispecific antibodies induced CD4+ and CD8+ T cell proliferation.

### Example 7

### Proliferation of Human CD4 Memory T Cells with OX40L Fusion Antibodies

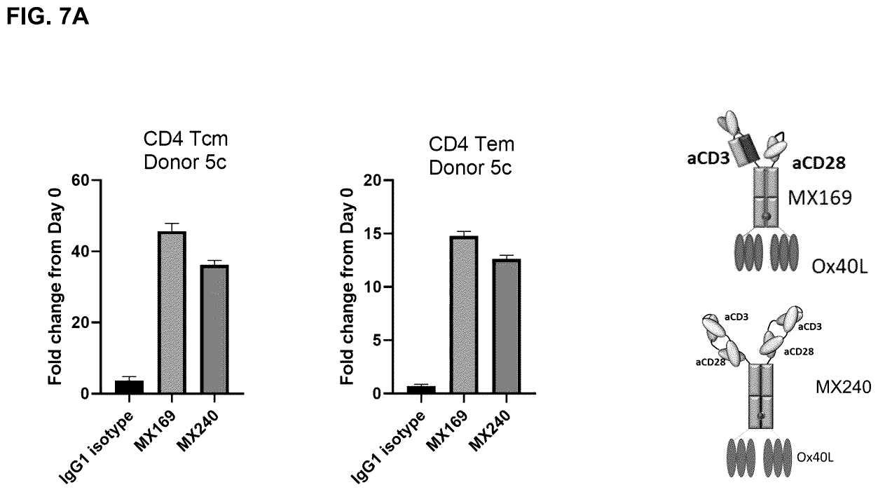

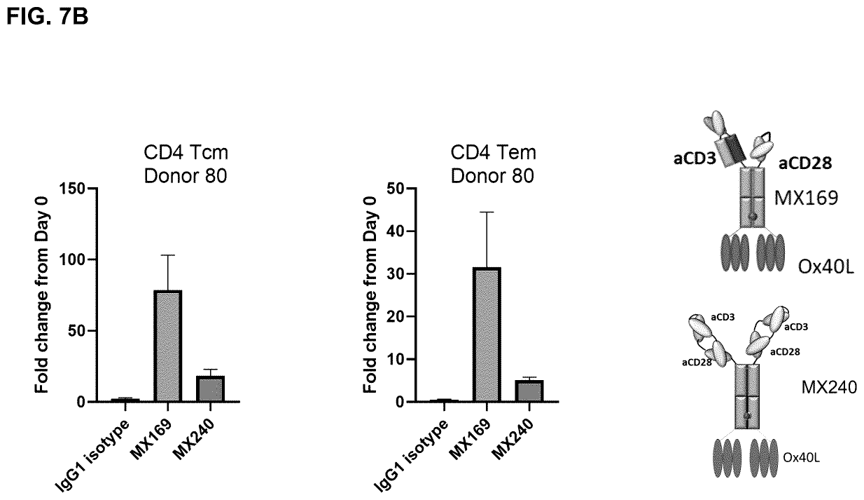

To assess the effect of multispecific antibodies on T cells, an in vitro T cell proliferation assay and flow cytometry were performed. MX169 was prepared as explained in Example 1. MX240 was also prepared as explained in Example 1 to contain a dimer of OX40L trimers and binding sites for CD3 and CD28, as shown in FIG. 7A-7B.

For the assay, purified human PBMCs (Blood Research Component, Brookline, MA, USA) were resuspended in culture medium (RPMI1640 with 10% FBS and supplemented with Penicillin Streptomycin)(Gibco) (2.5×10⁵ cells/ml). Alternatively, purified cynomolgus PBMC (Humancells Biosciences) were resuspended in culture medium (RPMI1640 with 10% FBS and supplemented with Penicillin Streptomycin and rIL-2 (50 units/mL)(Gibco). Serially-diluted multispecific and control antibodies (IgG1 isotype) were first coated onto 96-well flat-bottom culture plates by incubating 2-4 hours in a 37° C. tissue culture incubator. PBMC (200 μL) were then added to each well containing the antibodies and incubated for multiple time points (up to 10 days) in a 37° C. tissue culture incubator. The cell culture media was replaced after 4 days incubation. The cells were centrifuged, washed, and stained with fluorescent labeled antibodies for T cell markers, such as CD4 and CD8, along with viability dye and Precision Count Beads™ (Biolegend), before being acquired on an Attune flow cytometer (Thermo Fisher Scientific, USA). CD4 and CD8 T cells were identified and memory T cell subsets were characterized by CD45RA and CCR7 expressions. Fold change was calculated by dividing cell concentrations from Day 7 and Day 0. Data were analyzed using FlowJo software.

FIG. 7A-7B shows T cell proliferation, measured as the fold change of CD4 in PBMCs, caused by MX169 and MX240 from two donors (FIG. 7A and FIG. 7B, respectively). Results from a control antibody (IgG1 isotype) are also shown. These results show that both MX169 and MX240 induced CD4 memory T cell proliferation.

### Example 8

### Proliferation of Human CD4 Memory T Cells with OX40L Fusion Antibody Time Course

To assess the effect of multispecific antibodies on T cells, an in vitro T cell proliferation assay and flow cytometry were performed. MX169, MX368 and MX369 were prepared as explained in Example 1. The assay was performed as explained in Example 7. PBMC were incubated with multispecific antibodies for 3, 7, and 10 days.

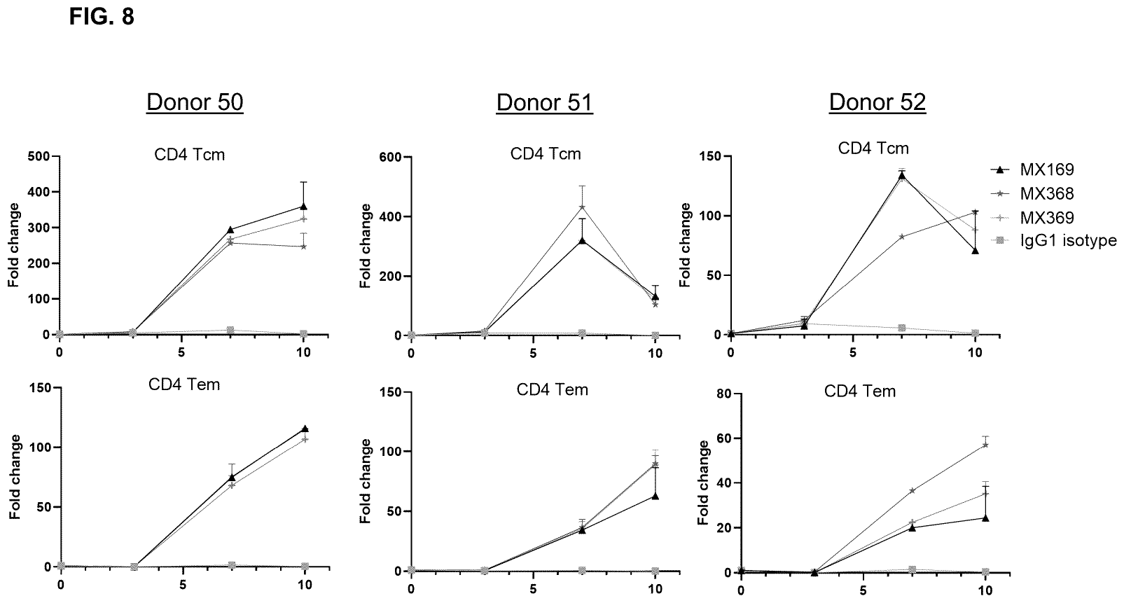

FIG. 8 shows T cell proliferation, measured as the fold change of CD4 in PBMCs, caused by MX169, MX368 and MX369 from three donors. Results from a control antibody (IgG1 isotype) are also shown. These results show that MX169, MX368 and MX369 induced CD4 memory T cell proliferation.

### Example 9

### Proliferation of Human CD8 Memory T Cells with OX40L Fusion Antibodies

To assess the effect of multispecific antibodies on T cells, an in vitro T cell proliferation assay and flow cytometry were performed. MX169 was prepared as explained in Example 1. MX240 was prepared as explained in Example 7. The assay was performed as explained in Example 7.

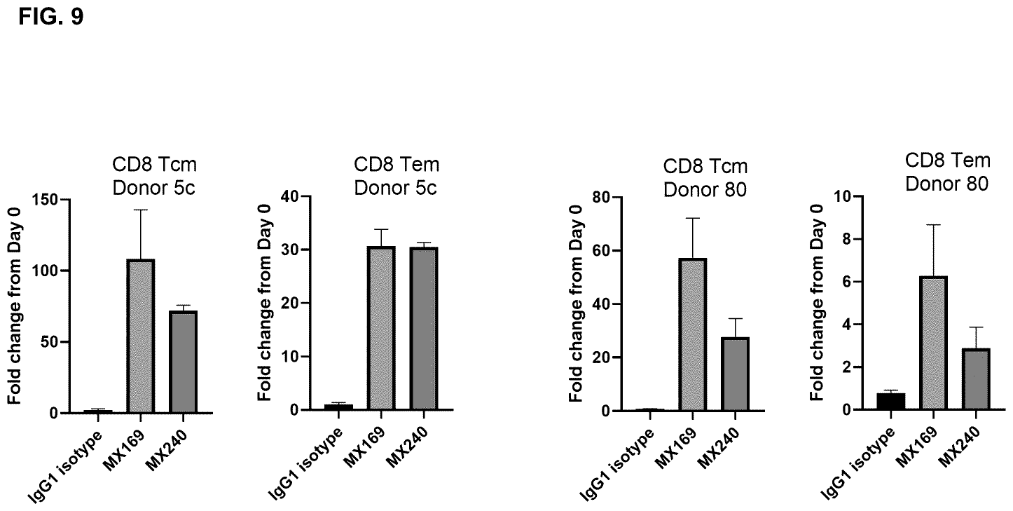

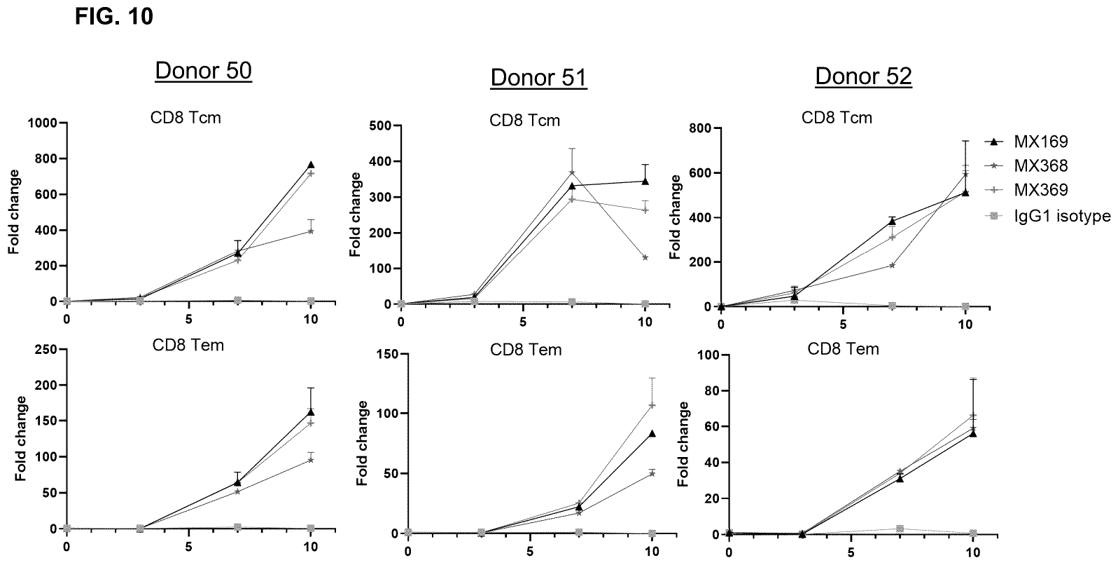

FIG. 9 shows T cell proliferation, measured as the fold change of CD8 in PBMCs, caused by MX169 and MX240 from two donors. Results from a control antibody (IgG1 isotype) are also shown. These results show that MX169 and MX240 induced CD8 memory T cell proliferation.

### Example 10

### Proliferation of Human CD8 Memory T Cells with OX40L Fusion Antibody Time Course

To assess the effect of multispecific antibodies on T cells, an in vitro T cell proliferation assay and flow cytometry were performed. MX169, MX368 and MX369 were prepared as explained in Example 1. The assay was performed as explained in Example 7. PBMC were incubated with multispecific antibodies for 3, 7, and 10 days.

FIG. 10 shows T cell proliferation, measured as the fold change of CD8 in PBMCs, caused by MX169, MX368 and MX369 from three donors. Results from a control antibody (IgG1 isotype) are also shown. These results show that MX169, MX368 and MX369 induced CD8 memory T cell proliferation.

### Example 11

### Proliferation of Human CD4 and CD8 Memory T Cells with 41BBL Fusion Antibodies

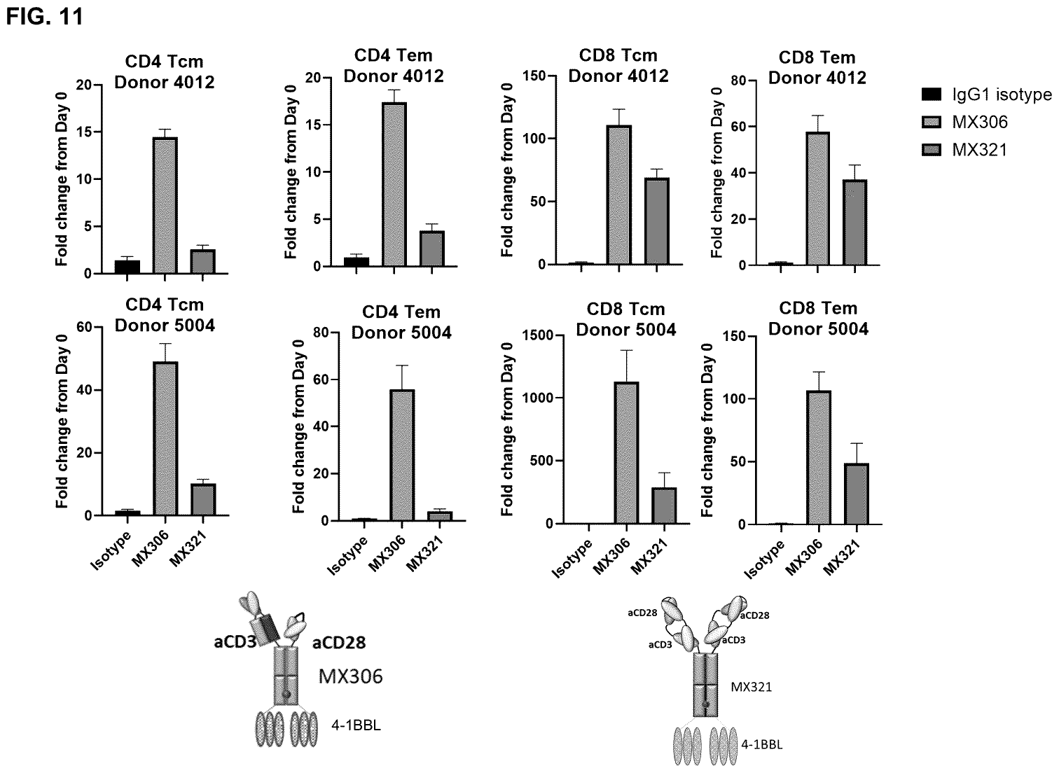

To assess the effect of multispecific antibodies on T cells, an in vitro T cell proliferation assay and flow cytometry were performed. MX306 was prepared as explained in Example 5. MX321 was also prepared as explained in Example 5 to contain a dimer of 4-1BBL trimers and binding sites for CD3 and CD28, as shown in FIG. 11. The assay was performed as explained in Example 7.

FIG. 11 shows T cell proliferation, measured as the fold change of CD4 or CD8 in PBMCs, caused by MX306 and MX321 from two donors. Results from a control antibody (IgG1 isotype) are also shown. These results show that MX306 and MX321 induced CD4 and CD8 memory T cell proliferation.

### Example 12

### Proliferation of Human CD4 Memory T Cells with 4-1BBL Fusion Antibody Time Course

To assess the effect of multispecific antibodies on T cells, an in vitro T cell proliferation assay and flow cytometry were performed. MX306, MX424 and MX425 were prepared as explained in Example 5. The assay was performed as explained in Example 7. PBMC were incubated with multispecific antibodies for 3, 7, and 10 days.

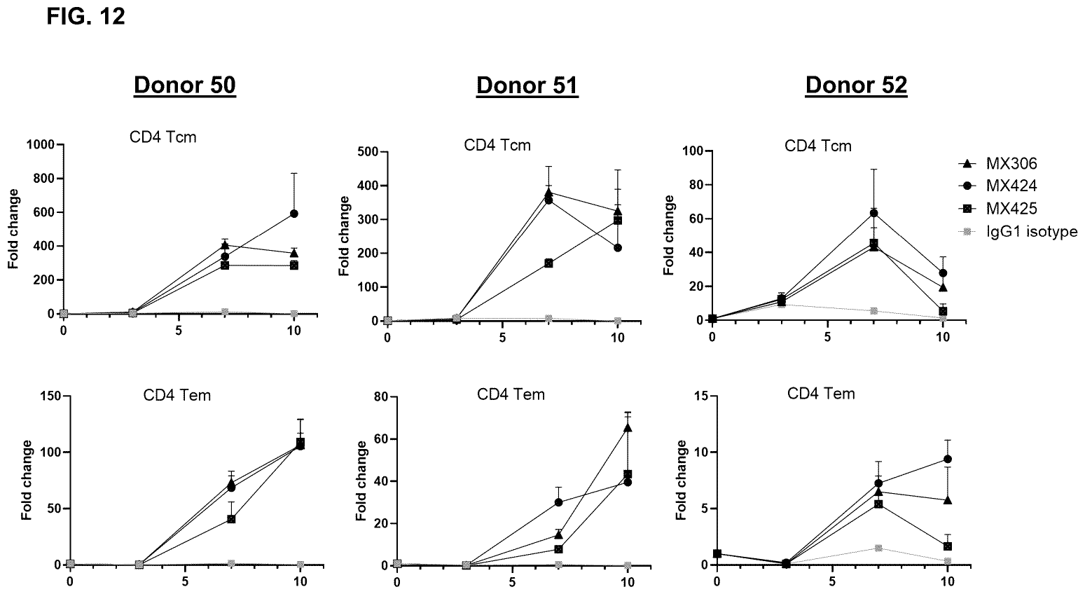

FIG. 12 shows T cell proliferation, measured as the fold change of CD4 in PBMCs, caused by MX306, MX424 and MX425 from three donors. Results from a control antibody (IgG1 isotype) are also shown. These results show that MX306, MX424 and MX425 induced CD4 memory T cell proliferation.

### Example 13

### Proliferation of Human CD8 Memory T Cells with 4-1BBL Fusion Antibody Time Course

To assess the effect of multispecific antibodies on T cells, an in vitro T cell proliferation assay and flow cytometry were performed. MX306, MX424 and MX425 were prepared as explained in Example 5. The assay was performed as explained in Example 7. PBMC were incubated with multispecific antibodies for 3, 7, and 10 days.

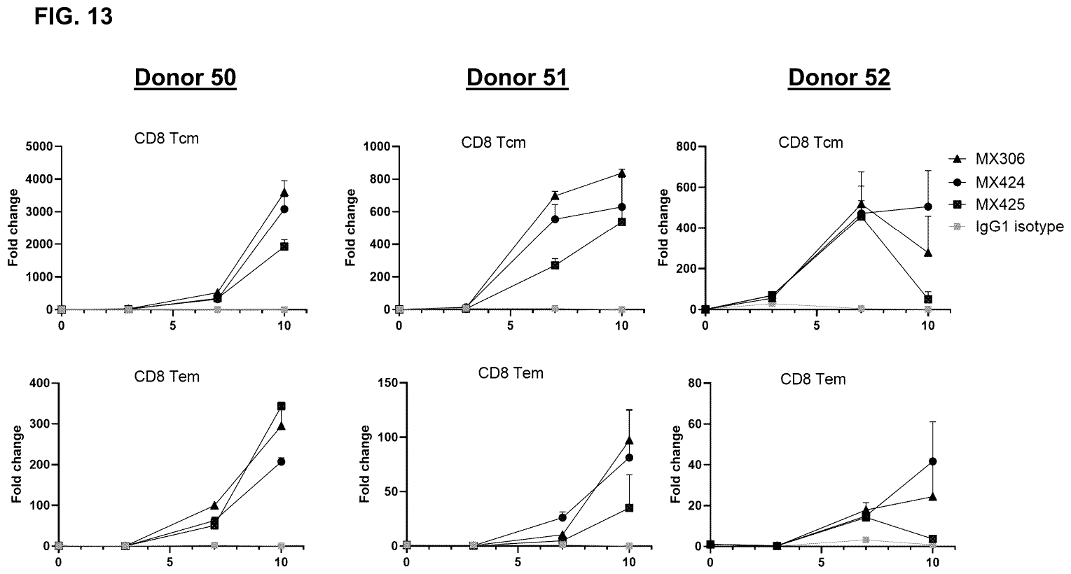

FIG. 13 shows T cell proliferation, measured as the fold change of CD8 in PBMCs, caused by MX306, MX424 and MX425 from three donors. Results from a control antibody (IgG1 isotype) are also shown. These results show that MX306, MX424 and MX425 induced CD4 memory T cell proliferation.

### Example 14

### Th1 Cytokine Release Concentrations from Primary Human T Cell Donor with OX40L Fusion Antibodies

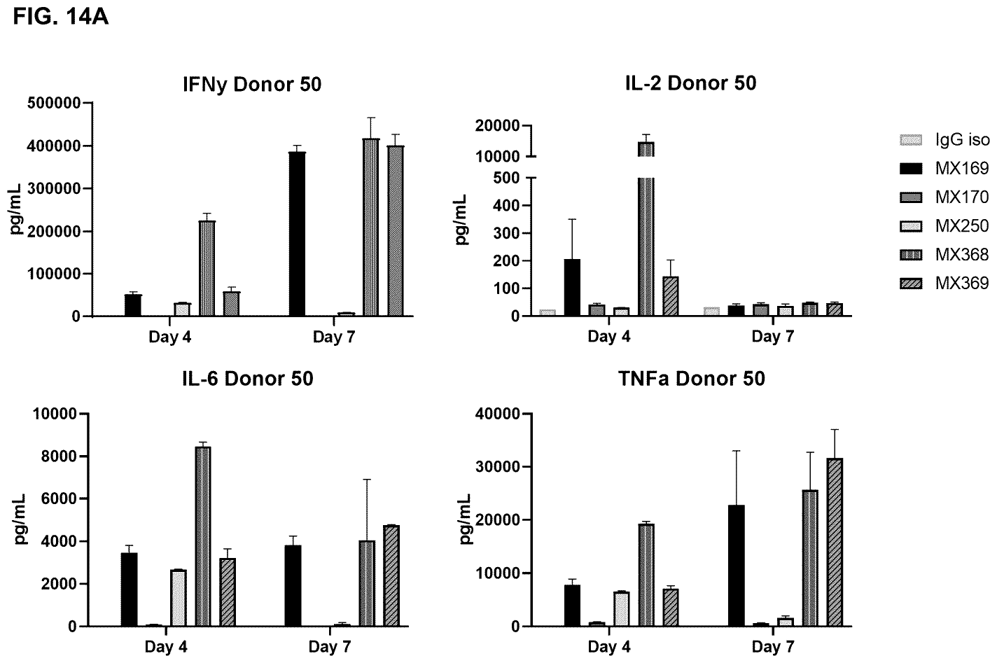

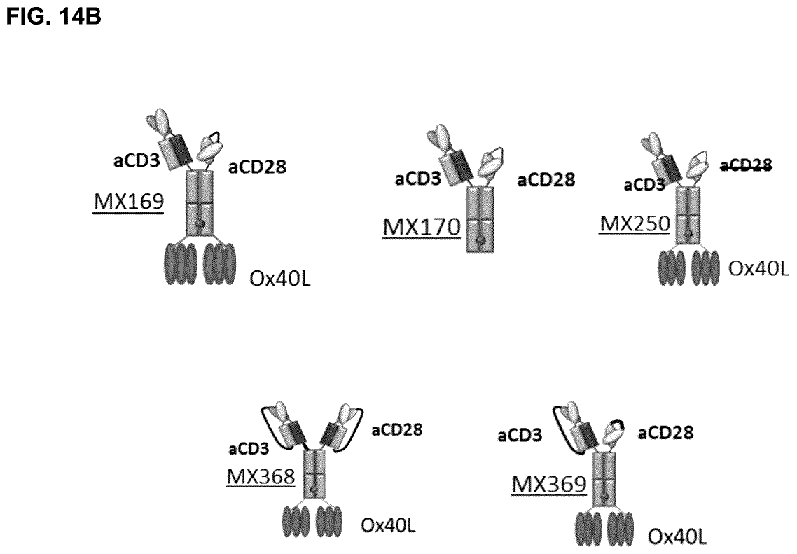

To assess the effect of multispecific antibodies on cytokine release, the following assay was performed. MX169, MX368 and MX369 were prepared as described above. MX170 and MX250 were also prepared using the methods explained above, with the resulting structures shown in FIG. 14B.

For the assay, cell culture supernatants were assayed using the MILLIPLEX® MAP multiplex assay (Millipore Sigma) with adoption of the Drop Array system (Curiox Biosystems, Singapore). Plasma samples were assayed using the ProcartaPlex™ immunoassay (Thermo Fisher) with adoption of the Drop Array system. In brief, magnetic analyte bead mixture was added to wells in the DropArray assay plate Standards, quality controls, and diluted samples were then added to the plate; all standards and quality controls were tested in duplicate, with samples tested in multiple replicates. The plate was placed on a magnetic stand in a humidified chamber and shaken overnight at 4° C. The plate was washed with a DropArray LT210 washing station (Curiox Biosystems). Detection antibody and streptavidin-PE substrate were added to each well and incubated with shaking. The plate was washed before reading by Luminex MAGPIX instrument. Data were analyzed using EMD Millipore Milliplex Analyst software or Thermofisher Procartaplex Analysis Tool.

FIG. 14A shows IFNgamma, IL-2, IL-6 and TNFa release from primary human T cells, caused by MX169, MX170, MX250, MX369 and MX369. Results from a control antibody (IgG1 iso) are also shown.

### Example 15

### Th2 Cytokine Release Concentrations from Primary Human T Cell Donor 50 with OX40L Fusion Antibodies

To assess the effect of multispecific antibodies on cytokine release, the following assay was performed. MX169, MX170, MX250, MX368 and MX369 were prepared as described above and assayed using the method described in Example 14. PBMCs were incubated with 1 nM multispecific antibodies for 4 and 7 days.

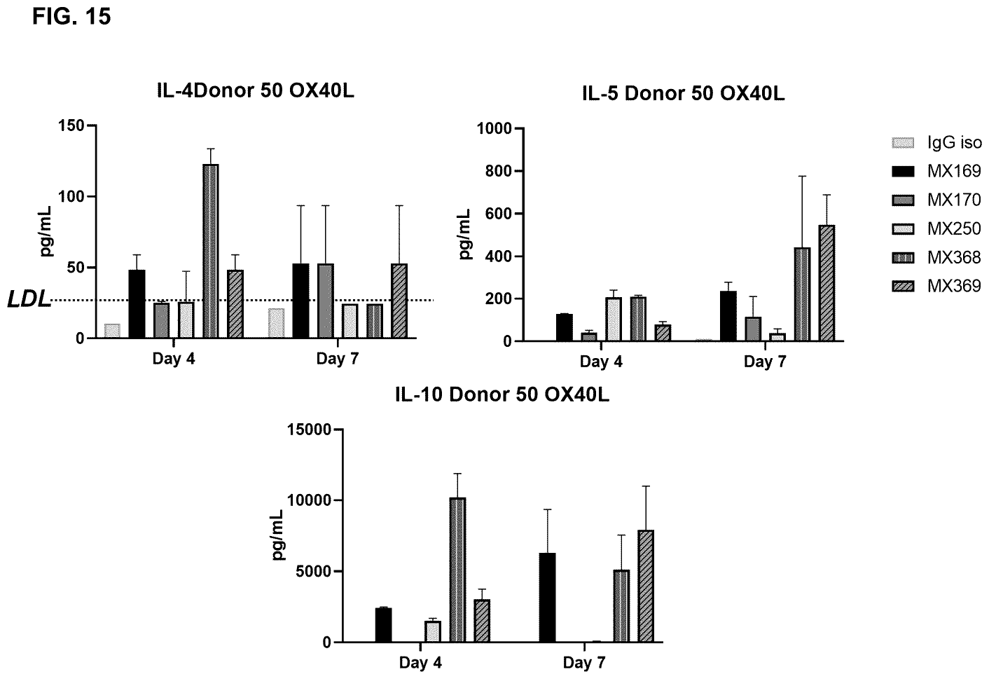

FIG. 15 shows IL-4, IL-5 and IL-10 release from primary human T cells, caused by MX169, MX170, MX250, MX368 and MX369. Results from a control antibody (IgG1 iso) are also shown.

### Example 16

### Th1 Cytokine Release Concentrations from Primary Human T Cell Donor 51 with OX40L Fusion Antibodies

To assess the effect of multispecific antibodies on cytokine release, the following assay was performed. MX169, MX170, MX250, MX368 and MX369 were prepared as described above and assayed using the method described in Example 14. PBMCs were incubated with 1 nM multispecific antibodies for 4 and 7 days.

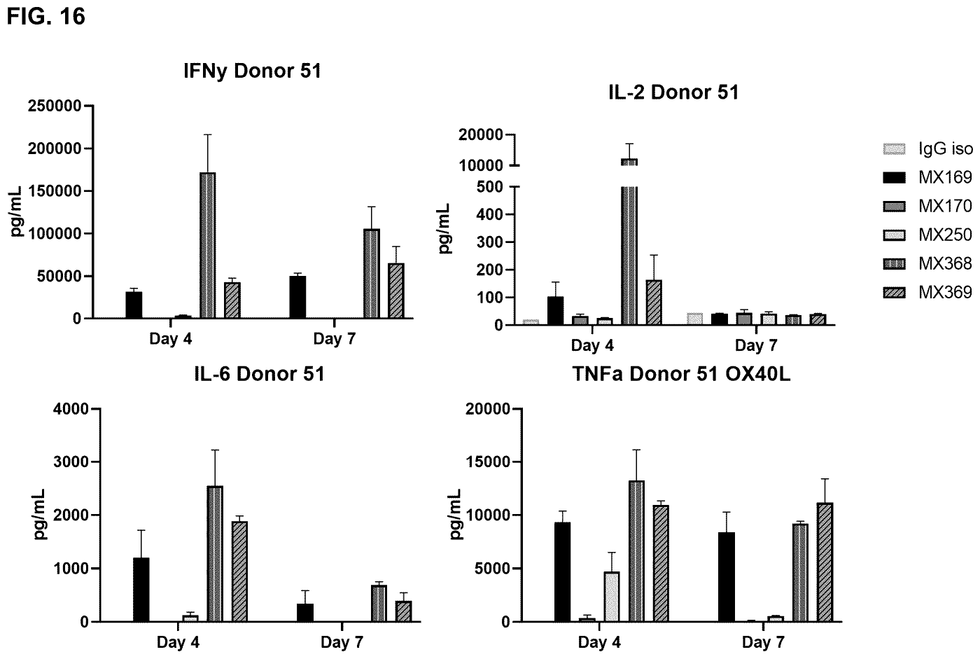

FIG. 16 shows IFNgamma, IL-2, IL-6 and TNFa release from primary human T cells, caused by MX169, MX170, MX250, MX368 and MX369. Results from a control antibody (IgG1 iso) are also shown.

### Example 17

### Th2 Cytokine Release Concentrations from Primary Human T Cell Donor 51 with OX40L Fusion Antibodies

To assess the effect of multispecific antibodies on cytokine release, the following assay was performed. MX169, MX170, MX250, MX368 and MX369 were prepared as described above and assayed using the method described in Example 14. PBMCs were incubated with 1 nM multispecific antibodies for 4 and 7 days.

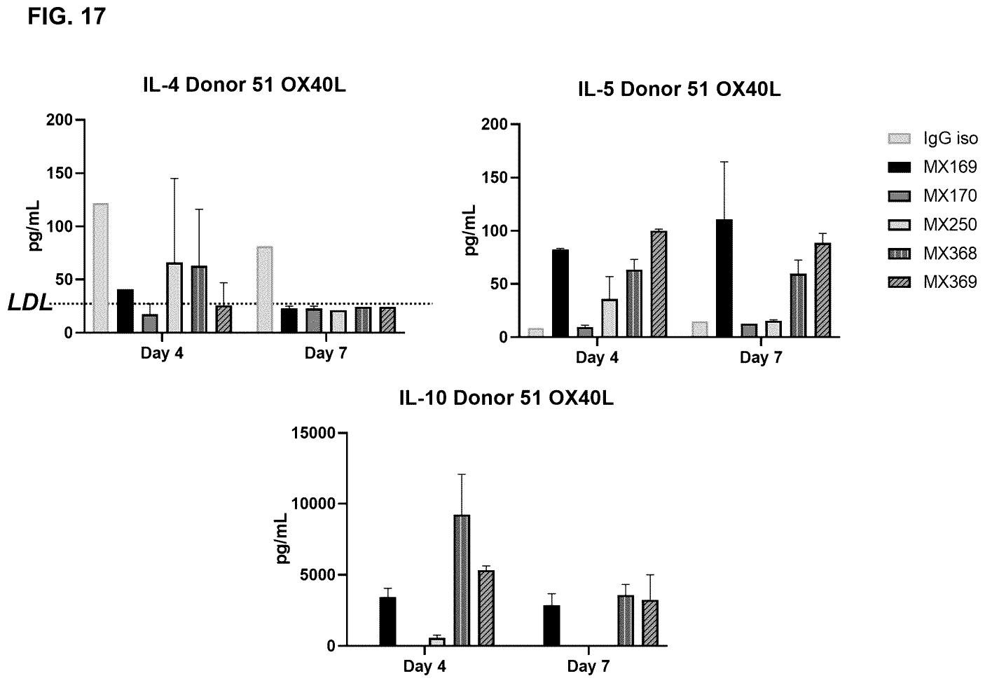

FIG. 17 shows IL-4, IL-5 and IL-10 release from primary human T cells, caused by MX169, MX170, MX250, MX368 and MX369. Results from a control antibody (IgG1 iso) are also shown.

### Example 18

### Th1 Cytokine Release Concentrations from Primary Human T Cell Donor 50 with 41BBL Fusion Antibodies

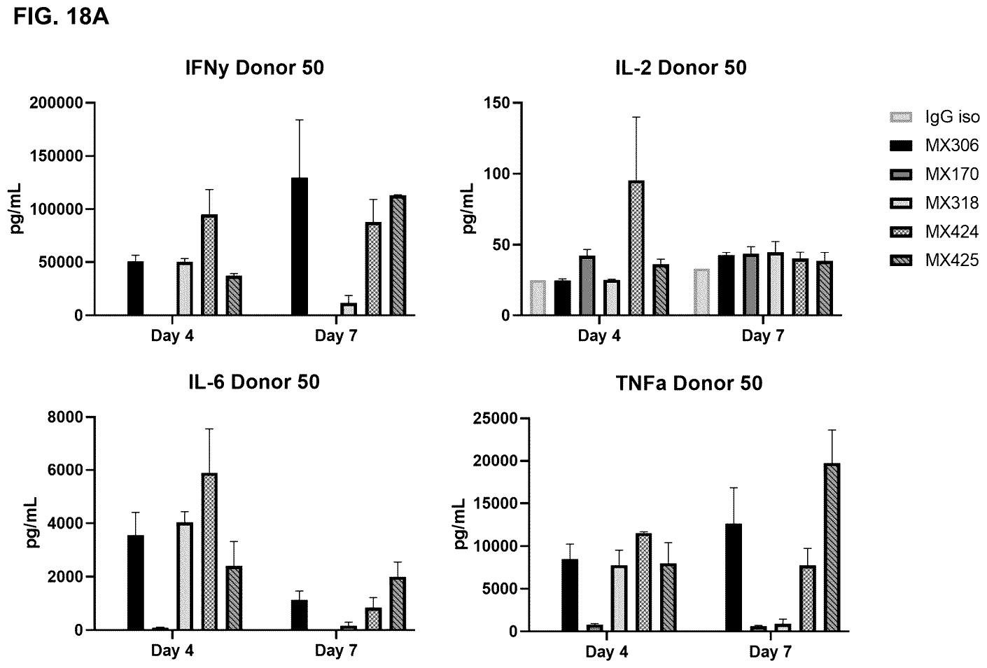

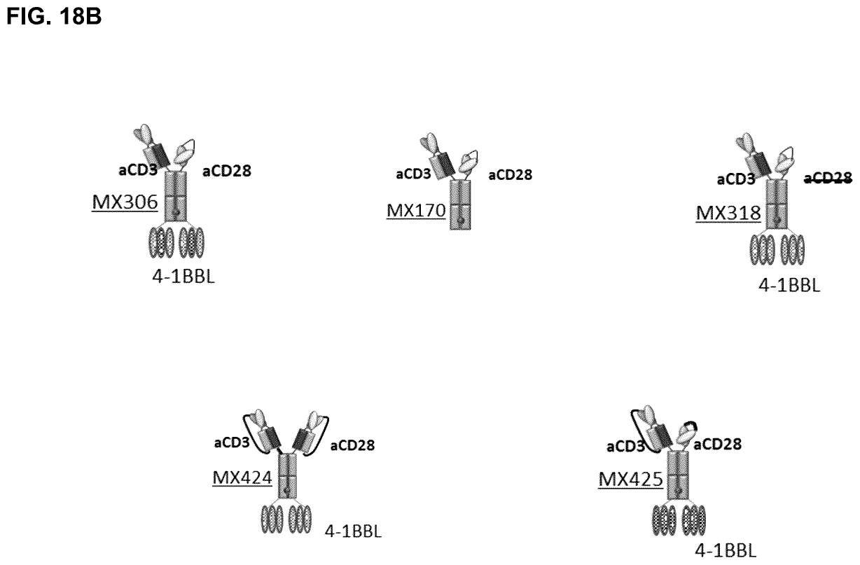

To assess the effect of multispecific antibodies cytokine release, the following assay was performed. MX306, MX170, MX424 and MX425 were prepared as described above. MX318 was also prepared using the methods explained above, with the resulting structure shown in FIG. 18B. Multispecific antibodies were assayed using the method described in Example 14. PBMCs were incubated with 1 nM multispecific antibodies for 4 and 7 days.

FIG. 18A shows IFNgamma, IL-2, IL-6 and TNFa release from primary human T cells, caused by MX306, MX170, MX318, MX424 and MX425. Results from a control antibody (IgG1 iso) are also shown.

### Example 19

### Th2 Cytokine Release Concentrations from Primary Human T Cell Donor 50 with 41BBL Fusion Antibodies

To assess the effect of multispecific antibodies on cytokine release, the following assay was performed. MX306, MX170, MX318, MX424 and MX425 were prepared as described above and assayed using the method described in Example 14. PBMCs were incubated with 1 nM multispecific antibodies for 4 and 7 days.

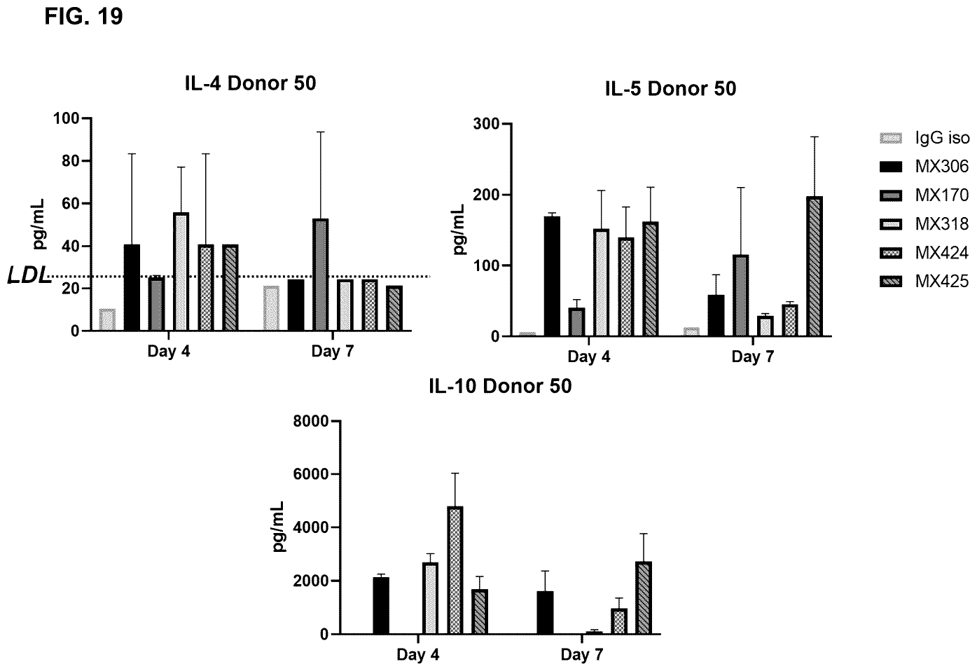

FIG. 19 shows IL-4, IL-5 and IL-10 release from primary human T cells, caused by MX306, MX170, MX318, MX424 and MX425. Results from a control antibody (IgG1 iso) are also shown.

### Example 20

### Th1 Cytokine Release Concentrations from Primary Human T Cells Donor 51 with 41BBL Fusion Antibodies

To assess the effect of multispecific antibodies on cytokine release, the following assay was performed. MX306, MX170, MX318, MX424 and MX425 were prepared as described above and assayed using the method described in Example 14. PBMCs were incubated with 1 nM multispecific antibodies for 4 and 7 days.

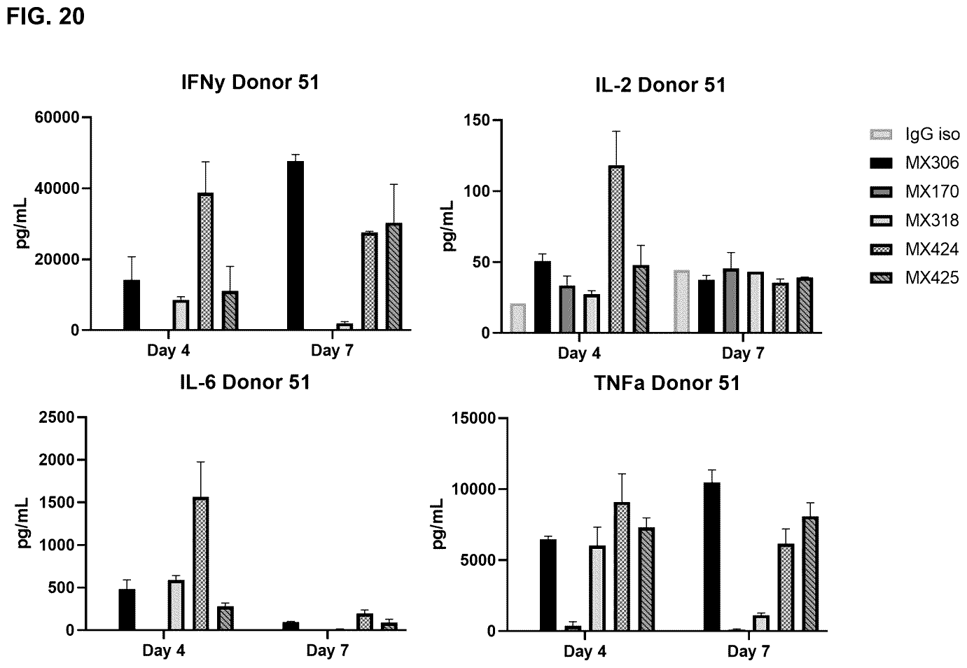

FIG. 20 shows IFNgamma, IL-2, IL-6, and TNFa release from primary human T cells, caused by MX306, MX170, MX318, MX424 and MX425. Results from a control antibody (IgG iso) are also shown.

### Example 21

### Th2 Cytokine Release Concentrations from Primary Human T Cell Donor 51 with 41BBL Fusion Antibodies

To assess the effect of multispecific antibodies on cytokine release, the following assay was performed. MX306, MX170, MX318, MX424 and MX425 were prepared as described above and assayed using the method described in Example 14. PBMCs were incubated with 1 nM multispecific antibodies for 4 and 7 days.

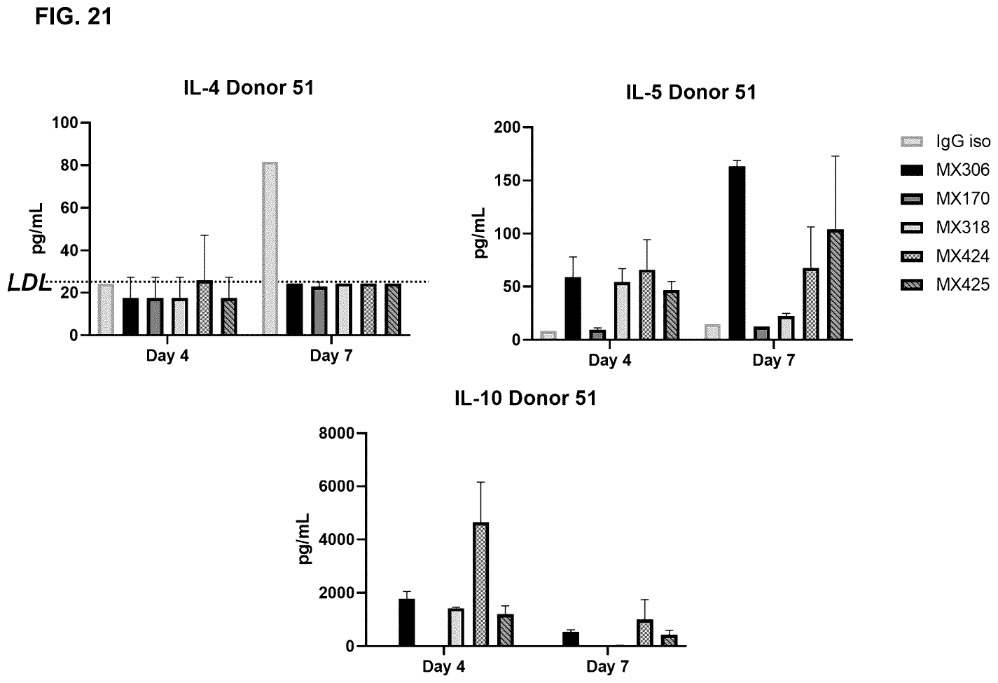

FIG. 21 shows IL-4, IL-5 and IL10 release from primary human T cells, caused by MX306, MX170, MX318, MX424 and MX425. Results from a control antibody (IgG iso) are also shown.

### Example 22

### Activation of Monkey T Cells with 41BBL Fusion Antibodies

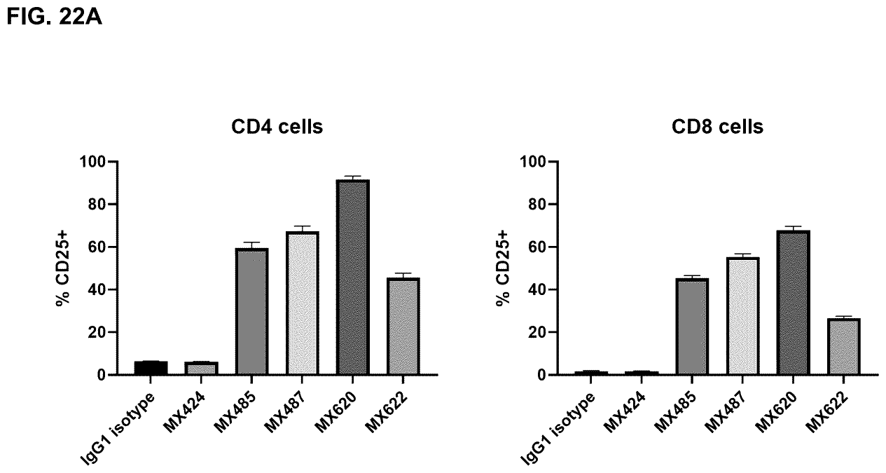

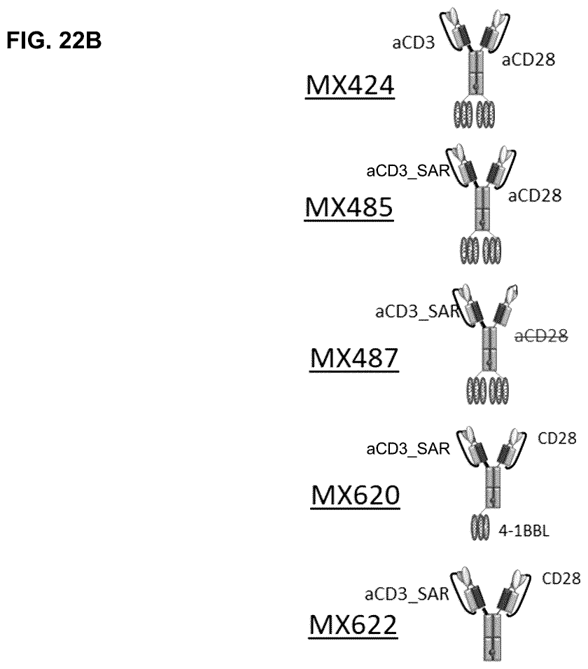

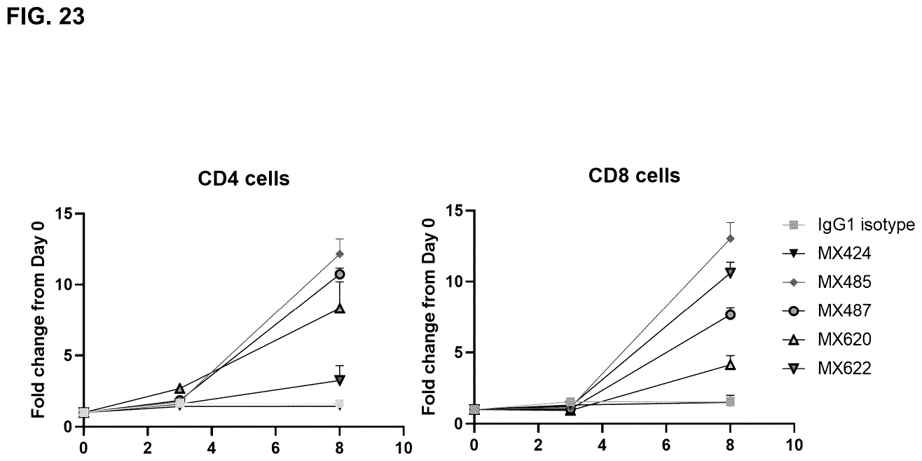

To assess the effect of multispecific antibodies on cytokine release, the following assay was performed. MX424 was prepared as described above. MX485, MX487, MX620 and MX622 were also prepared as described above, having structures shown in FIG. 22B. Multispecific antibodies were assayed using the method described in Example 14, with the following changes: NHP PBMCs were incubated with 1 nM multispecific antibodies for 3 days in culture media supplemented with rIL-2. The cells were then stained for flow cytometry. CD4 and CD8 cells were identified and their activation state was determined by percentage of CD25 expression. Only antibodies with NHP-reactive anti-CD3 demonstrated T cell activation.

FIG. 22A shows activation of monkey CD4 and CD8 T cells, caused by MX424, MX485, MX487, MX620 and MX622. Results from a control antibody (IgG1 isotype) are also shown.

### Example 23

### Proliferation of Monkey T Cells with 41BBL Fusion Antibodies

Proliferation of monkey T cells following treatment with MX424, MX485, MX487, MX620 and MX622 was also tested. Cynomolgus non-human primate (NHP) PBMCs were incubated with 1 nM multispecific antibodies for 8 days in culture media supplemented with rIL-2. The cells were then stained with flow cytometry. CD4 and CD8 cells were identified and their concentration were determined using Precision Count Beads™ (Biolegend). Fold change was calculated by dividing cell concentrations from each time point with Day 0. Only antibodies with NHP-reactive anti-CD3 demonstrated T cell proliferation. For the exploratory NHP study, cynomolgus monkeys were used with all animal procedures and experiments performed according to protocols approved by the Institutional Animal Care and Use Committee. MX487 and MX620 were administered via multiple 1-hour IV (10-30-100 μg/kg) infusion administration, or MX620 was administered via multiple 1-hour IV (100-300 μg/kg) infusion administration. Whole blood samples were collected prior to injection and at multiple time points as designed until day 14 after administration. Flow cytometry based immunophenotyping of circulating cells was carried out using a panel of cell surface markers to identify cell subpopulations (CD2, CD4, CD8, CD45RA, and CCR7).

FIG. 23 shows fold change of CD4 and CD8 cells, caused by MX424, MX485, MX487, MX620 and MX622. Results from a control antibody (IgG1 isotype) are also shown.

### Example 24

### Cytokine Release Concentrations from Monkey T Cells Incubated with 41BBL Fusion Antibodies

Cytokine release of monkey T cells following treatment with MX424, MX485, MX487, MX620 and MX622 was tested. Cynomolgus non-human primate (NHP) PBMC were incubated with 1 nM multispecific antibodies for 3 days in culture media supplemented with rIL-2. The supernatants were collected and multiplexed using Luminex® assay to determine cytokine concentrations. (rIL-2 supplement detected in this assay).

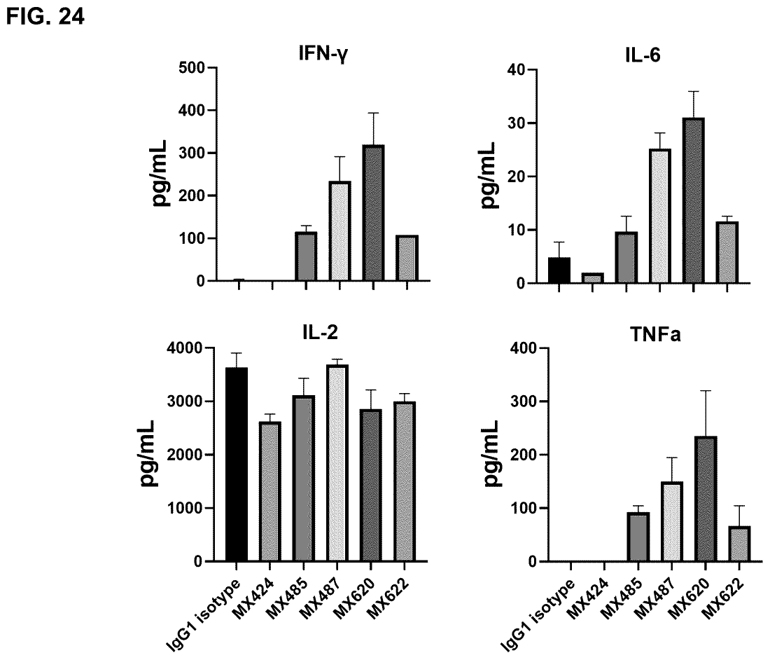

FIG. 24 shows release of IFNgamma, IL-6, IL-2 and TNFa, caused by MX424, MX485, MX487, MX620 and MX622. Results from a control antibody (IgG1 isotype) are also shown.

### Example 25

### Activation of Monkey T Cells with OX40L Fusion Antibodies

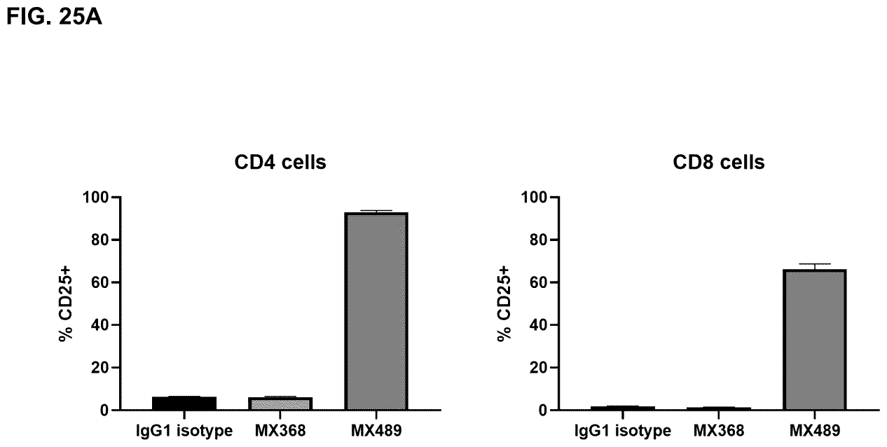

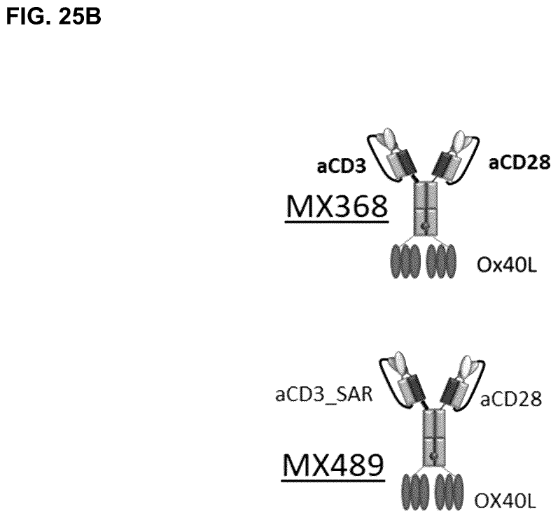

Activation of monkey T cells following treatment with MX368 and MX489 was tested. MX368 was prepared as discussed above. MX489 was also prepared as discussed above, having a structure shown in FIG. 25B. To test activation, cynomolgus non-human primate (NHP) PBMC were incubated with 1 nM multispecific antibodies for 3 days in culture media supplemented with rIL-2. The cells were then stained for flow cytometry. CD4 and CD8 cells were identified and their activation state was determined by percentage of CD25 expression. Only antibody with NHP-reactive anti-CD3 demonstrated T cell activation.

FIG. 25A shows activation of CD4 and CD8 monkey T cells caused by MX368 and MX489. Results from a control antibody (IgG1 isotype) are also shown.

### Example 26

### Proliferation of Monkey T Cells with OX40L Fusion Antibodies

Proliferation of monkey T cells following treatment with MX368 and MX489 was tested as explained above. Cynomolgus non-human primate (NHP) PBMC were incubated with 1 nM multispecific antibodies for 8 days in culture media supplemented with rIL-2. The cells were then stained for flow cytometry. CD4 and CD8 cells were identified and their concentration were determined using Precision Count Beads™ (Biolegend). Fold change was calculated by dividing cell concentrations from each time point with Day 0. Only antibody with NHP-reactive anti-CD3 demonstrated T cell proliferation.

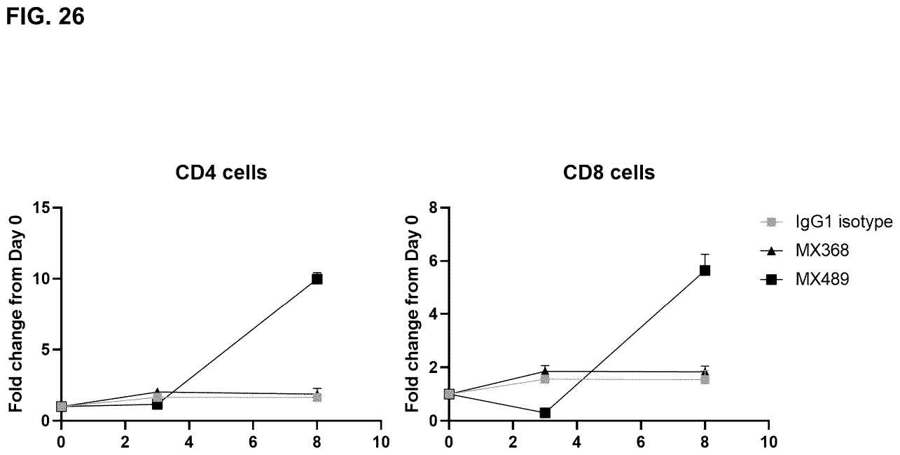

FIG. 26 shows proliferation of CD4 and CD8 monkey T cells caused by MX368 and MX489. Results from a control antibody (IgG1 isotype) are also shown.

### Example 27

### Cytokine Release Concentrations from Monkey T Cells Incubated with OX40L Fusion Antibodies

Cytokine release from monkey T cells following treatment with MX368 and MX489 was tested as explained above. Cynomolgus non-human primate (NHP) PBMC were incubated with 1 nM multispecific antibodies for 3 days in culture media. The supernatants were collected and multiplexed using Luminex® assay to determine cytokine concentrations.

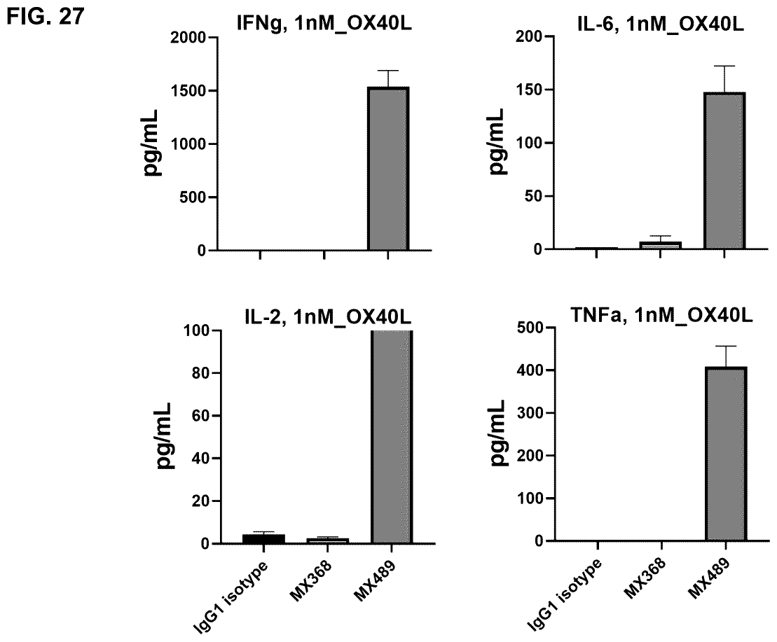

FIG. 27 shows release of IFNgamma (IFNg), IL-6, IL-2 and TNFa from monkey T cells caused by MX368 and MX489. Results from a control antibody (IgG1 isotype) are also shown.

### Example 28

### T Cells in NHP Expand and then Return to Baseline after Dosing with 41BBL Fusion Antibody MX487

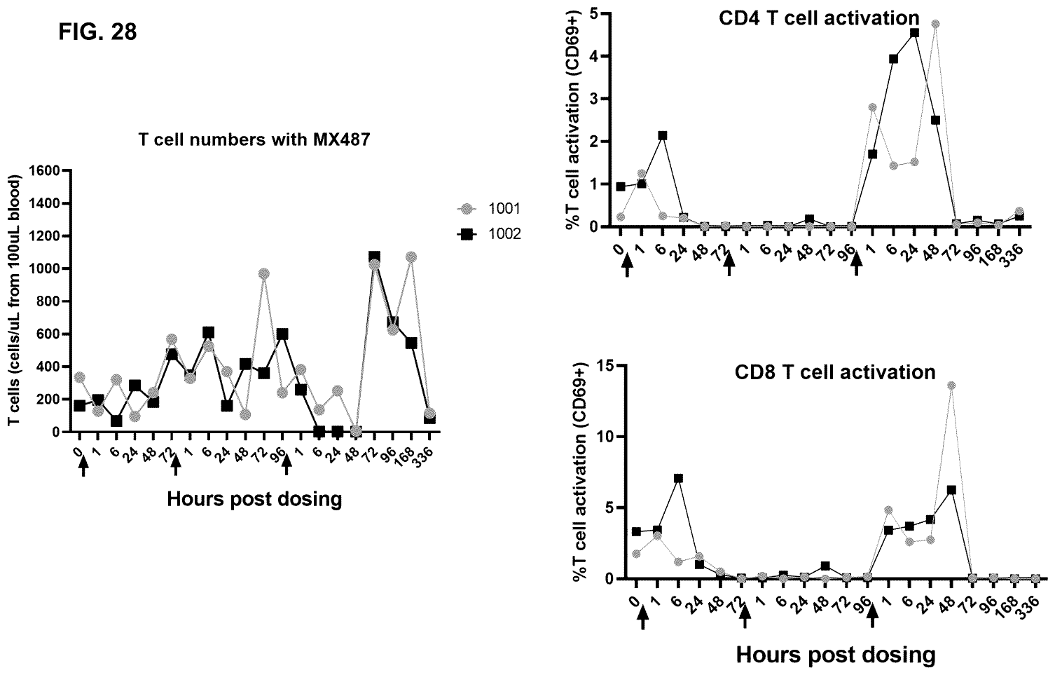

Two cynomolgus non-human primates (NHPs) were given 3 intra-animal escalating doses of 41BBL fusion antibody MX487, starting with 0.01, 0.03, and a final dose 0.1 mg/kg. Blood was collected at various time points and immunophenotyped by flow cytometry. Results are shown in FIG. 28. Arrows indicate antibody dosing. These results show that T cells in primates expand and then return to baseline after dosing with MX487.

### Example 29

### Naïve T Cell Memory Subset Dominates in NHP After Dosing with 41BBL Fusion Antibody MX487

Cynomolgus NHPs were given 3 intra-animal escalating doses of 41BBL fusion antibody MX487, with 0.01, 0.03, and 0.1 mg/kg as the final dose. Blood was collected 1 week after first dosing and immunophenotyped by flow cytometry. Memory T cell subsets were characterized by CD45RA and CCR7 expression.

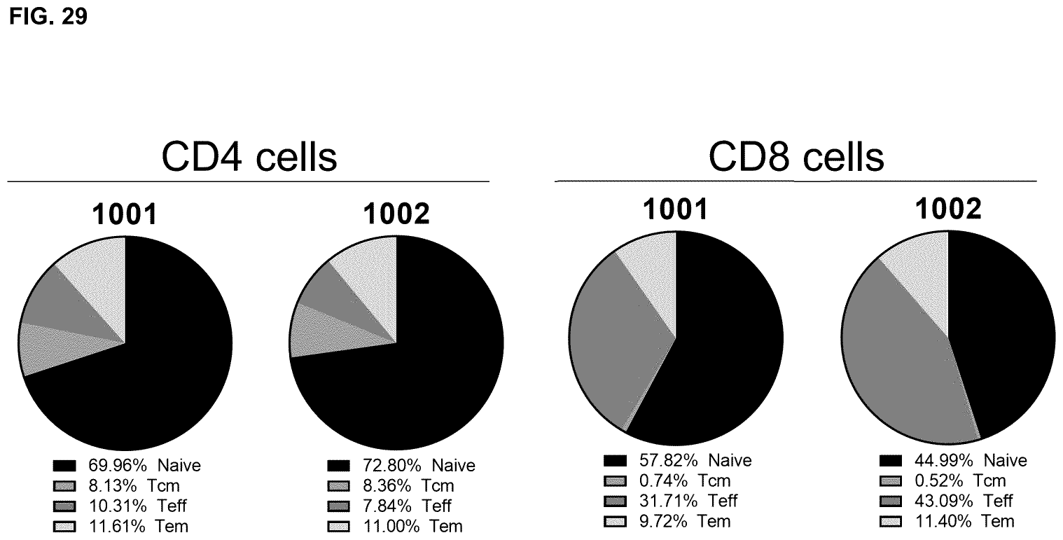

FIG. 29 shows the percentage of naïve, Tcm, Teff and Tem populations of CD4 and CD8 T cells from two different animals. These results show that naïve CD4 and CD8 memory T cells are the primary population in primates following treatment with MX487.

### Example 30

### T Cells in NHPs Greatly Expand after Dosing with 41BBL Fusion Antibody MX620

Cynomolgus NHPs were given 3 intra-animal escalating doses of 41BBL fusion antibody MX620, with 0.01, 0.03, and 0.1 mg/kg as the final dose. Blood was collected at various time points and immunophenotyped by flow cytometry.

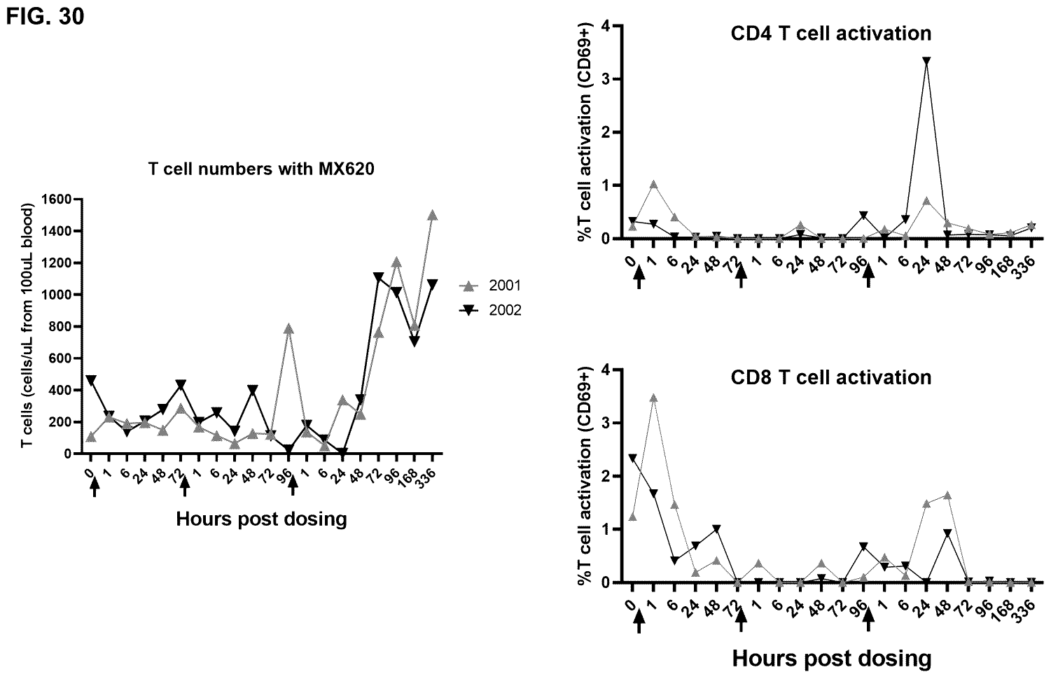

FIG. 30 shows the number of T cells and CD4 and CD8 T cell activation from two NHPs treated with MX620. Arrows indicate Ab dosing. These results show that T cells greatly expanded after treatment with MX620.

### Example 31

### Effector Memory T Cell Subset Dominates in NHPs After Dosing with 41BBL Fusion Antibody MX620

Cynomolgus NHPs were given 3 intra-animal escalating doses of 41BBL fusion antibody MX620, with 0.01, 0.03, and 0.1 mg/kg as the final dose. Blood was collected 1 week after first dosing and immunophenotyped by flow cytometry. Memory T cell subsets were characterized by CD45RA and CCR7 expression.

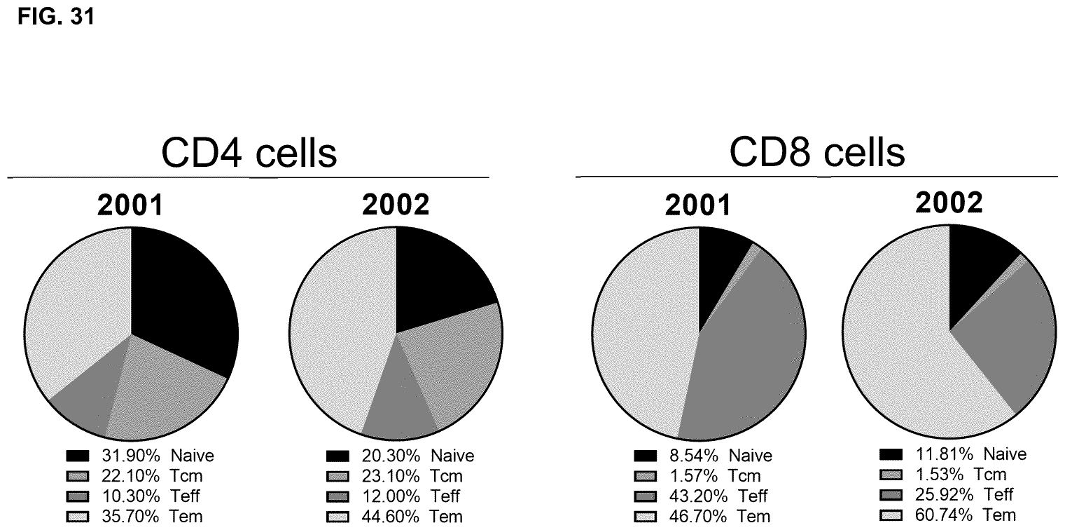

FIG. 31 shows the percentage of naïve, Tcm, Teff and Tem populations of CD4 and CD8 T cells from two different animals.

### Example 32

### T Cells in NHPs Expand after Dosing with 41BBL Fusion Antibody MX620

Cynomolgus NHPs were given two intra-animal escalating doses of 41BBL fusion antibody MX620, with 0.1 and 0.3 mg/kg. Blood was collected at various time points and immunophenotyped by flow cytometry. Cell concentration was determined using Precision Count Beads™ (Biolegend). Fold change was calculated by dividing cell concentrations from each time point with Day −2 value.

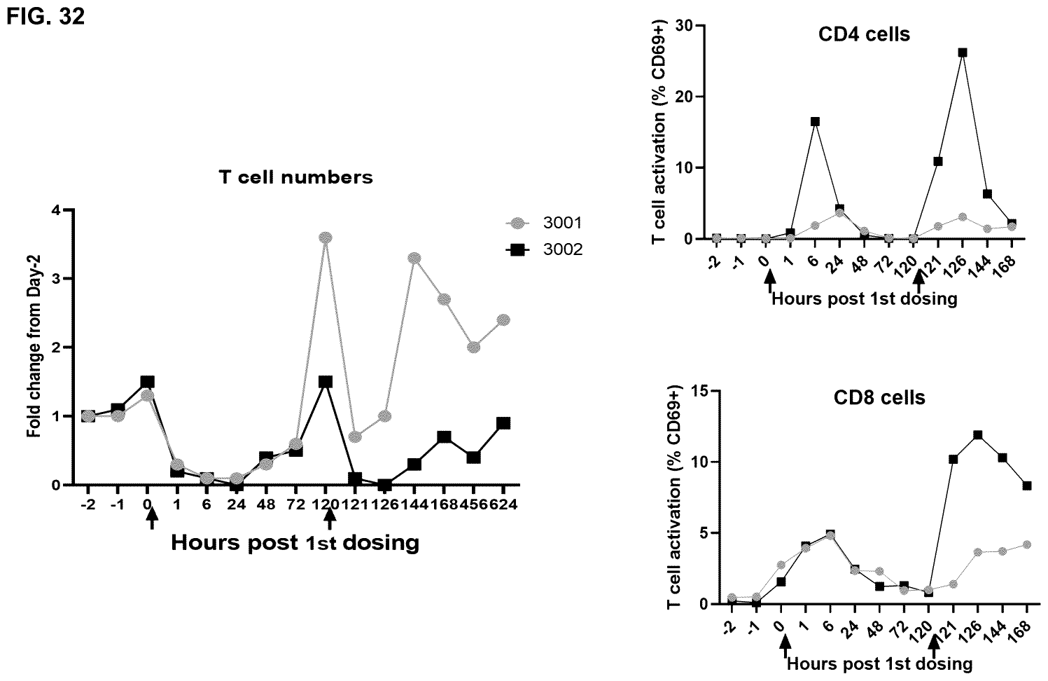

FIG. 32 shows the fold change in T cell number and CD4 and CD8 T cell activation from 2 NHPs, following treatment with MX620. Arrows indicate Ab dosing. These results show that T cells from NHPs expanded after dosing with MX620.

### Example 33

### Effector Memory CD8 T Cell Subset Dominates in NHPs After Dosing with 41BBL Fusion Antibody MX620

Cynomolgus NHPs were given two intra-animal escalating doses of 41BBL fusion antibody MX620, with 0.1 and 0.3 mg/kg. Blood was collected at various time points and immunophenotyped by flow cytometry. Memory T cell subsets were characterized by CD45RA and CCR7 expressions. Cell concentration was determined using Precision Count Beads™ (Biolegend). Fold change was calculated by dividing cell concentrations from each time point with Day −2 value.

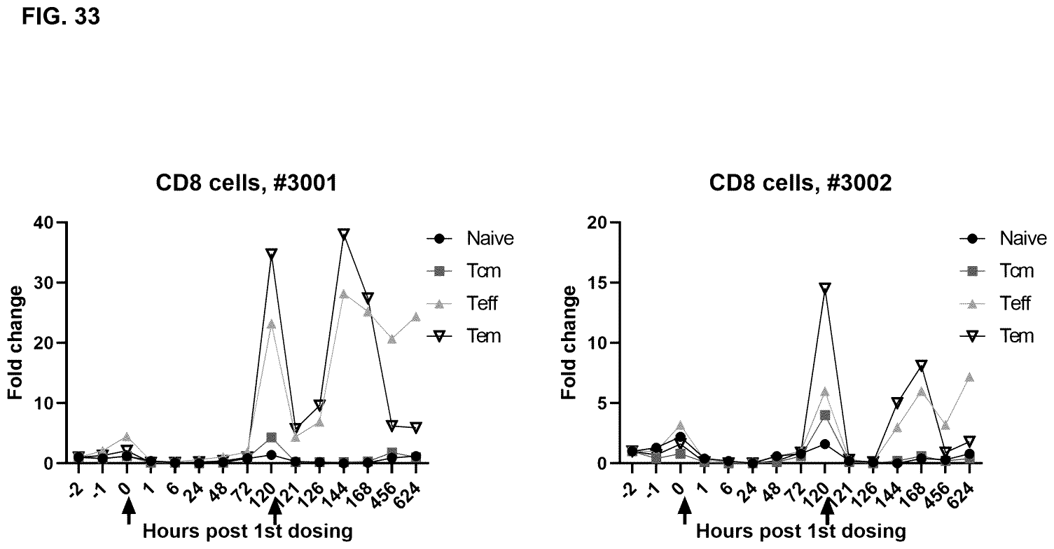

FIG. 33 shows the fold change in naïve, Tcm, Teff and Tem populations of CD8 T cells from two different NHPs. Arrows indicate Ab dosing.

### Example 34

### In Vitro Killing of Z138 Cells is Mediated by CD19×CD20/CD3_4-1BBL Fusion Antibodies

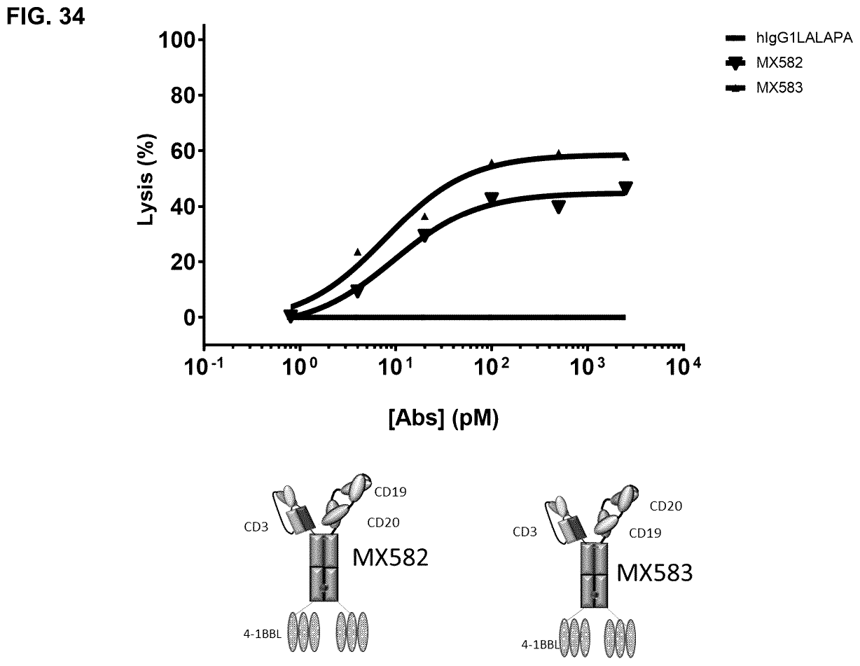

MX582 and MX583 fusion antibodies were prepared as described above to have the structure shown in FIG. 34. Killing of Z138 cells following treatment for 48 hours (E:T ratio of 3:1) with MX582 and MX583 was tested using the following assay. Z138 tumor target cells were labeled with PKH26 dye and then washed in culture media. Target cells were plated into a 96 well U-bottom plates at a final concentration of 2×10⁴ cells. Pan-T cells were prepared and added at a final concentration of 6×10⁴ cells/well at a E:T ratio of 3:1. Antibodies were serially diluted in media and added for a final concentration between 2.5 nM down to 0.8 pM. Plates were incubated 48 hrs at 37° C. in 5% CO₂. Cells were pelleted after incubation and stained with Violet dead stain 1:1000 for 20 minutes. Stain was washed and cells were resuspended in FACS fix buffer. Cells were analyzed by Attune Cytpix flow cytometer followed by analysis using FloJo software. Target cells were gated as PE positive. Dead target cells were identified as APC positive. The in vitro killing activation effect was calculated as percentage of cytolytic activity. Lysis %=100−(experimental live target cell/average of control antibody group live target cell)*100. The percentages of cytotoxicity were processed in GraphPad Prism.

FIG. 34 shows percent lysis of Z138 cells following treatment with increasing concentrations of MX582 and MX583. Treatment with a control antibody (hIgG1LALAPA) is also shown. These results show that MX582 and MX583 mediate cell killing against Z138 cells.

### Example 35

### In Vitro Killing of Z138 Cells is Mediated by CD19×CD20/CD3 of 4-1BBL Fusion Antibodies

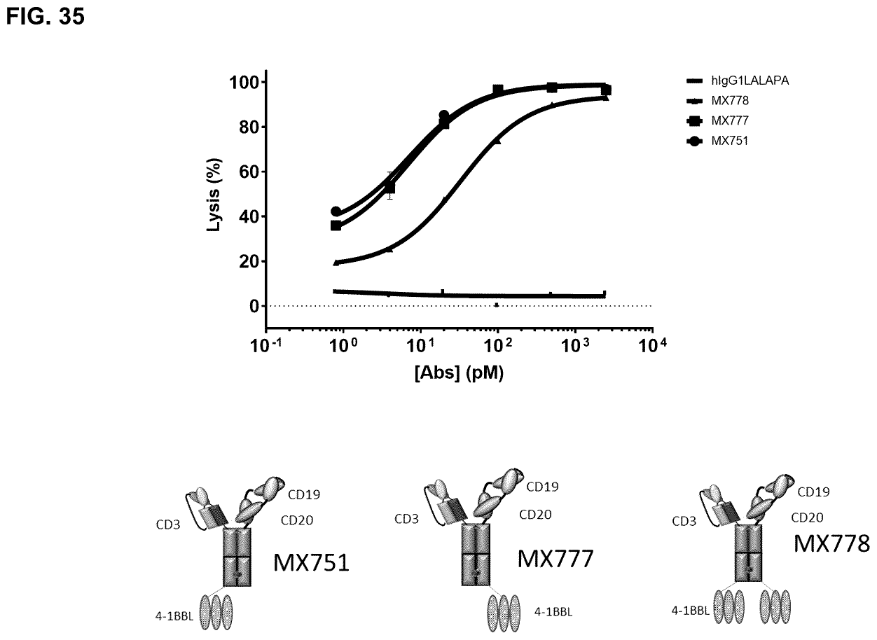

MX751, MX777 and MX778 fusion antibodies were prepared as described above to have the structure shown in FIG. 35. Killing of Z138 cells following treatment for 48 hours (E:T ratio of 3:1) with MX751, MX777 and MX778 was tested as described above.

FIG. 35 shows percent lysis of Z138 cells following treatment with increasing concentrations of MX751, MX777 and MX778. Treatment with a control antibody (hIgG1LALAPA) is also shown. These results show that MX751, MX777 and MX778 mediate cell killing against Z138 cells.

All publications, patents, patent applications and biological deposit mentioned in this application are herein incorporated in their entirety by reference into the specification, to the same extent as if each individual publication, patent, patent application or biological deposit was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An antigen binding polypeptide complex comprising a first polypeptide, a second polypeptide, and a third polypeptide;
(a) wherein the first polypeptide has a structure represented by:
VL1-L1-CL;
VL1-L1-CH1;
VH1-L1-CL; or
VH1-L1-CH1;
wherein:
(i) the second polypeptide has a structure represented by:
VH1-L2-CH1-L3-Fc-L4-TNF1-L5-TNF2-L6-TNF3;
VH1-L2-CL-L3-Fc-L4-TNF1-L5-TNF2-L6-TNF3;
VH1-L7-CH1-L8-Fc;
VH1-L7-CL-L8-Fc;
VL1-L2-CH1-L3-Fc-L4-TNF1-L5-TNF2-L6-TNF3;
VL1-L2-CL-L3-Fc-L4-TNF1-L5-TNF2-L6-TNF3;
VL1-L7-CH1-L8-Fc; or
VL1-L7-CL-L8-Fc; and
the third polypeptide has a structure represented by:
VL2-L9-VH2-L10-Fc-L11-TNF1-L12-TNF2-L13-TNF3; or
VH2-L14-VL2-L15-Fc-L16-TNF1-L17-TNF2-L18-TNF3;
or
(ii) the second polypeptide has a structure represented by:
VH1-L19-CH1-L20-Fc-L21-TNF1-L22-TNF2-L23-TNF3;
VH1-L19-CL-L20-Fc-L21-TNF1-L22-TNF2-L23-TNF3;
VL1-L19-CH1-L20-Fc-L21-TNF1-L22-TNF2-L23-TNF3; or
VL1-L19-CL-L20-Fc-L21-TNF1-L22-TNF2-L23-TNF3; and
the third polypeptide has a structure represented by:
VL2-L24-VH2-L25-Fc; or
VH2-L26-VL2-L27-Fc;
wherein:
VL1 is a first immunoglobulin light chain variable region;
VL2 is a second immunoglobulin light chain variable region;
VH1 is a first immunoglobulin heavy chain variable region;
VH2 is a second immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region;
TNF1 is a first extracellular domain of a tumor necrosis factor superfamily (TNFSF) ligand;
TNF2 is a second extracellular domain of a TNFSF ligand;
TNF3 is a third extracellular domain of a TNFSF ligand; and
L1-L27 are amino acid linkers; or
(b) wherein the first polypeptide has a structure represented by:
VL1-L1-CL;
VL1-L1-CH1;
VH1-L1-CL; or
VH1-L1-CH1;
wherein:
(i) the second polypeptide has a structure represented by:
VH1-CH1-L2-Fc-L3-TNF1-L4-TNF2-L5-TNF3;
VH1-CL-L2-Fc-L3-TNF1-L4-TNF2-L5-TNF3;
VH1-CH1-L6-Fc;
VH1-CL-L7-Fc;
VL1-CH1-L2-Fc-L3-TNF1-L4-TNF2-L5-TNF3;
VL1-CL-L2-Fc-L3-TNF1-L4-TNF2-L5-TNF3;
VL1-CH1-L6-Fc; or
VL1-CL-L7-Fc; and
the third polypeptide has a structure represented by:
VL2-L8-VH2-L9-Fc-L10-TNF1-L11-TNF2-L12-TNF3; or
VH2-L13-VL2-L14-Fc-L15-TNF1-L16-TNF2-L17-TNF3;
or
(ii) the second polypeptide has a structure represented by:
VH1-CH1-L18-Fc-L19-TNF1-L20-TNF2-L21-TNF3;
VH1-CL-L18-Fc-L19-TNF1-L20-TNF2-L21-TNF3;
VL1-CH1-L18-Fc-L19-TNF1-L20-TNF2-L21-TNF3; or
VL1-CL-L18-Fc-L19-TNF1-L20-TNF2-L21-TNF3; and
the third polypeptide has a structure represented by:
VL2-L22-VH2-L23-Fc; or
VH2-L24-VL2-L25-Fc;
wherein:
VL1 is a first immunoglobulin light chain variable region;
VL2 is a second immunoglobulin light chain variable region;
VH1 is a first immunoglobulin heavy chain variable region;
VH2 is a second immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region;
TNF1 is a first extracellular domain of a tumor necrosis factor superfamily (TNFSF) ligand;
TNF2 is a second extracellular domain of a TNFSF ligand;
TNF3 is a third extracellular domain of a TNFSF ligand; and
L1-L25 are amino acid linkers.

2. An antigen binding polypeptide complex comprising a first polypeptide and a second polypeptide;
wherein:
(i) the first polypeptide has a structure represented by:
VL1-L1-VH1-L2-Fc;
VH1-L3-VL1-L4-Fc;
VL1-L5-VH1-L6-CL-L7-CH1-L8-Fc;
VL1-L5-VH1-L6-CH1-L7-CL-L8-Fc;
VH1-L5-VL1-L6-CL-L7-CH1-L8-Fc;
VH1-L5-VL1-L6-CH1-L7-CL-L8-Fc;
VL1-L9-CL-L10-VH1-L11-CH1-L12-Fc;
VL1-L9-CH1-L10-VH1-L11-CL-L12-Fc;
VH1-L9-CL-L10-VL1-L11-CH1-L12-Fc;
VH1-L9-CH1-L10-VL1-L11-CL-L12-Fc;
VL1-L13-VH1-L14-Fc-L15-TNF1-L16-TNF2-L17-TNF3;
VH1-L18-VL1-L19-Fc-L20-TNF1-L21-TNF2-L22-TNF3;
VL1-L23-VH1-L24-CL-L25-CH1-L26-Fc-L27-TNF1-L28-TNF2-L29-TNF3;
VL1-L23-VH1-L24-CH1-L25-CL-L26-Fc-L27-TNF1-L28-TNF2-L29-TNF3;
VH1-L23-VL1-L24-CL-L25-CH1-L26-Fc-L27-TNF1-L28-TNF2-L29-TNF3;
VH1-L23-VL1-L24-CH1-L25-CL-L26-Fc-L27-TNF1-L28-TNF2-L29-TNF3;
VL1-VH1-L14-Fc-L15-TNF1-L16-TNF2-L17-TNF3;
VH1-VL1-L19-Fc-L20-TNF1-L21-TNF2-L22-TNF3;
VL1-VH1-L24-CL-L25-CH1-L26-Fc-L27-TNF1-L28-TNF2-L29-TNF3;
VL1-VH1-L24-CH1-L25-CL-L26-Fc-L27-TNF1-L28-TNF2-L29-TNF3;
VH1-VL1-L24-CL-L25-CH1-L26-Fc-L27-TNF1-L28-TNF2-L29-TNF3;
VH1-VL1-L24-CH1-L25-CL-L26-Fc-L27-TNF1-L28-TNF2-L29-TNF3;
VL1-L30-CL-L31-VH1-L32-CH1-L33-Fc-L34-TNF1-L35-TNF2-L36-TNF3;
VL1-L30-CH1-L31-VH1-L32-CL-L33-Fc-L34-TNF1-L35-TNF2-L36-TNF3;
VH1-L30-CL-L31-VL1-L32-CH1-L33-Fc-L34-TNF1-L35-TNF2-L36-TNF3; or
VH1-L30-CH1-L31-VL1-L32-CL-L33-Fc-L34-TNF1-L35-TNF2-L36-TNF3; and
the second polypeptide has a structure represented by:
VL2-L37-VH2-L38-Fc-L39-TNF1-L40-TNF2-L41-TNF3;
VH2-L42-VL2-L43-Fc-L44-TNF1-L45-TNF2-L46-TNF3;
VL2-L47-VH2-L48-CL-L49-CH1-L50-Fc-L51-TNF1-L52-TNF2-L53-TNF3;
VL2-L54-CL-L55-VH2-L56-CH1-L57-Fc-L58-TNF1-L59-TNF2-L60-TNF3;
VL2-L47-VH2-L48-CH1-L49-CL-L50-Fc-L51-TNF1-L52-TNF2-L53-TNF3;
VL2-L54-CH1-L55-VH2-L56-CL-L57-Fc-L58-TNF1-L59-TNF2-L60-TNF3;
VL2-VH2-L38-Fc-L39-TNF1-L40-TNF2-L41-TNF3;
VH2-VL2-L43-Fc-L44-TNF1-L45-TNF2-L46-TNF3;
VL2-VH2-L48-CL-L49-CH1-L50-Fc-L51-TNF1-L52-TNF2-L53-TNF3;
VL2-CL-L55-VH2-L56-CH1-L57-Fc-L58-TNF1-L59-TNF2-L60-TNF3;
VL2-VH2-L48-CH1-L49-CL-L50-Fc-L51-TNF1-L52-TNF2-L53-TNF3; or
VL2-CH1-L55-VH2-L56-CL-L57-Fc-L58-TNF1-L59-TNF2-L60-TNF3;
or
(ii) the first polypeptide has a structure represented by:
VL1-L61-VH1-L62-Fc-L63-TNF1-L64-TNF2-L65-TNF3;
VH1-L66-VL1-L67-Fc-L68-TNF1-L69-TNF2-L70-TNF3;
VL1-L71-VH1-L72-CL-L73-CH1-L74-Fc-L75-TNF1-L76-TNF2-L77-TNF3;
VL1-L71-VH1-L72-CH1-L73-CL-L74-Fc-L75-TNF1-L76-TNF2-L77-TNF3;
VL1-L78-CL-L79-VH1-L80-CH1-L81-Fc-L82-TNF1-L83-TNF2-L84-TNF3; or
VL1-L78-CH1-L79-VH1-L80-CL-L81-Fc-L82-TNF1-L83-TNF2-L84-TNF3; and
the second polypeptide has a structure represented by:
VL2-L85-VH2-L86-Fc;
VH2-L87-VL2-L88-Fc;
VL2-L89-VH2-L90-CL-L91-CH1-L92-Fc;
VL2-L89-VH2-L90-CH1-L91-CL-L92-Fc;
VL2-L93-CL-L94-VH2-L95-CH1-L96-Fc; or
VL2-L93-CH1-L94-VH2-L95-CL-L96-Fc;
wherein:
VL1 is a first immunoglobulin light chain variable region;
VL2 is a second immunoglobulin light chain variable region;
VH1 is a first immunoglobulin heavy chain variable region;
VH2 is a second immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region;
TNF1 is a first extracellular domain of a TNFSF ligand;
TNF2 is a second extracellular domain of a TNFSF ligand;
TNF3 is a third extracellular domain of a TNFSF ligand; and
L1-L96 are amino acid linkers.

3. An antigen binding polypeptide complex comprising a first polypeptide and a second polypeptide;
wherein:
(i) the first polypeptide has a structure represented by:
Fc;
VL1-L1-VL2-L2-VH2-L3-VH1-L4-Fc;
VH1-L5-VH2-L6-VL2-L7-VL1-L8-Fc;
Fc-L9-TNF1-L10-TNF2-L11-TNF3;
VL1-L12-VL2-L13-VH2-L14-VH1-L15-Fc-L16-TNF1-L17-TNF2-L18-TNF3; or
VH1-L19-VH2-L20-VL2-L21-VL1-L22-Fc-L23-TNF1-L24-TNF2-L25-TNF3; and
the second polypeptide has a structure represented by:
VL3-L26-VL4-L27-VH4-L28-VH3-L29-Fc-L30-TNF1-L31-TNF2-L32-TNF3; or
VH3-L33-VH4-L34-VL4-L35-VL3-L36-Fc-L37-TNF1-L38-TNF2-L39-TNF3;
or
(ii) the first polypeptide has a structure represented by:
Fc-L40-TNF1-L41-TNF2-L42-TNF3;
VL1-L43-VL2-L44-VH2-L45-VH1-L46-Fc-L47-TNF1-L48-TNF2-L49-TNF3; or
VH1-L50-VH2-L51-VL2-L52-VL1-L53-Fc-L54-TNF1-L55-TNF2-L56-TNF3; and
the second polypeptide has a structure represented by:
Fc;
VL3-L57-VL4-L58-VH4-L59-VH3-L60-Fc; or
VH3-L61-VH4-L62-VL4-L63-VL3-L64-Fc;
wherein:
VL1 is a first immunoglobulin light chain variable region;
VL2 is a second immunoglobulin light chain variable region;
VL3 is a third immunoglobulin light chain variable region;
VL4 is a fourth immunoglobulin light chain variable region;
VH1 is a first immunoglobulin heavy chain variable region;
VH2 is a second immunoglobulin heavy chain variable region;
VH3 is a third immunoglobulin heavy chain variable region;
VH4 is a fourth immunoglobulin heavy chain variable region;
Fc is a region comprising an immunoglobulin heavy chain constant region 2 (CH2), an immunoglobulin heavy chain constant region 3 (CH3), and optionally, an immunoglobulin hinge;
TNF1 is a first extracellular domain of a TNFSF ligand;
TNF2 is a second extracellular domain of a TNFSF ligand;
TNF3 is a third extracellular domain of a TNFSF ligand; and
L1-L64 are amino acid linkers.

4. The antigen binding polypeptide complex of claim 2, wherein one or more of linkers L1-L96 have a length of from about 0 amino acids to about 50 amino acids.

5-6. (canceled)

7. The antigen binding polypeptide complex of claim 2, wherein one or more of linkers L1-L96 comprise the amino acid sequence of any one of SEQ ID NOs:3-10 and 148-175 or a sequence having at least 50%, at least 60%, at least 70%, at least 80%, at least 90%, or at least 95% identity to any one of SEQ ID NOs:3-10 and 148-175.

8. The antigen binding polypeptide complex of claim 1, wherein VL1 and VH1 specifically bind to CD3.

9. The antigen binding polypeptide complex of claim 8,
wherein VL1 comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and a CDR3 comprising an amino acid sequence having at least 90% identity, at least 95% identity, or 100% identity to any one of SEQ ID NOs:24, 30, 187, 300 and 308; and
wherein VH1 comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and a CDR3 comprising an amino acid sequence having at least 90% identity, at least 95% identity, or 100% identity to any one of SEQ ID NOs:21, 27, 184, 296 and 304.

10. The antigen binding polypeptide complex of claim 9, wherein VL1 comprises an amino acid sequence having at least 80%, at least 85%, at least 90%, at least 95%, or 100% identity to SEQ ID NO:45, and VH1 comprises an amino acid sequence having at least 80%, at least 85%, at least 90%, at least 95%, or 100% identity to SEQ ID NO:43 or 44.

11. The antigen binding polypeptide complex of claim 1, wherein VL2 and VH2 specifically bind to a tumor-associated antigen (TAA) or an immune stimulatory receptor.

12. The antigen binding polypeptide complex of claim 11, wherein the tumor-associated antigen is tyrosine-protein kinase Met (cMet), trophoblast cell surface antigen 2 (Trop2), CD20, CD19, receptor tyrosine-protein kinase erbB-2 (HER2), receptor tyrosine-protein kinase erbB-3 (HER3), adenosine A2A receptor (A2AR), a proliferation-inducing ligand (APRIL), epidermal growth factor receptor (EGFR), fibroblast growth factor receptor (FGFR), B cell activating factor (BAFF), BAFF receptor (BAFFR), B cell maturation antigen (BCMA), Bruton's tyrosine kinase (BTK), B and T lymphocyte attenuator (BTLA), B7DC (programmed death ligand 2), B7 homolog 1 (B7H1), B7 homolog 4 (B7H4), delta-like ligand 3 (DLL3), ectonucleoside triphosphate diphosphohydrolase 1 (ENTPD1), Fc fragment of IgE receptor 1a (FCER1A), Fc fragment of IgE receptor 1 (FCER1), arachidonate 5-lipoxygenase-activating protein (FLAP), folate hydrolase 1 (FOLH1), mucin 1 (MUC-1), CD133, mucin 16 (MUC-16), lysosomal-associated membrane protein 1 (LAMP1), CD38, programmed death ligand 1 (PD-L1), CEA cell adhesion molecule 5 (CEACAM5), six-transmembrane epithelial antigen of prostate 1 (STEAP1), or epithelial cellular adhesion molecule (EpCAM).

13. The antigen binding polypeptide complex of claim 11, wherein the TAA is HER2 or the immune stimulatory receptor is CD28.

14. The antigen binding polypeptide complex of claim 1,
wherein VL2 comprises a CDR1 comprising an amino acid sequence having at least 90% identity, at least 95% identity, or 100% identity to SEQ ID NO:34, 40, 274, 282, 290, 314 or 322; a CDR2 comprising an amino acid sequence having at least 90% identity, at least 95% identity, or 100% identity to SEQ ID NO:35, 41, 275, 283, 291, 315 or 323; and a CDR3 comprising an amino acid sequence having at least 90% identity, at least 95% identity, or 100% identity to SEQ ID NO:36, 42, 276, 284, 292, 316 or 324; and
wherein VH2 comprises a CDR1 comprising an amino acid sequence having at least 90% identity, at least 95% identity, or 100% identity to SEQ ID NO:31, 37, 270, 278, 286, 310 or 318; a CDR2 comprising an amino acid sequence having at least 90% identity, at least 95% identity, or 100% identity to SEQ ID NO:32, 38, 271, 279, 287, 311 or 319; and a CDR3 comprising an amino acid sequence having at least 90% identity, at least 95% identity, or 100% identity to SEQ ID NO:33, 39, 272, 280, 288, 312 or 320.

15. The antigen binding polypeptide complex of claim 14, wherein VL2 comprises an amino acid sequence having at least 80%, at least 85%, at least 90%, at least 95%, or 100% identity to SEQ ID NO:47, 49, 273, 281, 289, 313 or 321, and VH2 comprises an amino acid sequence having at least 80%, at least 85%, at least 90%, at least 95%, or 100% identity to SEQ ID NO:46, 48, 269, 277, 285, 309 or 317.

16. The antigen binding polypeptide complex of claim 1, wherein VL2 and VH2 specifically bind to CD3.

17. The antigen binding polypeptide complex of claim 16, wherein VL1 and VH1 specifically bind to a TAA or an immune stimulatory receptor.

18. The antigen binding polypeptide complex of claim 17, wherein the TAA is HER2 or the immune stimulatory receptor is CD28.

19. The antigen binding polypeptide complex of claim 2, wherein VL1 and VH1 specifically bind to CD3.

20. The antigen binding polypeptide complex of claim 19,
wherein VL1 comprises a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and a CDR3 comprising an amino acid sequence having at least 90% identity, at least 95% identity, or 100% identity to any one of SEQ ID NOs:24, 30, 187, 300 and 308; and
wherein VH1 comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and a CDR3 comprising an amino acid sequence having at least 90% identity, at least 95% identity, or 100% identity to any one of SEQ ID NOs:21, 27, 184, 296 and 304.

21. The antigen binding polypeptide complex of claim 20, wherein VL1 comprises an amino acid sequence having at least 80%, at least 85%, at least 90%, at least 95%, or 100% identity to SEQ ID NO:45, 297 or 305, and VH1 comprises an amino acid sequence having at least 80%, at least 85%, at least 90%, at least 95%, or 100% identity to SEQ ID NO:43, 44, 188, 293 and 301.

22. The antigen binding polypeptide complex of claim 2, wherein VL2 and VH2 specifically bind to a TAA or an immune stimulatory receptor.

23. The antigen binding polypeptide complex of claim 22, wherein the TAA is cMet, Trop2, CD20, CD19, HER2, HER3, A2AR, APRIL, EGFR, FGFR, BAFF, BAFFR, BCMA, BTK, BTLA, B7DC, B7H1, B7H4, DLL3, ENTPD1, FCER1A, FCER1, FLAP, FOLH1, MUC-1, CD133, MUC-16, LAMP1, CD38, PD-L1, CEACAM5, STEAP1, or EpCAM.

24. The antigen binding polypeptide complex of claim 22, wherein the TAA is HER2 or the immune stimulatory receptor is CD28.

25. The antigen binding polypeptide complex of claim 2,
(i) wherein VL2 comprises a CDR1 comprising an amino acid sequence having at least 90% identity, at least 95% identity, or 100% identity to SEQ ID NO:34, 40, 274, 282, 290, 314 and 322; a CDR2 comprising an amino acid sequence having at least 90% identity, at least 95% identity, or 100% identity to any one of SEQ ID NO:35, 41, 275, 283, 291, 315 and 323; and a CDR3 comprising an amino acid sequence having at least 90% identity, at least 95% identity, or 100% identity to SEQ ID NO:36, 42, 276, 284, 292, 316 and 324; and
wherein VH2 comprises a CDR1 comprising an amino acid sequence having at least 90% identity, at least 95% identity, or 100% identity to SEQ ID NO:31, 37, 270, 278, 286, 310 and 318; a CDR2 comprising an amino acid sequence having at least 90% identity, at least 95% identity, or 100% identity to SEQ ID NO:32, 38 271, 279, 287, 311 and 319; and a CDR3 comprising an amino acid sequence having at least 90% identity, at least 95% identity, or 100% identity to SEQ ID NO:33, 39, 272, 280, 288, 312 and 320; or
(ii) wherein VL2 comprises an amino acid sequence having at least 80%, at least 85%, at least 90%, at least 95%, or 100% identity to SEQ ID NO:47, 49, 273, 281, 289, 313 or 321, and VH2 comprises an amino acid sequence having at least 80%, at least 85%, at least 90%, at least 95%, or 100% identity to SEQ ID NO:46, 48, 269, 277, 285, 309 or 317.

26. The antigen binding polypeptide complex of claim 2, wherein VL2 and VH2 specifically bind to CD3.

27. The antigen binding polypeptide complex of claim 2, wherein VL1 and VH1 specifically bind to a TAA or an immune stimulatory receptor.

28. The antigen binding polypeptide complex of claim 27, wherein the TAA is HER2 or the immune stimulatory receptor is CD28.

29. The antigen binding polypeptide complex of claim 2, wherein the VL1 and VH1 of the antigen binding polypeptide specifically bind to CD3 and the VL2 and VH2 specifically bind to a TAA or an immune stimulatory receptor; or the VL2 and VH2 of the antigen binding polypeptide specifically bind to CD3 and the VL1 and VH1 specifically bind to a TAA or an immune stimulatory receptor.

30. The antigen binding polypeptide complex of claim 3, wherein VL1, VH1, VL3 and VH3 specifically bind to CD3.

31. The antigen binding polypeptide complex of claim 3, wherein VL2, VH2, VL4 and VH4 specifically bind to a TAA or an immune stimulatory receptor.

32. The antigen binding polypeptide complex of claim 31, wherein the TAA is HER2 or the immune stimulatory receptor is CD28.

33. The antigen binding polypeptide complex of claim 3, wherein VL1, VH1, VL4 and VH4 specifically bind to CD3.

34. The antigen binding polypeptide complex of claim 3, wherein VL2, VH2, VL3 and VH3 specifically bind to a TAA or an immune stimulatory receptor.

35. The antigen binding polypeptide complex of claim 34, wherein the TAA is HER2 or the immune stimulatory receptor is CD28.

36. The antigen binding polypeptide complex of claim 3, wherein VL2, VH2, VL4 and VH4 specifically bind to CD3.

37. The antigen binding polypeptide complex of claim 3, wherein VL1, VH1, VL3 and VH3 specifically bind to a TAA or an immune stimulatory receptor.

38. The antigen binding polypeptide complex of claim 37, wherein the TAA is HER2 or the immune stimulatory receptor is CD28.

39. The antigen binding polypeptide complex of claim 3, wherein VL2, VH2, VL3 and VH3 specifically bind to CD3.

40. The antigen binding polypeptide complex of claim 3, wherein VL1, VH1, VL4 and VH4 specifically bind to a TAA or an immune stimulatory receptor.

41. The antigen binding polypeptide complex of claim 40, wherein the TAA is HER2 or the immune stimulatory receptor is CD28.

42. The antigen binding polypeptide complex of claim 30,
wherein the VLs specifically binding to CD3 comprise a CDR1 comprising an amino acid sequence having at least 90% identity, at least 95% identity, or 100% identity to any one of SEQ ID NOs:22, 28, 185, 298 and 306; a CDR2 comprising an amino acid sequence having at least 90% identity, at least 95% identity, or 100% identity to any one of SEQ ID NOs:23, 29, 186, 299 and 307; and a CDR3 comprising an amino acid sequence having at least 90% identity, at least 95% identity, or 100% identity to any one of SEQ ID NOs:24, 30, 187, 300 and 308; and
wherein the VHs specifically binding to CD3 comprises a CDR1 comprising an amino acid sequence having at least 90% identity, at least 95% identity, or 100% identity to any one of SEQ ID NOs:19, 25, 182, 294 and 302; a CDR2 comprising an amino acid sequence having at least 90% identity, at least 95% identity, or 100% identity to any one of SEQ ID NOs:20, 26, 183, 295 and 303; and a CDR3 comprising an amino acid sequence having at least 90% identity, at least 95% identity, or 100% identity to any one of SEQ ID NOs:21, 27, 184, 296 and 304.

43. The antigen binding polypeptide complex of claim 42, wherein the VLs specifically binding to CD3 comprise an amino acid sequence having at least 80%, at least 85%, at least 90%, at least 95%, or 100% identity to SEQ ID NO:45, 297 or 305, and the VHs specifically binding to CD3 comprise an amino acid sequence having at least 80%, at least 85%, at least 90%, at least 95%, or 100% identity to SEQ ID NO:43, 44, 188, 293 or 301.

44. The antigen binding polypeptide complex of claim 30,
wherein the VLs specifically binding to a TAA or immune stimulatory receptor comprise a CDR1 comprising an amino acid sequence having at least 90% identity, at least 95% identity, or 100% identity to SEQ ID NO:34, 40 274, 282, 290, 314 or 322; a CDR2 comprising an amino acid sequence having at least 90% identity, at least 95% identity, or 100% identity to SEQ ID NO:35, 41, 275, 283, 291, 315 or 323; and a CDR3 comprising an amino acid sequence having at least 90% identity, at least 95% identity, or 100% identity to SEQ ID NO:36, 42, 276, 284, 292, 316 or 324; and
wherein the VHs specifically binding to a TAA or immune stimulatory receptor comprises a CDR1 comprising an amino acid sequence having at least 90% identity, at least 95% identity, or 100% identity to SEQ ID NO:31, 37, 270, 278, 286, 310 or 318; a CDR2 comprising an amino acid sequence having at least 90% identity, at least 95% identity, or 100% identity to SEQ ID NO:32, 38, 271, 279, 287, 311 or 319; and a CDR3 comprising an amino acid sequence having at least 90% identity, at least 95% identity, or 100% identity to SEQ ID NO:33, 39, 272, 280, 288, 312 or 320.

45. The antigen binding polypeptide complex of claim 44, wherein the VLs specifically binding to a TAA or immune stimulatory receptor comprise an amino acid sequence having at least 80%, at least 85%, at least 90%, at least 95%, or 100% identity to SEQ ID NO:47, 49, 273, 281, 289, 313 or 321, and the VHs specifically binding to a TAA or an immune stimulatory receptor comprise an amino acid sequence having at least 80%, at least 85%, at least 90%, at least 95%, or 100% identity to SEQ ID NO:46, 48, 269, 277, 285, 309 or 317.

46. The antigen binding polypeptide complex of claim 2, wherein TNF1, TNF2 and TNF3 are each selected from the group consisting of OX40L (TNFSF4), 4-1BBL (TNFSF9), TNF, TNF-related apoptosis inducing ligand (TRAIL), CD40L (TNFSF5), CD27L (TNFSF7), CD30L (TNFSF8), FasL (TNFSF6), EDAM, LTA (TNFSF1), LTB (TNFSF3), CD153 (TNFSF8), RANKL (TNFSF11), TWEAK (TNFSF12), APRIL (TNF SF 13), BAFF (TNF SF 13B), LIGHT (TNF SF I4), VEGI (TNF SF 15), and GITRL (TNF SF 18).

47. The antigen binding polypeptide complex of claim 2, wherein TNF1, TNF2 and TNF3 are each OX40L.

48. The antigen binding polypeptide complex of claim 2, wherein TNF1, TNF2, and TNF3 are each 4-1BBL.

49. (canceled)

50. The antigen binding polypeptide complex of claim 2, wherein the immunoglobulin hinge comprises an upper hinge region, a middle hinge region, a lower hinge region, or a combination thereof.

51. The antigen binding polypeptide complex of claim 2, wherein the Fc region comprises at least one knob-into-hole modification.

52. The antigen binding polypeptide complex of claim 51, wherein the antigen binding polypeptide complex is an IgG1 or IgG4 antibody and the knob-into-hole modification comprises:
(i) knob substitutions of S354C and T366W and hole substitutions of Y349C, T366S, L368A and Y407V;
(ii) hole substitutions of L234A, L235A and P329A;
(iii) hole substitutions of L234A and L235A;
(iv) hole substitutions of M428L and N433 S;
(v) hole substitutions of M252Y, S254T and T256E; or
(vi) a combination thereof;
based on the EU numbering scheme.

53. An antibody or antigen binding fragment thereof comprising the antigen binding polypeptide complex of claim 2.

54. A pharmaceutical composition comprising the antigen binding polypeptide complex of claim 2, and a pharmaceutically acceptable carrier.

55. A method for inducing or enhancing an immune response, comprising administering to a subject in need thereof the antigen binding polypeptide complex of claim 2.

56. A method for overcoming cancer-mediated immune suppression, comprising administering to a subject in need thereof the antigen binding polypeptide complex of claim 2.
